SEQUENCE LISTING (618 sequences)

SEQ ID NO: 1 - protein, 20 aa, synthetic construct
  REGION 1..20: note = Description of Artificial Sequence: Synthetic peptide
GKPGSGKPGSGKPGSGKPGS

SEQ ID NO: 2 - protein, 5 aa, synthetic construct
  REGION 1..5: note = Description of Artificial Sequence: Synthetic peptide
GGGGS

SEQ ID NO: 3 - protein, 5 aa, synthetic construct
  REGION 1..5: note = Description of Artificial Sequence: Synthetic peptide
  REGION 1..5: note = See specification as filed for detailed description of substitutions and preferred embodiments
GSGGS

SEQ ID NO: 4 - protein, 4 aa, synthetic construct
  REGION 1..4: note = Description of Artificial Sequence: Synthetic peptide
  REGION 1..4: note = See specification as filed for detailed description of substitutions and preferred embodiments
GGGS

SEQ ID NO: 5 - protein, 15 aa, synthetic construct
  REGION 1..15: note = Description of Artificial Sequence: Synthetic peptide
GGGGSGGGGSGGGGS

SEQ ID NO: 6 - protein, 18 aa, synthetic construct
  REGION 1..18: note = Description of Artificial Sequence: Synthetic peptide
GSTSGSGKPGSGEGSTKG

SEQ ID NO: 7 - protein, 14 aa, synthetic construct
  REGION 1..14: note = Description of Artificial Sequence: Synthetic peptide
IRPRAIGGSKPRVA

SEQ ID NO: 8 - protein, 15 aa, synthetic construct
  REGION 1..15: note = Description of Artificial Sequence: Synthetic peptide
GKGGSGKGGSGKGGS

SEQ ID NO: 9 - protein, 15 aa, synthetic construct
  REGION 1..15: note = Description of Artificial Sequence: Synthetic peptide
GGKGSGGKGSGGKGS

SEQ ID NO: 10 - protein, 15 aa, synthetic construct
  REGION 1..15: note = Description of Artificial Sequence: Synthetic peptide
GGGKSGGGKSGGGKS

SEQ ID NO: 11 - protein, 15 aa, synthetic construct
  REGION 1..15: note = Description of Artificial Sequence: Synthetic peptide
GKGKSGKGKSGKGKS

SEQ ID NO: 12 - protein, 15 aa, synthetic construct
  REGION 1..15: note = Description of Artificial Sequence: Synthetic peptide
GGGKSGGKGSGKGGS

SEQ ID NO: 13 - protein, 15 aa, synthetic construct
  REGION 1..15: note = Description of Artificial Sequence: Synthetic peptide
GKPGSGKPGSGKPGS

SEQ ID NO: 14 - protein, 20 aa, synthetic construct
  REGION 1..20: note = Description of Artificial Sequence: Synthetic peptide
GKGKSGKGKSGKGKSGKGKS

SEQ ID NO: 15 - protein, 20 aa, synthetic construct
  REGION 1..20: note = Description of Artificial Sequence: Synthetic peptide
GGGGSGGGGSGGGGSGGGGS

SEQ ID NO: 16 - protein, 14 aa, synthetic construct
  REGION 1..14: note = Description of Artificial Sequence: Synthetic peptide
STAGDTHLGGEDFD

SEQ ID NO: 17 - protein, 15 aa, synthetic construct
  REGION 1..15: note = Description of Artificial Sequence: Synthetic peptide
GEGGSGEGGSGEGGS

SEQ ID NO: 18 - protein, 15 aa, synthetic construct
  REGION 1..15: note = Description of Artificial Sequence: Synthetic peptide
GGEGSGGEGSGGEGS

SEQ ID NO: 19 - protein, 15 aa, synthetic construct
  REGION 1..15: note = Description of Artificial Sequence: Synthetic peptide
GGGESGGGESGGGES

SEQ ID NO: 20 - protein, 15 aa, synthetic construct
  REGION 1..15: note = Description of Artificial Sequence: Synthetic peptide
GEGESGEGESGEGES

SEQ ID NO: 21 - protein, 15 aa, synthetic construct
  REGION 1..15: note = Description of Artificial Sequence: Synthetic peptide
GGGESGGEGSGEGGS

SEQ ID NO: 22 - protein, 20 aa, synthetic construct
  REGION 1..20: note = Description of Artificial Sequence: Synthetic peptide
GEGESGEGESGEGESGEGES

SEQ ID NO: 23 - protein, 19 aa, synthetic construct
  REGION 1..19: note = Description of Artificial Sequence: Synthetic peptide
PRGASKSGSASQTGSAPGS

SEQ ID NO: 24 - protein, 19 aa, synthetic construct
  REGION 1..19: note = Description of Artificial Sequence: Synthetic peptide
GTAAAGAGAAGGAAAGAAG

SEQ ID NO: 25 - protein, 19 aa, synthetic construct
  REGION 1..19: note = Description of Artificial Sequence: Synthetic peptide
GTSGSSGSGSGGSGSGGGG

SEQ ID NO: 26 - protein, 10 aa, synthetic construct
  REGION 1..10: note = Description of Artificial Sequence: Synthetic peptide
GGGGSGGGGS

SEQ ID NO: 27 - protein, 25 aa, synthetic construct
  REGION 1..25: note = Description of Artificial Sequence: Synthetic peptide
GGGGSGGGGSGGGGSGGGGSGGGGS

SEQ ID NO: 28 - protein, 30 aa, synthetic construct
  REGION 1..30: note = Description of Artificial Sequence: Synthetic polypeptide
GGGGSGGGGSGGGGSGGGGSGGGGSGGGGS

SEQ ID NO: 29 - protein, 35 aa, synthetic construct
  REGION 1..35: note = Description of Artificial Sequence: Synthetic polypeptide
GGGGSGGGGSGGGGSGGGGSGGGGSGGGGSGGGGS

SEQ ID NO: 30 - protein, 5 aa, synthetic construct
  REGION 1..5: note = Description of Artificial Sequence: Synthetic peptide
GGGGA

SEQ ID NO: 31 - protein, 10 aa, synthetic construct
  REGION 1..10: note = Description of Artificial Sequence: Synthetic peptide
GGGGAGGGGA

SEQ ID NO: 32 - protein, 15 aa, synthetic construct
  REGION 1..15: note = Description of Artificial Sequence: Synthetic peptide
GGGGAGGGGAGGGGA

SEQ ID NO: 33 - protein, 20 aa, synthetic construct
  REGION 1..20: note = Description of Artificial Sequence: Synthetic peptide
GGGGAGGGGAGGGGAGGGGA

SEQ ID NO: 34 - protein, 25 aa, synthetic construct
  REGION 1..25: note = Description of Artificial Sequence: Synthetic peptide
GGGGAGGGGAGGGGAGGGGAGGGGA

SEQ ID NO: 35 - protein, 30 aa, synthetic construct
  REGION 1..30: note = Description of Artificial Sequence: Synthetic polypeptide
GGGGAGGGGAGGGGAGGGGAGGGGAGGGGA

SEQ ID NO: 36 - protein, 35 aa, synthetic construct
  REGION 1..35: note = Description of Artificial Sequence: Synthetic polypeptide
GGGGAGGGGAGGGGAGGGGAGGGGAGGGGAGGGGA

SEQ ID NO: 37 - protein, 30 aa, synthetic construct
  REGION 1..30: note = Description of Artificial Sequence: Synthetic polypeptide
DPALVHQRPAPPGGGGSGGGGSGGGGSGGG

SEQ ID NO: 38 - protein, 5 aa, synthetic construct
  REGION 1..5: note = Description of Artificial Sequence: Synthetic peptide
GKPGS

SEQ ID NO: 39 - protein, 25 aa, synthetic construct
  REGION 1..25: note = Description of Artificial Sequence: Synthetic peptide
GKPGSGKPGSGKPGSGKPGSGKPGS

SEQ ID NO: 40 - protein, 30 aa, synthetic construct
  REGION 1..30: note = Description of Artificial Sequence: Synthetic polypeptide
GKPGSGKPGSGKPGSGKPGSGKPGSGKPGS

SEQ ID NO: 41 - protein, 5 aa, synthetic construct
  REGION 1..5: note = Description of Artificial Sequence: Synthetic peptide
GGGES

SEQ ID NO: 42 - protein, 9 aa, synthetic construct
  REGION 1..9: note = Description of Artificial Sequence: Synthetic peptide
KTHTCPPCP

SEQ ID NO: 43 - protein, 15 aa, synthetic construct
  REGION 1..15: note = Description of Artificial Sequence: Synthetic peptide
EPKSSDKTHTCPPCP

SEQ ID NO: 44 - protein, 19 aa, synthetic construct
  REGION 1..19: note = Description of Artificial Sequence: Synthetic peptide
GGGGSGGGGSKTHTCPPCP

SEQ ID NO: 45 - protein, 19 aa, synthetic construct
  REGION 1..19: note = Description of Artificial Sequence: Synthetic peptide
GKPGSGKPGSKTHTCPPCP

SEQ ID NO: 46 - protein, 14 aa, synthetic construct
  REGION 1..14: note = Description of Artificial Sequence: Synthetic peptide
GKPGSKTHTCPPCP

SEQ ID NO: 47 - protein, 329 aa, synthetic construct
  REGION 1..329: note = Description of Artificial Sequence: Synthetic polypeptide
  REGION 1..329: note = 1 + 1 Fab-scFv-Fc Backbone 1 Fab-Fc Side
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 48 - protein, 231 aa, synthetic construct
  REGION 1..231: note = Description of Artificial Sequence: Synthetic polypeptide
  REGION 1..231: note = 1 + 1 Fab-scFv-Fc Backbone 1 scFv-Fc Side
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 329 aa, synthetic construct; identical to SEQ ID NO: 47
  REGION 1..329: note = Description of Artificial Sequence: Synthetic polypeptide
  REGION 1..329: note = 1 + 1 Fab-scFv-Fc Backbone 2 Fab-Fc Side

SEQ ID NO: 50 - protein, 231 aa, synthetic construct
  REGION 1..231: note = Description of Artificial Sequence: Synthetic polypeptide
  REGION 1..231: note = 1 + 1 Fab-scFv-Fc Backbone 2 scFv-Fc Side
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 329 aa, synthetic construct
  REGION 1..329: note = Description of Artificial Sequence: Synthetic polypeptide
  REGION 1..329: note = 1 + 1 Fab-scFv-Fc Backbone 3 Fab-Fc Side
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 52 - protein, 231 aa, synthetic construct; identical to SEQ ID NO: 50
  REGION 1..231: note = Description of Artificial Sequence: Synthetic polypeptide
  REGION 1..231: note = 1 + 1 Fab-scFv-Fc Backbone 3 scFv-Fc Side

SEQ ID NO: 53 - protein, 329 aa, synthetic construct
  REGION 1..329: note = Description of Artificial Sequence: Synthetic polypeptide
  REGION 1..329: note = 1 + 1 Fab-scFv-Fc Backbone 4 Fab-Fc Side
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 54 - protein, 231 aa, synthetic construct
  REGION 1..231: note = Description of Artificial Sequence: Synthetic polypeptide
  REGION 1..231: note = 1 + 1 Fab-scFv-Fc Backbone 4 scFv-Fc Side
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 55 - protein, 329 aa, synthetic construct
  REGION 1..329: note = Description of Artificial Sequence: Synthetic polypeptide
  REGION 1..329: note = 1 + 1 Fab-scFv-Fc Backbone 5 Fab-Fc Side
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 56 - protein, 231 aa, synthetic construct
  REGION 1..231: note = Description of Artificial Sequence: Synthetic polypeptide
  REGION 1..231: note = 1 + 1 Fab-scFv-Fc Backbone 5 scFv-Fc Side
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 329 aa, synthetic construct
  REGION 1..329: note = Description of Artificial Sequence: Synthetic polypeptide
  REGION 1..329: note = 1 + 1 Fab-scFv-Fc Backbone 6 Fab-Fc Side
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 58 - protein, 231 aa, synthetic construct
  REGION 1..231: note = Description of Artificial Sequence: Synthetic polypeptide
  REGION 1..231: note = 1 + 1 Fab-scFv-Fc Backbone 6 scFv-Fc Side
EPKSSDKTHTCPPCPAPPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 329 aa, synthetic construct
  REGION 1..329: note = Description of Artificial Sequence: Synthetic polypeptide
  REGION 1..329: note = 1 + 1 Fab-scFv-Fc Backbone 7 Fab-Fc Side
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 60 - protein, 231 aa, synthetic construct
  REGION 1..231: note = Description of Artificial Sequence: Synthetic polypeptide
  REGION 1..231: note = 1 + 1 Fab-scFv-Fc Backbone 7 scFv-Fc Side
EPKSSDKTHTCPPCPAPPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 327 aa, synthetic construct
  REGION 1..327: note = Description of Artificial Sequence: Synthetic polypeptide
  REGION 1..327: note = 1 + 1 Fab-scFv-Fc Backbone 8 Fab-Fc Side
ASTKGPSVFPLAPCSRSTSESTAALGCLVKDYFPEPVTVSWNSGALTSGVHTFPAVLQSSGLYSLSSVVTVPSSSLGTKTYTCNVDHKPSDTKVDKRVESKYGPPCPPCPAPEFLGGPSVFLFPPKPKDTLMISRTPEVTCVVVDVSQEDPEVQFNWYVDGVEVHNAKTKPREEEFNSTYRVVSVLTVLHQDWLNGKEYKCKVSNKGLPSSIEKTISKAKGQPREPQVYTLPPSQEEMTKNQVSLTCDVSGFYPSDIAVEWESDGQPENNYKTTPPVLDSDGSFFLYSRLTVDKSRWEEGDVFSCSVMHEALHNHYTQKSLSLSLGK

SEQ ID NO: 62 - protein, 229 aa, synthetic construct
  REGION 1..229: note = Description of Artificial Sequence: Synthetic polypeptide
  REGION 1..229: note = 1 + 1 Fab-scFv-Fc Backbone 8 scFv-Fc Side
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEQMTKNQVKLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 326 aa, synthetic construct
  REGION 1..326: note = Description of Artificial Sequence: Synthetic polypeptide
  REGION 1..326: note = 1 + 1 Fab-scFv-Fc Backbone 9 Fab-Fc Side
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTVERKCCVECPPCPAPPVAGPSVFLFPPKPKDTLMISRTPEVTCVVVDVSHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 64 - protein, 228 aa, synthetic construct
  REGION 1..228: note = Description of Artificial Sequence: Synthetic polypeptide
  REGION 1..228: note = 1 + 1 Fab-scFv-Fc Backbone 9 scFv-Fc Side
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 65 - protein, 326 aa, synthetic construct
  REGION 1..326: note = Description of Artificial Sequence: Synthetic polypeptide
  REGION 1..326: note = 1 + 1 Fab-scFv-Fc Backbone 10 Fab-Fc Side
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTVERKCCVECPPCPAPPVAGPSVFLFPPKPKDTLMISRTPEVTCVVVDVKHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 66 - protein, 228 aa, synthetic construct
  REGION 1..228: note = Description of Artificial Sequence: Synthetic polypeptide
  REGION 1..228: note = 1 + 1 Fab-scFv-Fc Backbone 10 scFv-Fc Side
ERKCCVECPPCPAPPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - protein, 329 aa, synthetic construct
  REGION 1..329: note = Description of Artificial Sequence: Synthetic polypeptide
  REGION 1..329: note = 1 + 1 Fab-scFv-Fc Backbone 11 Fab-Fc Side
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 68 - protein, 231 aa, synthetic construct
  REGION 1..231: note = Description of Artificial Sequence: Synthetic polypeptide
  REGION 1..231: note = 1 + 1 Fab-scFv-Fc Backbone 11 scFv-Fc Side
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 69 - protein, 329 aa, synthetic construct
  REGION 1..329: note = Description of Artificial Sequence: Synthetic polypeptide
  REGION 1..329: note = 1 + 1 Fab-scFv-Fc Backbone 12 Fab-Fc Side
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCDVAGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 231 aa, synthetic construct
  REGION 1..231: note = Description of Artificial Sequence: Synthetic polypeptide
  REGION 1..231: note = 1 + 1 Fab-scFv-Fc Backbone 12 scFv-Fc Side
ERKSSDKTHTCPPRPAPPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 329 aa, synthetic construct; identical to SEQ ID NO: 47
  REGION 1..329: note = Description of Artificial Sequence: Synthetic polypeptide
  REGION 1..329: note = 2 + 1 Fab2-scFv-Fc Backbone 1 Fab-Fc Side

SEQ ID NO: 72 - protein, 216 aa, synthetic construct
  REGION 1..216: note = Description of Artificial Sequence: Synthetic polypeptide
  REGION 1..216: note = 2 + 1 Fab2-scFv-Fc Backbone 1 Fab-scFv-Fc Side
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 329 aa, synthetic construct; identical to SEQ ID NO: 47
  REGION 1..329: note = Description of Artificial Sequence: Synthetic polypeptide
  REGION 1..329: note = 2 + 1 Fab2-scFv-Fc Backbone 2 Fab-Fc Side

SEQ ID NO: 74 - protein, 216 aa, synthetic construct
  REGION 1..216: note = Description of Artificial Sequence: Synthetic polypeptide
  REGION 1..216: note = 2 + 1 Fab2-scFv-Fc Backbone 2 Fab-scFv-Fc Side
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 329 aa, synthetic construct; identical to SEQ ID NO: 51
  REGION 1..329: note = Description of Artificial Sequence: Synthetic polypeptide
  REGION 1..329: note = 2 + 1 Fab2-scFv-Fc Backbone 3 Fab-Fc Side

SEQ ID NO: 76 - protein, 216 aa, synthetic construct; identical to SEQ ID NO: 74
  REGION 1..216: note = Description of Artificial Sequence: Synthetic polypeptide
  REGION 1..216: note = 2 + 1 Fab2-scFv-Fc Backbone 3 Fab-scFv-Fc Side

SEQ ID NO: 77 - protein, 329 aa, synthetic construct; identical to SEQ ID NO: 53
  REGION 1..329: note = Description of Artificial Sequence: Synthetic polypeptide
  REGION 1..329: note = 2 + 1 Fab2-scFv-Fc Backbone 4 Fab-Fc Side

SEQ ID NO: 78 - protein, 216 aa, synthetic construct
  REGION 1..216: note = Description of Artificial Sequence: Synthetic polypeptide
  REGION 1..216: note = 2 + 1 Fab2-scFv-Fc Backbone 4 Fab-scFv-Fc Side
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 79 - protein, 329 aa, synthetic construct; identical to SEQ ID NO: 55
  REGION 1..329: note = Description of Artificial Sequence: Synthetic polypeptide
  REGION 1..329: note = 2 + 1 Fab2-scFv-Fc Backbone 5 Fab-Fc Side

SEQ ID NO: 80 - protein, 216 aa, synthetic construct
  REGION 1..216: note = Description of Artificial Sequence: Synthetic polypeptide
  REGION 1..216: note = 2 + 1 Fab2-scFv-Fc Backbone 5 Fab-scFv-Fc Side
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 329 aa, synthetic construct; identical to SEQ ID NO: 57
  REGION 1..329: note = Description of Artificial Sequence: Synthetic polypeptide
  REGION 1..329: note = 2 + 1 Fab2-scFv-Fc Backbone 6 Fab-Fc Side

SEQ ID NO: 82 - protein, 216 aa, synthetic construct
  REGION 1..216: note = Description of Artificial Sequence: Synthetic polypeptide
  REGION 1..216: note = 2 + 1 Fab2-scFv-Fc Backbone 6 Fab-scFv-Fc Side
APPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 329 aa, synthetic construct; identical to SEQ ID NO: 59
  REGION 1..329: note = Description of Artificial Sequence: Synthetic polypeptide
  REGION 1..329: note = 2 + 1 Fab2-scFv-Fc Backbone 7 Fab-Fc Side

SEQ ID NO: 84 - protein, 216 aa, synthetic construct
  REGION 1..216: note = Description of Artificial Sequence: Synthetic polypeptide
  REGION 1..216: note = 2 + 1 Fab2-scFv-Fc Backbone 7 Fab-scFv-Fc Side
APPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 329 aa, synthetic construct; identical to SEQ ID NO: 67
  REGION 1..329: note = Description of Artificial Sequence: Synthetic polypeptide
  REGION 1..329: note = 2 + 1 Fab2-scFv-Fc Backbone 8 Fab-Fc Side

SEQ ID NO: 86 - protein, 216 aa, synthetic construct
  REGION 1..216: note = Description of Artificial Sequence: Synthetic polypeptide
  REGION 1..216: note = 2 + 1 Fab2-scFv-Fc Backbone 8 Fab-scFv-Fc Side
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 87 - protein, 329 aa, synthetic construct; identical to SEQ ID NO: 69
  REGION 1..329: note = Description of Artificial Sequence: Synthetic polypeptide
  REGION 1..329: note = 2 + 1 Fab2-scFv-Fc Backbone 9 Fab-Fc Side

SEQ ID NO: 88 - protein, 216 aa, synthetic construct
  REGION 1..216: note = Description of Artificial Sequence: Synthetic polypeptide
  REGION 1..216: note = 2 + 1 Fab2-scFv-Fc Backbone 9 Fab-scFv-Fc Side
APPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 107 aa, synthetic construct [sequence omitted]

SEQ ID NO: 90 - protein, 106 aa, synthetic construct
  REGION 1..106: note = Description of Artificial Sequence: Synthetic polypeptide
  REGION 1..106: note = Constant Light Domain - Lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 91 - protein, 254 aa, synthetic construct
  REGION 1..254: note = Description of Artificial Sequence: Synthetic polypeptide
  REGION 1..254: note = CD3 High - [anti-CD3]_H1.30_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 92 - protein, 125 aa, synthetic construct
  REGION 1..125: note = Description of Artificial Sequence: Synthetic polypeptide
  REGION 1..125: note = CD3 High - [anti-CD3]_H1.30_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 93 - protein, 5 aa, synthetic construct
  REGION 1..5: note = Description of Artificial Sequence: Synthetic peptide
  REGION 1..5: note = CD3 High - [anti-CD3]_H1.30_L1.47_scFv vhCDR1
TYAMN

SEQ ID NO: 94 - protein, 19 aa, synthetic construct
  REGION 1..19: note = Description of Artificial Sequence: Synthetic peptide
  REGION 1..19: note = CD3 High - [anti-CD3]_H1.30_L1.47_scFv vhCDR2
RIRSKYNNYATYYADSVKG

SEQ ID NO: 95 - protein, 14 aa, synthetic construct
  REGION 1..14: note = Description of Artificial Sequence: Synthetic peptide
  REGION 1..14: note = CD3 High - [anti-CD3]_H1.30_L1.47_scFv vhCDR3
HGNFGDSYVSWFAY

SEQ ID NO: 96 - protein, 109 aa, synthetic construct
  REGION 1..109: note = Description of Artificial Sequence: Synthetic polypeptide
  REGION 1..109: note = CD3 High - [anti-CD3]_H1.30_L1.47_scFv Variable Light (vl) Domain
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 97 - protein, 14 aa, synthetic construct
  REGION 1..14: note = Description of Artificial Sequence: Synthetic peptide
  REGION 1..14: note = CD3 High - [anti-CD3]_H1.30_L1.47_scFv vlCDR1
GSSTGAVTTSNYAN

SEQ ID NO: 98 - protein, 7 aa, synthetic construct
  REGION 1..7: note = Description of Artificial Sequence: Synthetic peptide
  REGION 1..7: note = CD3 High - [anti-CD3]_H1.30_L1.47_scFv vlCDR2
GTNKRAP

SEQ ID NO: 99 - protein, 9 aa, synthetic construct
  REGION 1..9: note = Description of Artificial Sequence: Synthetic peptide
  REGION 1..9: note = CD3 High - [anti-CD3]_H1.30_L1.47_scFv vlCDR3
ALWYSNHWV

SEQ ID NO: 100 - protein, 20 aa, synthetic construct
  REGION 1..20: note = Description of Artificial Sequence: Synthetic peptide
  REGION 1..20: note = CD3 High - [anti-CD3]_H1.30_L1.47_scFv Linker
GKPGSGKPGSGKPGSGKPGS

SEQ ID NO: 101 - protein, 254 aa, synthetic construct
  REGION 1..254: note = Description of Artificial Sequence: Synthetic polypeptide
  REGION 1..254: note = CD3 High-Int #1 - [anti-CD3]_H1.32_L1.47_scFv scFv
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 102 - protein, 125 aa, synthetic construct
  REGION 1..125: note = Description of Artificial Sequence: Synthetic polypeptide
  REGION 1..125: note = CD3 High-Int #1 - [anti-CD3]_H1.32_L1.47_scFv Variable Heav y (vh) Domain
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 103 - protein, 5 aa, synthetic construct
  REGION 1..5: note = Description of Artificial Sequence: Synthetic peptide
  REGION 1..5: note = CD3 High-Int #1 - [anti-CD3]_H1.32_L1.47_scFv vhCDR1
TYAMN

SEQ ID NO: 104 - protein, 19 aa, synthetic construct
  REGION 1..19: note = Description of Artificial Sequence: Synthetic peptide
  REGION 1..19: note = CD3 High-Int #1 - [anti-CD3]_H1.32_L1.47_scFv vhCDR2
RIRSKANNYATYYADSVKG

SEQ ID NO: 105 - protein, 14 aa, synthetic construct
  REGION 1..14: note = Description of Artificial Sequence: Synthetic peptide
  REGION 1..14: note = CD3 High-Int #1 - [anti-CD3]_H1.32_L1.47_scFv vhCDR3
HGNFGDSYVSWFAY

SEQ ID NO: 106 - protein, 109 aa, synthetic construct; identical to SEQ ID NO: 96
  REGION 1..109: note = Description of Artificial Sequence: Synthetic polypeptide
  REGION 1..109: note = CD3 High-Int #1 - [anti-CD3]_H1.32_L1.47_scFv Variable Ligh t (vl) Domain

SEQ ID NO: 107 - protein, 14 aa, synthetic construct
  REGION 1..14: note = Description of Artificial Sequence: Synthetic peptide
  REGION 1..14: note = CD3 High-Int #1 - [anti-CD3]_H1.32_L1.47_scFv vlCDR1
GSSTGAVTTSNYAN

SEQ ID NO: 108 - protein, 7 aa, synthetic construct
  REGION 1..7: note = Description of Artificial Sequence: Synthetic peptide
  REGION 1..7: note = CD3 High-Int #1 - [anti-CD3]_H1.32_L1.47_scFv vlCDR2
GTNKRAP

SEQ ID NO: 109 - protein, 9 aa, synthetic construct
  REGION 1..9: note = Description of Artificial Sequence: Synthetic peptide
  REGION 1..9: note = CD3 High-Int #1 - [anti-CD3]_H1.32_L1.47_scFv vlCDR3
ALWYSNHWV

SEQ ID NO: 110 - protein, 20 aa, synthetic construct
  REGION 1..20: note = Description of Artificial Sequence: Synthetic peptide
  REGION 1..20: note = CD3 High-Int #1 - [anti-CD3]_H1.32_L1.47_scFv Linker
GKPGSGKPGSGKPGSGKPGS

SEQ ID NO: 111 - protein, 254 aa, synthetic construct
  REGION 1..254: note = Description of Artificial Sequence: Synthetic polypeptide
  REGION 1..254: note = CD3 High-Int #2 - [anti-CD3]_H1.89_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 112 - protein, 125 aa, synthetic construct
  REGION 1..125: note = Description of Artificial Sequence: Synthetic polypeptide
  REGION 1..125: note = CD3 High-Int #2 - [anti-CD3]_H1.89_L1.47_scFv Variable Heav y (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSS

SEQ ID NO: 113 - protein, 5 aa, synthetic construct
  REGION 1..5: note = Description of Artificial Sequence: Synthetic peptide
  REGION 1..5: note = CD3 High-Int #2 - [anti-CD3]_H1.89_L1.47_scFv vhCDR1
TYAMN

SEQ ID NO: 114 - protein, 19 aa, synthetic construct
  REGION 1..19: note = Description of Artificial Sequence: Synthetic peptide
  REGION 1..19: note = CD3 High-Int #2 - [anti-CD3]_H1.89_L1.47_scFv vhCDR2
RIRSKYNNYATYYADSVKG

SEQ ID NO: 115 - protein, 14 aa, synthetic construct
  REGION 1..14: note = Description of Artificial Sequence: Synthetic peptide
  REGION 1..14: note = CD3 High-Int #2 - [anti-CD3]_H1.89_L1.47_scFv vhCDR3
HGNFGDEYVSWFAY

SEQ ID NO: 116 - protein, 109 aa, synthetic construct; identical to SEQ ID NO: 96
  REGION 1..109: note = Description of Artificial Sequence: Synthetic polypeptide
  REGION 1..109: note = CD3 High-Int #2 - [anti-CD3]_H1.89_L1.47_scFv Variable Ligh t (vl) Domain

SEQ ID NO: 117 - protein, 14 aa, synthetic construct
  REGION 1..14: note = Description of Artificial Sequence: Synthetic peptide
  REGION 1..14: note = CD3 High-Int #2 - [anti-CD3]_H1.89_L1.47_scFv vlCDR1
GSSTGAVTTSNYAN

SEQ ID NO: 118 - protein, 7 aa, synthetic construct
  REGION 1..7: note = Description of Artificial Sequence: Synthetic peptide
  REGION 1..7: note = CD3 High-Int #2 - [anti-CD3]_H1.89_L1.47_scFv vlCDR2
GTNKRAP

SEQ ID NO: 119 - protein, 9 aa, synthetic construct
  REGION 1..9: note = Description of Artificial Sequence: Synthetic peptide
  REGION 1..9: note = CD3 High-Int #2 - [anti-CD3]_H1.89_L1.47_scFv vlCDR3
ALWYSNHWV

SEQ ID NO: 120 - protein, 20 aa, synthetic construct
  REGION 1..20: note = Description of Artificial Sequence: Synthetic peptide
  REGION 1..20: note = CD3 High-Int #2 - [anti-CD3]_H1.89_L1.47_scFv Linker
GKPGSGKPGSGKPGSGKPGS

SEQ ID NO: 121 - protein, 254 aa, synthetic construct
  REGION 1..254: note = Description of Artificial Sequence: Synthetic polypeptide
  REGION 1..254: note = CD3 High-Int - [anti-CD3]_H1.90_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 122 - protein, 125 aa, synthetic construct
  REGION 1..125: note = Description of Artificial Sequence: Synthetic polypeptide
  REGION 1..125: note = CD3 High-Int - [anti-CD3]_H1.90_L1.47_scFv Variable Heavy ( vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSS

SEQ ID NO: 123 - protein, 5 aa, synthetic construct
  REGION 1..5: note = Description of Artificial Sequence: Synthetic peptide
  REGION 1..5: note = CD3 High-Int - [anti-CD3]_H1.90_L1.47_scFv vhCDR1
TYAMN

SEQ ID NO: 124 - protein, 19 aa, synthetic construct
  REGION 1..19: note = Description of Artificial Sequence: Synthetic peptide
  REGION 1..19: note = CD3 High-Int - [anti-CD3]_H1.90_L1.47_scFv vhCDR2
RIRSKYNNYATYYADSVKG

SEQ ID NO: 125 - protein, 14 aa, synthetic construct
  REGION 1..14: note = Description of Artificial Sequence: Synthetic peptide
  REGION 1..14: note = CD3 High-Int - [anti-CD3]_H1.90_L1.47_scFv vhCDR3
HGNFGDPYVSWFAY

SEQ ID NO: 126 - protein, 109 aa, synthetic construct; identical to SEQ ID NO: 96
  REGION 1..109: note = Description of Artificial Sequence: Synthetic polypeptide
  REGION 1..109: note = CD3 High-Int - [anti-CD3]_H1.90_L1.47_scFv Variable Light ( vl) Domain

SEQ ID NO: 127 - protein, 14 aa, synthetic construct
  REGION 1..14: note = Description of Artificial Sequence: Synthetic peptide
  REGION 1..14: note = CD3 High-Int - [anti-CD3]_H1.90_L1.47_scFv vlCDR1
GSSTGAVTTSNYAN

SEQ ID NO: 128 - protein, 7 aa, synthetic construct
  REGION 1..7: note = Description of Artificial Sequence: Synthetic peptide
  REGION 1..7: note = CD3 High-Int - [anti-CD3]_H1.90_L1.47_scFv vlCDR2
GTNKRAP

SEQ ID NO: 129 - protein, 9 aa, synthetic construct
  REGION 1..9: note = Description of Artificial Sequence: Synthetic peptide
  REGION 1..9: note = CD3 High-Int - [anti-CD3]_H1.90_L1.47_scFv vlCDR3
ALWYSNHWV

SEQ ID NO: 130 - protein, 20 aa, synthetic construct
  REGION 1..20: note = Description of Artificial Sequence: Synthetic peptide
  REGION 1..20: note = CD3 High-Int - [anti-CD3]_H1.90_L1.47_scFv Linker
GKPGSGKPGSGKPGSGKPGS

SEQ ID NO: 131 - protein, 254 aa, synthetic construct
  REGION 1..254: note = Description of Artificial Sequence: Synthetic polypeptide
  REGION 1..254: note = Anti-CD3-Intermediate - [anti-CD3]_H1.33_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 132 - protein, 125 aa, synthetic construct
  REGION 1..125: note = Description of Artificial Sequence: Synthetic polypeptide
  REGION 1..125: note = Anti-CD3-Intermediate - [anti-CD3]_H1.33_L1.47_scFv Variabl e Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 133 - protein, 5 aa, synthetic construct
  REGION 1..5: note = Description of Artificial Sequence: Synthetic peptide
  REGION 1..5: note = Anti-CD3-Intermediate - [anti-CD3]_H1.33_L1.47_scFv vhCDR1
TYAMN

SEQ ID NO: 134 - protein, 19 aa, synthetic construct
  REGION 1..19: note = Description of Artificial Sequence: Synthetic peptide
  REGION 1..19: note = Anti-CD3-Intermediate - [anti-CD3]_H1.33_L1.47_scFv vhCDR2
RIRSKYNNYATYYADSVKG

SEQ ID NO: 135 - protein, 14 aa, synthetic construct
  REGION 1..14: note = Description of Artificial Sequence: Synthetic peptide
  REGION 1..14: note = Anti-CD3-Intermediate - [anti-CD3]_H1.33_L1.47_scFv vhCDR3
HGNFGDSYVSWFDY

SEQ ID NO: 136 - protein, 109 aa, synthetic construct; identical to SEQ ID NO: 96
  REGION 1..109: note = Description of Artificial Sequence: Synthetic polypeptide
  REGION 1..109: note = Anti-CD3-Intermediate - [anti-CD3]_H1.33_L1.47_scFv Variabl e Light (vl) Domain

SEQ ID NO: 137 - protein, 14 aa, synthetic construct
  REGION 1..14: note = Description of Artificial Sequence: Synthetic peptide
  REGION 1..14: note = Anti-CD3-Intermediate - [anti-CD3]_H1.33_L1.47_scFv vlCDR1
GSSTGAVTTSNYAN

SEQ ID NO: 138 - protein, 7 aa, synthetic construct
  REGION 1..7: note = Description of Artificial Sequence: Synthetic peptide
  REGION 1..7: note = Anti-CD3-Intermediate - [anti-CD3]_H1.33_L1.47_scFv vlCDR2
GTNKRAP

SEQ ID NO: 139 - protein, 9 aa, synthetic construct
  REGION 1..9: note = Description of Artificial Sequence: Synthetic peptide
  REGION 1..9: note = Anti-CD3-Intermediate - [anti-CD3]_H1.33_L1.47_scFv vlCDR3
ALWYSNHWV

SEQ ID NO: 140 - protein, 20 aa, synthetic construct
  REGION 1..20: note = Description of Artificial Sequence: Synthetic peptide
  REGION 1..20: note = Anti-CD3-Intermediate - [anti-CD3]_H1.33_L1.47_scFv Linker
GKPGSGKPGSGKPGSGKPGS

SEQ ID NO: 141 - protein, 254 aa, synthetic construct
  REGION 1..254: note = Description of Artificial Sequence: Synthetic polypeptide
  REGION 1..254: note = CD3 High-Int - [anti-CD3]_H1.31_L1.47_scFv scFv
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 142 - protein, 125 aa, synthetic construct
  REGION 1..125: note = Description of Artificial Sequence: Synthetic polypeptide
  REGION 1..125: note = CD3 High-Int - [anti-CD3]_H1.31_L1.47_scFv Variable Heavy ( vh) Domain
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 143 - protein, 5 aa, synthetic construct
  REGION 1..5: note = Description of Artificial Sequence: Synthetic peptide
  REGION 1..5: note = CD3 High-Int - [anti-CD3]_H1.31_L1.47_scFv vhCDR1
TYAMS

SEQ ID NO: 144 - protein, 19 aa, synthetic construct
  REGION 1..19: note = Description of Artificial Sequence: Synthetic peptide
  REGION 1..19: note = CD3 High-Int - [anti-CD3]_H1.31_L1.47_scFv vhCDR2
RIRSKYNNYATYYADSVKG

SEQ ID NO: 145 - protein, 14 aa, synthetic construct
  REGION 1..14: note = Description of Artificial Sequence: Synthetic peptide
  REGION 1..14: note = CD3 High-Int - [anti-CD3]_H1.31_L1.47_scFv vhCDR3
HGNFGDSYVSWFAY

SEQ ID NO: 146 - protein, 109 aa, synthetic construct; identical to SEQ ID NO: 96
  REGION 1..109: note = Description of Artificial Sequence: Synthetic polypeptide
  REGION 1..109: note = CD3 High-Int - [anti-CD3]_H1.31_L1.47_scFv Variable Light ( vl) Domain

SEQ ID NO: 147 - protein, 14 aa, synthetic construct
  REGION 1..14: note = Description of Artificial Sequence: Synthetic peptide
  REGION 1..14: note = CD3 High-Int - [anti-CD3]_H1.31_L1.47_scFv vlCDR1
GSSTGAVTTSNYAN

SEQ ID NO: 148 - protein, 7 aa, synthetic construct
  REGION 1..7: note = Description of Artificial Sequence: Synthetic peptide
  REGION 1..7: note = CD3 High-Int - [anti-CD3]_H1.31_L1.47_scFv vlCDR2
GTNKRAP

SEQ ID NO: 149 - protein, 9 aa, synthetic construct
  REGION 1..9: note = Description of Artificial Sequence: Synthetic peptide
  REGION 1..9: note = CD3 High-Int - [anti-CD3]_H1.31_L1.47_scFv vlCDR3
ALWYSNHWV

SEQ ID NO: 150 - protein, 20 aa, synthetic construct
  REGION 1..20: note = Description of Artificial Sequence: Synthetic peptide
  REGION 1..20: note = CD3 High-Int - [anti-CD3]_H1.31_L1.47_scFv Linker
GKPGSGKPGSGKPGSGKPGS

SEQ ID NO: 151 - protein, 254 aa, synthetic construct
  REGION 1..254: note = Description of Artificial Sequence: Synthetic polypeptide
  REGION 1..254: note = CD3 High[VL-VH] - [anti-CD3]_L1.47_ H1.30_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 152 - protein, 109 aa, synthetic construct; identical to SEQ ID NO: 96
  REGION 1..109: note = Description of Artificial Sequence: Synthetic polypeptide
  REGION 1..109: note = CD3 High[VL-VH] - [anti-CD3]_L1.47_ H1.30_scFv Variable Lig ht (vl) Domain

SEQ ID NO: 153 - protein, 14 aa, synthetic construct
  REGION 1..14: note = Description of Artificial Sequence: Synthetic peptide
  REGION 1..14: note = CD3 High[VL-VH] - [anti-CD3]_L1.47_ H1.30_scFv vlCDR1
GSSTGAVTTSNYAN

SEQ ID NO: 154 - protein, 7 aa, synthetic construct
  REGION 1..7: note = Description of Artificial Sequence: Synthetic peptide
  REGION 1..7: note = CD3 High[VL-VH] - [anti-CD3]_L1.47_ H1.30_scFv vlCDR2
GTNKRAP

SEQ ID NO: 155 - protein, 9 aa, synthetic construct
  REGION 1..9: note = Description of Artificial Sequence: Synthetic peptide
  REGION 1..9: note = CD3 High[VL-VH] - [anti-CD3]_L1.47_ H1.30_scFv vlCDR3
ALWYSNHWV

SEQ ID NO: 156 - protein, 125 aa, synthetic construct; identical to SEQ ID NO: 92
  REGION 1..125: note = Description of Artificial Sequence: Synthetic polypeptide
  REGION 1..125: note = CD3 High[VL-VH] - [anti-CD3]_L1.47_ H1.30_scFv Variable Hea vy (vh) Domain

SEQ ID NO: 157 - protein, 5 aa, synthetic construct
  REGION 1..5: note = Description of Artificial Sequence: Synthetic peptide
  REGION 1..5: note = CD3 High[VL-VH] - [anti-CD3]_L1.47_ H1.30_scFv vhCDR1
TYAMN

SEQ ID NO: 158 - protein, 19 aa, synthetic construct
  REGION 1..19: note = Description of Artificial Sequence: Synthetic peptide
  REGION 1..19: note = CD3 High[VL-VH] - [anti-CD3]_L1.47_ H1.30_scFv vhCDR2
RIRSKYNNYATYYADSVKG

SEQ ID NO: 159 - protein, 14 aa, synthetic construct
  REGION 1..14: note = Description of Artificial Sequence: Synthetic peptide
  REGION 1..14: note = CD3 High[VL-VH] - [anti-CD3]_L1.47_ H1.30_scFv vhCDR3
HGNFGDSYVSWFAY

SEQ ID NO: 160 - protein, 20 aa, synthetic construct
  REGION 1..20: note = Description of Artificial Sequence: Synthetic peptide
  REGION 1..20: note = CD3 High[VL-VH] - [anti-CD3]_L1.47_ H1.30_scFv Linker
GKPGSGKPGSGKPGSGKPGS

SEQ ID NO: 161 - protein, 254 aa, synthetic construct
  REGION 1..254: note = Description of Artificial Sequence: Synthetic polypeptide
  REGION 1..254: note = CD3 High-Int #1[VL-VH] - [anti-CD3]_L1.47_ H1.32_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 162 - protein, 109 aa, synthetic construct; identical to SEQ ID NO: 96
  REGION 1..109: note = Description of Artificial Sequence: Synthetic polypeptide
  REGION 1..109: note = CD3 High-Int #1[VL-VH] - [anti-CD3]_L1.47_ H1.32_scFv Varia ble Light (vl) Domain

SEQ ID NO: 163 - protein, 14 aa, synthetic construct
  REGION 1..14: note = Description of Artificial Sequence: Synthetic peptide
  REGION 1..14: note = CD3 High-Int #1[VL-VH] - [anti-CD3]_L1.47_ H1.32_scFv vlCDR 1
GSSTGAVTTSNYAN

SEQ ID NO: 164 - protein, 7 aa, synthetic construct
  REGION 1..7: note = Description of Artificial Sequence: Synthetic peptide
  REGION 1..7: note = CD3 High-Int #1[VL-VH] - [anti-CD3]_L1.47_ H1.32_scFv vlCDR 2
GTNKRAP

SEQ ID NO: 165 - protein, 9 aa, synthetic construct
  REGION 1..9: note = Description of Artificial Sequence: Synthetic peptide
  REGION 1..9: note = CD3 High-Int #1[VL-VH] - [anti-CD3]_L1.47_ H1.32_scFv vlCDR 3
ALWYSNHWV

SEQ ID NO: 166 - protein, 125 aa, synthetic construct; identical to SEQ ID NO: 102
  REGION 1..125: note = Description of Artificial Sequence: Synthetic polypeptide
  REGION 1..125: note = CD3 High-Int #1[VL-VH] - [anti-CD3]_L1.47_ H1.32_scFv Varia ble Heavy (vh) Domain

SEQ ID NO: 167 - protein, 5 aa, synthetic construct
  REGION 1..5: note = Description of Artificial Sequence: Synthetic peptide
  REGION 1..5: note = CD3 High-Int #1[VL-VH] - [anti-CD3]_L1.47_ H1.32_scFv vhCDR 1
TYAMN

SEQ ID NO: 168 - protein, 19 aa, synthetic construct
  REGION 1..19: note = Description of Artificial Sequence: Synthetic peptide
  REGION 1..19: note = CD3 High-Int #1[VL-VH] - [anti-CD3]_L1.47_ H1.32_scFv vhCDR 2
RIRSKANNYATYYADSVKG

SEQ ID NO: 169 - protein, 14 aa, synthetic construct
  REGION 1..14: note = Description of Artificial Sequence: Synthetic peptide
  REGION 1..14: note = CD3 High-Int #1[VL-VH] - [anti-CD3]_L1.47_ H1.32_scFv vhCDR 3
HGNFGDSYVSWFAY

SEQ ID NO: 170 - protein, 20 aa, synthetic construct
  REGION 1..20: note = Description of Artificial Sequence: Synthetic peptide
  REGION 1..20: note = CD3 High-Int #1[VL-VH] - [anti-CD3]_L1.47_ H1.32_scFv Linke r
GKPGSGKPGSGKPGSGKPGS

SEQ ID NO: 171 - protein, 254 aa, synthetic construct
  REGION 1..254: note = Description of Artificial Sequence: Synthetic polypeptide
  REGION 1..254: note = CD3 High-Int #2[VL-VH] - [anti-CD3]_L1.47_ H1.89_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSS

SEQ ID NO: 172 - protein, 109 aa, synthetic construct; identical to SEQ ID NO: 96
  REGION 1..109: note = Description of Artificial Sequence: Synthetic polypeptide
  REGION 1..109: note = CD3 High-Int #2[VL-VH] - [anti-CD3]_L1.47_ H1.89_scFv Varia ble Light (vl) Domain

SEQ ID NO: 173 - protein, 14 aa, synthetic construct
  REGION 1..14: note = Description of Artificial Sequence: Synthetic peptide
  REGION 1..14: note = CD3 High-Int #2[VL-VH] - [anti-CD3]_L1.47_ H1.89_scFv vlCDR 1
GSSTGAVTTSNYAN

SEQ ID NO: 174 - protein, 7 aa, synthetic construct
  REGION 1..7: note = Description of Artificial Sequence: Synthetic peptide
  REGION 1..7: note = CD3 High-Int #2[VL-VH] - [anti-CD3]_L1.47_ H1.89_scFv vlCDR 2
GTNKRAP

SEQ ID NO: 175 - protein, 9 aa, synthetic construct
  REGION 1..9: note = Description of Artificial Sequence: Synthetic peptide
  REGION 1..9: note = CD3 High-Int #2[VL-VH] - [anti-CD3]_L1.47_ H1.89_scFv vlCDR 3
ALWYSNHWV

SEQ ID NO: 176 - protein, 125 aa, synthetic construct; identical to SEQ ID NO: 112
  REGION 1..125: note = Description of Artificial Sequence: Synthetic polypeptide
  REGION 1..125: note = CD3 High-Int #2[VL-VH] - [anti-CD3]_L1.47_ H1.89_scFv Varia ble Heavy (vh) Domain

SEQ ID NO: 177 - protein, 5 aa, synthetic construct
  REGION 1..5: note = Description of Artificial Sequence: Synthetic peptide
  REGION 1..5: note = CD3 High-Int #2[VL-VH] - [anti-CD3]_L1.47_ H1.89_scFv vhCDR 1
TYAMN

SEQ ID NO: 178 - protein, 19 aa, synthetic construct
  REGION 1..19: note = Description of Artificial Sequence: Synthetic peptide
  REGION 1..19: note = CD3 High-Int #2[VL-VH] - [anti-CD3]_L1.47_ H1.89_scFv vhCDR 2
RIRSKYNNYATYYADSVKG

SEQ ID NO: 179 - protein, 14 aa, synthetic construct
  REGION 1..14: note = Description of Artificial Sequence: Synthetic peptide
  REGION 1..14: note = CD3 High-Int #2[VL-VH] - [anti-CD3]_L1.47_ H1.89_scFv vhCDR 3
HGNFGDEYVSWFAY

SEQ ID NO: 180 - protein, 20 aa, synthetic construct
  REGION 1..20: note = Description of Artificial Sequence: Synthetic peptide
  REGION 1..20: note = CD3 High-Int #2[VL-VH] - [anti-CD3]_L1.47_ H1.89_scFv Linke r
GKPGSGKPGSGKPGSGKPGS

SEQ ID NO: 181 - protein, 254 aa, synthetic construct
  REGION 1..254: note = Description of Artificial Sequence: Synthetic polypeptide
  REGION 1..254: note = CD3 High-Int[VL-VH] - [anti-CD3]_L1.47_ H1.90_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSS

SEQ ID NO: 182 - protein, 109 aa, synthetic construct; identical to SEQ ID NO: 96
  REGION 1..109: note = Description of Artificial Sequence: Synthetic polypeptide
  REGION 1..109: note = CD3 High-Int[VL-VH] - [anti-CD3]_L1.47_ H1.90_scFv Variable Light (vl) Domain

SEQ ID NO: 183 - protein, 14 aa, synthetic construct
  REGION 1..14: note = Description of Artificial Sequence: Synthetic peptide
  REGION 1..14: note = CD3 High-Int[VL-VH] - [anti-CD3]_L1.47_ H1.90_scFv vlCDR1
GSSTGAVTTSNYAN

SEQ ID NO: 184 - protein, 7 aa, synthetic construct
  REGION 1..7: note = Description of Artificial Sequence: Synthetic peptide
  REGION 1..7: note = CD3 High-Int[VL-VH] - [anti-CD3]_L1.47_ H1.90_scFv vlCDR2
GTNKRAP

SEQ ID NO: 185 - protein, 9 aa, synthetic construct
  REGION 1..9: note = Description of Artificial Sequence: Synthetic peptide
  REGION 1..9: note = CD3 High-Int[VL-VH] - [anti-CD3]_L1.47_ H1.90_scFv vlCDR3
ALWYSNHWV

SEQ ID NO: 186 - protein, 125 aa, synthetic construct; identical to SEQ ID NO: 122
  REGION 1..125: note = Description of Artificial Sequence: Synthetic polypeptide
  REGION 1..125: note = CD3 High-Int[VL-VH] - [anti-CD3]_L1.47_ H1.90_scFv Variable Heavy (vh) Domain

SEQ ID NO: 187 - protein, 5 aa, synthetic construct
  REGION 1..5: note = Description of Artificial Sequence: Synthetic peptide
  REGION 1..5: note = CD3 High-Int[VL-VH] - [anti-CD3]_L1.47_ H1.90_scFv vhCDR1
TYAMN

SEQ ID NO: 188 - protein, 19 aa, synthetic construct
  REGION 1..19: note = Description of Artificial Sequence: Synthetic peptide
  REGION 1..19: note = CD3 High-Int[VL-VH] - [anti-CD3]_L1.47_ H1.90_scFv vhCDR2
RIRSKYNNYATYYADSVKG

SEQ ID NO: 189 - protein, 14 aa, synthetic construct
  REGION 1..14: note = Description of Artificial Sequence: Synthetic peptide
  REGION 1..14: note = CD3 High-Int[VL-VH] - [anti-CD3]_L1.47_ H1.90_scFv vhCDR3
HGNFGDPYVSWFAY

SEQ ID NO: 190 - protein, 20 aa, synthetic construct
  REGION 1..20: note = Description of Artificial Sequence: Synthetic peptide
  REGION 1..20: note = CD3 High-Int[VL-VH] - [anti-CD3]_L1.47_ H1.90_scFv Linker
GKPGSGKPGSGKPGSGKPGS

SEQ ID NO: 191 - protein, 254 aa, synthetic construct
  REGION 1..254: note = Description of Artificial Sequence: Synthetic polypeptide
  REGION 1..254: note = Anti-CD3-Intermediate[VL-VH] - [anti-CD3]_L1.47_ H1.33_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 192 - protein, 109 aa, synthetic construct; identical to SEQ ID NO: 96
  REGION 1..109: note = Description of Artificial Sequence: Synthetic polypeptide
  REGION 1..109: note = Anti-CD3-Intermediate[VL-VH] - [anti-CD3]_L1.47_ H1.33_scFv Variable Light (vl) Domain

SEQ ID NO: 193 - protein, 14 aa, synthetic construct
  REGION 1..14: note = Description of Artificial Sequence: Synthetic peptide
  REGION 1..14: note = Anti-CD3-Intermediate[VL-VH] - [anti-CD3]_L1.47_ H1.33_scFv vlCDR1
GSSTGAVTTSNYAN

SEQ ID NO: 194 - protein, 7 aa, synthetic construct
  REGION 1..7: note = Description of Artificial Sequence: Synthetic peptide
  REGION 1..7: note = Anti-CD3-Intermediate[VL-VH] - [anti-CD3]_L1.47_ H1.33_scFv vlCDR2
GTNKRAP

SEQ ID NO: 195 - protein, 9 aa, synthetic construct
  REGION 1..9: note = Description of Artificial Sequence: Synthetic peptide
  REGION 1..9: note = Anti-CD3-Intermediate[VL-VH] - [anti-CD3]_L1.47_ H1.33_scFv vlCDR3
ALWYSNHWV

SEQ ID NO: 196 - protein, 125 aa, synthetic construct; identical to SEQ ID NO: 132
  REGION 1..125: note = Description of Artificial Sequence: Synthetic polypeptide
  REGION 1..125: note = Anti-CD3-Intermediate[VL-VH] - [anti-CD3]_L1.47_ H1.33_scFv Variable Heavy (vh) Domain

SEQ ID NO: 197 - protein, 5 aa, synthetic construct
  REGION 1..5: note = Description of Artificial Sequence: Synthetic peptide
  REGION 1..5: note = Anti-CD3-Intermediate[VL-VH] - [anti-CD3]_L1.47_ H1.33_scFv vhCDR1
TYAMN

SEQ ID NO: 198 - protein, 19 aa, synthetic construct
  REGION 1..19: note = Description of Artificial Sequence: Synthetic peptide
  REGION 1..19: note = Anti-CD3-Intermediate[VL-VH] - [anti-CD3]_L1.47_ H1.33_scFv vhCDR2
RIRSKYNNYATYYADSVKG

SEQ ID NO: 199 - protein, 14 aa, synthetic construct
  REGION 1..14: note = Description of Artificial Sequence: Synthetic peptide
  REGION 1..14: note = Anti-CD3-Intermediate[VL-VH] - [anti-CD3]_L1.47_ H1.33_scFv vhCDR3
HGNFGDSYVSWFDY

SEQ ID NO: 200 - protein, 20 aa, synthetic construct
  REGION 1..20: note = Description of Artificial Sequence: Synthetic peptide
  REGION 1..20: note = Anti-CD3-Intermediate[VL-VH] - [anti-CD3]_L1.47_ H1.33_scFv Linker
GKPGSGKPGSGKPGSGKPGS

SEQ ID NO: 201 - protein, 254 aa, synthetic construct
  REGION 1..254: note = Description of Artificial Sequence: Synthetic polypeptide
  REGION 1..254: note = CD3 High-Int[VL-VH] - [anti-CD3]_L1.47_ H1.31_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 202 - protein, 109 aa, synthetic construct; identical to SEQ ID NO: 96
  REGION 1..109: note = Description of Artificial Sequence: Synthetic polypeptide
  REGION 1..109: note = CD3 High-Int[VL-VH] - [anti-CD3]_L1.47_ H1.31_scFv Variable Light (vl) Domain

SEQ ID NO: 203 - protein, 14 aa, synthetic construct
  REGION 1..14: note = Description of Artificial Sequence: Synthetic peptide
  REGION 1..14: note = CD3 High-Int[VL-VH] - [anti-CD3]_L1.47_ H1.31_scFv vlCDR1
GSSTGAVTTSNYAN

SEQ ID NO: 204 - protein, 7 aa, synthetic construct
  REGION 1..7: note = Description of Artificial Sequence: Synthetic peptide
  REGION 1..7: note = CD3 High-Int[VL-VH] - [anti-CD3]_L1.47_ H1.31_scFv vlCDR2
GTNKRAP

SEQ ID NO: 205 - protein, 9 aa, synthetic construct
  REGION 1..9: note = Description of Artificial Sequence: Synthetic peptide
  REGION 1..9: note = CD3 High-Int[VL-VH] - [anti-CD3]_L1.47_ H1.31_scFv vlCDR3
ALWYSNHWV

SEQ ID NO: 206 - protein, 125 aa, synthetic construct; identical to SEQ ID NO: 142
  REGION 1..125: note = Description of Artificial Sequence: Synthetic polypeptide
  REGION 1..125: note = CD3 High-Int[VL-VH] - [anti-CD3]_L1.47_ H1.31_scFv Variable Heavy (vh) Domain

SEQ ID NO: 207 - protein, 5 aa, synthetic construct
  REGION 1..5: note = Description of Artificial Sequence: Synthetic peptide
  REGION 1..5: note = CD3 High-Int[VL-VH] - [anti-CD3]_L1.47_ H1.31_scFv vhCDR1
TYAMS

SEQ ID NO: 208 - protein, 19 aa, synthetic construct
  REGION 1..19: note = Description of Artificial Sequence: Synthetic peptide
  REGION 1..19: note = CD3 High-Int[VL-VH] - [anti-CD3]_L1.47_ H1.31_scFv vhCDR2
RIRSKYNNYATYYADSVKG

SEQ ID NO: 209 - protein, 14 aa, synthetic construct
  REGION 1..14: note = Description of Artificial Sequence: Synthetic peptide
  REGION 1..14: note = CD3 High-Int[VL-VH] - [anti-CD3]_L1.47_ H1.31_scFv vhCDR3
HGNFGDSYVSWFAY

SEQ ID NO: 210 - protein, 20 aa, synthetic construct
  REGION 1..20: note = Description of Artificial Sequence: Synthetic peptide
  REGION 1..20: note = CD3 High-Int[VL-VH] - [anti-CD3]_L1.47_ H1.31_scFv Linker
GKPGSGKPGSGKPGSGKPGS

SEQ ID NO: 211 - protein, 875 aa, Homo sapiens [sequence omitted]

SEQ ID NO: 212 - protein, 845 aa, Homo sapiens
  REGION 1..845: note = sp|O14638|31-875
LLVIMSLGLGLGLGLRKLEKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 213 - protein, 874 aa, Mus musculus
  REGION 1..874: note = sp|Q6DYE8
MDSRLALATEEPIKKDSLKKYKILCVVLLALLVIVSLGLGLGLGLRKPEEQGSCRKKCFDSSHRGLEGCRCDSGCTGRGDCCWDFEDTCVKSTQIWTCNLFRCGENRLETALCSCADDCLQRKDCCADYKTVCQGESPWVTEACASSQEPQCPPGFDLPPVILFSMDGFRAEYLQTWSTLLPNINKLKTCGIHSKYMRAMYPTKTFPNHYTIVTGLYPESHGIIDNNMYDVHLNKNFSLSSVEKSNPAWWSGQPIWLTAMYQGLKAACYYWPGSDVAVNGSFPTIYRNYSNSVPYERRITTLLQWLDLPKADRPSFYTIYVEEPDSAGHSSGPVSAGVIKALQSVDNAFGMLMEGLKQRNLHNCVNIIVLADHGMDQTSCDRVEYMTDYFPKINFYMYQGPAPRIRTRNIPQDFFTFNSEEIVRNLSCRKPDQHFKPYLTPDLPKRLHYAKNVRIDKAHLMVDRQWLAFRSKGSSNCGGGTHGYNNEFKSMEAIFLAHGPSFIEKTVIEPFENIEVYNLLCDLLHIEPAPNNGTHGSLNHLLKTPFYKPSHAGELSTPADCGFTTPLPTDPLDCSCPALQNTPGLEEQANQRLNLSEGEVAATVKANLPFGRPRVMQKNGDHCLLYHRDYISGYGKAMKMPMWSSYTVLKPGDTSSLPPTVPDCLRADVRVAPSESQKCSFYLADKNITHGFLYPAIKGTNESRYDALITSNLVPMYKEFKKMWDYFHEVLLIKYAIERNGLNVVSGPIFDYNYDGHFDAPDEITQYVAGTDVPIPTHYFVVLTSCKDQTHTPDSCPGWLDVLPFIVPHRPTNIESCSENKTEDLWVEERFQAHAARVRDVELLTGLDFYQEKAQPVSQILQLKTYLPTFETII

SEQ ID NO: 214 - protein, 844 aa, Mus musculus
  REGION 1..844: note = sp|Q6DYE8|31-874
LLVIVSLGLGLGLGLRKPEEQGSCRKKCFDSSHRGLEGCRCDSGCTGRGDCCWDFEDTCVKSTQIWTCNLFRCGENRLETALCSCADDCLQRKDCCADYKTVCQGESPWVTEACASSQEPQCPPGFDLPPVILFSMDGFRAEYLQTWSTLLPNINKLKTCGIHSKYMRAMYPTKTFPNHYTIVTGLYPESHGIIDNNMYDVHLNKNFSLSSVEKSNPAWWSGQPIWLTAMYQGLKAACYYWPGSDVAVNGSFPTIYRNYSNSVPYERRITTLLQWLDLPKADRPSFYTIYVEEPDSAGHSSGPVSAGVIKALQSVDNAFGMLMEGLKQRNLHNCVNIIVLADHGMDQTSCDRVEYMTDYFPKINFYMYQGPAPRIRTRNIPQDFFTFNSEEIVRNLSCRKPDQHFKPYLTPDLPKRLHYAKNVRIDKAHLMVDRQWLAFRSKGSSNCGGGTHGYNNEFKSMEAIFLAHGPSFIEKTVIEPFENIEVYNLLCDLLHIEPAPNNGTHGSLNHLLKTPFYKPSHAGELSTPADCGFTTPLPTDPLDCSCPALQNTPGLEEQANQRLNLSEGEVAATVKANLPFGRPRVMQKNGDHCLLYHRDYISGYGKAMKMPMWSSYTVLKPGDTSSLPPTVPDCLRADVRVAPSESQKCSFYLADKNITHGFLYPAIKGTNESRYDALITSNLVPMYKEFKKMWDYFHEVLLIKYAIERNGLNVVSGPIFDYNYDGHFDAPDEITQYVAGTDVPIPTHYFVVLTSCKDQTHTPDSCPGWLDVLPFIVPHRPTNIESCSENKTEDLWVEERFQAHAARVRDVELLTGLDFYQEKAQPVSQILQLKTYLPTFETII

SEQ ID NO: 215 - protein, 874 aa, Macaca fascicularis
  REGION 1..874: note = tr|A0A2K5TKP4
MESMLTLATEQPVKKNTLKKYKIACIVLLALLVIMSLGLGLGLGLRKLEKQGSCRKKCFDASFRGLENCRCDVACEDRGDCCWDFEDTCVESTRIWTCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSEEQNNPAWWHGQPMWLTAMYQGLKAATYFWPGSEVAINGSFPSIYMPYNRSVPYEERISTLLKWLDLPKAERPSFYTMYFEEPDSSGHAGGPVSARVIKALQVVDHAFGMLMEGLKQRNLHNCVNIILLADHGMDQTYCNKMEYMTDYFPRINFYMYEGPAPRIRALNVPHDFFSCKYEDTYMKKRCRKPDQHFKPYLTPDLPKRLHYAKNVRIDKVHLFVDRQWLAVGSKSNTNCGGGNHGYNNEFRSMEAIFLAHGPSFKEKTEVEPFENIEVYNLMCDLLRIQPAPNNGTRGSLNHLLKVPFYEPSHAEEVSKFSVCGFANPLPTNNLSCLCPHLQNSIQLEQVNQMLNLTQEEITATVKVNLPFGRPRVLQKNVDNCLLYHREYVSGFGKAMRMPMWSSYTVPQLGDTSPLPPTVPDCLRADVRVPPSESQKCSFYLADENITHGFLYPPAINRTSDSQYDALIMSNLVPMYEEFRKMWDYFHSVLLIKHATERNGVNVVSGPIFDYNYDGHFDAPEEITKHIANTDIPIPTHYFVVLTSCKNKSHTPENCPGWLDVLPFIIPHRPTNVESCPEGKPEALWVEERFTAHIARVRDVELLTGLDFYQDKAQPVSEILQLKTYLPTFETTI

SEQ ID NO: 216 - protein, 844 aa, Macaca fascicularis
  REGION 1..844: note = tr|A0A2K5TKP4|31-874
LLVIMSLGLGLGLGLRKLEKQGSCRKKCFDASFRGLENCRCDVACEDRGDCCWDFEDTCVESTRIWTCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSEEQNNPAWWHGQPMWLTAMYQGLKAATYFWPGSEVAINGSFPSIYMPYNRSVPYEERISTLLKWLDLPKAERPSFYTMYFEEPDSSGHAGGPVSARVIKALQVVDHAFGMLMEGLKQRNLHNCVNIILLADHGMDQTYCNKMEYMTDYFPRINFYMYEGPAPRIRALNVPHDFFSCKYEDTYMKKRCRKPDQHFKPYLTPDLPKRLHYAKNVRIDKVHLFVDRQWLAVGSKSNTNCGGGNHGYNNEFRSMEAIFLAHGPSFKEKTEVEPFENIEVYNLMCDLLRIQPAPNNGTRGSLNHLLKVPFYEPSHAEEVSKFSVCGFANPLPTNNLSCLCPHLQNSIQLEQVNQMLNLTQEEITATVKVNLPFGRPRVLQKNVDNCLLYHREYVSGFGKAMRMPMWSSYTVPQLGDTSPLPPTVPDCLRADVRVPPSESQKCSFYLADENITHGFLYPPAINRTSDSQYDALIMSNLVPMYEEFRKMWDYFHSVLLIKHATERNGVNVVSGPIFDYNYDGHFDAPEEITKHIANTDIPIPTHYFVVLTSCKNKSHTPENCPGWLDVLPFIIPHRPTNVESCPEGKPEALWVEERFTAHIARVRDVELLTGLDFYQDKAQPVSEILQLKTYLPTFETTI

SEQ ID NO: 217 - protein, 124 aa, synthetic construct
  REGION 1..124: note = Description of Artificial Sequence: Synthetic polypeptide
  REGION 1..124: note = MESO-A[MSLN]_H1.1_L1 Variable heavy (vh) domain
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSS

SEQ ID NO: 218 - protein, 5 aa, synthetic construct
  REGION 1..5: note = Description of Artificial Sequence: Synthetic peptide
  REGION 1..5: note = MESO-A[MSLN]_H1.1_L1 vhCDR1
RYWMS

SEQ ID NO: 219 - protein, 17 aa, synthetic construct
  REGION 1..17: note = Description of Artificial Sequence: Synthetic peptide
  REGION 1..17: note = MESO-A[MSLN]_H1.1_L1 vhCDR2
EINPDSSTIVYTPSVKG

SEQ ID NO: 220 - protein, 15 aa, synthetic construct
  REGION 1..15: note = Description of Artificial Sequence: Synthetic peptide
  REGION 1..15: note = MESO-A[MSLN]_H1.1_L1 vhCDR3
RGSHYYGYRTGYFDV

SEQ ID NO: 221 - protein, 106 aa, synthetic construct
  REGION 1..106: note = Description of Artificial Sequence: Synthetic polypeptide
  REGION 1..106: note = MESO-A[MSLN]_H1.1_L1 Variable light (vl) domain
DVVLTQSPATLSLSPGERATLSCRASSSVSYMYWHQQKPDQSPKLLIYDTSNLASGVPVRFSGSGSGTDYTLTISSLEPEDFAVYYCQQWSSYPPTFGGGTKLEIK

SEQ ID NO: 222 - protein, 10 aa, synthetic construct
  REGION 1..10: note = Description of Artificial Sequence: Synthetic peptide
  REGION 1..10: note = MESO-A[MSLN]_H1.1_L1 vlCDR1
RASSSVSYMY

SEQ ID NO: 223 - protein, 7 aa, synthetic construct
  REGION 1..7: note = Description of Artificial Sequence: Synthetic peptide
  REGION 1..7: note = MESO-A[MSLN]_H1.1_L1 vlCDR2
DTSNLAS

SEQ ID NO: 224 - protein, 9 aa, synthetic construct
  REGION 1..9: note = Description of Artificial Sequence: Synthetic peptide
  REGION 1..9: note = MESO-A[MSLN]_H1.1_L1 vlCDR3
QQWSSYPPT

SEQ ID NO: 225 - protein, 454 aa, synthetic construct
  REGION 1..454: note = Description of Artificial Sequence: Synthetic polypeptide
  REGION 1..454: note = XENP16249 Chain 1 - MESO-A[MSLN]_H1.1_IgG1 Heavy Chain
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 213 aa, synthetic construct
  REGION 1..213: note = Description of Artificial Sequence: Synthetic polypeptide
  REGION 1..213: note = XENP16249 Chain 2 - MESO-A[MSLN]_L1 Light Chain
DVVLTQSPATLSLSPGERATLSCRASSSVSYMYWHQQKPDQSPKLLIYDTSNLASGVPVRFSGSGSGTDYTLTISSLEPEDFAVYYCQQWSSYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 453 aa, synthetic construct
  REGION 1..453: note = Description of Artificial Sequence: Synthetic polypeptide
  REGION 1..453: note = XENP16250 Chain 1 - MESO-A[MSLN]_H1.1_IgG1_PVA_/S267K Heav y Chain
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 213 aa, synthetic construct; identical to SEQ ID NO: 226
  REGION 1..213: note = Description of Artificial Sequence: Synthetic polypeptide
  REGION 1..213: note = XENP16250 Chain 2 - MESO-A[MSLN]_L1 Light Chain

SEQ ID NO: 229 - protein, 453 aa, synthetic construct
  REGION 1..453: note = Description of Artificial Sequence: Synthetic polypeptide
  REGION 1..453: note = XENP16253 Chain 1 - MESO-A[MSLN]_H1.1_IgG1_pI(-)_Isosteric_ A_/PVA_/S267K/L368D/K370S Heavy Chain
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 230 - protein, 213 aa, synthetic construct; identical to SEQ ID NO: 226
  REGION 1..213: note = Description of Artificial Sequence: Synthetic polypeptide
  REGION 1..213: note = XENP16253 Chain 2 - MESO-A[MSLN]_L1 Light Chain

SEQ ID NO: 231 - protein, 123 aa, synthetic construct
  REGION 1..123: note = Description of Artificial Sequence: Synthetic polypeptide
  REGION 1..123: note = MESO-B[MSLN]_H0L0 Variable heavy (vh) domain
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSS

SEQ ID NO: 232 - protein, 7 aa, synthetic construct
  REGION 1..7: note = Description of Artificial Sequence: Synthetic peptide
  REGION 1..7: note = MESO-B[MSLN]_H0L0 vhCDR1
SNSATWN

SEQ ID NO: 233 - protein, 18 aa, synthetic construct
  REGION 1..18: note = Description of Artificial Sequence: Synthetic peptide
  REGION 1..18: note = MESO-B[MSLN]_H0L0 vhCDR2
RTYYRSKWYNDYAVSVKS

SEQ ID NO: 234 - protein, 11 aa, synthetic construct
  REGION 1..11: note = Description of Artificial Sequence: Synthetic peptide
  REGION 1..11: note = MESO-B[MSLN]_H0L0 vhCDR3
GMMTYYYGMDV

SEQ ID NO: 235 - protein, 115 aa, synthetic construct
  REGION 1..115: note = Description of Artificial Sequence: Synthetic polypeptide
  REGION 1..115: note = MESO-B[MSLN]_H0L0 Variable light (vl) domain
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVL

SEQ ID NO: 236 - protein, 14 aa, synthetic construct
  REGION 1..14: note = Description of Artificial Sequence: Synthetic peptide
  REGION 1..14: note = MESO-B[MSLN]_H0L0 vlCDR1
TLRSGINVGPYRIY

SEQ ID NO: 237 - protein, 7 aa, synthetic construct
  REGION 1..7: note = Description of Artificial Sequence: Synthetic peptide
  REGION 1..7: note = MESO-B[MSLN]_H0L0 vlCDR2
SDKQQGS

SEQ ID NO: 238 - protein, 9 aa, synthetic construct
  REGION 1..9: note = Description of Artificial Sequence: Synthetic peptide
  REGION 1..9: note = MESO-B[MSLN]_H0L0 vlCDR3
MIWHSSAAV

SEQ ID NO: 239 - protein, 453 aa, synthetic construct
  REGION 1..453: note = Description of Artificial Sequence: Synthetic polypeptide
  REGION 1..453: note = XENP16827 Chain 1 - MESO-B[MSLN]_H0_IgG1 Heavy Chain
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 221 aa, synthetic construct
  REGION 1..221: note = Description of Artificial Sequence: Synthetic polypeptide
  REGION 1..221: note = XENP16827 Chain 2 - MESO-B[MSLN]_L0 Light Chain
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 241 - protein, 120 aa, synthetic construct
  REGION 1..120: note = Description of Artificial Sequence: Synthetic polypeptide
  REGION 1..120: note = MESO-C[MSLN]_H1L1 Variable heavy (vh) domain
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSS

SEQ ID NO: 242 - protein, 5 aa, synthetic construct
  REGION 1..5: note = Description of Artificial Sequence: Synthetic peptide
  REGION 1..5: note = MESO-C[MSLN]_H1L1 vhCDR1
SYWMH

SEQ ID NO: 243 - protein, 17 aa, synthetic construct
  REGION 1..17: note = Description of Artificial Sequence: Synthetic peptide
  REGION 1..17: note = MESO-C[MSLN]_H1L1 vhCDR2
MIHPNSDNTIYYEKFQG

SEQ ID NO: 244 - protein, 11 aa, synthetic construct
  REGION 1..11: note = Description of Artificial Sequence: Synthetic peptide
  REGION 1..11: note = MESO-C[MSLN]_H1L1 vhCDR3
IITPVVPKFDY

SEQ ID NO: 245 - protein, 107 aa, synthetic construct
  REGION 1..107: note = Description of Artificial Sequence: Synthetic polypeptide
  REGION 1..107: note = MESO-C[MSLN]_H1L1 Variable light (vl) domain
DIVMTQSPDSLAVSLGERATINCKASHDVGTSVAWYQQKPGQSPKLLIYWASTRHTGVPDRFTGSGSGTDFTLTISSLQAEDVAVYFCQQYSSYPLTFGAGTKLEIK

SEQ ID NO: 246 - protein, 11 aa, synthetic construct
  REGION 1..11: note = Description of Artificial Sequence: Synthetic peptide
  REGION 1..11: note = MESO-C[MSLN]_H1L1 vlCDR1
KASHDVGTSVA

SEQ ID NO: 247 - protein, 7 aa, synthetic construct
  REGION 1..7: note = Description of Artificial Sequence: Synthetic peptide
  REGION 1..7: note = MESO-C[MSLN]_H1L1 vlCDR2
WASTRHT

SEQ ID NO: 248 - protein, 9 aa, synthetic construct
  REGION 1..9: note = Description of Artificial Sequence: Synthetic peptide
  REGION 1..9: note = MESO-C[MSLN]_H1L1 vlCDR3
QQYSSYPLT

SEQ ID NO: 249 - protein, 449 aa, synthetic construct
  REGION 1..449: note = Description of Artificial Sequence: Synthetic polypeptide
  REGION 1..449: note = XENP31693 Chain 1 - MESO-C[MSLN]_H1_IgG1_PVA_/S267K Heavy C hain
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 214 aa, synthetic construct
  REGION 1..214: note = Description of Artificial Sequence: Synthetic polypeptide
  REGION 1..214: note = XENP31693 Chain 2 - MESO-C[MSLN]_L1 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - protein, 120 aa, synthetic construct
  REGION 1..120: note = Description of Artificial Sequence: Synthetic polypeptide
  REGION 1..120: note = MESO-C[MSLN]_H1.1 Variable Heavy
QVQLVQSGAEVKKPGASVKVSCKASGYTFTGYWMHWVRQAPGQGLEWMGMIHPNSDNTIYYEKFQGRVTMTVDKSISTAYMELSRLRSDDTAVYYCAIIITPVVPKFDYWGQGTLVTVSS

SEQ ID NO: 252 - protein, 120 aa, synthetic construct
  REGION 1..120: note = Description of Artificial Sequence: Synthetic polypeptide
  REGION 1..120: note = MESO-C[MSLN]_H1.2 Variable Heavy
QVQLVQSGAEVKKPGASVKVSCKASGYTFTSYYMHWVRQAPGQGLEWMGMIHPNSDNTIYYEKFQGRVTMTVDKSISTAYMELSRLRSDDTAVYYCAIIITPVVPKFDYWGQGTLVTVSS

SEQ ID NO: 253 - protein, 120 aa, synthetic construct
  REGION 1..120: note = Description of Artificial Sequence: Synthetic polypeptide
  REGION 1..120: note = MESO-C[MSLN]_H1.3 Variable Heavy
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSS

SEQ ID NO: 254 - protein, 120 aa, synthetic construct
  REGION 1..120: note = Description of Artificial Sequence: Synthetic polypeptide
  REGION 1..120: note = MESO-C[MSLN]_H1.4 Variable Heavy
QVQLVQSGAEVKKPGASVKVSCKASGYTFTSYWMHWVRQAPGQGLEWMGMINPNSDNTIYYEKFQGRVTMTVDKSISTAYMELSRLRSDDTAVYYCAIIITPVVPKFDYWGQGTLVTVSS

SEQ ID NO: 255 - protein, 120 aa, synthetic construct
  REGION 1..120: note = Description of Artificial Sequence: Synthetic polypeptide
  REGION 1..120: note = MESO-C[MSLN]_H1.5 Variable Heavy
QVQLVQSGAEVKKPGASVKVSCKASGYTFTSYWMHWVRQAPGQGLEWMGMIHPQSDNTIYYEKFQGRVTMTVDKSISTAYMELSRLRSDDTAVYYCAIIITPVVPKFDYWGQGTLVTVSS

SEQ ID NO: 256 - protein, 120 aa, synthetic construct
  REGION 1..120: note = Description of Artificial Sequence: Synthetic polypeptide
  REGION 1..120: note = MESO-C[MSLN]_H1.6 Variable Heavy
QVQLVQSGAEVKKPGASVKVSCKASGYTFTSYWMHWVRQAPGQGLEWMGMIHPNSGNTIYYEKFQGRVTMTVDKSISTAYMELSRLRSDDTAVYYCAIIITPVVPKFDYWGQGTLVTVSS

SEQ ID NO: 257 - protein, 120 aa, synthetic construct
  REGION 1..120: note = Description of Artificial Sequence: Synthetic polypeptide
  REGION 1..120: note = MESO-C[MSLN]_H1.7 Variable Heavy
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCARIITPVVPKFDYWGQGTLVTVSS

SEQ ID NO: 258 - protein, 120 aa, synthetic construct
  REGION 1..120: note = Description of Artificial Sequence: Synthetic polypeptide
  REGION 1..120: note = MESO-C[MSLN]_H1.8 Variable Heavy
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIISPVVPKFDYWGQGTLVTVSS

SEQ ID NO: 259 - protein, 120 aa, synthetic construct
  REGION 1..120: note = Description of Artificial Sequence: Synthetic polypeptide
  REGION 1..120: note = MESO-C[MSLN]_H1.9 Variable Heavy
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPRFDYWGQGTLVTVSS

SEQ ID NO: 260 - protein, 120 aa, synthetic construct
  REGION 1..120: note = Description of Artificial Sequence: Synthetic polypeptide
  REGION 1..120: note = MESO-C[MSLN]_H1.12 Variable Heavy
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSS

SEQ ID NO: 261 - protein, 120 aa, synthetic construct
  REGION 1..120: note = Description of Artificial Sequence: Synthetic polypeptide
  REGION 1..120: note = MESO-C[MSLN]_H1.13 Variable Heavy
QVQLVQSGAEVKKPGASVKVSCKASGYTFTSYWMHWVRQAPGQGLEWMGMIHPNSDNTIYYYKFQGRVTMTVDKSISTAYMELSRLRSDDTAVYYCAIIITPVVPKFDYWGQGTLVTVSS

SEQ ID NO: 262 - protein, 107 aa, synthetic construct
  REGION 1..107: note = Description of Artificial Sequence: Synthetic polypeptide
  REGION 1..107: note = MESO-C[MSLN]_L1.1 Variable Light
DIVMTQSPDSLAVSLGERATINCKSSHDVGTSVAWYQQKPGQSPKLLIYWASTRHTGVPDRFTGSGSGTDFTLTISSLQAEDVAVYFCQQYSSYPLTFGAGTKLEIK

SEQ ID NO: 263 - protein, 107 aa, synthetic construct
  REGION 1..107: note = Description of Artificial Sequence: Synthetic polypeptide
  REGION 1..107: note = MESO-C[MSLN]_L1.2 Variable Light
DIVMTQSPDSLAVSLGERATINCKASQDVGTSVAWYQQKPGQSPKLLIYWASTRHTGVPDRFTGSGSGTDFTLTISSLQAEDVAVYFCQQYSSYPLTFGAGTKLEIK

SEQ ID NO: 264 - protein, 107 aa, synthetic construct
  REGION 1..107: note = Description of Artificial Sequence: Synthetic polypeptide
  REGION 1..107: note = MESO-C[MSLN]_L1.3 Variable Light
DIVMTQSPDSLAVSLGERATINCKASHSVGTSVAWYQQKPGQSPKLLIYWASTRHTGVPDRFTGSGSGTDFTLTISSLQAEDVAVYFCQQYSSYPLTFGAGTKLEIK

SEQ ID NO: 265 - protein, 107 aa, synthetic construct
  REGION 1..107: note = Description of Artificial Sequence: Synthetic polypeptide
  REGION 1..107: note = MESO-C[MSLN]_L1.4 Variable Light
DIVMTQSPDSLAVSLGERATINCKASHDVLTSVAWYQQKPGQSPKLLIYWASTRHTGVPDRFTGSGSGTDFTLTISSLQAEDVAVYFCQQYSSYPLTFGAGTKLEIK

SEQ ID NO: 266 - protein, 107 aa, synthetic construct
  REGION 1..107: note = Description of Artificial Sequence: Synthetic polypeptide
  REGION 1..107: note = MESO-C[MSLN]_L1.5 Variable Light
DIVMTQSPDSLAVSLGERATINCKASHDVGTYVAWYQQKPGQSPKLLIYWASTRHTGVPDRFTGSGSGTDFTLTISSLQAEDVAVYFCQQYSSYPLTFGAGTKLEIK

SEQ ID NO: 267 - protein, 107 aa, synthetic construct
  REGION 1..107: note = Description of Artificial Sequence: Synthetic polypeptide
  REGION 1..107: note = MESO-C[MSLN]_L1.6 Variable Light
DIVMTQSPDSLAVSLGERATINCKASHDVGTSLAWYQQKPGQSPKLLIYWASTRHTGVPDRFTGSGSGTDFTLTISSLQAEDVAVYFCQQYSSYPLTFGAGTKLEIK

SEQ ID NO: 268 - protein, 107 aa, synthetic construct
  REGION 1..107: note = Description of Artificial Sequence: Synthetic polypeptide
  REGION 1..107: note = MESO-C[MSLN]_L1.7 Variable Light
DIVMTQSPDSLAVSLGERATINCKASHDVGTSVAWYQQKPGQSPKLLIYWASTRETGVPDRFTGSGSGTDFTLTISSLQAEDVAVYFCQQYSSYPLTFGAGTKLEIK

SEQ ID NO: 269 - protein, 107 aa, synthetic construct
  REGION 1..107: note = Description of Artificial Sequence: Synthetic polypeptide
  REGION 1..107: note = MESO-C[MSLN]_L1.8 Variable Light
DIVMTQSPDSLAVSLGERATINCKASHDVGTSVAWYQQKPGQSPKLLIYWASTRHSGVPDRFTGSGSGTDFTLTISSLQAEDVAVYFCQQYSSYPLTFGAGTKLEIK

SEQ ID NO: 270 - protein, 107 aa, synthetic construct
  REGION 1..107: note = Description of Artificial Sequence: Synthetic polypeptide
  REGION 1..107: note = MESO-C[MSLN]_L1.9 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYYSYPLTFGAGTKLEIK

SEQ ID NO: 271 - protein, 107 aa, synthetic construct
  REGION 1..107: note = Description of Artificial Sequence: Synthetic polypeptide
  REGION 1..107: note = MESO-C[MSLN]_L1.10 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSTPLTFGAGTKLEIK

SEQ ID NO: 272 - protein, 107 aa, synthetic construct
  REGION 1..107: note = Description of Artificial Sequence: Synthetic polypeptide
  REGION 1..107: note = MESO-C[MSLN]_L1.13 Variable Light
DIVMTQSPDSLAVSLGERATINCKASHDVGQSVAWYQQKPGQSPKLLIYWASTRHTGVPDRFTGSGSGTDFTLTISSLQAEDVAVYFCQQYSSYPLTFGAGTKLEIK

SEQ ID NO: 273 - protein, 107 aa, synthetic construct
  REGION 1..107: note = Description of Artificial Sequence: Synthetic polypeptide
  REGION 1..107: note = MESO-C[MSLN]_L1.14 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSFPLTFGAGTKLEIK

SEQ ID NO: 274 - protein, 107 aa, synthetic construct
  REGION 1..107: note = Description of Artificial Sequence: Synthetic polypeptide
  REGION 1..107: note = MESO-C[MSLN]_L1.15 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSLPLTFGAGTKLEIK

SEQ ID NO: 275 - protein, 107 aa, synthetic construct
  REGION 1..107: note = Description of Artificial Sequence: Synthetic polypeptide
  REGION 1..107: note = MESO-C[MSLN]_L1.16 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSVPLTFGAGTKLEIK

SEQ ID NO: 276 - protein, 107 aa, synthetic construct
  REGION 1..107: note = Description of Artificial Sequence: Synthetic polypeptide
  REGION 1..107: note = MESO-C[MSLN]_L1.17 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSIPLTFGAGTKLEIK

SEQ ID NO: 277 - protein, 107 aa, synthetic construct
  REGION 1..107: note = Description of Artificial Sequence: Synthetic polypeptide
  REGION 1..107: note = MESO-C[MSLN]_L1.18 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYSSHPLTFGAGTKLEIK

SEQ ID NO: 278 - protein, 107 aa, synthetic construct
  REGION 1..107: note = Description of Artificial Sequence: Synthetic polypeptide
  REGION 1..107: note = MESO-C[MSLN]_L1.19 Variable Light
DIVMTQSPDSLAVSLGERATINCKASHDVGTYVAWYQQKPGQSPKLLIYWASTRHTGVPDRFTGSGSGTDFTLTISSLQAEDVAVYFCQQYYSYPLTFGAGTKLEIK

SEQ ID NO: 279 - protein, 107 aa, synthetic construct
  REGION 1..107: note = Description of Artificial Sequence: Synthetic polypeptide
  REGION 1..107: note = MESO-C[MSLN]_L1.20 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYYSFPLTFGAGTKLEIK

SEQ ID NO: 280 - protein, 107 aa, synthetic construct
  REGION 1..107: note = Description of Artificial Sequence: Synthetic polypeptide
  REGION 1..107: note = MESO-C[MSLN]_L1.21 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYYSVPLTFGAGTKLEIK

SEQ ID NO: 281 - protein, 107 aa, synthetic construct
  REGION 1..107: note = Description of Artificial Sequence: Synthetic polypeptide
  REGION 1..107: note = MESO-C[MSLN]_L1.22 Variable Light
DIVMTQSPDSLAVSLGERATINCKASHDVGTSVAWYQQKPGQSPKLLIYWASTRHTGVPDRFTGSGSGTDFTLTISSLQAEDVAVYFCQQYYSIPLTFGAGTKLEIK

SEQ ID NO: 282 - protein, 453 aa, synthetic construct; identical to SEQ ID NO: 229
  REGION 1..453: note = Description of Artificial Sequence: Synthetic polypeptide
  REGION 1..453: note = XENP16248 Chain 1 - MESO-A[MSLN]_H1.1_IgG1_pI(-)_Isosteric_ A_/PVA_/S267K/L368D/K370S

SEQ ID NO: 283 - protein, 485 aa, synthetic construct
  REGION 1..485: note = Description of Artificial Sequence: Synthetic polypeptide
  REGION 1..485: note = XENP16248 Chain 2 - [CD3]_H1.30_L1.47_scFv(GKPGS)4_Fc(216)_ IgG1_C220S/PVA_/S267K/S364K/E357Q
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 284 - protein, 213 aa, synthetic construct; identical to SEQ ID NO: 226
  REGION 1..213: note = Description of Artificial Sequence: Synthetic polypeptide
  REGION 1..213: note = XENP16248 Chain 3 - MESO-A[MSLN]_L1 Light Chain

SEQ ID NO: 285 - protein, 452 aa, synthetic construct
  REGION 1..452: note = Description of Artificial Sequence: Synthetic polypeptide
  REGION 1..452: note = XENP16833 Chain 1 - MESO-B[MSLN]_H0L0_Fab_IgG1_pI(-)_Isoste ric_A_/PVA_/S267K/L368D/K370S
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 286 - protein, 485 aa, synthetic construct; identical to SEQ ID NO: 283
  REGION 1..485: note = Description of Artificial Sequence: Synthetic polypeptide
  REGION 1..485: note = XENP16833 Chain 2 - [CD3]_H1.30_L1.47_scFv(GKPGS)4_Fc(216)_ IgG1_C220S/PVA_/S267K/S364K/E357Q

SEQ ID NO: 287 - protein, 221 aa, synthetic construct; identical to SEQ ID NO: 240
  REGION 1..221: note = Description of Artificial Sequence: Synthetic polypeptide
  REGION 1..221: note = XENP16833 Chain 3 - MESO-B[MSLN]_L0 Light Chain

SEQ ID NO: 288 - protein, 449 aa, synthetic construct
  REGION 1..449: note = Description of Artificial Sequence: Synthetic polypeptide
  REGION 1..449: note = XENP31697 Chain 1 - MESO-C[MSLN]_H1_IgG1_pI(-)_Isosteric_A_ /PVA_/S267K/L368D/K370S
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 289 - protein, 485 aa, synthetic construct; identical to SEQ ID NO: 283
  REGION 1..485: note = Description of Artificial Sequence: Synthetic polypeptide
  REGION 1..485: note = XENP31697 Chain 2 - [CD3]_H1.30_L1.47_scFv(GKPGS)4_Fc(216)_ IgG1_C220S/PVA_/S267K/S364K/E357Q

SEQ ID NO: 290 - protein, 214 aa, synthetic construct; identical to SEQ ID NO: 250
  REGION 1..214: note = Description of Artificial Sequence: Synthetic polypeptide
  REGION 1..214: note = XENP31697 Chain 3 - MESO-C[MSLN]_L1 Light Chain

SEQ ID NO: 291 - protein, 449 aa, synthetic construct
  REGION 1..449: note = Description of Artificial Sequence: Synthetic polypeptide
  REGION 1..449: note = XENP32550 Chain 1 - MESO-C[MSLN]_H1.1_IgG1_pI(-)_Isosteric_ A_/PVA_/S267K/L368D/K370S
QVQLVQSGAEVKKPGASVKVSCKASGYTFTGYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 292 - protein, 485 aa, synthetic construct; identical to SEQ ID NO: 283
  REGION 1..485: note = Description of Artificial Sequence: Synthetic polypeptide
  REGION 1..485: note = XENP32550 Chain 2 - MESO-C[MSLN]_H1.1_[CD3]_H1.30_L1.47_scF v(GKPGS)4_Fc(216)_IgG1_C220S/PVA_/S267K/S364K/E357Q

SEQ ID NO: 293 - protein, 214 aa, synthetic construct; identical to SEQ ID NO: 250
  REGION 1..214: note = Description of Artificial Sequence: Synthetic polypeptide
  REGION 1..214: note = XENP32550 Chain 3 - MESO-C[MSLN]_L1 Light Chain

SEQ ID NO: 294 - protein, 449 aa, synthetic construct
  REGION 1..449: note = Description of Artificial Sequence: Synthetic polypeptide
  REGION 1..449: note = XENP32551 Chain 1 - MESO-C[MSLN]_H1.2_IgG1_pI(-)_Isosteric_ A_/PVA_/S267K/L368D/K370S
QVQLVQSGAEVKKPGASVKVSCKASGYTFTSYYMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 295 - protein, 485 aa, synthetic construct; identical to SEQ ID NO: 283
  REGION 1..485: note = Description of Artificial Sequence: Synthetic polypeptide
  REGION 1..485: note = XENP32551 Chain 2 - MESO-C[MSLN]_H1.2_[CD3]_H1.30_L1.47_scF v(GKPGS)4_Fc(216)_IgG1_C220S/PVA_/S267K/S364K/E357Q

SEQ ID NO: 296 - protein, 214 aa, synthetic construct; identical to SEQ ID NO: 250
  REGION 1..214: note = Description of Artificial Sequence: Synthetic polypeptide
  REGION 1..214: note = XENP32551 Chain 3 - MESO-C[MSLN]_L1 Light Chain

SEQ ID NO: 297 - protein, 449 aa, synthetic construct
  REGION 1..449: note = Description of Artificial Sequence: Synthetic polypeptide
  REGION 1..449: note = XENP32552 Chain 1 - MESO-C[MSLN]_H1.3_IgG1_pI(-)_Isosteric_ A_/PVA_/S267K/L368D/K370S
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 298 - protein, 485 aa, synthetic construct; identical to SEQ ID NO: 283
  REGION 1..485: note = Description of Artificial Sequence: Synthetic polypeptide
  REGION 1..485: note = XENP32552 Chain 2 - MESO-C[MSLN]_H1.3_[CD3]_H1.30_L1.47_scF v(GKPGS)4_Fc(216)_IgG1_C220S/PVA_/S267K/S364K/E357Q

SEQ ID NO: 299 - protein, 214 aa, synthetic construct; identical to SEQ ID NO: 250
  REGION 1..214: note = Description of Artificial Sequence: Synthetic polypeptide
  REGION 1..214: note = XENP32552 Chain 3 - MESO-C[MSLN]_L1 Light Chain

SEQ ID NO: 300 - protein, 449 aa, synthetic construct
  REGION 1..449: note = Description of Artificial Sequence: Synthetic polypeptide
  REGION 1..449: note = XENP32553 Chain 1 - MESO-C[MSLN]_H1.4_IgG1_pI(-)_Isosteric_ A_/PVA_/S267K/L368D/K370S
QVQLVQSGAEVKKPGASVKVSCKASGYTFTSYWMHWVRQAPGQGLEWMGMINPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 301 - protein, 485 aa, synthetic construct; identical to SEQ ID NO: 283
  REGION 1..485: note = Description of Artificial Sequence: Synthetic polypeptide
  REGION 1..485: note = XENP32553 Chain 2 - MESO-C[MSLN]_H1.4_[CD3]_H1.30_L1.47_scF v(GKPGS)4_Fc(216)_IgG1_C220S/PVA_/S267K/S364K/E357Q

SEQ ID NO: 302 - protein, 214 aa, synthetic construct; identical to SEQ ID NO: 250
  REGION 1..214: note = Description of Artificial Sequence: Synthetic polypeptide
  REGION 1..214: note = XENP32553 Chain 3 - MESO-C[MSLN]_L1 Light Chain

SEQ ID NO: 303 - protein, 449 aa, synthetic construct
  REGION 1..449: note = Description of Artificial Sequence: Synthetic polypeptide
  REGION 1..449: note = XENP32554 Chain 1 - MESO-C[MSLN]_H1.5_IgG1_pI(-)_Isosteric_ A_/PVA_/S267K/L368D/K370S
QVQLVQSGAEVKKPGASVKVSCKASGYTFTSYWMHWVRQAPGQGLEWMGMIHPQSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 304 - protein, 485 aa, synthetic construct; identical to SEQ ID NO: 283
  REGION 1..485: note = Description of Artificial Sequence: Synthetic polypeptide
  REGION 1..485: note = XENP32554 Chain 2 - MESO-C[MSLN]_H1.5_[CD3]_H1.30_L1.47_scF v(GKPGS)4_Fc(216)_IgG1_C220S/PVA_/S267K/S364K/E357Q

SEQ ID NO: 305 - protein, 214 aa, synthetic construct; identical to SEQ ID NO: 250
  REGION 1..214: note = Description of Artificial Sequence: Synthetic polypeptide
  REGION 1..214: note = XENP32554 Chain 3 - MESO-C[MSLN]_L1 Light Chain

SEQ ID NO: 306 - protein, 449 aa, synthetic construct
  REGION 1..449: note = Description of Artificial Sequence: Synthetic polypeptide
  REGION 1..449: note = XENP32555 Chain 1 - MESO-C[MSLN]_H1.6_IgG1_pI(-)_Isosteric_ A_/PVA_/S267K/L368D/K370S
QVQLVQSGAEVKKPGASVKVSCKASGYTFTSYWMHWVRQAPGQGLEWMGMIHPNSGNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 307 - protein, 485 aa, synthetic construct; identical to SEQ ID NO: 283
  REGION 1..485: note = Description of Artificial Sequence: Synthetic polypeptide
  REGION 1..485: note = XENP32555 Chain 2 - MESO-C[MSLN]_H1.6_[CD3]_H1.30_L1.47_scF v(GKPGS)4_Fc(216)_IgG1_C220S/PVA_/S267K/S364K/E357Q

SEQ ID NO: 308 - protein, 214 aa, synthetic construct; identical to SEQ ID NO: 250
  REGION 1..214: note = Description of Artificial Sequence: Synthetic polypeptide
  REGION 1..214: note = XENP32555 Chain 3 - MESO-C[MSLN]_L1 Light Chain

SEQ ID NO: 309 - protein, 449 aa, synthetic construct
  REGION 1..449: note = Description of Artificial Sequence: Synthetic polypeptide
  REGION 1..449: note = XENP32556 Chain 1 - MESO-C[MSLN]_H1.7_IgG1_pI(-)_Isosteric_ A_/PVA_/S267K/L368D/K370S
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCARIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 310 - protein, 485 aa, synthetic construct; identical to SEQ ID NO: 283
  REGION 1..485: note = Description of Artificial Sequence: Synthetic polypeptide
  REGION 1..485: note = XENP32556 Chain 2 - MESO-C[MSLN]_H1.7_[CD3]_H1.30_L1.47_scF v(GKPGS)4_Fc(216)_IgG1_C220S/PVA_/S267K/S364K/E357Q

SEQ ID NO: 311 - protein, 214 aa, synthetic construct; identical to SEQ ID NO: 250
  REGION 1..214: note = Description of Artificial Sequence: Synthetic polypeptide
  REGION 1..214: note = XENP32556 Chain 3 - MESO-C[MSLN]_L1 Light Chain

SEQ ID NO: 312 - protein, 449 aa, synthetic construct
  REGION 1..449: note = Description of Artificial Sequence: Synthetic polypeptide
  REGION 1..449: note = XENP32557 Chain 1 - MESO-C[MSLN]_H1.8_IgG1_pI(-)_Isosteric_ A_/PVA_/S267K/L368D/K370S
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIISPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 313 - protein, 485 aa, synthetic construct; identical to SEQ ID NO: 283
  REGION 1..485: note = Description of Artificial Sequence: Synthetic polypeptide
  REGION 1..485: note = XENP32557 Chain 2 - MESO-C[MSLN]_H1.8_[CD3]_H1.30_L1.47_scF v(GKPGS)4_Fc(216)_IgG1_C220S/PVA_/S267K/S364K/E357Q

SEQ ID NO: 314 - protein, 214 aa, synthetic construct; identical to SEQ ID NO: 250
  REGION 1..214: note = Description of Artificial Sequence: Synthetic polypeptide
  REGION 1..214: note = XENP32557 Chain 3 - MESO-C[MSLN]_L1 Light Chain

SEQ ID NO: 315 - protein, 449 aa, synthetic construct
  REGION 1..449: note = Description of Artificial Sequence: Synthetic polypeptide
  REGION 1..449: note = XENP32558 Chain 1 - MESO-C[MSLN]_H1.9_IgG1_pI(-)_Isosteric_ A_/PVA_/S267K/L368D/K370S
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 316 - protein, 485 aa, synthetic construct; identical to SEQ ID NO: 283
  REGION 1..485: note = Description of Artificial Sequence: Synthetic polypeptide
  REGION 1..485: note = XENP32558 Chain 2 - MESO-C[MSLN]_H1.9_[CD3]_H1.30_L1.47_scF v(GKPGS)4_Fc(216)_IgG1_C220S/PVA_/S267K/S364K/E357Q

SEQ ID NO: 317 - protein, 214 aa, synthetic construct; identical to SEQ ID NO: 250
  REGION 1..214: note = Description of Artificial Sequence: Synthetic polypeptide
  REGION 1..214: note = XENP32558 Chain 3 - MESO-C[MSLN]_L1 Light Chain

SEQ ID NO: 318 - protein, 449 aa, synthetic construct; identical to SEQ ID NO: 288
  REGION 1..449: note = Description of Artificial Sequence: Synthetic polypeptide
  REGION 1..449: note = XENP32559 Chain 1 - MESO-C[MSLN]_H1_IgG1_pI(-)_Isosteric_A_ /PVA_/S267K/L368D/K370S

SEQ ID NO: 319 - protein, 485 aa, synthetic construct; identical to SEQ ID NO: 283
  REGION 1..485: note = Description of Artificial Sequence: Synthetic polypeptide
  REGION 1..485: note = XENP32559 Chain 2 - MESO-C[MSLN]_H1_[CD3]_H1.30_L1.47_scFv( GKPGS)4_Fc(216)_IgG1_C220S/PVA_/S267K/S364K/E357Q

SEQ ID NO: 320 - protein, 214 aa, synthetic construct
  REGION 1..214: note = Description of Artificial Sequence: Synthetic polypeptide
  REGION 1..214: note = XENP32559 Chain 3 - MESO-C[MSLN]_L1.1 Light Chain
DIVMTQSPDSLAVSLGERATINCKSSHDVGTSVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 321 - protein, 449 aa, synthetic construct; identical to SEQ ID NO: 288
  REGION 1..449: note = Description of Artificial Sequence: Synthetic polypeptide
  REGION 1..449: note = XENP32560 Chain 1 - MESO-C[MSLN]_H1_IgG1_pI(-)_Isosteric_A_ /PVA_/S267K/L368D/K370S

SEQ ID NO: 322 - protein, 485 aa, synthetic construct; identical to SEQ ID NO: 283
  REGION 1..485: note = Description of Artificial Sequence: Synthetic polypeptide
  REGION 1..485: note = XENP32560 Chain 2 - MESO-C[MSLN]_H1_[CD3]_H1.30_L1.47_scFv( GKPGS)4_Fc(216)_IgG1_C220S/PVA_/S267K/S364K/E357Q

SEQ ID NO: 323 - protein, 214 aa, synthetic construct
  REGION 1..214: note = Description of Artificial Sequence: Synthetic polypeptide
  REGION 1..214: note = XENP32560 Chain 3 - MESO-C[MSLN]_L1.2 Light Chain
DIVMTQSPDSLAVSLGERATINCKASQDVGTSVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 324 - protein, 449 aa, synthetic construct; identical to SEQ ID NO: 288
  REGION 1..449: note = Description of Artificial Sequence: Synthetic polypeptide
  REGION 1..449: note = XENP32561 Chain 1 - MESO-C[MSLN]_H1_IgG1_pI(-)_Isosteric_A_ /PVA_/S267K/L368D/K370S

SEQ ID NO: 325 - protein, 485 aa, synthetic construct; identical to SEQ ID NO: 283
  REGION 1..485: note = Description of Artificial Sequence: Synthetic polypeptide
  REGION 1..485: note = XENP32561 Chain 2 - MESO-C[MSLN]_H1_[CD3]_H1.30_L1.47_scFv( GKPGS)4_Fc(216)_IgG1_C220S/PVA_/S267K/S364K/E357Q

SEQ ID NO: 326 - protein, 214 aa, synthetic construct
  REGION 1..214: note = Description of Artificial Sequence: Synthetic polypeptide
  REGION 1..214: note = XENP32561 Chain 3 - MESO-C[MSLN]_L1.3 Light Chain
DIVMTQSPDSLAVSLGERATINCKASHSVGTSVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 327 - protein, 449 aa, synthetic construct; identical to SEQ ID NO: 288
  REGION 1..449: note = Description of Artificial Sequence: Synthetic polypeptide
  REGION 1..449: note = XENP32562 Chain 1 - MESO-C[MSLN]_H1_IgG1_pI(-)_Isosteric_A_ /PVA_/S267K/L368D/K370S

SEQ ID NO: 328 - protein, 485 aa, synthetic construct; identical to SEQ ID NO: 283
  REGION 1..485: note = Description of Artificial Sequence: Synthetic polypeptide
  REGION 1..485: note = XENP32562 Chain 2 - MESO-C[MSLN]_H1_[CD3]_H1.30_L1.47_scFv( GKPGS)4_Fc(216)_IgG1_C220S/PVA_/S267K/S364K/E357Q

SEQ ID NO: 329 - protein, 214 aa, synthetic construct
  REGION 1..214: note = Description of Artificial Sequence: Synthetic polypeptide
  REGION 1..214: note = XENP32562 Chain 3 - MESO-C[MSLN]_L1.4 Light Chain
DIVMTQSPDSLAVSLGERATINCKASHDVLTSVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - protein, 449 aa, synthetic construct; identical to SEQ ID NO: 288
  REGION 1..449: note = Description of Artificial Sequence: Synthetic polypeptide
  REGION 1..449: note = XENP32563 Chain 1 - MESO-C[MSLN]_H1_IgG1_pI(-)_Isosteric_A_ /PVA_/S267K/L368D/K370S

SEQ ID NO: 331 - protein, 485 aa, synthetic construct; identical to SEQ ID NO: 283
  REGION 1..485: note = Description of Artificial Sequence: Synthetic polypeptide
  REGION 1..485: note = XENP32563 Chain 2 - MESO-C[MSLN]_H1_[CD3]_H1.30_L1.47_scFv( GKPGS)4_Fc(216)_IgG1_C220S/PVA_/S267K/S364K/E357Q

SEQ ID NO: 332 - protein, 214 aa, synthetic construct
  REGION 1..214: note = Description of Artificial Sequence: Synthetic polypeptide
  REGION 1..214: note = XENP32563 Chain 3 - MESO-C[MSLN]_L1.5 Light Chain
DIVMTQSPDSLAVSLGERATINCKASHDVGTYVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 449 aa, synthetic construct; identical to SEQ ID NO: 288
  REGION 1..449: note = Description of Artificial Sequence: Synthetic polypeptide
  REGION 1..449: note = XENP32564 Chain 1 - MESO-C[MSLN]_H1_IgG1_pI(-)_Isosteric_A_ /PVA_/S267K/L368D/K370S

SEQ ID NO: 334 - protein, 485 aa, synthetic construct; identical to SEQ ID NO: 283
  REGION 1..485: note = Description of Artificial Sequence: Synthetic polypeptide
  REGION 1..485: note = XENP32564 Chain 2 - MESO-C[MSLN]_H1_[CD3]_H1.30_L1.47_scFv( GKPGS)4_Fc(216)_IgG1_C220S/PVA_/S267K/S364K/E357Q

SEQ ID NO: 335 - protein, 214 aa, synthetic construct
  REGION 1..214: note = Description of Artificial Sequence: Synthetic polypeptide
  REGION 1..214: note = XENP32564 Chain 3 - MESO-C[MSLN]_L1.6 Light Chain
DIVMTQSPDSLAVSLGERATINCKASHDVGTSLAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 336 - protein, 449 aa, synthetic construct; identical to SEQ ID NO: 288
  REGION 1..449: note = Description of Artificial Sequence: Synthetic polypeptide
  REGION 1..449: note = XENP32565 Chain 1 - MESO-C[MSLN]_H1_IgG1_pI(-)_Isosteric_A_ /PVA_/S267K/L368D/K370S

SEQ ID NO: 337 - protein, 485 aa, synthetic construct; identical to SEQ ID NO: 283
  REGION 1..485: note = Description of Artificial Sequence: Synthetic polypeptide
  REGION 1..485: note = XENP32565 Chain 2 - MESO-C[MSLN]_H1_[CD3]_H1.30_L1.47_scFv( GKPGS)4_Fc(216)_IgG1_C220S/PVA_/S267K/S364K/E357Q

SEQ ID NO: 338 - protein, 214 aa, synthetic construct
  REGION 1..214: note = Description of Artificial Sequence: Synthetic polypeptide
  REGION 1..214: note = XENP32565 Chain 3 - MESO-C[MSLN]_L1.7 Light Chain
DIVMTQSPDSLAVSLGERATINCKASHDVGTSVAWYQQKPGQSPKLLIYWASTRETGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 339 - protein, 449 aa, synthetic construct; identical to SEQ ID NO: 288
  REGION 1..449: note = Description of Artificial Sequence: Synthetic polypeptide
  REGION 1..449: note = XENP32566 Chain 1 - MESO-C[MSLN]_H1_IgG1_pI(-)_Isosteric_A_ /PVA_/S267K/L368D/K370S

SEQ ID NO: 340 - protein, 485 aa, synthetic construct; identical to SEQ ID NO: 283
  REGION 1..485: note = Description of Artificial Sequence: Synthetic polypeptide
  REGION 1..485: note = XENP32566 Chain 2 - MESO-C[MSLN]_H1_[CD3]_H1.30_L1.47_scFv( GKPGS)4_Fc(216)_IgG1_C220S/PVA_/S267K/S364K/E357Q

SEQ ID NO: 341 - protein, 214 aa, synthetic construct
  REGION 1..214: note = Description of Artificial Sequence: Synthetic polypeptide
  REGION 1..214: note = XENP32566 Chain 3 - MESO-C[MSLN]_L1.8 Light Chain
DIVMTQSPDSLAVSLGERATINCKASHDVGTSVAWYQQKPGQSPKLLIYWASTRHSGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 449 aa, synthetic construct; identical to SEQ ID NO: 288
  REGION 1..449: note = Description of Artificial Sequence: Synthetic polypeptide
  REGION 1..449: note = XENP32567 Chain 1 - MESO-C[MSLN]_H1_IgG1_pI(-)_Isosteric_A_ /PVA_/S267K/L368D/K370S

SEQ ID NO: 343 - protein, 485 aa, synthetic construct; identical to SEQ ID NO: 283
  REGION 1..485: note = Description of Artificial Sequence: Synthetic polypeptide
  REGION 1..485: note = XENP32567 Chain 2 - MESO-C[MSLN]_H1_[CD3]_H1.30_L1.47_scFv( GKPGS)4_Fc(216)_IgG1_C220S/PVA_/S267K/S364K/E357Q

SEQ ID NO: 344 - protein, 214 aa, synthetic construct
  REGION 1..214: note = Description of Artificial Sequence: Synthetic polypeptide
  REGION 1..214: note = XENP32567 Chain 3 - MESO-C[MSLN]_L1.9 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYYSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - protein, 449 aa, synthetic construct; identical to SEQ ID NO: 288
  REGION 1..449: note = Description of Artificial Sequence: Synthetic polypeptide
  REGION 1..449: note = XENP32568 Chain 1 - MESO-C[MSLN]_H1_IgG1_pI(-)_Isosteric_A_ /PVA_/S267K/L368D/K370S

SEQ ID NO: 346 - protein, 485 aa, synthetic construct; identical to SEQ ID NO: 283
  REGION 1..485: note = Description of Artificial Sequence: Synthetic polypeptide
  REGION 1..485: note = XENP32568 Chain 2 - MESO-C[MSLN]_H1_[CD3]_H1.30_L1.47_scFv( GKPGS)4_Fc(216)_IgG1_C220S/PVA_/S267K/S364K/E357Q

SEQ ID NO: 347 - protein, 214 aa, synthetic construct
  REGION 1..214: note = Description of Artificial Sequence: Synthetic polypeptide
  REGION 1..214: note = XENP32568 Chain 3 - MESO-C[MSLN]_L1.10 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYSSTPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 348 - protein, 449 aa, synthetic construct; identical to SEQ ID NO: 303
  REGION 1..449: note = Description of Artificial Sequence: Synthetic polypeptide
  REGION 1..449: note = XENP32569 Chain 1 - MESO-C[MSLN]_H1.5_IgG1_pI(-)_Isosteric_ A_/PVA_/S267K/L368D/K370S

SEQ ID NO: 349 - protein, 485 aa, synthetic construct; identical to SEQ ID NO: 283
  REGION 1..485: note = Description of Artificial Sequence: Synthetic polypeptide
  REGION 1..485: note = XENP32569 Chain 2 - MESO-C[MSLN]_H1.5_[CD3]_H1.30_L1.47_scF v(GKPGS)4_Fc(216)_IgG1_C220S/PVA_/S267K/S364K/E357Q

SEQ ID NO: 350 - protein, 214 aa, synthetic construct; identical to SEQ ID NO: 326
  REGION 1..214: note = Description of Artificial Sequence: Synthetic polypeptide
  REGION 1..214: note = XENP32569 Chain 3 - MESO-C[MSLN]_L1.3 Light Chain

SEQ ID NO: 351 - protein, 449 aa, synthetic construct; identical to SEQ ID NO: 306
  REGION 1..449: note = Description of Artificial Sequence: Synthetic polypeptide
  REGION 1..449: note = XENP32570 Chain 1 - MESO-C[MSLN]_H1.6_IgG1_pI(-)_Isosteric_ A_/PVA_/S267K/L368D/K370S

SEQ ID NO: 352 - protein, 485 aa, synthetic construct; identical to SEQ ID NO: 283
  REGION 1..485: note = Description of Artificial Sequence: Synthetic polypeptide
  REGION 1..485: note = XENP32570 Chain 2 - MESO-C[MSLN]_H1.6_[CD3]_H1.30_L1.47_scF v(GKPGS)4_Fc(216)_IgG1_C220S/PVA_/S267K/S364K/E357Q

SEQ ID NO: 353 - protein, 214 aa, synthetic construct; identical to SEQ ID NO: 326
  REGION 1..214: note = Description of Artificial Sequence: Synthetic polypeptide
  REGION 1..214: note = XENP32570 Chain 3 - MESO-C[MSLN]_L1.3 Light Chain

SEQ ID NO: 354 - protein, 449 aa, synthetic construct
  REGION 1..449: note = Description of Artificial Sequence: Synthetic polypeptide
  REGION 1..449: note = XENP32937 Chain 1 - MESO-C[MSLN]_H1.12_IgG1_pI(-)_Isosteric _A_/PVA_/S267K/L368D/K370S
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 355 - protein, 485 aa, synthetic construct; identical to SEQ ID NO: 283
  REGION 1..485: note = Description of Artificial Sequence: Synthetic polypeptide
  REGION 1..485: note = XENP32937 Chain 2 - MESO-C[MSLN]_H1.12_[CD3]_H1.30_L1.47_sc Fv(GKPGS)4-IgG1_pI (-)_Isosteric_A_/PVA_/S267K/L368D/K370S-F c(216)_IgG1_C220S/PVA_/S267K/S364K/E357Q

SEQ ID NO: 356 - protein, 214 aa, synthetic construct; identical to SEQ ID NO: 344
  REGION 1..214: note = Description of Artificial Sequence: Synthetic polypeptide
  REGION 1..214: note = XENP32937 Chain 3 - MESO-C[MSLN]_L1.9 Light Chain

SEQ ID NO: 357 - protein, 449 aa, synthetic construct
  REGION 1..449: note = Description of Artificial Sequence: Synthetic polypeptide
  REGION 1..449: note = XENP32938 Chain 1 - MESO-C[MSLN]_H1.13_IgG1_pI(-)_Isosteric _A_/PVA_/S267K/L368D/K370S
QVQLVQSGAEVKKPGASVKVSCKASGYTFTSYWMHWVRQAPGQGLEWMGMIHPNSDNTIYYYKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 358 - protein, 485 aa, synthetic construct; identical to SEQ ID NO: 283
  REGION 1..485: note = Description of Artificial Sequence: Synthetic polypeptide
  REGION 1..485: note = XENP32938 Chain 2 - MESO-C[MSLN]_H1.13_[CD3]_H1.30_L1.47_sc Fv(GKPGS)4-IgG1_pI (-)_Isosteric_A_/PVA_/S267K/L368D/K370S-F c(216)_IgG1_C220S/PVA_/S267K/S364K/E357Q

SEQ ID NO: 359 - protein, 214 aa, synthetic construct; identical to SEQ ID NO: 344
  REGION 1..214: note = Description of Artificial Sequence: Synthetic polypeptide
  REGION 1..214: note = XENP32938 Chain 3 - MESO-C[MSLN]_L1.9 Light Chain

SEQ ID NO: 360 - protein, 449 aa, synthetic construct; identical to SEQ ID NO: 297
  REGION 1..449: note = Description of Artificial Sequence: Synthetic polypeptide
  REGION 1..449: note = XENP32939 Chain 1 - MESO-C[MSLN]_H1.3_IgG1_pI(-)_Isosteric_ A_/PVA_/S267K/L368D/K370S

SEQ ID NO: 361 - protein, 485 aa, synthetic construct; identical to SEQ ID NO: 283
  REGION 1..485: note = Description of Artificial Sequence: Synthetic polypeptide
  REGION 1..485: note = XENP32939 Chain 2 - MESO-C[MSLN]_H1.3_[CD3]_H1.30_L1.47_scF v(GKPGS)4-IgG1_pI( -)_Isosteric_A_/PVA_/S267K/L368D/K370S-Fc (216)_IgG1_C220S/PVA_/S267K/S364K/E357Q

SEQ ID NO: 362 - protein, 214 aa, synthetic construct
  REGION 1..214: note = Description of Artificial Sequence: Synthetic polypeptide
  REGION 1..214: note = XENP32939 Chain 3 - MESO-C[MSLN]_L1.13 Light Chain
DIVMTQSPDSLAVSLGERATINCKASHDVGQSVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 363 - protein, 449 aa, synthetic construct; identical to SEQ ID NO: 297
  REGION 1..449: note = Description of Artificial Sequence: Synthetic polypeptide
  REGION 1..449: note = XENP32940 Chain 1 - MESO-C[MSLN]_H1.3_IgG1_pI(-)_Isosteric_ A_/PVA_/S267K/L368D/K370S

SEQ ID NO: 364 - protein, 485 aa, synthetic construct; identical to SEQ ID NO: 283
  REGION 1..485: note = Description of Artificial Sequence: Synthetic polypeptide
  REGION 1..485: note = XENP32940 Chain 2 - MESO-C[MSLN]_H1.3_[CD3]_H1.30_L1.47_scF v(GKPGS)4-IgG1_pI( -)_Isosteric_A_/PVA_/S267K/L368D/K370S-Fc (216)_IgG1_C220S/PVA_/S267K/S364K/E357Q

SEQ ID NO: 365 - protein, 214 aa, synthetic construct
  REGION 1..214: note = Description of Artificial Sequence: Synthetic polypeptide
  REGION 1..214: note = XENP32940 Chain 3 - MESO-C[MSLN]_L1.14 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYSSFPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 366 - protein, 449 aa, synthetic construct; identical to SEQ ID NO: 297
  REGION 1..449: note = Description of Artificial Sequence: Synthetic polypeptide
  REGION 1..449: note = XENP32941 Chain 1 - MESO-C[MSLN]_H1.3_IgG1_pI(-)_Isosteric_ A_/PVA_/S267K/L368D/K370S

SEQ ID NO: 367 - protein, 485 aa, synthetic construct; identical to SEQ ID NO: 283
  REGION 1..485: note = Description of Artificial Sequence: Synthetic polypeptide
  REGION 1..485: note = XENP32941 Chain 2 - MESO-C[MSLN]_H1.3_[CD3]_H1.30_L1.47_scF v(GKPGS)4-IgG1_pI( -)_Isosteric_A_/PVA_/S267K/L368D/K370S-Fc (216)_IgG1_C220S/PVA_/S267K/S364K/E357Q

SEQ ID NO: 368 - protein, 214 aa, synthetic construct
  REGION 1..214: note = Description of Artificial Sequence: Synthetic polypeptide
  REGION 1..214: note = XENP32941 Chain 3 - MESO-C[MSLN]_L1.15 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYSSLPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 369 - protein, 449 aa, synthetic construct; identical to SEQ ID NO: 297
  REGION 1..449: note = Description of Artificial Sequence: Synthetic polypeptide
  REGION 1..449: note = XENP32942 Chain 1 - MESO-C[MSLN]_H1.3_IgG1_pI(-)_Isosteric_ A_/PVA_/S267K/L368D/K370S

SEQ ID NO: 370 - protein, 485 aa, synthetic construct; identical to SEQ ID NO: 283
  REGION 1..485: note = Description of Artificial Sequence: Synthetic polypeptide
  REGION 1..485: note = XENP32942 Chain 2 - MESO-C[MSLN]_H1.3_[CD3]_H1.30_L1.47_scF v(GKPGS)4_Fc(216)_IgG1_C220S/PVA_/S267K/S364K/E357Q

SEQ ID NO: 371 - protein, 214 aa, synthetic construct
  REGION 1..214: note = Description of Artificial Sequence: Synthetic polypeptide
  REGION 1..214: note = XENP32942 Chain 3 - MESO-C[MSLN]_L1.16 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYSSVPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 372 - protein, 449 aa, synthetic construct; identical to SEQ ID NO: 297
  REGION 1..449: note = Description of Artificial Sequence: Synthetic polypeptide
  REGION 1..449: note = XENP32943 Chain 1 - MESO-C[MSLN]_H1.3_IgG1_pI(-)_Isosteric_ A_/PVA_/S267K/L368D/K370S

SEQ ID NO: 373 - protein, 485 aa, synthetic construct; identical to SEQ ID NO: 283
  REGION 1..485: note = Description of Artificial Sequence: Synthetic polypeptide
  REGION 1..485: note = XENP32943 Chain 2 - MESO-C[MSLN]_H1.3_[CD3]_H1.30_L1.47_scF v(GKPGS)4-IgG1_pI( -)_Isosteric_A_/PVA_/S267K/L368D/K370S-Fc (216)_IgG1_C220S/PVA_/S267K/S364K/E357Q

SEQ ID NO: 374 - protein, 214 aa, synthetic construct
  REGION 1..214: note = Description of Artificial Sequence: Synthetic polypeptide
  REGION 1..214: note = XENP32943 Chain 3 - MESO-C[MSLN]_L1.17 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYSSIPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 449 aa, synthetic construct; identical to SEQ ID NO: 297
  REGION 1..449: note = Description of Artificial Sequence: Synthetic polypeptide
  REGION 1..449: note = XENP32944 Chain 1 - MESO-C[MSLN]_H1.3_IgG1_pI(-)_Isosteric_ A_/PVA_/S267K/L368D/K370S

SEQ ID NO: 376 - protein, 485 aa, synthetic construct; identical to SEQ ID NO: 283
  REGION 1..485: note = Description of Artificial Sequence: Synthetic polypeptide
  REGION 1..485: note = XENP32944 Chain 2 - MESO-C[MSLN]_H1.3_[CD3]_H1.30_L1.47_scF v(GKPGS)4-IgG1_pI( -)_Isosteric_A_/PVA_/S267K/L368D/K370S-Fc (216)_IgG1_C220S/PVA_/S267K/S364K/E357Q

SEQ ID NO: 377 - protein, 214 aa, synthetic construct
  REGION 1..214: note = Description of Artificial Sequence: Synthetic polypeptide
  REGION 1..214: note = XENP32944 Chain 3 - MESO-C[MSLN]_L1.18 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYSSHPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 378 - protein, 449 aa, synthetic construct; identical to SEQ ID NO: 297
  REGION 1..449: note = Description of Artificial Sequence: Synthetic polypeptide
  REGION 1..449: note = XENP32945 Chain 1 - MESO-C[MSLN]_H1.3_IgG1_pI(-)_Isosteric_ A_/PVA_/S267K/L368D/K370S

SEQ ID NO: 379 - protein, 485 aa, synthetic construct; identical to SEQ ID NO: 283
  REGION 1..485: note = Description of Artificial Sequence: Synthetic polypeptide
  REGION 1..485: note = XENP32945 Chain 2 - MESO-C[MSLN]_H1.3_[CD3]_H1.30_L1.47_scF v(GKPGS)4-IgG1_pI( -)_Isosteric_A_/PVA_/S267K/L368D/K370S-Fc (216)_IgG1_C220S/PVA_/S267K/S364K/E357Q

SEQ ID NO: 380 - protein, 214 aa, synthetic construct; identical to SEQ ID NO: 344
  REGION 1..214: note = Description of Artificial Sequence: Synthetic polypeptide
  REGION 1..214: note = XENP32945 Chain 3 - MESO-C[MSLN]_L1.9 Light Chain

SEQ ID NO: 381 - protein, 449 aa, synthetic construct; identical to SEQ ID NO: 297
  REGION 1..449: note = Description of Artificial Sequence: Synthetic polypeptide
  REGION 1..449: note = XENP32946 Chain 1 - MESO-C[MSLN]_H1.3_IgG1_pI(-)_Isosteric_ A_/PVA_/S267K/L368D/K370S

SEQ ID NO: 382 - protein, 485 aa, synthetic construct; identical to SEQ ID NO: 283
  REGION 1..485: note = Description of Artificial Sequence: Synthetic polypeptide
  REGION 1..485: note = XENP32946 Chain 2 - MESO-C[MSLN]_H1.3_[CD3]_H1.30_L1.47_scF v(GKPGS)4-IgG1_pI( -)_Isosteric_A_/PVA_/S267K/L368D/K370S-Fc (216)_IgG1_C220S/PVA_/S267K/S364K/E357Q

SEQ ID NO: 383 - protein, 214 aa, synthetic construct; identical to SEQ ID NO: 332
  REGION 1..214: note = Description of Artificial Sequence: Synthetic polypeptide
  REGION 1..214: note = XENP32946 Chain 3 - MESO-C[MSLN]_L1.5 Light Chain

SEQ ID NO: 384 - protein, 485 aa, synthetic construct; identical to SEQ ID NO: 283
  REGION 1..485: note = Description of Artificial Sequence: Synthetic polypeptide
  REGION 1..485: note = XENP33888 Chain 1 - MESO-C[MSLN]_H1_IgG1_pI(-)_Isosteric_A_ /PVA_/S267K/L368D/K370S

SEQ ID NO: 385 - protein, 449 aa, synthetic construct; identical to SEQ ID NO: 288
  REGION 1..449: note = Description of Artificial Sequence: Synthetic polypeptide
  REGION 1..449: note = XENP33888 Chain 2 - [CD3]_H1.30_L1.47_scFv(GKPGS)4_Fc(216)_ IgG1_C220S/PVA_/S267K/S364K/E357Q

SEQ ID NO: 386 - protein, 214 aa, synthetic construct
  REGION 1..214: note = Description of Artificial Sequence: Synthetic polypeptide
  REGION 1..214: note = XENP33888 Chain 3 - MESO-C[MSLN]_L1.20 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYYSFPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 387 - protein, 485 aa, synthetic construct; identical to SEQ ID NO: 283
  REGION 1..485: note = Description of Artificial Sequence: Synthetic polypeptide
  REGION 1..485: note = XENP33889 Chain 1 - MESO-C[MSLN]_H1.12_IgG1_pI(-)_Isosteric _A_/PVA_/S267K/L368D/K370S

SEQ ID NO: 388 - protein, 449 aa, synthetic construct; identical to SEQ ID NO: 354
  REGION 1..449: note = Description of Artificial Sequence: Synthetic polypeptide
  REGION 1..449: note = XENP33889 Chain 2 - [CD3]_H1.30_L1.47_scFv(GKPGS)4_Fc(216)_ IgG1_C220S/PVA_/S267K/S364K/E357Q

SEQ ID NO: 389 - protein, 214 aa, synthetic construct; identical to SEQ ID NO: 386
  REGION 1..214: note = Description of Artificial Sequence: Synthetic polypeptide
  REGION 1..214: note = XENP33889 Chain 3 - MESO-C[MSLN]_L1.20 Light Chain

SEQ ID NO: 390 - protein, 453 aa, synthetic construct; identical to SEQ ID NO: 229
  REGION 1..453: note = Description of Artificial Sequence: Synthetic polypeptide
  REGION 1..453: note = XENP30764 Chain 1 - MESO-A[MSLN]_H1.1_L1_Fab_IgG1_pI(-)_Iso steric_A_/PVA_/S267K/L368D/K370S

SEQ ID NO: 391 - protein, 485 aa, synthetic construct
  REGION 1..485: note = Description of Artificial Sequence: Synthetic polypeptide
  REGION 1..485: note = XENP30764 Chain 2 - [CD3]_H1.32_L1.47_scFv(GKPGS)4_Fc(216)_ IgG1_C220S/PVA_/S267K/S364K/E357Q
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 392 - protein, 213 aa, synthetic construct; identical to SEQ ID NO: 226
  REGION 1..213: note = Description of Artificial Sequence: Synthetic polypeptide
  REGION 1..213: note = XENP30764 Chain 3 - MESO-A[MSLN]_L1 Light Chain

SEQ ID NO: 393 - protein, 452 aa, synthetic construct; identical to SEQ ID NO: 285
  REGION 1..452: note = Description of Artificial Sequence: Synthetic polypeptide
  REGION 1..452: note = XENP30765 Chain 1 - MESO-B[MSLN]_H0L0_Fab_IgG1_pI(-)_Isoste ric_A_/PVA_/S267K/L368D/K370S

SEQ ID NO: 394 - protein, 485 aa, synthetic construct; identical to SEQ ID NO: 391
  REGION 1..485: note = Description of Artificial Sequence: Synthetic polypeptide
  REGION 1..485: note = XENP30765 Chain 2 - [CD3]_H1.32_L1.47_scFv(GKPGS)4_Fc(216)_ IgG1_C220S/PVA_/S267K/S364K/E357Q

SEQ ID NO: 395 - protein, 221 aa, synthetic construct; identical to SEQ ID NO: 240
  REGION 1..221: note = Description of Artificial Sequence: Synthetic polypeptide
  REGION 1..221: note = XENP30765 Chain 3 - MESO-B[MSLN]_L0 Light Chain

SEQ ID NO: 396 - protein, 449 aa, synthetic construct; identical to SEQ ID NO: 288
  REGION 1..449: note = Description of Artificial Sequence: Synthetic polypeptide
  REGION 1..449: note = XENP31701 Chain 1 - MESO-C[MSLN]_H1_IgG1_pI(-)_Isosteric_A_ /PVA_/S267K/L368D/K370S

SEQ ID NO: 397 - protein, 485 aa, synthetic construct; identical to SEQ ID NO: 391
  REGION 1..485: note = Description of Artificial Sequence: Synthetic polypeptide
  REGION 1..485: note = XENP31701 Chain 2 - [CD3]_H1.32_L1.47_scFv(GKPGS)4_Fc(216)_ IgG1_C220S/PVA_/S267K/S364K/E357Q

SEQ ID NO: 398 - protein, 214 aa, synthetic construct; identical to SEQ ID NO: 250
  REGION 1..214: note = Description of Artificial Sequence: Synthetic polypeptide
  REGION 1..214: note = XENP31701 Chain 3 - MESO-C[MSLN]_L1 Light Chain

SEQ ID NO: 399 - protein, 449 aa, synthetic construct
  REGION 1..449: note = Description of Artificial Sequence: Synthetic polypeptide
  REGION 1..449: note = XENP33969 Chain 1 - MESO-C[MSLN]_H1.14_IgG1_pI(-)_Isosteric _A_PVA_/S267K/L368D/K370S
QVQLVQSGAEVKKPGASVKVSCKASGYTFTSYWMHWVRQAPGQGLEWMGWIHPQSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 400 - protein, 485 aa, synthetic construct
  REGION 1..485: note = Description of Artificial Sequence: Synthetic polypeptide
  REGION 1..485: note = XENP33969 Chain 2 - [CD3]_L1.47_H1.32_scFv(GKPGS)4_Fc(216)_ IgG1_C220S/PVA_/S267K/S364K/E357Q
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 401 - protein, 214 aa, synthetic construct; identical to SEQ ID NO: 365
  REGION 1..214: note = Description of Artificial Sequence: Synthetic polypeptide
  REGION 1..214: note = XENP33969 Chain 3 - MESO-C[MSLN]_L1.14 Light Chain

SEQ ID NO: 402 - protein, 449 aa, synthetic construct
  REGION 1..449: note = Description of Artificial Sequence: Synthetic polypeptide
  REGION 1..449: note = XENP33971 Chain 1 - MESO-C[MSLN]_H1.15_IgG1_pI(-)_Isosteric _A_PVA_/S267K/L368D/K370S
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCARIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 403 - protein, 485 aa, synthetic construct; identical to SEQ ID NO: 400
  REGION 1..485: note = Description of Artificial Sequence: Synthetic polypeptide
  REGION 1..485: note = XENP33971 Chain 2 - [CD3]_L1.47_H1.32_scFv(GKPGS)4_Fc(216)_ IgG1_C220S/PVA_/S267K/S364K/E357Q

SEQ ID NO: 404 - protein, 214 aa, synthetic construct; identical to SEQ ID NO: 365
  REGION 1..214: note = Description of Artificial Sequence: Synthetic polypeptide
  REGION 1..214: note = XENP33971 Chain 3 - MESO-C[MSLN]_L1.14 Light Chain

SEQ ID NO: 405 - protein, 449 aa, synthetic construct
  REGION 1..449: note = Description of Artificial Sequence: Synthetic polypeptide
  REGION 1..449: note = XENP33973 Chain 1 - MESO-C[MSLN]_H1.16_IgG1_pI(-)_Isosteric _A_PVA_/S267K/L368D/K370S
QVQLVQSGAEVKKPGASVKVSCKASGYTFTSYWMHWVRQAPGQGLEWMGWIHPQSDNTIYYEKFQGRVTMTVDKSISTAYMELSRLRSDDTAVYYCARIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 406 - protein, 485 aa, synthetic construct; identical to SEQ ID NO: 400
  REGION 1..485: note = Description of Artificial Sequence: Synthetic polypeptide
  REGION 1..485: note = XENP33973 Chain 2 - [CD3]_L1.47_H1.32_scFv(GKPGS)4_Fc(216)_ IgG1_C220S/PVA_/S267K/S364K/E357Q

SEQ ID NO: 407 - protein, 214 aa, synthetic construct; identical to SEQ ID NO: 365
  REGION 1..214: note = Description of Artificial Sequence: Synthetic polypeptide
  REGION 1..214: note = XENP33973 Chain 3 - MESO-C[MSLN]_L1.14 Light Chain

SEQ ID NO: 408 - protein, 453 aa, synthetic construct; identical to SEQ ID NO: 229
  REGION 1..453: note = Description of Artificial Sequence: Synthetic polypeptide
  REGION 1..453: note = XENP16251 Chain 1 - MESO-A[MSLN]_H1.1_L1_Fab_IgG1_pI(-)_Iso steric_A_/PVA_/S267K/L368D/K370S

SEQ ID NO: 409 - protein, 485 aa, synthetic construct
  REGION 1..485: note = Description of Artificial Sequence: Synthetic polypeptide
  REGION 1..485: note = XENP16251 Chain 2 - [CD3]_H1.33_L1.47_scFv(GKPGS)4_Fc(216)_ IgG1_C220S/PVA_/S267K/S364K/E357Q
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 410 - protein, 213 aa, synthetic construct; identical to SEQ ID NO: 226
  REGION 1..213: note = Description of Artificial Sequence: Synthetic polypeptide
  REGION 1..213: note = XENP16251 Chain 3 - MESO-A[MSLN]_L1 Light Chain

SEQ ID NO: 411 - protein, 452 aa, synthetic construct; identical to SEQ ID NO: 285
  REGION 1..452: note = Description of Artificial Sequence: Synthetic polypeptide
  REGION 1..452: note = XENP16834 Chain 1 - MESO-B[MSLN]_H0L0_Fab_IgG1_pI(-)_Isoste ric_A_/PVA_/S267K/L368D/K370S

SEQ ID NO: 412 - protein, 485 aa, synthetic construct; identical to SEQ ID NO: 409
  REGION 1..485: note = Description of Artificial Sequence: Synthetic polypeptide
  REGION 1..485: note = XENP16834 Chain 2 - [CD3]_H1.33_L1.47_scFv(GKPGS)4_Fc(216)_ IgG1_C220S/PVA_/S267K/S364K/E357Q

SEQ ID NO: 413 - protein, 221 aa, synthetic construct; identical to SEQ ID NO: 240
  REGION 1..221: note = Description of Artificial Sequence: Synthetic polypeptide
  REGION 1..221: note = XENP16834 Chain 3 - MESO-B[MSLN]_L0 Light Chain

SEQ ID NO: 414 - protein, 453 aa, synthetic construct; identical to SEQ ID NO: 229
  REGION 1..453: note = Description of Artificial Sequence: Synthetic polypeptide
  REGION 1..453: note = XENP16252 Chain 1 - MESO-A[MSLN]_H1.1_L1_Fab_IgG1_pI(-)_Iso steric_A_/PVA_/S267K/L368D/K370S

SEQ ID NO: 415 - protein, 485 aa, synthetic construct
  REGION 1..485: note = Description of Artificial Sequence: Synthetic polypeptide
  REGION 1..485: note = XENP16252 Chain 2 - [CD3]_H1.31_L1.47_scFv(GKPGS)4_Fc(216)_ IgG1_C220S/PVA_/S267K/S364K/E357Q
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 416 - protein, 213 aa, synthetic construct; identical to SEQ ID NO: 226
  REGION 1..213: note = Description of Artificial Sequence: Synthetic polypeptide
  REGION 1..213: note = XENP16252 Chain 3 - MESO-A[MSLN]_L1 Light Chain

SEQ ID NO: 417 - protein, 452 aa, synthetic construct; identical to SEQ ID NO: 285
  REGION 1..452: note = Description of Artificial Sequence: Synthetic polypeptide
  REGION 1..452: note = XENP16835 Chain 1 - MESO-B[MSLN]_H0L0_Fab_IgG1_pI(-)_Isoste ric_A_/PVA_/S267K/L368D/K370S

SEQ ID NO: 418 - protein, 485 aa, synthetic construct; identical to SEQ ID NO: 415
  REGION 1..485: note = Description of Artificial Sequence: Synthetic polypeptide
  REGION 1..485: note = XENP16835 Chain 2 - [CD3]_H1.31_L1.47_scFv(GKPGS)4_Fc(216)_ IgG1_C220S/PVA_/S267K/S364K/E357Q

SEQ ID NO: 419 - protein, 221 aa, synthetic construct; identical to SEQ ID NO: 240
  REGION 1..221: note = Description of Artificial Sequence: Synthetic polypeptide
  REGION 1..221: note = XENP16835 Chain 3 - MESO-B[MSLN]_L0 Light Chain

SEQ ID NO: 420 - protein, 453 aa, synthetic construct; identical to SEQ ID NO: 229
  REGION 1..453: note = Description of Artificial Sequence: Synthetic polypeptide
  REGION 1..453: note = XENP31063 Chain 1 - MESO-A[MSLN]_H1.1_IgG1_pI(-)_Isosteric_ A_/PVA_/S267K/L368D/K370S

SEQ ID NO: 421 - protein, 726 aa, synthetic construct
  REGION 1..726: note = Description of Artificial Sequence: Synthetic polypeptide
  REGION 1..726: note = XENP31063 Chain 2 - MESO-A[MSLN]_H1.1_[CD3]_H1.30_L1.47_scF v(GKPGS)4_Fc(216)_IgG1_C220S/PVA_/S267K/S364K/E357Q
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 422 - protein, 213 aa, synthetic construct; identical to SEQ ID NO: 226
  REGION 1..213: note = Description of Artificial Sequence: Synthetic polypeptide
  REGION 1..213: note = XENP31063 Chain 3 - MESO-A[MSLN]_L1 Light Chain

SEQ ID NO: 423 - protein, 452 aa, synthetic construct; identical to SEQ ID NO: 285
  REGION 1..452: note = Description of Artificial Sequence: Synthetic polypeptide
  REGION 1..452: note = XENP31067 Chain 1 - MESO-B[MSLN]_H0_IgG1_pI(-)_Isosteric_A_ /PVA_/S267K/L368D/K370S

SEQ ID NO: 424 - protein, 725 aa, synthetic construct
  REGION 1..725: note = Description of Artificial Sequence: Synthetic polypeptide
  REGION 1..725: note = XENP31067 Chain 2 - MESO-B[MSLN]_H0_[CD3]_H1.30_L1.47_scFv( GKPGS)4_Fc(216)_IgG1_C220S/PVA_/S267K/S364K/E357Q
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 425 - protein, 221 aa, synthetic construct; identical to SEQ ID NO: 240
  REGION 1..221: note = Description of Artificial Sequence: Synthetic polypeptide
  REGION 1..221: note = XENP31067 Chain 3 - MESO-B[MSLN]_L0 Light Chain

SEQ ID NO: 426 - protein, 449 aa, synthetic construct; identical to SEQ ID NO: 288
  REGION 1..449: note = Description of Artificial Sequence: Synthetic polypeptide
  REGION 1..449: note = XENP31705 Chain 1 - MESO-C[MSLN]_H1_IgG1_pI(-)_Isosteric_A_ /PVA_/S267K/L368D/K370S

SEQ ID NO: 427 - protein, 722 aa, synthetic construct
  REGION 1..722: note = Description of Artificial Sequence: Synthetic polypeptide
  REGION 1..722: note = XENP31705 Chain 2 - MESO-C[MSLN]_H1_[CD3]_H1.30_L1.47_scFv( GKPGS)4_Fc(216)_IgG1_C220S/PVA_/S267K/S364K/E357Q
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 428 - protein, 214 aa, synthetic construct; identical to SEQ ID NO: 250
  REGION 1..214: note = Description of Artificial Sequence: Synthetic polypeptide
  REGION 1..214: note = XENP31705 Chain 3 - MESO-C[MSLN]_L1 Light Chain

SEQ ID NO: 429 - protein, 453 aa, synthetic construct; identical to SEQ ID NO: 229
  REGION 1..453: note = Description of Artificial Sequence: Synthetic polypeptide
  REGION 1..453: note = XENP31451 Chain 1 - MESO-A[MSLN]_H1.1_IgG1_pI(-)_Isosteric_ A_/PVA_/S267K/L368D/K370S

SEQ ID NO: 430 - protein, 726 aa, synthetic construct
  REGION 1..726: note = Description of Artificial Sequence: Synthetic polypeptide
  REGION 1..726: note = XENP31451 Chain 2 - MESO-A[MSLN]_H1.1_(G4S)2_[CD3]_L1.47_H1 .30_scFv(GKPGS)4_Fc(216)_IgG1_C220S/PVA_/S267K/S364K/E357Q
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 431 - protein, 213 aa, synthetic construct; identical to SEQ ID NO: 226
  REGION 1..213: note = Description of Artificial Sequence: Synthetic polypeptide
  REGION 1..213: note = XENP31451 Chain 3 - MESO-A[MSLN]_L1 Light Chain

SEQ ID NO: 432 - protein, 452 aa, synthetic construct; identical to SEQ ID NO: 285
  REGION 1..452: note = Description of Artificial Sequence: Synthetic polypeptide
  REGION 1..452: note = XENP31452 Chain 1 - MESO-B[MSLN]_H0_IgG1_pI(-)_Isosteric_A_ /PVA_/S267K/L368D/K370S

SEQ ID NO: 433 - protein, 725 aa, synthetic construct
  REGION 1..725: note = Description of Artificial Sequence: Synthetic polypeptide
  REGION 1..725: note = XENP31452 Chain 2 - MESO-B[MSLN]_H0_(G4S)2_[CD3]_L1.47_H1.3 0_scFv(GKPGS)4_Fc(216)_IgG1_C220S/PVA_/S267K/S364K/E357Q
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 434 - protein, 221 aa, synthetic construct; identical to SEQ ID NO: 240
  REGION 1..221: note = Description of Artificial Sequence: Synthetic polypeptide
  REGION 1..221: note = XENP31452 Chain 3 - MESO-B[MSLN]_L0 Light Chain

SEQ ID NO: 435 - protein, 449 aa, synthetic construct; identical to SEQ ID NO: 288
  REGION 1..449: note = Description of Artificial Sequence: Synthetic polypeptide
  REGION 1..449: note = XENP31713 Chain 1 - MESO-C[MSLN]_H1_IgG1_pI(-)_Isosteric_A_ /PVA_/S267K/L368D/K370S

SEQ ID NO: 436 - protein, 722 aa, synthetic construct
  REGION 1..722: note = Description of Artificial Sequence: Synthetic polypeptide
  REGION 1..722: note = XENP31713 Chain 2 - MESO-C[MSLN]_H1_[CD3]_L1.47_H1.30_scFv( GKPGS)4_Fc(216)_IgG1_C220S/PVA_/S267K/S364K/E357Q
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 437 - protein, 214 aa, synthetic construct; identical to SEQ ID NO: 250
  REGION 1..214: note = Description of Artificial Sequence: Synthetic polypeptide
  REGION 1..214: note = XENP31713 Chain 3 - MESO-C[MSLN]_L1 Light Chain

SEQ ID NO: 438 - protein, 453 aa, synthetic construct; identical to SEQ ID NO: 229
  REGION 1..453: note = Description of Artificial Sequence: Synthetic polypeptide
  REGION 1..453: note = XENP31064 Chain 1 - MESO-A[MSLN]_H1.1_IgG1_pI(-)_Isosteric_ A_/PVA_/S267K/L368D/K370S

SEQ ID NO: 439 - protein, 726 aa, synthetic construct
  REGION 1..726: note = Description of Artificial Sequence: Synthetic polypeptide
  REGION 1..726: note = XENP31064 Chain 2 - MESO-A[MSLN]_H1.1_[CD3]_H1.32_L1.47_scF v(GKPGS)4_Fc(216)_IgG1_C220S/PVA_/S267K/S364K/E357Q
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 440 - protein, 213 aa, synthetic construct; identical to SEQ ID NO: 226
  REGION 1..213: note = Description of Artificial Sequence: Synthetic polypeptide
  REGION 1..213: note = XENP31064 Chain 3 - MESO-A[MSLN]_L1 Light Chain

SEQ ID NO: 441 - protein, 452 aa, synthetic construct; identical to SEQ ID NO: 285
  REGION 1..452: note = Description of Artificial Sequence: Synthetic polypeptide
  REGION 1..452: note = XENP31068 Chain 1 - MESO-B[MSLN]_H0_IgG1_pI(-)_Isosteric_A_ /PVA_/S267K/L368D/K370S

SEQ ID NO: 442 - protein, 725 aa, synthetic construct
  REGION 1..725: note = Description of Artificial Sequence: Synthetic polypeptide
  REGION 1..725: note = XENP31068 Chain 2 - MESO-B[MSLN]_H0_[CD3]_H1.32_L1.47_scFv( GKPGS)4_Fc(216)_IgG1_C220S/PVA_/S267K/S364K/E357Q
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 443 - protein, 221 aa, synthetic construct; identical to SEQ ID NO: 240
  REGION 1..221: note = Description of Artificial Sequence: Synthetic polypeptide
  REGION 1..221: note = XENP31068 Chain 3 - MESO-B[MSLN]_L0 Light Chain

SEQ ID NO: 444 - protein, 449 aa, synthetic construct; identical to SEQ ID NO: 288
  REGION 1..449: note = Description of Artificial Sequence: Synthetic polypeptide
  REGION 1..449: note = XENP31709 Chain 1 - MESO-C[MSLN]_H1_IgG1_pI(-)_Isosteric_A_ /PVA_/S267K/L368D/K370S

SEQ ID NO: 445 - protein, 722 aa, synthetic construct
  REGION 1..722: note = Description of Artificial Sequence: Synthetic polypeptide
  REGION 1..722: note = XENP31709 Chain 2 - MESO-C[MSLN]_H1_[CD3]_H1.32_L1.47_scFv( GKPGS)4_Fc(216)_IgG1_C220S/PVA_/S267K/S364K/E357Q
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 446 - protein, 214 aa, synthetic construct; identical to SEQ ID NO: 250
  REGION 1..214: note = Description of Artificial Sequence: Synthetic polypeptide
  REGION 1..214: note = XENP31709 Chain 3 - MESO-C[MSLN]_L1 Light Chain

SEQ ID NO: 447 - protein, 449 aa, synthetic construct; identical to SEQ ID NO: 288
  REGION 1..449: note = Description of Artificial Sequence: Synthetic polypeptide
  REGION 1..449: note = XENP32744 Chain 1 - MESO-C[MSLN]_H1_IgG1_pI(-)_Isosteric_A_ /PVA_/S267K/L368D/K370S

SEQ ID NO: 448 - protein, 722 aa, synthetic construct; identical to SEQ ID NO: 445
  REGION 1..722: note = Description of Artificial Sequence: Synthetic polypeptide
  REGION 1..722: note = XENP32744 Chain 2 - MESO-C[MSLN]_H1_(G4S)2_[CD3]_H1.32_L1.4 7_scFv(GKPGS)4_Fc(216)_IgG1_C220S/PVA_/S267K/S364K/E357Q

SEQ ID NO: 449 - protein, 214 aa, synthetic construct; identical to SEQ ID NO: 326
  REGION 1..214: note = Description of Artificial Sequence: Synthetic polypeptide
  REGION 1..214: note = XENP32744 Chain 3 - MESO-C[MSLN]_L1.3 Light Chain

SEQ ID NO: 450 - protein, 449 aa, synthetic construct; identical to SEQ ID NO: 288
  REGION 1..449: note = Description of Artificial Sequence: Synthetic polypeptide
  REGION 1..449: note = XENP32747 Chain 1 - MESO-C[MSLN]_H1_IgG1_pI(-)_Isosteric_A_ /PVA_/S267K/L368D/K370S

SEQ ID NO: 451 - protein, 722 aa, synthetic construct; identical to SEQ ID NO: 445
  REGION 1..722: note = Description of Artificial Sequence: Synthetic polypeptide
  REGION 1..722: note = XENP32747 Chain 2 - MESO-C[MSLN]_H1_(G4S)2_[CD3]_H1.32_L1.4 7_scFv(GKPGS)4_Fc(216)_IgG1_C220S/PVA_/S267K/S364K/E357Q

SEQ ID NO: 452 - protein, 214 aa, synthetic construct; identical to SEQ ID NO: 329
  REGION 1..214: note = Description of Artificial Sequence: Synthetic polypeptide
  REGION 1..214: note = XENP32747 Chain 3 - MESO-C[MSLN]_L1.4 Light Chain

SEQ ID NO: 453 - protein, 449 aa, synthetic construct; identical to SEQ ID NO: 288
  REGION 1..449: note = Description of Artificial Sequence: Synthetic polypeptide
  REGION 1..449: note = XENP32750 Chain 1 - MESO-C[MSLN]_H1_IgG1_pI(-)_Isosteric_A_ /PVA_/S267K/L368D/K370S

SEQ ID NO: 454 - protein, 722 aa, synthetic construct; identical to SEQ ID NO: 445
  REGION 1..722: note = Description of Artificial Sequence: Synthetic polypeptide
  REGION 1..722: note = XENP32750 Chain 2 - MESO-C[MSLN]_H1_(G4S)2_[CD3]_H1.32_L1.4 7_scFv(GKPGS)4_Fc(216)_IgG1_C220S/PVA_/S267K/S364K/E357Q

SEQ ID NO: 455 - protein, 214 aa, synthetic construct; identical to SEQ ID NO: 332
  REGION 1..214: note = Description of Artificial Sequence: Synthetic polypeptide
  REGION 1..214: note = XENP32750 Chain 3 - MESO-C[MSLN]_L1.5 Light Chain

SEQ ID NO: 456 - protein, 449 aa, synthetic construct; identical to SEQ ID NO: 288
  REGION 1..449: note = Description of Artificial Sequence: Synthetic polypeptide
  REGION 1..449: note = XENP32851 Chain 1 - MESO-C[MSLN]_H1_IgG1_pI(-)_Isosteric_A_ /PVA_/S267K/L368D/K370S

SEQ ID NO: 457 - protein, 722 aa, synthetic construct; identical to SEQ ID NO: 445
  REGION 1..722: note = Description of Artificial Sequence: Synthetic polypeptide
  REGION 1..722: note = XENP32851 Chain 2 - MESO-C[MSLN]_H1_(G4S)2_[CD3]_H1.32_L1.4 7_scFv(GKPGS)4_Fc(216)_IgG1_C220S/PVA_/S267K/S364K/E357Q

SEQ ID NO: 458 - protein, 214 aa, synthetic construct; identical to SEQ ID NO: 344
  REGION 1..214: note = Description of Artificial Sequence: Synthetic polypeptide
  REGION 1..214: note = XENP32851 Chain 3 - MESO-C[MSLN]_L1.9 Light Chain

SEQ ID NO: 459 - protein, 453 aa, synthetic construct; identical to SEQ ID NO: 229
  REGION 1..453: note = Description of Artificial Sequence: Synthetic polypeptide
  REGION 1..453: note = XENP31453 Chain 1 - MESO-A[MSLN]_H1.1_IgG1_pI(-)_Isosteric_ A_/PVA_/S267K/L368D/K370S

SEQ ID NO: 460 - protein, 726 aa, synthetic construct
  REGION 1..726: note = Description of Artificial Sequence: Synthetic polypeptide
  REGION 1..726: note = XENP31453 Chain 2 - MESO-A[MSLN]_H1.1_(G4S)2_[CD3]_L1.47_H1 .32_scFv(GKPGS)4_Fc(216)_IgG1_C220S/PVA_/S267K/S364K/E357Q
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 461 - protein, 213 aa, synthetic construct; identical to SEQ ID NO: 226
  REGION 1..213: note = Description of Artificial Sequence: Synthetic polypeptide
  REGION 1..213: note = XENP31453 Chain 3 - MESO-A[MSLN]_L1 Light Chain

SEQ ID NO: 462 - protein, 452 aa, synthetic construct; identical to SEQ ID NO: 285
  REGION 1..452: note = Description of Artificial Sequence: Synthetic polypeptide
  REGION 1..452: note = XENP31454 Chain 1 - MESO-B[MSLN]_H0_IgG1_pI(-)_Isosteric_A_ /PVA_/S267K/L368D/K370S

SEQ ID NO: 463 - protein, 725 aa, synthetic construct
  REGION 1..725: note = Description of Artificial Sequence: Synthetic polypeptide
  REGION 1..725: note = XENP31454 Chain 2 - MESO-B[MSLN]_H0_(G4S)2_[CD3]_L1.47_H1.3 2_scFv(GKPGS)4_Fc(216)_IgG1_C220S/PVA_/S267K/S364K/E357Q
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 464 - protein, 221 aa, synthetic construct; identical to SEQ ID NO: 240
  REGION 1..221: note = Description of Artificial Sequence: Synthetic polypeptide
  REGION 1..221: note = XENP31454 Chain 3 - MESO-B[MSLN]_L0 Light Chain

SEQ ID NO: 465 - protein, 449 aa, synthetic construct; identical to SEQ ID NO: 288
  REGION 1..449: note = Description of Artificial Sequence: Synthetic polypeptide
  REGION 1..449: note = XENP31717 Chain 1 - MESO-C[MSLN]_H1_IgG1_pI(-)_Isosteric_A_ /PVA_/S267K/L368D/K370S

SEQ ID NO: 466 - protein, 722 aa, synthetic construct
  REGION 1..722: note = Description of Artificial Sequence: Synthetic polypeptide
  REGION 1..722: note = XENP31717 Chain 2 - MESO-C[MSLN]_H1_[CD3]_L1.47_H1.32_scFv( GKPGS)4_Fc(216)_IgG1_C220S/PVA_/S267K/S364K/E357Q
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 467 - protein, 214 aa, synthetic construct; identical to SEQ ID NO: 250
  REGION 1..214: note = Description of Artificial Sequence: Synthetic polypeptide
  REGION 1..214: note = XENP31717 Chain 3 - MESO-C[MSLN]_L1 Light Chain

SEQ ID NO: 468 - protein, 449 aa, synthetic construct; identical to SEQ ID NO: 288
  REGION 1..449: note = Description of Artificial Sequence: Synthetic polypeptide
  REGION 1..449: note = XENP32745 Chain 1 - MESO-C[MSLN]_H1_IgG1_pI(-)_Isosteric_A_ /PVA_/S267K/L368D/K370S

SEQ ID NO: 469 - protein, 722 aa, synthetic construct; identical to SEQ ID NO: 466
  REGION 1..722: note = Description of Artificial Sequence: Synthetic polypeptide
  REGION 1..722: note = XENP32745 Chain 2 - MESO-C[MSLN]_H1_(G4S)2_[CD3]_L1.47_H1.3 2_scFv(GKPGS)4_Fc(216)_IgG1_C220S/PVA_/S267K/S364K/E357Q

SEQ ID NO: 470 - protein, 214 aa, synthetic construct; identical to SEQ ID NO: 326
  REGION 1..214: note = Description of Artificial Sequence: Synthetic polypeptide
  REGION 1..214: note = XENP32745 Chain 3 - MESO-C[MSLN]_L1.3 Light Chain

SEQ ID NO: 471 - protein, 449 aa, synthetic construct; identical to SEQ ID NO: 288
  REGION 1..449: note = Description of Artificial Sequence: Synthetic polypeptide
  REGION 1..449: note = XENP32748 Chain 1 - MESO-C[MSLN]_H1_IgG1_pI(-)_Isosteric_A_ /PVA_/S267K/L368D/K370S

SEQ ID NO: 472 - protein, 722 aa, synthetic construct; identical to SEQ ID NO: 466
  REGION 1..722: note = Description of Artificial Sequence: Synthetic polypeptide
  REGION 1..722: note = XENP32748 Chain 2 - MESO-C[MSLN]_H1_(G4S)2_[CD3]_L1.47_H1.3 2_scFv(GKPGS)4_Fc(216)_IgG1_C220S/PVA_/S267K/S364K/E357Q

SEQ ID NO: 473 - protein, 214 aa, synthetic construct; identical to SEQ ID NO: 329
  REGION 1..214: note = Description of Artificial Sequence: Synthetic polypeptide
  REGION 1..214: note = XENP32748 Chain 3 - MESO-C[MSLN]_L1.4 Light Chain

SEQ ID NO: 474 - protein, 449 aa, synthetic construct; identical to SEQ ID NO: 288
  REGION 1..449: note = Description of Artificial Sequence: Synthetic polypeptide
  REGION 1..449: note = XENP32751 Chain 1 - MESO-C[MSLN]_H1_IgG1_pI(-)_Isosteric_A_ /PVA_/S267K/L368D/K370S

SEQ ID NO: 475 - protein, 722 aa, synthetic construct; identical to SEQ ID NO: 466
  REGION 1..722: note = Description of Artificial Sequence: Synthetic polypeptide
  REGION 1..722: note = XENP32751 Chain 2 - MESO-C[MSLN]_H1_(G4S)2_[CD3]_L1.47_H1.3 2_scFv(GKPGS)4_Fc(216)_IgG1_C220S/PVA_/S267K/S364K/E357Q

SEQ ID NO: 476 - protein, 213 aa, synthetic construct
  REGION 1..213: note = Description of Artificial Sequence: Synthetic polypeptide
  REGION 1..213: note = XENP32751 Chain 3 - MESO-C[MSLN]_L1.5 Light Chain
DIVMTQSPDSLAVSLGERATINCKASHDVGTYVAWYQQKPGQSPKLLIYWASTRHTGVPDRFTGSGSGTDFTLTISSLQAEDVAVYFCQQYSSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 477 - protein, 449 aa, synthetic construct; identical to SEQ ID NO: 288
  REGION 1..449: note = Description of Artificial Sequence: Synthetic polypeptide
  REGION 1..449: note = XENP32852 Chain 1 - MESO-C[MSLN]_H1_IgG1_pI(-)_Isosteric_A_ /PVA_/S267K/L368D/K370S

SEQ ID NO: 478 - protein, 722 aa, synthetic construct; identical to SEQ ID NO: 466
  REGION 1..722: note = Description of Artificial Sequence: Synthetic polypeptide
  REGION 1..722: note = XENP32852 Chain 2 - MESO-C[MSLN]_H1_(G4S)2_[CD3]_L1.47_H1.3 2_scFv(GKPGS)4_Fc(216)_IgG1_C220S/PVA_/S267K/S364K/E357Q

SEQ ID NO: 479 - protein, 214 aa, synthetic construct; identical to SEQ ID NO: 344
  REGION 1..214: note = Description of Artificial Sequence: Synthetic polypeptide
  REGION 1..214: note = XENP32852 Chain 3 - MESO-C[MSLN]_L1.9 Light Chain

SEQ ID NO: 480 - protein, 449 aa, synthetic construct; identical to SEQ ID NO: 297
  REGION 1..449: note = Description of Artificial Sequence: Synthetic polypeptide
  REGION 1..449: note = XENP32947 Chain 1 - MESO-C[MSLN]_H1.3_IgG1_pI(-)_Isosteric_ A_/PVA_/S267K/L368D/K370S

SEQ ID NO: 481 - protein, 722 aa, synthetic construct
  REGION 1..722: note = Description of Artificial Sequence: Synthetic polypeptide
  REGION 1..722: note = XENP32947 Chain 2 - MESO-C[MSLN]_H1.3_(G4S)2_[CD3]_L1.47_H1 .32_scFv(GKPGS)4_Fc(216)_IgG1_C220S/PVA_/S267K/S364K/E357Q
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 482 - protein, 214 aa, synthetic construct; identical to SEQ ID NO: 250
  REGION 1..214: note = Description of Artificial Sequence: Synthetic polypeptide
  REGION 1..214: note = XENP32947 Chain 3 - MESO-C[MSLN]_L1 Light Chain

SEQ ID NO: 483 - protein, 449 aa, synthetic construct; identical to SEQ ID NO: 354
  REGION 1..449: note = Description of Artificial Sequence: Synthetic polypeptide
  REGION 1..449: note = XENP32948 Chain 1 - MESO-C[MSLN]_H1.12_IgG1_pI(-)_Isosteric _A_/PVA_/S267K/L368D/K370S

SEQ ID NO: 484 - protein, 722 aa, synthetic construct
  REGION 1..722: note = Description of Artificial Sequence: Synthetic polypeptide
  REGION 1..722: note = XENP32948 Chain 2 - MESO-C[MSLN]_H1.12_(G4S)2_[CD3]_L1.47_H 1.32_scFv(GKPGS)4_ Fc(216)_IgG1_C220S/PVA_/S267K/S364K/E357Q
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 485 - protein, 214 aa, synthetic construct; identical to SEQ ID NO: 344
  REGION 1..214: note = Description of Artificial Sequence: Synthetic polypeptide
  REGION 1..214: note = XENP32948 Chain 3 - MESO-C[MSLN]_L1.9 Light Chain

SEQ ID NO: 486 - protein, 449 aa, synthetic construct; identical to SEQ ID NO: 357
  REGION 1..449: note = Description of Artificial Sequence: Synthetic polypeptide
  REGION 1..449: note = XENP32949 Chain 1 - MESO-C[MSLN]_H1.13_IgG1_pI(-)_Isosteric _A_/PVA_/S267K/L368D/K370S

SEQ ID NO: 487 - protein, 722 aa, synthetic construct
  REGION 1..722: note = Description of Artificial Sequence: Synthetic polypeptide
  REGION 1..722: note = XENP32949 Chain 2 - MESO-C[MSLN]_H1.13_(G4S)2_[CD3]_L1.47_H 1.32_scFv(GKPGS)4_ Fc(216)_IgG1_C220S/PVA_/S267K/S364K/E357Q
QVQLVQSGAEVKKPGASVKVSCKASGYTFTSYWMHWVRQAPGQGLEWMGMIHPNSDNTIYYYKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 488 - protein, 214 aa, synthetic construct; identical to SEQ ID NO: 344
  REGION 1..214: note = Description of Artificial Sequence: Synthetic polypeptide
  REGION 1..214: note = XENP32949 Chain 3 - MESO-C[MSLN]_L1.9 Light Chain

SEQ ID NO: 489 - protein, 449 aa, synthetic construct; identical to SEQ ID NO: 297
  REGION 1..449: note = Description of Artificial Sequence: Synthetic polypeptide
  REGION 1..449: note = XENP32950 Chain 1 - MESO-C[MSLN]_H1.3_IgG1_pI(-)_Isosteric_ A_/PVA_/S267K/L368D/K370S

SEQ ID NO: 490 - protein, 722 aa, synthetic construct; identical to SEQ ID NO: 481
  REGION 1..722: note = Description of Artificial Sequence: Synthetic polypeptide
  REGION 1..722: note = XENP32950 Chain 2 - MESO-C[MSLN]_H1.3_(G4S)2_[CD3]_L1.47_H1 .32_scFv(GKPGS)4_Fc(216)_IgG1_C220S/PVA_/S267K/S364K/E357Q

SEQ ID NO: 491 - protein, 214 aa, synthetic construct; identical to SEQ ID NO: 344
  REGION 1..214: note = Description of Artificial Sequence: Synthetic polypeptide
  REGION 1..214: note = XENP32950 Chain 3 - MESO-C[MSLN]_L1.9 Light Chain

SEQ ID NO: 492 - protein, 449 aa, synthetic construct; identical to SEQ ID NO: 297
  REGION 1..449: note = Description of Artificial Sequence: Synthetic polypeptide
  REGION 1..449: note = XENP32951 Chain 1 - MESO-C[MSLN]_H1.3_IgG1_pI(-)_Isosteric_ A_/PVA_/S267K/L368D/K370S

SEQ ID NO: 493 - protein, 722 aa, synthetic construct; identical to SEQ ID NO: 481
  REGION 1..722: note = Description of Artificial Sequence: Synthetic polypeptide
  REGION 1..722: note = XENP32951 Chain 2 - MESO-C[MSLN]_H1.3_(G4S)2_[CD3]_L1.47_H1 .32_scFv(GKPGS)4_Fc(216)_IgG1_C220S/PVA_/S267K/S364K/E357Q

SEQ ID NO: 494 - protein, 214 aa, synthetic construct; identical to SEQ ID NO: 332
  REGION 1..214: note = Description of Artificial Sequence: Synthetic polypeptide
  REGION 1..214: note = XENP32951 Chain 3 - MESO-C[MSLN]_L1.5 Light Chain

SEQ ID NO: 495 - protein, 449 aa, synthetic construct; identical to SEQ ID NO: 297
  REGION 1..449: note = Description of Artificial Sequence: Synthetic polypeptide
  REGION 1..449: note = XENP33366 Chain 1 - MESO-C[MSLN]_H1.3_IgG1_pI(-)_Isosteric_ A_/PVA_/S267K/L368D/K370S

SEQ ID NO: 496 - protein, 722 aa, synthetic construct; identical to SEQ ID NO: 481
  REGION 1..722: note = Description of Artificial Sequence: Synthetic polypeptide
  REGION 1..722: note = XENP33366 Chain 2 - MESO-C[MSLN]_H1.3_(G4S)2_[CD3]_L1.47_H1 .32_scFv(GKPGS)4_Fc(216)_IgG1_C220S/PVA_/S267K/S364K/E357Q

SEQ ID NO: 497 - protein, 214 aa, synthetic construct; identical to SEQ ID NO: 362
  REGION 1..214: note = Description of Artificial Sequence: Synthetic polypeptide
  REGION 1..214: note = XENP33366 Chain 3 - MESO-C[MSLN]_L1.13 Light Chain

SEQ ID NO: 498 - protein, 449 aa, synthetic construct; identical to SEQ ID NO: 297
  REGION 1..449: note = Description of Artificial Sequence: Synthetic polypeptide
  REGION 1..449: note = XENP33367 Chain 1 - MESO-C[MSLN]_H1.3_IgG1_pI(-)_Isosteric_ A_/PVA_/S267K/L368D/K370S

SEQ ID NO: 499 - protein, 722 aa, synthetic construct; identical to SEQ ID NO: 481
  REGION 1..722: note = Description of Artificial Sequence: Synthetic polypeptide
  REGION 1..722: note = XENP33367 Chain 2 - MESO-C[MSLN]_H1.3_(G4S)2_[CD3]_L1.47_H1 .32_scFv(GKPGS)4_Fc(216)_IgG1_C220S/PVA_/S267K/S364K/E357Q

SEQ ID NO: 500 - protein, 214 aa, synthetic construct; identical to SEQ ID NO: 365
  REGION 1..214: note = Description of Artificial Sequence: Synthetic polypeptide
  REGION 1..214: note = XENP33367 Chain 3 - MESO-C[MSLN]_L1.14 Light Chain

SEQ ID NO: 501 - protein, 449 aa, synthetic construct; identical to SEQ ID NO: 288
  REGION 1..449: note = Description of Artificial Sequence: Synthetic polypeptide
  REGION 1..449: note = XENP33460 Chain 1 - MESO-C[MSLN]_H1_IgG1_pI(-)_Isosteric_A_ /PVA_/S267K/L368D/K370S

SEQ ID NO: 502 - protein, 722 aa, synthetic construct; identical to SEQ ID NO: 466
  REGION 1..722: note = Description of Artificial Sequence: Synthetic polypeptide
  REGION 1..722: note = XENP33460 Chain 2 - MESO-C[MSLN]_H1_(G4S)2_[CD3]_L1.47_H1.3 2_scFv(GKPGS)4_Fc(216)_IgG1_C220S/PVA_/S267K/S364K/E357Q

SEQ ID NO: 503 - protein, 214 aa, synthetic construct
  REGION 1..214: note = Description of Artificial Sequence: Synthetic polypeptide
  REGION 1..214: note = XENP33460 Chain 3 - MESO-C[MSLN]_L1.19 Light Chain
DIVMTQSPDSLAVSLGERATINCKASHDVGTYVAWYQQKPGQSPKLLIYWASTRHTGVPDRFTGSGSGTDFTLTISSLQAEDVAVYFCQQYYSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 504 - protein, 449 aa, synthetic construct; identical to SEQ ID NO: 288
  REGION 1..449: note = Description of Artificial Sequence: Synthetic polypeptide
  REGION 1..449: note = XENP33461 Chain 1 - MESO-C[MSLN]_H1_IgG1_pI(-)_Isosteric_A_ /PVA_/S267K/L368D/K370S

SEQ ID NO: 505 - protein, 722 aa, synthetic construct; identical to SEQ ID NO: 466
  REGION 1..722: note = Description of Artificial Sequence: Synthetic polypeptide
  REGION 1..722: note = XENP33461 Chain 2 - MESO-C[MSLN]_H1_(G4S)2_[CD3]_L1.47_H1.3 2_scFv(GKPGS)4_Fc(216)_IgG1_C220S/PVA_/S267K/S364K/E357Q

SEQ ID NO: 506 - protein, 214 aa, synthetic construct; identical to SEQ ID NO: 386
  REGION 1..214: note = Description of Artificial Sequence: Synthetic polypeptide
  REGION 1..214: note = XENP33461 Chain 3 - MESO-C[MSLN]_L1.20 Light Chain

SEQ ID NO: 507 - protein, 449 aa, synthetic construct; identical to SEQ ID NO: 288
  REGION 1..449: note = Description of Artificial Sequence: Synthetic polypeptide
  REGION 1..449: note = XENP33462 Chain 1 - MESO-C[MSLN]_H1_IgG1_pI(-)_Isosteric_A_ /PVA_/S267K/L368D/K370S

SEQ ID NO: 508 - protein, 722 aa, synthetic construct; identical to SEQ ID NO: 466
  REGION 1..722: note = Description of Artificial Sequence: Synthetic polypeptide
  REGION 1..722: note = XENP33462 Chain 2 - MESO-C[MSLN]_H1_(G4S)2_[CD3]_L1.47_H1.3 2_scFv(GKPGS)4_Fc(216)_IgG1_C220S/PVA_/S267K/S364K/E357Q

SEQ ID NO: 509 - protein, 214 aa, synthetic construct
  REGION 1..214: note = Description of Artificial Sequence: Synthetic polypeptide
  REGION 1..214: note = XENP33462 Chain 3 - MESO-C[MSLN]_L1.21 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYYSVPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 510 - protein, 449 aa, synthetic construct; identical to SEQ ID NO: 288
  REGION 1..449: note = Description of Artificial Sequence: Synthetic polypeptide
  REGION 1..449: note = XENP33463 Chain 1 - MESO-C[MSLN]_H1_IgG1_pI(-)_Isosteric_A_ /PVA_/S267K/L368D/K370S

SEQ ID NO: 511 - protein, 722 aa, synthetic construct; identical to SEQ ID NO: 466
  REGION 1..722: note = Description of Artificial Sequence: Synthetic polypeptide
  REGION 1..722: note = XENP33463 Chain 2 - MESO-C[MSLN]_H1_(G4S)2_[CD3]_L1.47_H1.3 2_scFv(GKPGS)4_Fc(216)_IgG1_C220S/PVA_/S267K/S364K/E357Q

SEQ ID NO: 512 - protein, 214 aa, synthetic construct
  REGION 1..214: note = Description of Artificial Sequence: Synthetic polypeptide
  REGION 1..214: note = XENP33463 Chain 3 - MESO-C[MSLN]_L1.22 Light Chain
DIVMTQSPDSLAVSLGERATINCKASHDVGTSVAWYQQKPGQSPKLLIYWASTRHTGVPDRFTGSGSGTDFTLTISSLQAEDVAVYFCQQYYSIPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 513 - protein, 449 aa, synthetic construct; identical to SEQ ID NO: 354
  REGION 1..449: note = Description of Artificial Sequence: Synthetic polypeptide
  REGION 1..449: note = XENP33464 Chain 1 - MESO-C[MSLN]_H1.12_IgG1_pI(-)_Isosteric _A_/PVA_/S267K/L368D/K370S

SEQ ID NO: 514 - protein, 722 aa, synthetic construct; identical to SEQ ID NO: 484
  REGION 1..722: note = Description of Artificial Sequence: Synthetic polypeptide
  REGION 1..722: note = XENP33464 Chain 2 - MESO-C[MSLN]_H1.12_(G4S)2_[CD3]_L1.47_H 1.32_scFv(GKPGS)4_ Fc(216)_IgG1_C220S/PVA_/S267K/S364K/E357Q

SEQ ID NO: 515 - protein, 214 aa, synthetic construct; identical to SEQ ID NO: 503
  REGION 1..214: note = Description of Artificial Sequence: Synthetic polypeptide
  REGION 1..214: note = XENP33464 Chain 3 - MESO-C[MSLN]_L1.19 Light Chain

SEQ ID NO: 516 - protein, 449 aa, synthetic construct; identical to SEQ ID NO: 354
  REGION 1..449: note = Description of Artificial Sequence: Synthetic polypeptide
  REGION 1..449: note = XENP33465 Chain 1 - MESO-C[MSLN]_H1.12_IgG1_pI(-)_Isosteric _A_/PVA_/S267K/L368D/K370S

SEQ ID NO: 517 - protein, 722 aa, synthetic construct; identical to SEQ ID NO: 484
  REGION 1..722: note = Description of Artificial Sequence: Synthetic polypeptide
  REGION 1..722: note = XENP33465 Chain 2 - MESO-C[MSLN]_H1.12_(G4S)2_[CD3]_L1.47_H 1.32_scFv(GKPGS)4_ Fc(216)_IgG1_C220S/PVA_/S267K/S364K/E357Q

SEQ ID NO: 518 - protein, 214 aa, synthetic construct; identical to SEQ ID NO: 386
  REGION 1..214: note = Description of Artificial Sequence: Synthetic polypeptide
  REGION 1..214: note = XENP33465 Chain 3 - MESO-C[MSLN]_L1.20 Light Chain

SEQ ID NO: 519 - protein, 449 aa, synthetic construct; identical to SEQ ID NO: 354
  REGION 1..449: note = Description of Artificial Sequence: Synthetic polypeptide
  REGION 1..449: note = XENP33466 Chain 1 - MESO-C[MSLN]_H1.12_IgG1_pI(-)_Isosteric _A_/PVA_/S267K/L368D/K370S

SEQ ID NO: 520 - protein, 722 aa, synthetic construct; identical to SEQ ID NO: 484
  REGION 1..722: note = Description of Artificial Sequence: Synthetic polypeptide
  REGION 1..722: note = XENP33466 Chain 2 - MESO-C[MSLN]_H1.12_(G4S)2_[CD3]_L1.47_H 1.32_scFv(GKPGS)4_ Fc(216)_IgG1_C220S/PVA_/S267K/S364K/E357Q

SEQ ID NO: 521 - protein, 214 aa, synthetic construct; identical to SEQ ID NO: 509
  REGION 1..214: note = Description of Artificial Sequence: Synthetic polypeptide
  REGION 1..214: note = XENP33466 Chain 3 - MESO-C[MSLN]_L1.21 Light Chain

SEQ ID NO: 522 - protein, 449 aa, synthetic construct; identical to SEQ ID NO: 354
  REGION 1..449: note = Description of Artificial Sequence: Synthetic polypeptide
  REGION 1..449: note = XENP33467 Chain 1 - MESO-C[MSLN]_H1.12_IgG1_pI(-)_Isosteric _A_/PVA_/S267K/L368D/K370S

SEQ ID NO: 523 - protein, 722 aa, synthetic construct; identical to SEQ ID NO: 484
  REGION 1..722: note = Description of Artificial Sequence: Synthetic polypeptide
  REGION 1..722: note = XENP33467 Chain 2 - MESO-C[MSLN]_H1.12_(G4S)2_[CD3]_L1.47_H 1.32_scFv(GKPGS)4_ Fc(216)_IgG1_C220S/PVA_/S267K/S364K/E357Q

SEQ ID NO: 524 - protein, 214 aa, synthetic construct; identical to SEQ ID NO: 512
  REGION 1..214: note = Description of Artificial Sequence: Synthetic polypeptide
  REGION 1..214: note = XENP33467 Chain 3 - MESO-C[MSLN]_L1.22 Light Chain

SEQ ID NO: 525 - protein, 449 aa, synthetic construct; identical to SEQ ID NO: 399
  REGION 1..449: note = Description of Artificial Sequence: Synthetic polypeptide
  REGION 1..449: note = XENP3396 Chain 1 - MESO-C[MSLN]_H1.14_IgG1_pI(-)_Isosteric_ A_PVA_/S267K/L368 D/K370S-(G4S)2_IgG1_PVA_/S267K/S364K/E357Q

SEQ ID NO: 526 - protein, 722 aa, synthetic construct
  REGION 1..722: note = Description of Artificial Sequence: Synthetic polypeptide
  REGION 1..722: note = XENP3396 Chain 2 - MESO-C[MSLN]_H1.14_(G4S)2_[CD3]_(G4S)2_I gG1_PVA_/S267K/S364K/E357Q
QVQLVQSGAEVKKPGASVKVSCKASGYTFTSYWMHWVRQAPGQGLEWMGWIHPQSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 527 - protein, 214 aa, synthetic construct; identical to SEQ ID NO: 365
  REGION 1..214: note = Description of Artificial Sequence: Synthetic polypeptide
  REGION 1..214: note = XENP3396 Chain 3 - MESO-C[MSLN]_L1.14 Light Chain

SEQ ID NO: 528 - protein, 449 aa, synthetic construct; identical to SEQ ID NO: 402
  REGION 1..449: note = Description of Artificial Sequence: Synthetic polypeptide
  REGION 1..449: note = XENP33970 Chain 1 - MESO-C[MSLN]_H1.15_IgG1_pI(-)_Isosteric _A_PVA_/S267K/L368 D/K370S-(G4S)2_IgG1_PVA_/S267K/S364K/E357Q

SEQ ID NO: 529 - protein, 722 aa, synthetic construct
  REGION 1..722: note = Description of Artificial Sequence: Synthetic polypeptide
  REGION 1..722: note = XENP33970 Chain 2 - MESO-C[MSLN]_H1.15_(G4S)2_[CD3]_(G4S)2_ IgG1_PVA_/S267K/S364K/E357Q
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCARIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 530 - protein, 214 aa, synthetic construct; identical to SEQ ID NO: 365
  REGION 1..214: note = Description of Artificial Sequence: Synthetic polypeptide
  REGION 1..214: note = XENP33970 Chain 3 - MESO-C[MSLN]_L1.14 Light Chain

SEQ ID NO: 531 - protein, 449 aa, synthetic construct; identical to SEQ ID NO: 405
  REGION 1..449: note = Description of Artificial Sequence: Synthetic polypeptide
  REGION 1..449: note = XENP33972 Chain 1 - MESO-C[MSLN]_H1.16_IgG1_pI(-)_Isosteric _A_PVA_/S267K/L368 D/K370S-(G4S)2_IgG1_PVA_/S267K/S364K/E357Q

SEQ ID NO: 532 - protein, 722 aa, synthetic construct
  REGION 1..722: note = Description of Artificial Sequence: Synthetic polypeptide
  REGION 1..722: note = XENP33972 Chain 2 - MESO-C[MSLN]_H1.16_(G4S)2_[CD3]_(G4S)2_ IgG1_PVA_/S267K/S364K/E357Q
QVQLVQSGAEVKKPGASVKVSCKASGYTFTSYWMHWVRQAPGQGLEWMGWIHPQSDNTIYYEKFQGRVTMTVDKSISTAYMELSRLRSDDTAVYYCARIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 533 - protein, 214 aa, synthetic construct; identical to SEQ ID NO: 365
  REGION 1..214: note = Description of Artificial Sequence: Synthetic polypeptide
  REGION 1..214: note = XENP33972 Chain 3 - MESO-C[MSLN]_L1.14 Light Chain

SEQ ID NO: 534 - protein, 449 aa, synthetic construct
  REGION 1..449: note = Description of Artificial Sequence: Synthetic polypeptide
  REGION 1..449: note = XENP34155 Chain 1 - MESO-C[MSLN]_H1.3_IgG1_pI(-)_Isosteric_ A_/PVA_/S267K/L368D/K370S/M248L/N434S
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 535 - protein, 722 aa, synthetic construct
  REGION 1..722: note = Description of Artificial Sequence: Synthetic polypeptide
  REGION 1..722: note = XENP34155 Chain 2 - MESO-C[MSLN]_H1.3_(G4S)2_[CD3]_L1.47_H1 .32_scFv(GKPGS)4_F c(216)_IgG1_C220S/PVA_/S267K/S364K/E357Q/ M248L/N434S
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 536 - protein, 214 aa, synthetic construct; identical to SEQ ID NO: 365
  REGION 1..214: note = Description of Artificial Sequence: Synthetic polypeptide
  REGION 1..214: note = XENP34155 Chain 3 - MESO-C[MSLN]_L1.14 Light Chain

SEQ ID NO: 537 - protein, 449 aa, synthetic construct; identical to SEQ ID NO: 534
  REGION 1..449: note = Description of Artificial Sequence: Synthetic polypeptide
  REGION 1..449: note = XENP34156 Chain 1 - MESO-C[MSLN]_H1.3_IgG1_pI(-)_Isosteric_ A_/PVA_/S267K/L368D/K370S/M248L/N434S

SEQ ID NO: 538 - protein, 722 aa, synthetic construct; identical to SEQ ID NO: 535
  REGION 1..722: note = Description of Artificial Sequence: Synthetic polypeptide
  REGION 1..722: note = XENP34156 Chain 2 - MESO-C[MSLN]_H1.3_(G4S)2_[CD3]_L1.47_H1 .32_scFv(GKPGS)4_F c(216)_IgG1_C220S/PVA_/S267K/S364K/E357Q/ M248L/N434S

SEQ ID NO: 539 - protein, 214 aa, synthetic construct; identical to SEQ ID NO: 362
  REGION 1..214: note = Description of Artificial Sequence: Synthetic polypeptide
  REGION 1..214: note = XENP34156 Chain 3 - MESO-C[MSLN]_L1.13 Light Chain

SEQ ID NO: 540 - protein, 449 aa, synthetic construct; identical to SEQ ID NO: 534
  REGION 1..449: note = Description of Artificial Sequence: Synthetic polypeptide
  REGION 1..449: note = XENP34157 Chain 1 - MESO-C[MSLN]_H1.3_IgG1_pI(-)_Isosteric_ A_/PVA_/S267K/L368D/K370S/M248L/N434S

SEQ ID NO: 541 - protein, 722 aa, synthetic construct; identical to SEQ ID NO: 535
  REGION 1..722: note = Description of Artificial Sequence: Synthetic polypeptide
  REGION 1..722: note = XENP34157 Chain 2 - MESO-C[MSLN]_H1.3_(G4S)2_[CD3]_L1.47_H1 .32_scFv(GKPGS)4_F c(216)_IgG1_C220S/PVA_/S267K/S364K/E357Q/ M248L/N434S

SEQ ID NO: 542 - protein, 214 aa, synthetic construct; identical to SEQ ID NO: 250
  REGION 1..214: note = Description of Artificial Sequence: Synthetic polypeptide
  REGION 1..214: note = XENP34157 Chain 3 - MESO-C[MSLN]_L1 Light Chain

SEQ ID NO: 543 - protein, 449 aa, synthetic construct
  REGION 1..449: note = Description of Artificial Sequence: Synthetic polypeptide
  REGION 1..449: note = XENP34158 Chain 1 - MESO-C[MSLN]_H1.12_IgG1_pI(-)_Isosteric _A_/PVA_/S267K/L368D/K370S/M248L/N434S
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 544 - protein, 722 aa, synthetic construct
  REGION 1..722: note = Description of Artificial Sequence: Synthetic polypeptide
  REGION 1..722: note = XENP34158 Chain 2 - MESO-C[MSLN]_H1.12_(G4S)2_[CD3]_L1.47_H 1.32_scFv(GKPGS)4_ Fc(216)_IgG1_C220S/PVA_/S267K/S364K/E357Q /M248L/N434S
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 545 - protein, 214 aa, synthetic construct; identical to SEQ ID NO: 344
  REGION 1..214: note = Description of Artificial Sequence: Synthetic polypeptide
  REGION 1..214: note = XENP34158 Chain 3 - MESO-C[MSLN]_L1.9 Light Chain

SEQ ID NO: 546 - protein, 453 aa, synthetic construct; identical to SEQ ID NO: 229
  REGION 1..453: note = Description of Artificial Sequence: Synthetic polypeptide
  REGION 1..453: note = XENP31455 Chain 1 - MESO-A[MSLN]_H1.1_IgG1_pI(-)_Isosteric_ A_/PVA_/S267K/L368D/K370S

SEQ ID NO: 547 - protein, 726 aa, synthetic construct
  REGION 1..726: note = Description of Artificial Sequence: Synthetic polypeptide
  REGION 1..726: note = XENP31455 Chain 2 - MESO-A[MSLN]_H1.1_(G4S)2_[CD3]_(G4S)2_I gG1_C220S/PVA_/S267K/S364K/E357Q
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 548 - protein, 213 aa, synthetic construct; identical to SEQ ID NO: 226
  REGION 1..213: note = Description of Artificial Sequence: Synthetic polypeptide
  REGION 1..213: note = XENP31455 Chain 3 - MESO-A[MSLN]_L1 Light Chain

SEQ ID NO: 549 - protein, 452 aa, synthetic construct; identical to SEQ ID NO: 285
  REGION 1..452: note = Description of Artificial Sequence: Synthetic polypeptide
  REGION 1..452: note = XENP31456 Chain 1 - MESO-B[MSLN]_H0_IgG1_pI(-)_Isosteric_A_ /PVA_/S267K/L368D/K370S

SEQ ID NO: 550 - protein, 725 aa, synthetic construct
  REGION 1..725: note = Description of Artificial Sequence: Synthetic polypeptide
  REGION 1..725: note = XENP31456 Chain 2 - MESO-B[MSLN]_H0_(G4S)2_[CD3]_(G4S)2_IgG 1_C220S/PVA_/S267K/S364K/E357Q
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 551 - protein, 221 aa, synthetic construct; identical to SEQ ID NO: 240
  REGION 1..221: note = Description of Artificial Sequence: Synthetic polypeptide
  REGION 1..221: note = XENP31456 Chain 3 - MESO-B[MSLN]_L0 Light Chain

SEQ ID NO: 552 - protein, 453 aa, synthetic construct; identical to SEQ ID NO: 229
  REGION 1..453: note = Description of Artificial Sequence: Synthetic polypeptide
  REGION 1..453: note = XENP32249 Chain 1 - MESO-A[MSLN]_H1.1_IgG1_pI(-)_Isosteric_ A_/PVA_/S267K/L368D/K370S

SEQ ID NO: 553 - protein, 722 aa, synthetic construct
  REGION 1..722: note = Description of Artificial Sequence: Synthetic polypeptide
  REGION 1..722: note = XENP32249 Chain 2 - MESO-A[MSLN]_H1.1_(G4S)2_[CD3]_L1.47_H1 .89_scFv(GKPGS)4_Fc(216)_IgG1_C220S/PVA_/S267K/S364K/E357Q
EVQLVESGGGLVQPGGSLKLSCAASGFDFSRYWMSWVRQAPGKGLEWVSEINPDSSTIVYTPSVKGRFTISRDNAKNSLYLQMNSLRAEDTAVYYCARRGSHYYGYRTGYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 554 - protein, 213 aa, synthetic construct; identical to SEQ ID NO: 226
  REGION 1..213: note = Description of Artificial Sequence: Synthetic polypeptide
  REGION 1..213: note = XENP32249 Chain 3 - MESO-A[MSLN]_L1 Light Chain

SEQ ID NO: 555 - protein, 452 aa, synthetic construct; identical to SEQ ID NO: 285
  REGION 1..452: note = Description of Artificial Sequence: Synthetic polypeptide
  REGION 1..452: note = XENP32250 Chain 1 - MESO-B[MSLN]_H0_IgG1_pI(-)_Isosteric_A_ /PVA_/S267K/L368D/K370S

SEQ ID NO: 556 - protein, 721 aa, synthetic construct
  REGION 1..721: note = Description of Artificial Sequence: Synthetic polypeptide
  REGION 1..721: note = XENP32250 Chain 2 - MESO-B[MSLN]_H0_(G4S)2_[CD3]_L1.47_H1.8 9_scFv(GKPGS)4_Fc(216)_IgG1_C220S/PVA_/S267K/S364K/E357Q
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 557 - protein, 221 aa, synthetic construct; identical to SEQ ID NO: 240
  REGION 1..221: note = Description of Artificial Sequence: Synthetic polypeptide
  REGION 1..221: note = XENP32250 Chain 3 - MESO-B[MSLN]_L0 Light Chain

SEQ ID NO: 558 - protein, 449 aa, synthetic construct; identical to SEQ ID NO: 288
  REGION 1..449: note = Description of Artificial Sequence: Synthetic polypeptide
  REGION 1..449: note = XENP32251 Chain 1 - MESO-C[MSLN]_H1_IgG1_pI(-)_Isosteric_A_ /PVA_/S267K/L368D/K370S

SEQ ID NO: 559 - protein, 718 aa, synthetic construct
  REGION 1..718: note = Description of Artificial Sequence: Synthetic polypeptide
  REGION 1..718: note = XENP32251 Chain 2 - MESO-C[MSLN]_H1_[CD3]_L1.47_H1.89_scFv( GKPGS)4_Fc(216)_IgG1_C220S/PVA_/S267K/S364K/E357Q
QVQLVQSGAEVKKPGASVKVSCKASGYTFTSYWMHWVRQAPGQGLEWMGMIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 560 - protein, 214 aa, synthetic construct; identical to SEQ ID NO: 250
  REGION 1..214: note = Description of Artificial Sequence: Synthetic polypeptide
  REGION 1..214: note = XENP32251 Chain 3 - MESO-C[MSLN]_L1 Light Chain

SEQ ID NO: 561 - protein, 449 aa, synthetic construct; identical to SEQ ID NO: 288
  REGION 1..449: note = Description of Artificial Sequence: Synthetic polypeptide
  REGION 1..449: note = XENP32746 Chain 1 - MESO-C[MSLN]_H1_IgG1_pI(-)_Isosteric_A_ /PVA_/S267K/L368D/K370S

SEQ ID NO: 562 - protein, 718 aa, synthetic construct; identical to SEQ ID NO: 559
  REGION 1..718: note = Description of Artificial Sequence: Synthetic polypeptide
  REGION 1..718: note = XENP32746 Chain 2 - MESO-C[MSLN]_H1_(G4S)2_[CD3]_L1.47_H1.8 9_scFv(GKPGS)4_Fc(216)_IgG1_C220S/PVA_/S267K/S364K/E357Q

SEQ ID NO: 563 - protein, 214 aa, synthetic construct; identical to SEQ ID NO: 326
  REGION 1..214: note = Description of Artificial Sequence: Synthetic polypeptide
  REGION 1..214: note = XENP32746 Chain 3 - MESO-C[MSLN]_L1.3 Light Chain

SEQ ID NO: 564 - protein, 449 aa, synthetic construct; identical to SEQ ID NO: 288
  REGION 1..449: note = Description of Artificial Sequence: Synthetic polypeptide
  REGION 1..449: note = XENP32749 Chain 1 - MESO-C[MSLN]_H1_IgG1_pI(-)_Isosteric_A_ /PVA_/S267K/L368D/K370S

SEQ ID NO: 565 - protein, 718 aa, synthetic construct; identical to SEQ ID NO: 559
  REGION 1..718: note = Description of Artificial Sequence: Synthetic polypeptide
  REGION 1..718: note = XENP32749 Chain 2 - MESO C[MSLN]_H1_(G4S)2_[CD3]_L1.47_H1.8 9_scFv(GKPGS)4_Fc(216)_IgG1_C220S/PVA_/S267K/S364K/E357Q

SEQ ID NO: 566 - protein, 214 aa, synthetic construct; identical to SEQ ID NO: 329
  REGION 1..214: note = Description of Artificial Sequence: Synthetic polypeptide
  REGION 1..214: note = XENP32749 Chain 3 - MESO-C[MSLN]_L1.4 Light Chain

SEQ ID NO: 567 - protein, 449 aa, synthetic construct; identical to SEQ ID NO: 288
  REGION 1..449: note = Description of Artificial Sequence: Synthetic polypeptide
  REGION 1..449: note = XENP32752 Chain 1 - MESO-C[MSLN]_H1_IgG1_pI(-)_Isosteric_A_ /PVA_/S267K/L368D/K370S

SEQ ID NO: 568 - protein, 718 aa, synthetic construct; identical to SEQ ID NO: 559
  REGION 1..718: note = Description of Artificial Sequence: Synthetic polypeptide
  REGION 1..718: note = XENP32752 Chain 2 - MESO-C[MSLN]_H1_(G4S)2_[CD3]_L1.47_H1.8 9_scFv(GKPGS)4_Fc(216)_IgG1_C220S/PVA_/S267K/S364K/E357Q

SEQ ID NO: 569 - protein, 214 aa, synthetic construct; identical to SEQ ID NO: 332
  REGION 1..214: note = Description of Artificial Sequence: Synthetic polypeptide
  REGION 1..214: note = XENP32752 Chain 3 - MESO-C[MSLN]_L1.5 Light Chain

SEQ ID NO: 570 - protein, 449 aa, synthetic construct; identical to SEQ ID NO: 288
  REGION 1..449: note = Description of Artificial Sequence: Synthetic polypeptide
  REGION 1..449: note = XENP32853 Chain 1 - MESO-C[MSLN]_H1_IgG1_pI(-)_Isosteric_A_ /PVA_/S267K/L368D/K370S

SEQ ID NO: 571 - protein, 718 aa, synthetic construct; identical to SEQ ID NO: 559
  REGION 1..718: note = Description of Artificial Sequence: Synthetic polypeptide
  REGION 1..718: note = XENP32853 Chain 2 - MESO-C[MSLN]_H1_(G4S)2_[CD3]_L1.47_H1.8 9_scFv(GKPGS)4_Fc(216)_IgG1_C220S/PVA_/S267K/S364K/E357Q

SEQ ID NO: 572 - protein, 214 aa, synthetic construct; identical to SEQ ID NO: 344
  REGION 1..214: note = Description of Artificial Sequence: Synthetic polypeptide
  REGION 1..214: note = XENP32853 Chain 3 - MESO-C[MSLN]_L1.9 Light Chain

SEQ ID NO: 573 - protein, 718 aa, synthetic construct
  REGION 1..718: note = Description of Artificial Sequence: Synthetic polypeptide
  REGION 1..718: note = XENP33974 Chain 1 - MESO-C[MSLN]_H1.3_IgG1_pI(-)_Isosteric_ A_PVA_/S267K/L368D/K370S
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 574 - protein, 449 aa, synthetic construct; identical to SEQ ID NO: 297
  REGION 1..449: note = Description of Artificial Sequence: Synthetic polypeptide
  REGION 1..449: note = XENP33974 Chain 2 - MESO-C[MSLN]_H1.3_(G4S)2_[CD3]_L1.47_H1 .89_scFv(GKPGS)4_Fc(216)_IgG1_PVA_/S267K/S364K/E357Q

SEQ ID NO: 575 - protein, 214 aa, synthetic construct; identical to SEQ ID NO: 365
  REGION 1..214: note = Description of Artificial Sequence: Synthetic polypeptide
  REGION 1..214: note = XENP33974 Chain 3 - MESO-C[MSLN]_L1.14 Light Chain

SEQ ID NO: 576 - protein, 718 aa, synthetic construct; identical to SEQ ID NO: 559
  REGION 1..718: note = Description of Artificial Sequence: Synthetic polypeptide
  REGION 1..718: note = XENP33975 Chain 1 - MESO-C[MSLN]_H1_IgG1_pI(-)_Isosteric_A_ PVA_/S267K/L368D/K370S

SEQ ID NO: 577 - protein, 449 aa, synthetic construct; identical to SEQ ID NO: 288
  REGION 1..449: note = Description of Artificial Sequence: Synthetic polypeptide
  REGION 1..449: note = XENP33975 Chain 2 - MESO-C[MSLN]_H1_(G4S)2_[CD3]_L1.47_H1.8 9_scFv(GKPGS)4_Fc(216)_IgG1_PVA_/S267K/S364K/E357Q

SEQ ID NO: 578 - protein, 214 aa, synthetic construct; identical to SEQ ID NO: 386
  REGION 1..214: note = Description of Artificial Sequence: Synthetic polypeptide
  REGION 1..214: note = XENP33975 Chain 3 - MESO-C[MSLN]_L1.20 Light Chain

SEQ ID NO: 579 - protein, 718 aa, synthetic construct
  REGION 1..718: note = Description of Artificial Sequence: Synthetic polypeptide
  REGION 1..718: note = XENP33976 Chain 1 - MESO-C[MSLN]_H1.12_IgG1_pI(-)_Isosteric _A_PVA_/S267K/L368D/K370S
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 580 - protein, 449 aa, synthetic construct; identical to SEQ ID NO: 354
  REGION 1..449: note = Description of Artificial Sequence: Synthetic polypeptide
  REGION 1..449: note = XENP33976 Chain 2 - MESO-C[MSLN]_H1.12_(G4S)2_[CD3]_L1.47_H 1.89_scFv(GKPGS)4_Fc(216)_IgG1_PVA_/S267K/S364K/E357Q

SEQ ID NO: 581 - protein, 214 aa, synthetic construct; identical to SEQ ID NO: 386
  REGION 1..214: note = Description of Artificial Sequence: Synthetic polypeptide
  REGION 1..214: note = XENP33976 Chain 3 - MESO-C[MSLN]_L1.20 Light Chain

SEQ ID NO: 582 - protein, 718 aa, synthetic construct; identical to SEQ ID NO: 573
  REGION 1..718: note = Description of Artificial Sequence: Synthetic polypeptide
  REGION 1..718: note = XENP33977 Chain 1 - MESO-C[MSLN]_H1.3_IgG1_pI(-)_Isosteric_ A_PVA_/S267K/L368D/K370S

SEQ ID NO: 583 - protein, 449 aa, synthetic construct; identical to SEQ ID NO: 297
  REGION 1..449: note = Description of Artificial Sequence: Synthetic polypeptide
  REGION 1..449: note = XENP33977 Chain 2 - MESO-C[MSLN]_H1.3_(G4S)2_[CD3]_L1.47_H1 .89_scFv(GKPGS)4_Fc(216)_IgG1_PVA_/S267K/S364K/E357Q

SEQ ID NO: 584 - protein, 214 aa, synthetic construct; identical to SEQ ID NO: 362
  REGION 1..214: note = Description of Artificial Sequence: Synthetic polypeptide
  REGION 1..214: note = XENP33977 Chain 3 - MESO-C[MSLN]_L1.13 Light Chain

SEQ ID NO: 585 - protein, 718 aa, synthetic construct; identical to SEQ ID NO: 573
  REGION 1..718: note = Description of Artificial Sequence: Synthetic polypeptide
  REGION 1..718: note = XENP33978 Chain 1 - MESO-C[MSLN]_H1.3_IgG1_pI(-)_Isosteric_ A_PVA_/S267K/L368D/K370S

SEQ ID NO: 586 - protein, 449 aa, synthetic construct; identical to SEQ ID NO: 297
  REGION 1..449: note = Description of Artificial Sequence: Synthetic polypeptide
  REGION 1..449: note = XENP33978 Chain 2 - MESO-C[MSLN]_H1.3_(G4S)2_[CD3]_L1.47_H1 .89_scFv(GKPGS)4_Fc(216)_IgG1_PVA_/S267K/S364K/E357Q

SEQ ID NO: 587 - protein, 214 aa, synthetic construct; identical to SEQ ID NO: 250
  REGION 1..214: note = Description of Artificial Sequence: Synthetic polypeptide
  REGION 1..214: note = XENP33978 Chain 3 - MESO-C[MSLN]_L1 Light Chain

SEQ ID NO: 588 - protein, 718 aa, synthetic construct; identical to SEQ ID NO: 559
  REGION 1..718: note = Description of Artificial Sequence: Synthetic polypeptide
  REGION 1..718: note = XENP33979 Chain 1 - MESO-C[MSLN]_H1_IgG1_pI(-)_Isosteric_A_ PVA_/S267K/L368D/K370S

SEQ ID NO: 589 - protein, 449 aa, synthetic construct; identical to SEQ ID NO: 288
  REGION 1..449: note = Description of Artificial Sequence: Synthetic polypeptide
  REGION 1..449: note = XENP33979 Chain 2 - MESO-C[MSLN]_H1_(G4S)2_[CD3]_L1.47_H1.8 9_scFv(GKPGS)4_Fc(216)_IgG1_PVA_/S267K/S364K/E357Q

SEQ ID NO: 590 - protein, 214 aa, synthetic construct; identical to SEQ ID NO: 344
  REGION 1..214: note = Description of Artificial Sequence: Synthetic polypeptide
  REGION 1..214: note = XENP33979 Chain 3 - MESO-C[MSLN]_L1.9 Light Chain

SEQ ID NO: 591 - protein, 718 aa, synthetic construct; identical to SEQ ID NO: 579
  REGION 1..718: note = Description of Artificial Sequence: Synthetic polypeptide
  REGION 1..718: note = XENP33980 Chain 1 - MESO-C[MSLN]_H1.12_IgG1_pI(-)_Isosteric _A_PVA_/S267K/L368D/K370S

SEQ ID NO: 592 - protein, 449 aa, synthetic construct; identical to SEQ ID NO: 354
  REGION 1..449: note = Description of Artificial Sequence: Synthetic polypeptide
  REGION 1..449: note = XENP33980 Chain 2 - MESO-C[MSLN]_H1.12_(G4S)2_[CD3]_L1.47_H 1.89_scFv(GKPGS)4_Fc(216)_IgG1_PVA_/S267K/S364K/E357Q

SEQ ID NO: 593 - protein, 214 aa, synthetic construct; identical to SEQ ID NO: 344
  REGION 1..214: note = Description of Artificial Sequence: Synthetic polypeptide
  REGION 1..214: note = XENP33980 Chain 3 - MESO-C[MSLN]_L1.9 Light Chain

SEQ ID NO: 594 - protein, 449 aa, synthetic construct; identical to SEQ ID NO: 297
  REGION 1..449: note = Description of Artificial Sequence: Synthetic polypeptide
  REGION 1..449: note = XENP34151 Chain 1 - MESO-C[MSLN]_H1.3_IgG1_pI(-)_Isosteric_ A_/PVA_/S267K/L368D/K370S

SEQ ID NO: 595 - protein, 722 aa, synthetic construct
  REGION 1..722: note = Description of Artificial Sequence: Synthetic polypeptide
  REGION 1..722: note = XENP34151 Chain 2 - MESO-C[MSLN]_H1.3_(G4S)2_[CD3]_L1.47_H1 .89_scFv(GKPGS)4_Fc(216)_IgG1_C220S/PVA_/S267K/S364K/E357Q
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 596 - protein, 214 aa, synthetic construct; identical to SEQ ID NO: 365
  REGION 1..214: note = Description of Artificial Sequence: Synthetic polypeptide
  REGION 1..214: note = XENP34151 Chain 3 - MESO-C[MSLN]_L1.14 Light Chain

SEQ ID NO: 597 - protein, 449 aa, synthetic construct; identical to SEQ ID NO: 297
  REGION 1..449: note = Description of Artificial Sequence: Synthetic polypeptide
  REGION 1..449: note = XENP34152 Chain 1 - MESO-C[MSLN]_H1.3_IgG1_pI(-)_Isosteric_ A_/PVA_/S267K/L368D/K370S

SEQ ID NO: 598 - protein, 722 aa, synthetic construct; identical to SEQ ID NO: 595
  REGION 1..722: note = Description of Artificial Sequence: Synthetic polypeptide
  REGION 1..722: note = XENP34152 Chain 2 - MESO-C[MSLN]_H1.3_(G4S)2_[CD3]_L1.47_H1 .89_scFv(GKPGS)4_Fc(216)_IgG1_C220S/PVA_/S267K/S364K/E357Q

SEQ ID NO: 599 - protein, 214 aa, synthetic construct; identical to SEQ ID NO: 362
  REGION 1..214: note = Description of Artificial Sequence: Synthetic polypeptide
  REGION 1..214: note = XENP34152 Chain 3 - MESO-C[MSLN]_L1.13 Light Chain

SEQ ID NO: 600 - protein, 449 aa, synthetic construct; identical to SEQ ID NO: 297
  REGION 1..449: note = Description of Artificial Sequence: Synthetic polypeptide
  REGION 1..449: note = XENP34153 Chain 1 - MESO-C[MSLN]_H1.3_IgG1_pI(-)_Isosteric_ A_/PVA_/S267K/L368D/K370S

SEQ ID NO: 601 - protein, 722 aa, synthetic construct; identical to SEQ ID NO: 595
  REGION 1..722: note = Description of Artificial Sequence: Synthetic polypeptide
  REGION 1..722: note = XENP34153 Chain 2 - MESO-C[MSLN]_H1.3_(G4S)2_[CD3]_L1.47_H1 .89_scFv(GKPGS)4_Fc(216)_IgG1_C220S/PVA_/S267K/S364K/E357Q

SEQ ID NO: 602 - protein, 214 aa, synthetic construct; identical to SEQ ID NO: 250
  REGION 1..214: note = Description of Artificial Sequence: Synthetic polypeptide
  REGION 1..214: note = XENP34153 Chain 3 - MESO-C[MSLN]_L1 Light Chain

SEQ ID NO: 603 - protein, 449 aa, synthetic construct; identical to SEQ ID NO: 354
  REGION 1..449: note = Description of Artificial Sequence: Synthetic polypeptide
  REGION 1..449: note = XENP34154 Chain 1 - MESO-C[MSLN]_H1.12_IgG1_pI(-)_Isosteric _A_/PVA_/S267K/L368D/K370S

SEQ ID NO: 604 - protein, 722 aa, synthetic construct
  REGION 1..722: note = Description of Artificial Sequence: Synthetic polypeptide
  REGION 1..722: note = XENP34154 Chain 2 - MESO-C[MSLN]_H1.12_(G4S)2_[CD3]_L1.47_H 1.89_scFv(GKPGS)4_ Fc(216)_IgG1_C220S/PVA_/S267K/S364K/E357Q
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 605 - protein, 214 aa, synthetic construct; identical to SEQ ID NO: 344
  REGION 1..214: note = Description of Artificial Sequence: Synthetic polypeptide
  REGION 1..214: note = XENP34154 Chain 3 - MESO-C[MSLN]_L1.9 Light Chain

SEQ ID NO: 606 - protein, 449 aa, synthetic construct; identical to SEQ ID NO: 534
  REGION 1..449: note = Description of Artificial Sequence: Synthetic polypeptide
  REGION 1..449: note = XENP34159 Chain 1 - MESO-C[MSLN]_H1.3_IgG1_pI(-)_Isosteric_ A_/PVA_/S267K/L368D/K370S/M248L/N434S

SEQ ID NO: 607 - protein, 722 aa, synthetic construct
  REGION 1..722: note = Description of Artificial Sequence: Synthetic polypeptide
  REGION 1..722: note = XENP34159 Chain 2 - MESO-C[MSLN]_H1.3_(G4S)2_[CD3]_L1.47_H1 .89_scFv(GKPGS)4_F c(216)_IgG1_C220S/PVA_/S267K/S364K/E357Q/ M248L/N434S
QVQLVQSGAEVKKPGASVKVSCKASGYTFTSYWMHWVRQAPGQGLEWMGWIHPNSDNTIYYEKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 608 - protein, 214 aa, synthetic construct; identical to SEQ ID NO: 365
  REGION 1..214: note = Description of Artificial Sequence: Synthetic polypeptide
  REGION 1..214: note = XENP34159 Chain 3 - MESO-C[MSLN]_L1.14 Light Chain

SEQ ID NO: 609 - protein, 449 aa, synthetic construct; identical to SEQ ID NO: 534
  REGION 1..449: note = Description of Artificial Sequence: Synthetic polypeptide
  REGION 1..449: note = XENP34160 Chain 1 - MESO-C[MSLN]_H1.3_IgG1_pI(-)_Isosteric_ A_/PVA_/S267K/L368D/K370S/M248L/N434S

SEQ ID NO: 610 - protein, 722 aa, synthetic construct; identical to SEQ ID NO: 607
  REGION 1..722: note = Description of Artificial Sequence: Synthetic polypeptide
  REGION 1..722: note = XENP34160 Chain 2 - MESO-C[MSLN]_H1.3_(G4S)2_[CD3]_L1.47_H1 .89_scFv(GKPGS)4_F c(216)_IgG1_C220S/PVA_/S267K/S364K/E357Q/ M248L/N434S

SEQ ID NO: 611 - protein, 214 aa, synthetic construct; identical to SEQ ID NO: 362
  REGION 1..214: note = Description of Artificial Sequence: Synthetic polypeptide
  REGION 1..214: note = XENP34160 Chain 3 - MESO-C[MSLN]_L1.13 Light Chain

SEQ ID NO: 612 - protein, 449 aa, synthetic construct; identical to SEQ ID NO: 534
  REGION 1..449: note = Description of Artificial Sequence: Synthetic polypeptide
  REGION 1..449: note = XENP34161 Chain 1 - MESO-C[MSLN]_H1.3_IgG1_pI(-)_Isosteric_ A_/PVA_/S267K/L368D/K370S/M248L/N434S

SEQ ID NO: 613 - protein, 722 aa, synthetic construct; identical to SEQ ID NO: 607
  REGION 1..722: note = Description of Artificial Sequence: Synthetic polypeptide
  REGION 1..722: note = XENP34161 Chain 2 - MESO-C[MSLN]_H1.3_(G4S)2_[CD3]_L1.47_H1 .89_scFv(GKPGS)4_F c(216)_IgG1_C220S/PVA_/S267K/S364K/E357Q/ M248L/N434S

SEQ ID NO: 614 - protein, 214 aa, synthetic construct; identical to SEQ ID NO: 250
  REGION 1..214: note = Description of Artificial Sequence: Synthetic polypeptide
  REGION 1..214: note = XENP34161 Chain 3 - MESO-C[MSLN]_L1 Light Chain

SEQ ID NO: 615 - protein, 449 aa, synthetic construct; identical to SEQ ID NO: 543
  REGION 1..449: note = Description of Artificial Sequence: Synthetic polypeptide
  REGION 1..449: note = XENP34162 Chain 1 - MESO-C[MSLN]_H1.12_IgG1_pI(-)_Isosteric _A_/PVA_/S267K/L368D/K370S/M248L/N434S

SEQ ID NO: 616 - protein, 722 aa, synthetic construct
  REGION 1..722: note = Description of Artificial Sequence: Synthetic polypeptide
  REGION 1..722: note = XENP34162 Chain 2 - MESO-C[MSLN]_H1.12_(G4S)2_[CD3]_L1.47_H 1.89_scFv(GKPGS)4_ Fc(216)_IgG1_C220S/PVA_/S267K/S364K/E357Q /M248L/N434S
QVQLVQSGAEVKKPGASVKVSCKASGYTFTSYWMHWVRQAPGQGLEWMGMIHPNSDNTIYYQKFQGRVTMTVDKSISTAYMELSRLRSDDTAVYYCAIIITPVVPKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 617 - protein, 214 aa, synthetic construct; identical to SEQ ID NO: 344
  REGION 1..214: note = Description of Artificial Sequence: Synthetic polypeptide
  REGION 1..214: note = XENP34162 Chain 3 - MESO-C[MSLN]_L1.9 Light Chain

SEQ ID NO: 618 - protein, 5 aa, synthetic construct
  REGION 1..5: note = Description of Artificial Sequence: Synthetic peptide
  REGION 1..5: note = See specification as filed for detailed description of substitutions and preferred embodiments
GGGGS

(19) United States
(12) Patent Application Publication
(10) Pub. No.: US 2024/0279356 A1
(43) Pub. Date: Aug. 22, 2024

(54) HETERODIMERIC ANTIBODIES THAT BIND MSLN AND CD3

(71) Applicant: Xencor, Inc., Pasadena, CA (US)

(72) Inventors: John R. Desjarlais, Pasadena, CA (US); Matthew Bernett, Monrovia, CA (US); Michael Hedvat, Encino, CA (US); Veronica Gusti Zeng, Monrovia, CA (US); Matthew S. Faber, Monrovia, CA (US)

(21) Appl. No.: 18/652,669

(22) Filed: May 1, 2024

Related U.S. Application Data
(63) Continuation of application No. 17/321,272, filed on May 14, 2021, now abandoned.
(60) Provisional application No. 63/025,021, filed on May 14, 2020.

(51) Int. Cl. C07K 16/30; A61K 39/00; C07K 16/28

(52) U.S. Cl. CPC C07K 16/3069; C07K 16/2809; A61K 39/00; C07K 2317/31; C07K 2317/56; C07K 2317/565; C07K 2317/622

(57) ABSTRACT

The present invention is directed to antibodies, including novel antigen binding domains and heterodimeric antibodies, that bind Mesothelin (MSLN).

### PRIORITY

This application is a continuation of U.S. patent application Ser. No. 17/321,272, filed May 14, 2021, which claims priority to U.S. Provisional Patent Application No. 63/025,021, filed May 14, 2020, which is hereby incorporated by reference in its entirety.

### SEQUENCE LISTING INCORPORATION PARAGRAPH

The instant application contains a Sequence Listing which has been submitted electronically in XML format and is hereby incorporated by reference in its entirety. Said XML file, created on May 1, 2024, is named 067461-5268-US01.xml and is 1,210,988 bytes in size.

### BACKGROUND

Antibody-based therapeutics have been used successfully to treat a variety of diseases, including cancer. An increasingly prevalent avenue being explored is the engineering of single immunoglobulin molecules that co-engage two different antigens. Such alternate antibody formats that engage two different antigens are often referred to as bispecific antibodies. Because the considerable diversity of the antibody variable region (Fv) makes it possible to produce an Fv that recognizes virtually any molecule, the typical approach to bispecific antibody generation is the introduction of new variable regions into the antibody.

A particularly useful approach for bispecific antibodies is to engineer a first binding domain which engages CD3 and a second binding domain which engages an antigen associated with or upregulated on cancer cells so that the bispecific antibody redirects CD3⁺ T cells to destroy the cancer cells. Mesothelin (MSLN) has previously been reported to be highly expressed in ovarian tumors. Current treatment options for ovarian cancer have only modest efficacy and there remains a large unmet need for new targeted therapies. The present invention provides novel bispecific antibodies to CD3 and MSLN that are capable of localizing CD3⁺ effector T cells to MSLN expressing tumors such as in ovarian cancer.

### BRIEF SUMMARY

Accordingly, provided herein are MSLN antigen binding domains and anti-MSLN antibodies (e.g., bispecific antibodies).

In one aspect, provided herein is a composition that includes a Mesothelin (MSLN) binding domain that includes the variable heavy complementary determining regions 1-3 (vhCDR1-3) and the variable light complementary determining regions (vlCDR1-3) of any of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In another aspect, provided herein is a composition that includes a Mesothelin (MSLN) binding domain that includes a variable heavy domain and a variable light domain of any of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In another aspect, the present invention provides a composition that includes a Mesothelin (MSLN) binding domain selected from the following MSLN binding domains: MESO-A[MSLN]_H1.1_L1, MESO-B[MSLN]_H0L0, MESO-C[MSLN]_HIL1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In another aspect, the present invention provides a nucleic acid composition that includes: a) a first nucleic acid encoding a variable heavy domain that includes the variable heavy complementary determining regions 1-3 (vhCDR1-3) of a MSLN binding domain; and b) a second nucleic acid encoding a variable light domain that includes the variable light complementary determining regions 1-3 (vlCDR1-3) of the MSLN binding domain, wherein the MSLN binding domain is one of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In another aspect, the present invention provides a nucleic acid composition that includes: a) a first nucleic acid encoding a variable heavy domain that includes the variable heavy domain of a MSLN binding domain; and b) a second nucleic acid encoding a variable light domain that includes the variable light domain of the MSLN binding domain, wherein the MSLN binding domain is any one of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In some embodiments, the present invention provides an expression vector composition that includes: a) a first expression vector that includes the first nucleic acid b) a second expression vector that includes a second nucleic acid. In further embodiments, the present invention provides a host cell that includes the expression vector composition.

In some embodiments, the present invention provides a method of making a Mesothelin (MSLN) binding domain that includes culturing the host cell under conditions wherein the MSLN binding domain is expressed, and recovering the MSLN binding domain.

In another aspect, the present invention provides an anti-MSLN antibody that includes an Mesothelin (MSLN) binding domain, the MSLN binding domain includes the variable heavy complementary determining regions 1-3 (vhCDR1-3) and the variable light complementary determining regions (vlCDR1-3) of any of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In some embodiments, the vhCDR1-3 and vlCDR1-3 are selected from the vhCDR1-3 and vlCDR1-3 of any of the following MSLN binding domains in FIGS. 15-21.

In another aspect, the present invention provides an anti-MSLN antibody that includes an Mesothelin (MSLN) binding domain, the MSLN binding domain includes a variable heavy domain and a variable light domain of any of the following MSLN binding domains: MESO-A[MSLN]_H1.1_L1, MESO-B[MSLN]_H0L0, MESO-C[MSLN]_HIL1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In another aspect, provided herein is an anti-MSLN antibody that includes an Mesothelin (MSLN) binding domain selected from any one of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21). In some embodiments, the antibody includes: a) a first monomer that includes a first antigen binding domain and a first constant domain; and b) a second monomer that includes a second antigen binding domain and a second constant domain, wherein either of the first antigen binding domain or second antigen binding domain is the MSLN binding domain. In further embodiments, first antigen binding domain and the second antigen binding domain bind different antigens. In further embodiments, the first antigen binding domain is the MSLN binding domain and the second antigen binding domain is a CD3 binding domain. In further embodiments, the CD3 binding domain includes the vhCDR1-3, and vlCDR1-3 of any of the following CD3 binding domains: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F). In further embodiments, the vhCDR1-3 and vlCDR1-3 of the CD3 binding domain are selected from the vhCDR1-3 and vlCDR1-3 in FIGS. 10A-10F.

In some embodiments, the CD3 binding domain includes the variable heavy domain and variable light domain of any of the following CD3 binding domains: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

In some embodiments, the CD3 binding domain is an anti-CD3 scFv.

In some embodiments, wherein the first and second constant domains each includes CH2-CH3.

In some embodiments, the first and second constant domains each includes CH1-hinge-CH2-CH3.

In some embodiments, the first and second constant domains each are a variant constant domain.

In some embodiments, the first and second monomers include a set of heterodimerization variants are any one of the variants depicted in FIGS. 1A-1E. In some embodiments, the set of heterodimerization variants includes one of the follow set of variants: S364K/E357Q: L368D/K370S; S364K: L368D/K370S; S364K: L368E/K370S; D401K: T411E/K360E/Q362E; and T366W: T366S/L368A/Y407V.

In some embodiments, the first and second monomers each further include an ablation variant. In further embodiments, the ablation variant is E233P/L234V/L235A/G236del/S267K.

In some embodiments, the at least one of the first or second monomer further includes a pI variant. In further embodiments, the pI variant is N208D/Q295E/N384D/Q418E/N421D. In some embodiments, the scFv includes a charged scFv linker.

In some embodiments, the present invention provides a nucleic acid composition including nucleic acids encoding the anti-MSLN. In some embodiments, the composition including nucleic acids encoding first and second monomers. In some embodiments, the present invention provides expression vectors that include the nucleic acids. In some embodiments, the present invention provides a host cell transformed with the expression vector.

In some embodiments, the present invention provides a method of making an anti-MSLN antibody according to any one of claims B1 to B21. The method includes culturing the host cell according to claim B25 under conditions wherein the anti-MSLN antibody is expressed, and recovering the anti-MSLN antibody. In some embodiments, the present invention provides a method of treating a cancer that includes administering to a patient in need thereof the antibody.

In another aspect, the present invention provides a heterodimeric antibody that includes: a) a first monomer that includes: i) an anti-CD3 scFv that includes a first variable light domain, an scFv linker and a first variable heavy domain; and ii) a first Fc domain, wherein the scFv is covalently attached to the N-terminus of the first Fc domain using a domain linker; b) a second monomer that includes a VH2-CH1-hinge-CH2-CH3 monomer, wherein VH is a second variable heavy domain and CH2-CH3 is a second Fc domain; and c) a light chain that includes a second variable light domain, wherein the second variable heavy domain and the second variable light domain form a MSLN binding domain.

In some embodiments, the MSLN binding domain includes the vhCDR1-3 and vlCDR1-3 of any of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In some embodiments, the vhCDR1-3 and vlCDR1-3 of the MSLN binding domain are selected from the vhCDR1-3 and vlCDR1-3 sequences of the MSLN binding domains provided in FIGS. 15-21.

In some embodiments, the second heavy variable domain includes a heavy variable domain and the second light variable domain includes a variable light domain of any of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In some embodiments, the anti-CD3 scFv includes the vhCDR1-3 and the vlCDR1-3 of any of the following CD3 binding domains: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

In some embodiments, the vhCDR1-3 and vlCDR1-3 of the anti-CD3 scFv are selected from the vhCDR1-3 and vlCDR1-3 in FIGS. 10A-10F.

In some embodiments, the anti-CD3 scFv includes the variable heavy domain and variable light domain of any of the following CD3 binding domains: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

In some embodiments, the first variable light domain is covalently attached to the N-terminus of the first Fc domain using a domain linker.

In some embodiments, the first variable heavy domain is covalently attached to the N-terminus of the first Fc domain using a domain linker.

In some embodiments, the scFv linker is a charged scFv linker.

In some embodiments, the first and second Fc domains are variant Fc domains.

In some embodiments, the first and second monomers includes a set of heterodimerization variants selected from any of the heterodimerization variants in FIGS. 1A-1E. In some embodiments, the set of heterodimerization variants selected is from following: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering.

In some embodiments, the first and second monomers further includes an ablation variant. In some embodiments, the ablation variant is E233P/L234V/L235A/G236del/S267K, wherein numbering is according to EU numbering.

In some embodiments, one of the first or second monomer includes a pI variant.

In some embodiments, the pI variant is N208D/Q295E/N384D/Q418E/N421D, wherein numbering is according to EU numbering.

In some embodiments, the first monomer includes amino acid variants S364K/E357Q/E233P/L234V/L235A/G236del/S267K, wherein the second monomer includes amino acid variants
L368D/K370S/N208D/Q295E/N384D/Q418E/N421D/E233P/L234V/L235A/G236del/S267 K, and wherein numbering is according to EU numbering.

In some embodiments, the scFv linker is a charged scFv linker having the amino acid sequence (GKPGS)₄ (SEQ ID NO: 1).

In some embodiments, the first and second monomers each further include amino acid variants 428/434S.

In some embodiments, the heterodimeric antibody includes the following heterodimeric antibodies: XENP16248, XENP16833, XENP31697, XENP32550, XENP32551, XENP32552, XENP32553, XENP32554, XENP32555, XENP32556, XENP32557, XENP32558, XENP32559, XENP32560, XENP32561, XENP32562, XENP32563, XENP32564, XENP32565, XENP32566, XENP32567, XENP32568, XENP32569, XENP32570, XENP32937, XENP32938, XENP32939, XENP32940, XENP32941, XENP32942, XENP32943, XENP32944, XENP32945, XENP32946, XENP33888, XENP33889, XENP30764, XENP30765, XENP31701, XENP33969, XENP33971, XENP33973, XENP16251, XENP16834, XENP16252, and XENP16835 (FIGS. 23-27).

In another aspect, the present invention provides a heterodimeric antibody that includes: a) a first monomer that includes from N-terminus to C-terminus, a scFv-linker-CH2-CH3, wherein scFv is an anti-CD3 scFV and CH2-CH3 is a first Fc domain; b) a second monomer that includes from N-terminus to C-terminus a VH-CH1-hinge-CH2-CH3, wherein CH2-CH3 is a second Fc domain; and c) a light chain that includes a VL-CL; wherein the first variant Fc domain includes amino acid variants S364K/E357Q, wherein the second variant Fc domain includes amino acid variants L368D/K370S, wherein the first and second variant Fc domains each include amino acid variants E233P/L234V/L235A/G236del/S267K, wherein the hinge-CH2-CH3 of the second monomer includes amino acid variants N208D/Q295E/N384D/Q418E/N421D, wherein the VH and VL form a MSLN binding domain that includes the variable heavy domain and the variable light domain, respectively, of a MSLN binding domain selected from: MESO-A[MSLN]_H1.1_L1, MESO-B[MSLN]_H0L0, MESO-C[MSLN]_HIL1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-MESO-C[MSLN]_H1.12_L1.21, MESO-C[MSLN]_H1.12_L1.22, and MESO-C[MSLN]_H1.12_L1.9 (FIGS. 15-21), wherein the anti-CD3 scFv includes the variable heavy domain and the variable light domain of a CD3 binding domain selected from H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F), and wherein numbering is according to EU numbering.

In some embodiments, the scFv includes a charged scFv linker having the amino acid sequence (GKPGS)₄ (SEQ ID NO: 1).

In some embodiments, the first and second variant Fc domains each further include amino acid variants 428/434S, wherein numbering is according to EU numbering.

In some embodiments, the present invention provides a nucleic acid composition that includes nucleic acids encoding the first and second monomers and the light chain of the antibody.

In some embodiments, the present invention provides an expression vector that includes the nucleic acids. In some embodiments, the present invention provides a host cell transformed with the expression vector.

In some embodiments, the present invention provides a method of treating a MSLN associated cancer that includes administering to a patient in need thereof any one of the antibodies provided herein.

In another aspect, the present invention provides a heterodimeric antibody that includes: a) a first monomer that includes from N-terminus to C-terminus, a VH1-CH1-linker 1-scFv-linker 2-CH2-CH3, wherein VH1 is a first variable heavy domain, scFv is an anti-CD3 scFV, linker 1 and linker 2 are a first domain linker and second domain linker, respectively, and CH2-CH3 is a first Fc domain; b) a second monomer that includes from N-terminus to C-terminus a VH2-CH1-hinge-CH2-CH3, wherein VH2 is a second variable heavy domain and CH2-CH3 is a second Fc domain; and c) a common light chain that includes a variable light domain; wherein the first variable heavy domain and the variable light domain form a first MSLN binding domain, and the second variable heavy domain and the variable light domain form a second MSLN binding domain.

In some embodiments, the first and second MSLN binding domains each includes the vhCDR1-3 and vlCDR1-3 of any of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21),

In some embodiments, the vhCDR1-3 and vlCDR1-3 of the first and second MSLN binding domains are selected from the vhCDR1-3 and vlCDR1-3 provided in FIGS. 15-21.

In some embodiments, the first and second variable heavy domain each include a variable heavy domain of a MSLN binding domain, and the first and second variable light domain each include a variable light domain of the MSLN binding domain, wherein the MSLN binding domain is any of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20,_MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In some embodiments, the scFv includes the vhCDR1-3 and the vlCDR1-3 of any of the following CD3 binding domains: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

In some embodiments, the vhCDR1-3 and vlCDR1-3 of the scFv are selected from the vhCDR1-3 and vlCDR1-3 in FIGS. 10A-10F.

In some embodiments, the scFv includes the variable heavy domain and variable light domain of any of the following CD3 binding domains: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

In some embodiments, the scFv includes an scFv variable heavy domain, an scFv variable light domain and an scFv linker that connects the scFv variable heavy domain and the scFv variable light domain.

In some embodiments, the scFv variable heavy domain is attached to the C-terminus of the CH1 of the first monomer using the first domain linker and the scFv variable light domain is covalently attached to the N-terminus of the first Fc domain using the second domain linker.

In some embodiments, the scFv variable light domain is attached to the C-terminus of the CH1 of the first monomer using the first domain linker and the scFv variable heavy domain is covalently attached to the N-terminus of the first Fc domain using the second domain linker.

In some embodiments, the scFv linker is a charged scFv linker.

In some embodiments, the first and second Fc domains are variant Fc domains.

In some embodiments, the first and second monomers includes a set of heterodimerization variants selected from those depicted in FIGS. 1A-1E.

In some embodiments, the set of heterodimerization variants selected is from the following: S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering.

In some embodiments, the first and second monomers further include an ablation variant.

In some embodiments, the ablation variant is E233P/L234V/L235A/G236del/S267K, wherein numbering is according to EU numbering.

In some embodiments, one of the first or second monomer further includes a pI variant.

In some embodiments, the pI variant is N208D/Q295E/N384D/Q418E/N421D, wherein numbering is according to EU numbering.

In some embodiments, first variant Fc domain includes amino acid variants S364K/E357Q/E233P/L234V/L235A/G236del/S267K, wherein the second variant Fc domain includes amino acid variants
L368D/K370S/N208D/Q295E/N384D/Q418E/N421D/E233P/L234V/L235A/G236del/S267 K, and wherein numbering is according to EU numbering.

In some embodiments, the scFv linker is a charged scFv linker having the amino acid sequence (GKPGS)₄ (SEQ ID NO: 1).

In some embodiments, the first and second variant Fc domains each further include amino acid variants 428/434S, wherein numbering is according to EU numbering.

In some embodiments, the heterodimeric antibody includes the following heterodimeric antibodies: XENP31063, XENP31067, XENP31705, XENP31451, XENP31452, XENP31713, XENP31064, XENP31068, XENP31709, ENP32744, XENP32747, XENP32750, XENP32851, XENP31453, XENP31454, XENP31717, XENP32745, XENP32748, XENP32751, XENP32852, XENP32947, XENP32948, XENP32949, XENP32950, XENP32951, XENP33366, XENP33367, XENP33460, XENP33461, XENP33462, XENP33463, XENP33464, XENP33465, XENP33466, XENP33467, XENP33968, XENP33970, XENP33972, XENP34155, XENP34156, XENP34157, XENP34158, XENP31455, XENP31456, XENP32249, XENP32250, XENP32251, XENP32746, XENP32749, XENP32752, XENP32853, XENP33974, XENP33975, XENP33976, XENP33977, XENP33978, XENP33979, XENP33980, XENP34151, XENP34152, XENP34153, XENP34154, XENP34159, XENP34160, XENP34161, and XENP34162 (FIGS. 28-32).

In another aspect, the heterodimeric antibody includes: a) a first monomer that includes from N-terminus to C-terminus, a VH1-CH1-linker 1-scFv-linker 2-CH2-CH3, wherein scFv is an anti-CD3 scFV and CH2-CH3 is a first Fc domain; b) a second monomer that includes from N-terminus to C-terminus a VH1-CH1-hinge-CH2-CH3, wherein CH2-CH3 is a second Fc domain; and c) a common light chain that includes VL-CL; wherein the first variant Fc domain includes amino acid variants S364K/E357Q, wherein the second variant Fc domain includes amino acid variants L368D/K370S, wherein the first and second variant Fc domains each include amino acid variants E233P/L234V/L235A/G236del/S267K, wherein the hinge-CH2-CH3 of the second monomer includes amino acid variants N208D/Q295E/N384D/Q418E/N421D, wherein said VH and VL include the variable heavy domain and the variable light domain of a MSLN binding domain selected from: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21), wherein the anti-CD3 scFv includes the variable heavy domain and the variable light domain of a CD3 binding domain selected from H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F), and wherein numbering is according to EU numbering.

In some embodiments, the scFv includes a charged scFv linker having the amino acid sequence (GKPGS)₄ (SEQ ID NO: 1).

In some embodiments, the first and second variant Fc domains each further include amino acid variants 428/434S.

In some embodiments, the first and second monomers and the common light chain of the antibody XENP34156. In some embodiments, the present invention provides an expression vector that includes the nucleic acids. In some embodiments, the present invention provides a host cell transformed with the expression vector. In some embodiments, the present invention provides treating a MSLN associated cancer includes administering to a patient in need thereof the antibody.

In another aspect, the present invention provides a heterodimeric antibody including the following heterodimeric antibodies: XENP16248, XENP16833, XENP31697, XENP32550, XENP32551, XENP32552, XENP32553, XENP32554, XENP32555, XENP32556, XENP32557, XENP32558, XENP32559, XENP32560, XENP32561, XENP32562, XENP32563, XENP32564, XENP32565, XENP32566, XENP32567, XENP32568, XENP32569, XENP32570, XENP32937, XENP32938, XENP32939, XENP32940, XENP32941, XENP32942, XENP32943, XENP32944, XENP32945, XENP32946, XENP33888, XENP33889, XENP30764, XENP30765, XENP31701, XENP33969, XENP33971, XENP33973, XENP16251, XENP16834, XENP16252, and XENP16835 (FIGS. 23-27).

In another aspect, the present invention provides a heterodimeric antibody including the following heterodimeric antibodies: XENP31063, XENP31067, XENP31705, XENP31451, XENP31452, XENP31713, XENP31064, XENP31068, XENP31709, ENP32744, XENP32747, XENP32750, XENP32851, XENP31453, XENP31454, XENP31717, XENP32745, XENP32748, XENP32751, XENP32852, XENP32947, XENP32948, XENP32949, XENP32950, XENP32951, XENP33366, XENP33367, XENP33460, XENP33461, XENP33462, XENP33463, XENP33464, XENP33465, XENP33466, XENP33467, XENP33968, XENP33970, XENP33972, XENP34155, XENP34156, XENP34157, XENP34158, XENP31455, XENP31456, XENP32249, XENP32250, XENP32251, XENP32746, XENP32749, XENP32752, XENP32853, XENP33974, XENP33975, XENP33976, XENP33977, XENP33978, XENP33979, XENP33980, XENP34151, XENP34152, XENP34153, XENP34154, XENP34159, XENP34160, XENP34161, and XENP34162 (FIGS. 28-32).

In some embodiments, the present invention provides nucleic acid composition that includes the nucleic acids encoding the heterodimeric antibody. In some embodiments, the present invention provides an expression vector includes the nucleic acids. In some embodiments, the present invention provides a host cell transformed with the expression vector.

In some embodiments, the present method provides a method of treating a MSLN related cancer that includes administering to a patient in need thereof any one of the heterodimeric antibodies provided herein.

### BRIEF DESCRIPTION OF THE DRAWINGS

FIG. 1A-1E depict useful pairs of Fc heterodimerization variant sets (including skew and pI variants). There are variants for which there are no corresponding “monomer 2” variants; these are pI variants which can be used alone on either monomer.

FIG. 2 depicts a list of isosteric variant antibody constant regions and their respective substitutions. pI_(−) indicates lower pI variants, while pI_(+) indicates higher pI variants. These can be optionally and independently combined with other heterodimerization variants of the inventions (and other variant types as well, as outlined herein.)

FIG. 3 depicts useful ablation variants that ablate FcγR binding (sometimes referred to as “knock outs” or “KO” variants). Generally, ablation variants are found on both monomers, although in some cases they may be on only one monomer.

FIG. 4 depicts particularly useful embodiments of “non-Fv” components of the antibodies described herein.

FIG. 5 depicts a number of charged scFv linkers that find use in increasing or decreasing the pI of the subject heterodimeric bsAbs that utilize one or more scFv as a component, as described herein. The (+H) positive linker finds particular use herein, particularly with anti-CD3 V_{L} and VH sequences shown herein. A single prior art scFv linker with a single charge is referenced as “Whitlow”, from Whitlow et al., Protein Engineering 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab₂-scFv-Fc formats).

FIG. 6 depicts a number of exemplary domain linkers. In some embodiments, these linkers find use linking a single-chain Fv to an Fc chain. In some embodiments, these linkers may be combined. For example, a GGGGS linker (SEQ ID NO: 2) may be combined with a “half hinge” linker.

FIGS. 7A-7D depict the sequences of several useful 1+1 Fab-scFv-Fc bispecific antibody format heavy chain backbones based on human IgG1, without the Fv sequences (e.g. the scFv and the VH for the Fab side). Backbone 1 is based on human IgG1 (356E/358M allotype), and includes the S364K/E357Q: L368D/K370S skew variants, C220S on the chain with the S364K/E357Q skew variants, the N208D/Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes S364K: L368D/K370S skew variants, C220S on the chain with the S364K skew variant, the N208D/Q295E/N384D/Q418E/N421D pI variants on the chain with L368D/K370S skew variants, and the E233P/L234V/L235A/G236del/S267K ablation variants on both chains. Backbone 3 is based on human IgG1 (356E/358M allotype), and includes S364K: L368E/K370S skew variants, C220S on the chain with the S364K skew variant, the N208D/Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes D401K: K360E/Q362E/T411E skew variants, C220S on the chain with the D401K skew variant, the N208D/Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes S364K/E357Q: L368D/K370S skew variants, C220S on the chain with the S364K/E357Q skew variants, the N208D/Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes S364K/E357Q: L368D/K370S skew variants, C220S on the chain with the S364K/E357Q skew variants, N208D/Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Backbone 8 is based on human IgG4, and includes the S364K/E357Q: L368D/K370S skew variants, the N208D/Q295E/N384D/Q418E/N421D pI variants on the chain with L368D/K370S skew variants, as well as a S228P (EU numbering, this is S241P in Kabat) variant on both chains that ablates Fab arm exchange as is known in the art. Backbone 9 is based on human IgG2, and includes the S364K/E357Q: L368D/K370S skew variants, the N208D/Q295E/N384D/Q418E/N421D pI variants on the chain with L368D/K370S skew variants. Backbone 10 is based on human IgG2, and includes the S364K/E357Q: L368D/K370S skew variants, the N208D/Q295E/N384D/Q418E/N421D pI variants on the chain with L368D/K370S skew variants as well as a S267K variant on both chains. Backbone 11 is identical to backbone 1, except it includes M428L/N434S Xtend™ mutations. Backbone 12 is based on human IgG1 (356E/358M allotype), and includes S364K/E357Q: L368D/K370S skew variants, C220S and the P217R/P229R/N276K pI variants on the chain with S364K/E357Q skew variants and the E233P/L234V/L235A/G236del/S267K ablation variants on both chains. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIGS. 8A-8C depicts the sequences of several useful 2+1 Fab₂-scFv-Fc bispecific antibody format heavy chain backbones based on human IgG1, without the Fv sequences (e.g. the scFv and the VH for the Fab side). Backbone 1 is based on human IgG1 (356E/358M allotype), and includes the S364K/E357Q: L368D/K370S skew variants, the N208D/Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes S364K: L368D/K370S skew variants, the N208D/Q295E/N384D/Q418E/N421D pI variants on the chain with L368D/K370S skew variants, and the E233P/L234V/L235A/G236del/S267K ablation variants on both chains. Backbone 3 is based on human IgG1 (356E/358M allotype), and includes S364K: L368E/K370S skew variants, the N208D/Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes D401K: K360E/Q362E/T411E skew variants, the N208D/Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes S364K/E357Q: L368D/K370S skew variants, the N208D/Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes S364K/E357Q: L368D/K370S skew variants, N208D/Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Backbone 8 is identical to backbone 1, except it includes M428L/N434S Xtend™ mutations. Backbone 9 is based on human IgG1 (356E/358M allotype), and includes S364K/E357Q: L368D/K370S skew variants, the P217R/P229R/N276K pI variants on the chain with S364K/E357Q skew variants and the E233P/L234V/L235A/G236del/S267K ablation variants on both chains. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIG. 9 depicts the sequences of several useful constant light domain backbones based on human IgG1, without the Fv sequences (e.g. the scFv or the Fab). Included herein are constant light backbone sequences that are 90, 95, 98 and 99% identical (as defined herein) to the recited sequences, and/or contain from 1, 2, 3, 4, 5, 6, 7, 8, 9 or 10 additional amino acid modifications.

FIGS. 10A-10F depicts sequences for exemplary anti-CD3 scFvs suitable for use in the bispecific antibodies of the invention. The CDRs are underlined, the scFv linker is double underlined (in the sequences, the scFv linker is a positively charged scFv (GKPGS)₄ linker (SEQ ID NO: 1), although as will be appreciated by those in the art, this linker can be replaced by other linkers, including uncharged or negatively charged linkers, some of which are depicted in FIG. 5), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 11 depicts illustrative IHC of ovarian cancer biopsy cores and adjacent normal tissue showing mesothelin expression.

FIG. 12 depicts breakdown of IHC scores of 191 ovarian cancer biopsy cores showing mesothelin expression.

FIG. 13 depicts endogenous mesothelin expression on cancer cell lines as determined by IHC and flow.

FIGS. 14A-14B depict the antigen sequences for a number of antigens of use in the antibodies described herein, including both human and cyno, to facilitate the development of antigen binding domains that bind to both for ease of clinical development.

FIGS. 15A-15B depict the variable heavy and variable light chain sequences for an exemplary MSLN binding domain referred to herein as MESO-A, as well as the sequences for XENP16249, XENP16250, and XENP16253, anti-MSLN mAbs based on MESO-A and IgG1 backbone. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 16 depicts the variable heavy and variable light chain sequences for an exemplary MSLN binding domain referred to herein as MESO-B, as well as the sequences for XENP16827, an anti-MSLN mAb based on MESO-B and IgG1 backbone. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 17 depicts the variable heavy and variable light chain sequences for an exemplary MSLN binding domain referred to herein as MESO-C, as well as the sequences for XENP31693, an anti-MSLN mAb based on MESO-C and IgG1 backbone. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 18 depicts the variable heavy chain sequences for MESO-C variants engineered with the aim to tune binding affinity for human MSLN. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within V_{H} domains using other numbering systems. Further, as for all the sequences in the Figures, these V_{L} sequences can be used either in a scFv format or in a Fab format. Each of the variable heavy domains depicted herein can be paired with any other αMSLN variable light domain.

FIGS. 19A-19B depict the variable light chain sequences for MESO-C variants engineered with the aim to tune binding affinity for human MSLN. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within V_{L} domains using other numbering systems. Further, as for all the sequences in the Figures, these V_{L} sequences can be used either in a scFv format or in a Fab format. Each of the variable light domains depicted herein can be paired with any other αMSLN variable heavy domain.

FIG. 20 depicts BLI-response, dissociation constant (KD), association rate (kₐ), and dissociation rate (k_{d}) of affinity-engineered MESO-C variants (in the context of 1+1 Fab-scFv-Fc anti-MSLN×anti-CD3 bispecific antibodies) for human and cynomolgus MSLN as determined by Octet®. Substitutions in variable regions are based on Kabat numbering. It should be noted that for Kabat numbering, some positions are numbered using letters as well; for example, in Kabat numbering, there is one amino acid at position 100b (K100b) but a different amino acid at position 100a (P100a); that is, the inclusion of the small letters denotes a position, not a particular amino acid in that position.

FIG. 21 depicts BLI-response, dissociation constant (K_{D}), association rate (kₐ), and dissociation rate (k_{d}) of affinity-engineered MESO-C variants (in the context of 1+1 Fab-scFv-Fc anti-MSLN×anti-CD3 bispecific antibodies) for human MSLN as determined by Octet® (multiple concentration Octet® run). Substitutions in variable regions are based on Kabat numbering.

FIGS. 22A-22B depict a couple of formats of the present invention. FIG. 22A depicts the “1+1 Fab-scFv-Fc” format, with a first Fab arm binding MSLN and a second scFv arm binding CD3. FIG. 22B depicts the “2+1 Fab₂-scFv-Fc” format, with a first Fab arm binding MSLN and a second Fab-scFv arm, wherein the Fab binds MSLN and the scFv binds CD3.

FIGS. 23A-23R depict the sequences for illustrative αMSLN×αCD3 bsAbs in the 1+1 Fab-scFv-Fc format and comprising a H1.30_L1.47 anti-CD3 scFv (a.k.a. CD3 High[VHVL]). CDRs are underlined and slashes indicate the border(s) between the variable regions and other chain components (e.g. constant region and domain linkers). It should be noted that the αMSLN×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 24A-24B depict the sequences for illustrative αMSLN×αCD3 bsAbs in the 1+1 Fab-scFv-Fc format and comprising a H1.32_L1.47 anti-CD3 scFv (a.k.a. CD3 High-Int #1[VHVL]). CDRs are underlined and slashes indicate the border(s) between the variable regions and other chain components (e.g. constant region and domain linkers). It should be noted that the αMSLN×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 25A-25B depict the sequences for illustrative αMSLN×αCD3 bsAbs in the 1+1 Fab-scFv-Fc format and comprising a L1.47_ H1.32 anti-CD3 scFv (a.k.a. CD3 High-Int #1[VLVH]). CDRs are underlined and slashes indicate the border(s) between the variable regions and other chain components (e.g. constant region and domain linkers). It should be noted that the αMSLN×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIG. 26 depict the sequences for illustrative αMSLN×αCD3 bsAbs in the 1+1 Fab-scFv-Fc format and comprising a H1.33_L1.47 anti-CD3 scFv (a.k.a. CD3 Intermediate[VHVL]). CDRs are underlined and slashes indicate the border(s) between the variable regions and other chain components (e.g. constant region and domain linkers). It should be noted that the αMSLN×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIG. 27 depicts the sequences for illustrative αMSLN×αCD3 bsAbs in the 1+1 Fab-scFv-Fc format and comprising a H1.31_L1.47 anti-CD3 scFv (a.k.a. CD3 Low[VHVL]). CDRs are underlined and slashes indicate the border(s) between the variable regions and other chain components (e.g. constant region and domain linkers). It should be noted that the αMSLN×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 28A-28B depict the sequences for illustrative αMSLN×αCD3 bsAbs in the 2+1 Fab₂-scFv-Fc format and comprising a H1.30_L1.47 anti-CD3 scFv (a.k.a. CD3 High[VHVL]). CDRs are underlined and slashes indicate the border(s) between the variable regions and other chain components (e.g. constant region and domain linkers). It should be noted that the αMSLN×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 29A-29B depict the sequences for illustrative αMSLN×αCD3 bsAbs in the 2+1 Fab₂-scFv-Fc format and comprising a L1.47_ H1.30 anti-CD3 scFv (a.k.a. CD3 High[VLVH]). CDRs are underlined and slashes indicate the border(s) between the variable regions and other chain components (e.g. constant region and domain linkers). It should be noted that the αMSLN×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 30A-30E depict the sequences for illustrative αMSLN×αCD3 bsAbs in the 2+1 Fab₂-scFv-Fc format and comprising a H1.32_L1.47 anti-CD3 scFv (a.k.a. CD3 High-Int #1[VHVL]). CDRs are underlined and slashes indicate the border(s) between the variable regions and other chain components (e.g. constant region and domain linkers). It should be noted that the αMSLN×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 31A-31T depict the sequences for illustrative αMSLN×αCD3 bsAbs in the 2+1 Fab₂-scFv-Fc format and comprising a L1.47_H1.32 anti-CD3 scFv (a.k.a. CD3 High-Int #1[VLVH]). CDRs are underlined and slashes indicate the border(s) between the variable regions and other chain components (e.g. constant region and domain linkers). It should be noted that the αMSLN×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 32A-32P depict the sequences for illustrative αMSLN×αCD3 bsAbs in the 2+1 Fab₂-scFv-Fc format and comprising a L1.47_H1.89 anti-CD3 scFv (a.k.a. CD3 High-Int #2[VLVH]). CDRs are underlined and slashes indicate the border(s) between the variable regions and other chain components (e.g. constant region and domain linkers). It should be noted that the αMSLN×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIG. 33 depicts activation of CD4⁺ T cells, as indicated by A) CD107a degranulation, B) CD25 expression, and C) CD69 expression following incubation of T cells with Kuramochi cells and the indicated test articles. The data show that anti-MSLN×anti-CD3 bsAbs in the 2+1 Fab2-scFv-Fc format (XENP31063, XENP31064, XENP31067, and XENP31068) promoted activity of CD4+ T cells more potently than corresponding 1+1 Fab-scFv-Fc bsAbs (XENP16248, XENP30764, XENP16833, and XENP30765).

FIG. 34 depicts activation of CD8+ T cells, as indicated by A) CD107a degranulation, B) CD25 expression, and C) CD69 expression following incubation of T cells with Kuramochi cells and the indicated test articles. The data show that anti-MSLN×anti-CD3 bsAbs in the 2+1 Fab2-scFv-Fc format (XENP31063, XENP31064, XENP31067, and XENP31068) promoted activity of CD8+ T cells more potently than corresponding 1+1 Fab-scFv-Fc bsAbs (XENP16248, XENP30764, XENP16833, and XENP30765).

FIGS. 35A-35D depict the binding to OVCAR8 cells by A) XENP31697 vs. XENP31717 (both having 0.28 nM MSLN K_{D}), B) XENP32561 vs. XENP32745 (both having 0.45 nM MSLN K_{D}), C) XENP32562 vs. XENP32748 (both having 0.74 nM MSLN K_{D}), D) XENP32567 vs. XENP32852 vs. XENP32853 (each having 3.9 nM MSLN K_{D}), E) XENP32552 vs. XENP32947 (both having 9.2 nM MSLN K_{D}), F) XENP32937 vs. XENP32948 (both having 10.2 nM MSLN K_{D}), and G) XENP32751 vs. XENP32563 (both having ˜12.8 nM MSLN K_{D}). The data show that as the MSLN binding affinity is decreased, the 2+1 Fab2-scFv-Fc format provides more potent binding to OVCAR8 cells than the 1+1 Fab-scFv-Fc format. Further, XENP32852 and XENP32853 which differ only in CD3 binding affinity have identical binding to OVCAR8 cells confirming that avid binding is due to the MSLN binding domain.

FIG. 36 depicts the RTCC on A549, HT29, MCF7, OVCAR8, and SKOV3 cells by A) XENP31717 (having 0.28 nM MSLN K_{D}), B) XENP32852 (having 3.9 nM MSLN K_{D}), C) XENP32948 (having 10.2 nM MSLN K_{D}), and D) XENP32947 (having 9.2 nM MSLN K_{D}). The data show that at high MSLN binding affinity, the 2+1 bsAbs induced potent RTCC on MSLN^{high} OVCAR8 cell lines as well as MSLN^{low} SKOV3 and HT29 cell lines. As MSLN binding affinity is reduced, the RTCC potency of the 2+1 bsAbs on the MSLN^{low} cell lines is reduced.

FIG. 37 depicts induction of RTCC on luciferase-transfected cell lines with varying surface MSLN densities by A) XENP31717, B) XENP33366, and C) XENP33367. XENP33366 having 90.4 nM MSLN binding affinity enabled improved selectivity for MSLN^{high} OVCAR8, as indicated by reduced potency in killing of SKOV3 cells and more potent killing of OVCAR8. XENP33367 having 246 nM MSLN binding affinity demonstrated little to no RTCC activity on MSLN^{low} cells, but was also less potent on OVCAR8 than XENP33366.

FIG. 38 depicts induction of RTCC (as determined by xCELLigence® Real Time Cell Analysis instrument) on cell lines with varying surface MSLN densities by A) XENP31717, B) XENP33366, and C) XENP33367. XENP33366 having 90.4 nM MSLN binding affinity enabled improved selectivity for MSLN^{high} OVCAR8, as indicated by reduced potency in killing of SKOV3 cells and more potent killing of OVCAR8. XENP33367 having 246 nM MSLN binding affinity demonstrated little to no RTCC activity on MSLN^{low} cells, but was also less potent on OVCAR8 than XENP33366. RTCC activity on ASPC-1, a cancer cell line with ˜36K surface MSLN expression and a lower cancer IHC score, was similar to RTCC activity on OVCAR8 cell.

FIG. 39 depicts induction of T cell proliferation (as indicated by percentage T cells expressing Ki67) by A) XENP31717, B) XENP33366, and C) XENP33367 in the presence of cell lines with varying surface MSLN densities. Each of the three bispecific antibodies enabled proliferation in the presence of Score 3 and Score 2 cell lines, but not in the presence of Score 1 cell lines.

FIG. 40 depicts induction of T cell proliferation (as indicated by percentage T cells expressing Ki67) by A) XENP31717, B) XENP33366, and C) XENP33367 in the presence of cell lines with varying surface MSLN densities. Reduction in MSLN binding affinity reduced T cell degranulation in the presence of Score 1 cell lines.

FIG. 41 depicts induction of T cell proliferation (as indicated by percentage T cells expressing Ki67) by A) XENP31717, B) XENP33366, and C) XENP33367 in the presence of cell lines with varying surface MSLN densities. Reduction in MSLN binding affinity reduced T cell activation in the presence of Score 1 cell lines.

FIG. 42 induction of RTCC on luciferase-transfected cell lines with varying surface MSLN densities by A) XENP31717, B) XENP32852, C) XENP32251, and D) XENP32853. The data show that bsAbs with lower CD3 binding affinity (e.g. CD3 High-Int #2) enabled enhanced selectivity for OVCAR8 (MSLN^{high}) over bsAbs with higher CD3 binding affinity (e.g. CD3 High-Int #1).

FIGS. 43A-43K depict several formats for use in the anti-MSLN×anti-CD3 bispecific antibodies disclosed herein. The first is the “1+1 Fab-scFv-Fc” format (also referred to as the “bottle opener” or “Triple F” format), with a first antigen binding domain that is a Fab domain and a second anti-antigen binding domain that is an scFv domain. Additionally, “mAb-Fv,” “mAb-scFv,” “2+1 Fab2-scFv-Fc” (also referred to as the “central scFv” or “central-scFv” format”), “central-Fv,” “one armed central-scFv,” “one scFv-mAb,” “scFv-mAb,” “dual scFv,” “trident,” and non-heterodimeric bispecific formats are all shown. The scFv domains can be either, from N- to C-terminus orientation: variable heavy-(optional linker)-variable light, or variable light-(optional linker)-variable heavy. In addition, for the one armed scFv-mAb, the scFv can be attached either to the N-terminus of a heavy chain monomer or to the N-terminus of the light chain. In certain embodiments, “Anti-antigen 1” in FIGS. 43A-43K refers to a MSLN binding domain. In certain embodiments, “Anti-antigen 1” in FIGS. 43A-43K refers to a CD3 binding domain. In certain embodiments, “Anti-antigen 2” in FIGS. 43A-43K refers to a MSLN binding domain. In certain embodiments “Anti-antigen 2” in FIGS. 43A-43K refers to a CD3 binding domain. In some embodiments, “Anti-antigen 1” in FIGS. 43A-43K refers to a MSLN binding domain and “Anti-antigen 2” in FIGS. 43A-43K refers to a CD3 binding domain. In some embodiments, “Anti-antigen 1” in FIGS. 43A-43K refers to a CD3 binding domain and “Anti-antigen 2” in FIGS. 43A-43K refers to a MSLN binding domain. Any of the MSLN binding domains and CD3 binding domains disclosed can be included in the bispecific formats of FIGS. 43A-43K.

### DETAILED DESCRIPTION OF THE INVENTION

### I. Overview

Anti-bispecific antibodies that co-engage CD3 and a tumor antigen target are used to redirect T cells to attack and lyse targeted tumor cells. Examples include the BiTE® and DART® antibody formats, which monovalently engage CD3 and a tumor antigen. While the CD3-targeting approach has shown considerable promise, a common side effect of such therapies is the associated production of cytokines, often leading to toxic cytokine release syndrome. Because the anti-CD3 binding domain of the bispecific antibody engages all T cells, the high cytokine-producing CD4 T cell subset is recruited. Moreover, the CD4 T cell subset includes regulatory T cells, whose recruitment and expansion can potentially lead to immune suppression and have a negative impact on long-term tumor suppression. In addition, these formats do not contain Fc domains and show very short serum half-lives in patients.

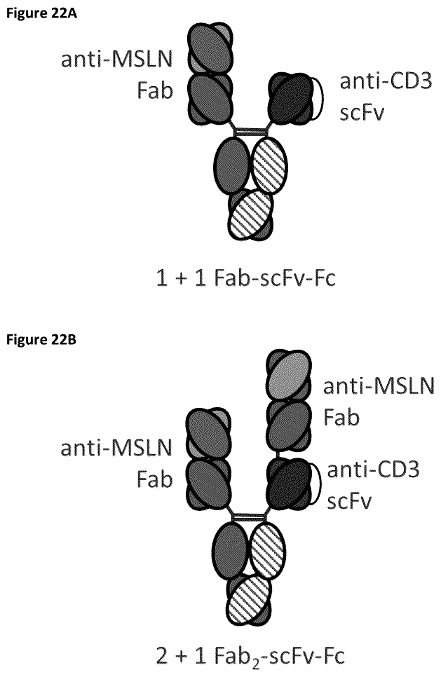

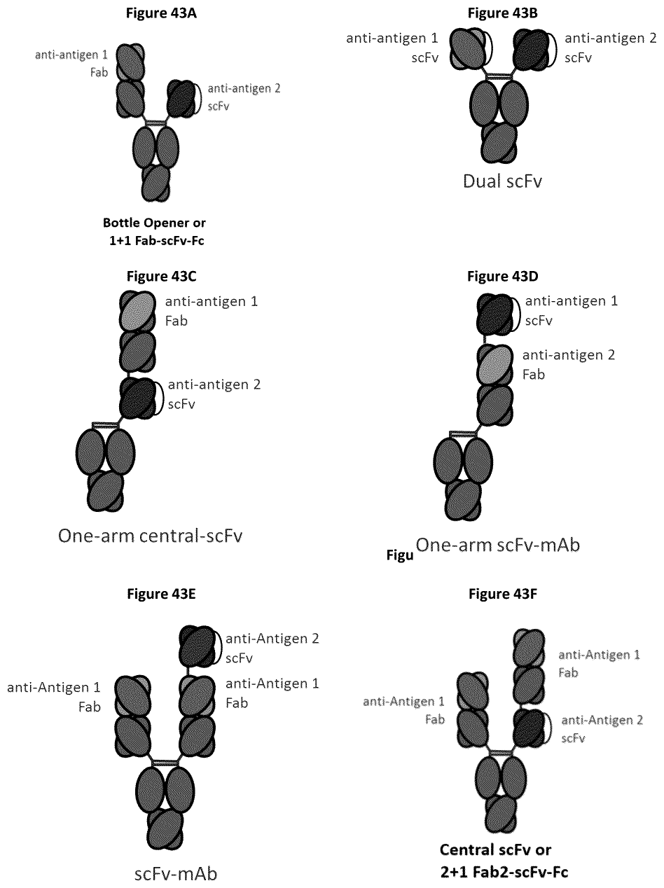

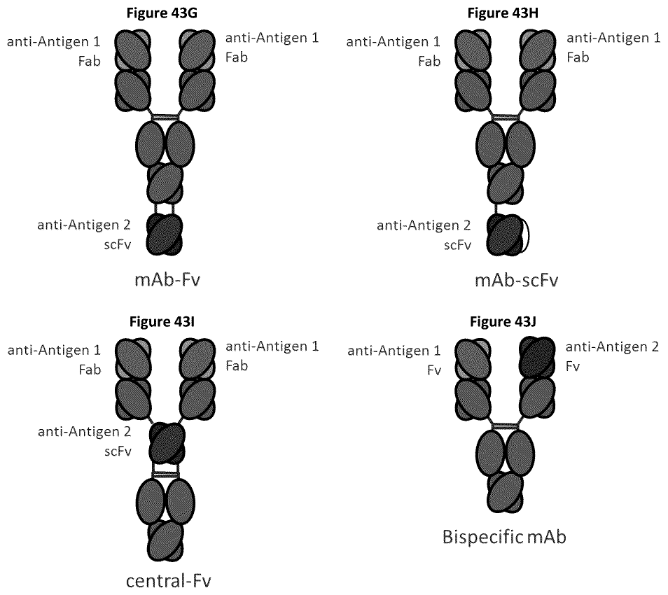

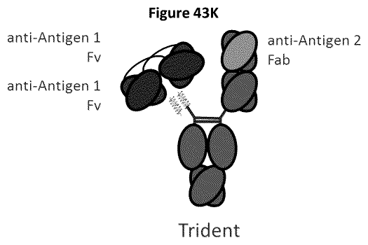

Provided herein are novel anti-CD3×anti-MSLN (also referred to as anti-MSLN×anti-CD3, αCD3×αMSLN, or αMSLN×αCD3) heterodimeric bispecific antibodies and methods of using such antibodies for the treatment of cancers. In particular, provided herein are anti-CD3, anti-MSLN bispecific antibodies in a variety of formats such as those depicted in FIGS. 22A, 22B and 43A-43K. These bispecific antibodies are useful for the treatment of cancers, particularly those with increased MSLN expression such as ovarian cancer, colon cancer, breast cancer, hepatocellular carcinoma, lung cancer, mesothelioma, pancreatic cancer, non-small cell lung cancer (such as lung adenocarcinoma) and endometrial cancer. Such antibodies are used to direct CD3+ effector T cells to MSLN+ tumors, thereby allowing the CD3+ effector T cells to attack and lyse the MSLN+ tumors.

Additionally, in some embodiments, the disclosure provides bispecific antibodies that have different binding affinities to human CD3 that can alter or reduce the potential side effects of anti-CD3 therapy. That is, in some embodiments the antibodies described herein provide antibody constructs comprising anti-CD3 antigen binding domains that are “strong” or “high affinity” binders to CD3 (e.g. one example are heavy and light variable domains depicted as H1.30_L1.47 (optionally including a charged linker as appropriate)) and also bind to MSLN. In other embodiments, the antibodies described herein provide antibody constructs comprising anti-CD3 antigen binding domains that are “lite” or “lower affinity” binders to CD3. Additional embodiments provides antibody constructs comprising anti-CD3 antigen binding domains that have intermediate or “medium” affinity to CD3 that also bind to MSLN. While a very large number of anti-CD3 antigen binding domains (ABDs) can be used, particularly useful embodiments use 6 different anti-CD3 ABDs, although they can be used in two scFv orientations as discussed herein. Affinity is generally measured using a Biacore™ assay.

It should be appreciated that the “high, medium, low” anti-CD3 sequences provided herein can be used in a variety of heterodimerization formats as depicted in FIGS. 22A, 22B, and 43A-43K. In general, due to the potential side effects of T cell recruitment, exemplary embodiments utilize formats that only bind CD3 monovalently, such as depicted in FIGS. 22A and 22B, and in the formats depicted herein, it is the CD3 ABD that is a scFv as more fully described herein. In contrast, the subject bispecific antibodies can bind MSLN either monovalently (e.g. FIG. 22A) or bivalently (e.g. FIG. 22B).

Provided herein are compositions that include MSLN binding domains, including antibodies with such MSLN binding domains (e.g., MSLN×CD3 bispecific antibodies). Subject antibodies that include such MSLN binding domains advantageously elicit a range of different immune responses, depending on the particular MSLN binding domain used. For example, the subject antibodies exhibit differences in selectivity for cells with different MSLN expression, potencies for MSLN expressing cells, ability to elicit cytokine release, and sensitivity to soluble MSLN. Such MSLN binding domains and related antibodies find use, for example, in the treatment of MSLN associated cancers.

Accordingly, in one aspect, provided herein are heterodimeric antibodies that bind to two different antigens, e.g. the antibodies are “bispecific”, in that they bind two different target antigens, generally MSLN and CD3 as described herein. These heterodimeric antibodies can bind these target antigens either monovalently (e.g. there is a single antigen binding domain such as a variable heavy and variable light domain pair) or bivalently (there are two antigen binding domains that each independently bind the antigen). In some embodiments, the heterodimeric antibody provided herein includes one CD3 binding domain and one MSLN binding domain (e.g., heterodimeric antibodies in the “1+1 Fab-scFv-Fc” format described herein). In other embodiments, the heterodimeric antibody provided herein includes one CD3 binding domain and two MSLN binding domains (e.g., heterodimeric antibodies in the “2+1 Fab2-scFv-Fc” formats described herein). The heterodimeric antibodies provided herein are based on the use different monomers which contain amino acid substitutions that “skew” formation of heterodimers over homodimers, as is more fully outlined below, coupled with “pI variants” that allow simple purification of the heterodimers away from the homodimers, as is similarly outlined below. The heterodimeric bispecific antibodies provided generally rely on the use of engineered or variant Fc domains that can self-assemble in production cells to produce heterodimeric proteins, and methods to generate and purify such heterodimeric proteins.

### II. Nomenclature

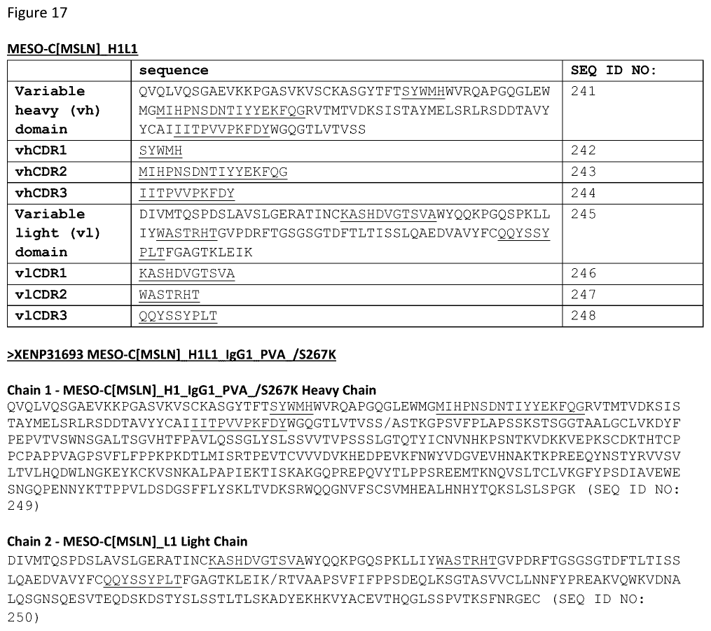

The antibodies provided herein are listed in several different formats. In some instances, each monomer of a particular antibody is given a unique “XENP” number, although as will be appreciated in the art, a longer sequence might contain a shorter one. For example, a “scFv-Fc” monomer of a 1+1 Fab-scFv-Fc format antibody may have a first XENP number, while the scFv domain itself will have a different XENP number. Some molecules have three polypeptides, so the XENP number, with the components, is used as a name. Thus, the molecule XENP31063, which is in 2+1 Fab₂-scFv-Fc format, comprises three sequences (see FIG. 28A) a “Fab-Fc Heavy Chain” monomer; 2) a “Fab-scFv-Fc Heavy Chain” monomer; and 3) a “Light Chain” monomer or equivalents, although one of skill in the art would be able to identify these easily through sequence alignment. These XENP numbers are in the sequence listing as well as identifiers, and used in the Figures. In addition, one molecule, comprising the three components, gives rise to multiple sequence identifiers. For example, the listing of the Fab includes, the full heavy chain sequence, the variable heavy domain sequence and the three CDRs of the variable heavy domain sequence, the full light chain sequence, a variable light domain sequence and the three CDRs of the variable light domain sequence. A Fab-scFv-Fc monomer includes a full length sequence, a variable heavy domain sequence, 3 heavy CDR sequences, and an scFv sequence (include scFv variable heavy domain sequence, scFv variable light domain sequence and scFv linker). Note that some molecules herein with a scFv domain use a single charged scFv linker (+H), although others can be used. In addition, the naming nomenclature of particular antigen binding domains (e.g., MSLN and CD3 binding domains) use a “Hx.xx_Ly.yy” type of format, with the numbers being unique identifiers to particular variable chain sequences. Thus, the variable domain of the Fab side of MSLN binding domain MESO-C[MSLN] H1L1 (e.g., FIG. 17) is “H1 L1”, which indicates that the variable heavy domain, H1, was combined with the light domain L1. In the case that these sequences are used as scFvs, the designation “H1 L1”, indicates that the variable heavy domain, H1 is combined with the light domain, L1, and is in VH-linker-VL orientation, from N- to C-terminus. This molecule with the identical sequences of the heavy and light variable domains but in the reverse order (VL-linker-VH orientation, from N- to C-terminus) would be designated “L1_H1.1”. Similarly, different constructs may “mix and match” the heavy and light chains as will be evident from the sequence listing and the figures.

### III. Definitions

In order that the application may be more completely understood, several definitions are set forth below. Such definitions are meant to encompass grammatical equivalents.

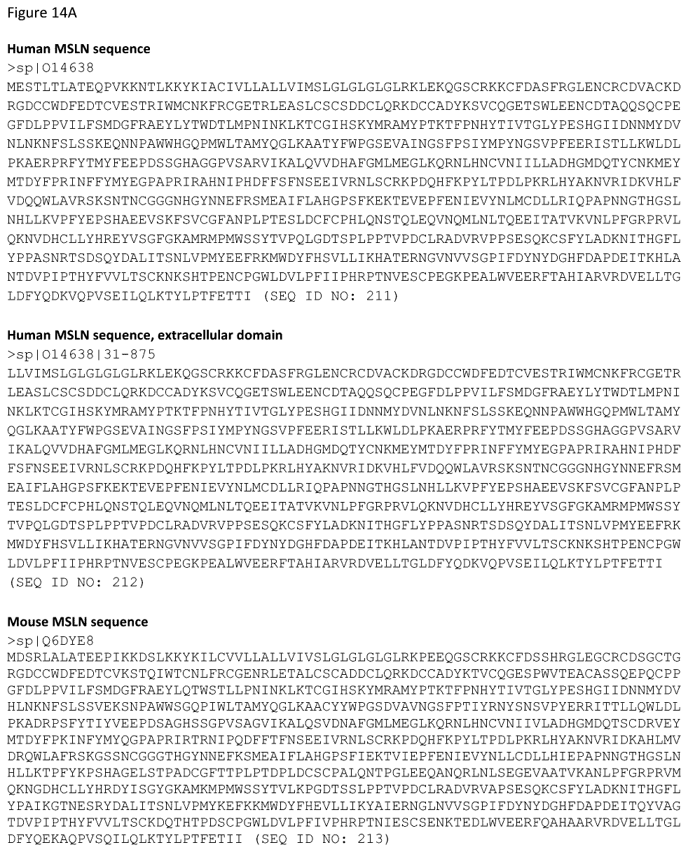

By “MSLN” or “Mesothelin” or “MESO” (e.g., FIG. 14A-14B) herein is meant a protein belonging to a series of ectoenzymes that are involved in hydrolysis of extracellular nucleotides. MSLN sequences are depicted, for example, in FIGS. 14A and 14B. MSLN is expressed in particular cancers, including ovarian cancer, colon cancer, breast cancer, hepatocellular carcinoma, lung cancer, mesothelioma, pancreatic cancer, non-small cell lung cancer (such as lung adenocarcinoma) and endometrial cancer.

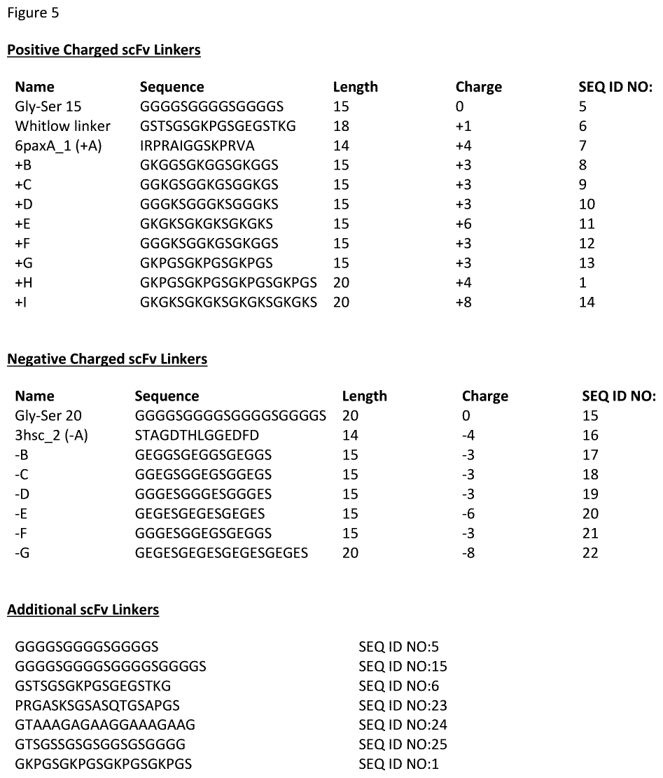

By “ablation” herein is meant a decrease or removal of activity. Thus for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore™, SPR or BLI assay. Of particular use in the ablation of FcγR binding are those shown in FIG. 5, which generally are added to both monomers.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, term “antibody” is used generally. Antibodies described herein can take on a number of formats as described herein, including traditional antibodies as well as antibody derivatives, fragments and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

Other useful antibody formats include, but are not limited to, the 1+1 Fab-scFv-Fc format and 2+1 Fab-scFv-Fc antibody formats described herein, as well as “mAb-Fv,” “mAb-scFv,” “central-Fv”, “one armed scFv-mAb,” “scFv-mAb,” “dual scFv,” and “trident” format antibodies, as shown in FIG. 43A-43K.

Antibody heavy chains typically include a variable heavy (VH) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM. Thus, “isotype” as used herein is meant any of the subclasses of immunoglobulins defined by the chemical and antigenic characteristics of their constant regions. It should be understood that therapeutic antibodies can also comprise hybrids of isotypes and/or subclasses. For example, as shown in US Publication 2009/0163699, incorporated by reference, the antibodies described herein include the use of human IgG1/G2 hybrids.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the antibodies described herein are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356D/358M allotype, however the other allotype is included herein. That is, any sequence inclusive of an IgG1 Fc domain included herein can have 356E/358L replacing the 356D/358M allotype. It should be understood that therapeutic antibodies can also comprise hybrids of isotypes and/or subclasses. For example, as shown in US Publication 2009/0163699, incorporated by reference, the present antibodies, in some embodiments, include IgG1/IgG2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminal, CH2-CH3 and hinge-CH2-CH3. In some embodiments, the Fc domain is that from IgG1, IgG2, IgG3 or lgG4, with IgG1 hinge-CH2-CH3 and IgG4 hinge-CH2-CH3 finding particular use in many embodiments. Additionally, in the case of human IgG1 Fc domains, frequently the hinge includes a C220S amino acid substitution. Furthermore, in the case of human IgG4 Fc domains, frequently the hinge includes a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of Ig domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy constant region domains can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL), which includes light chain CDRs vlCDR1-3, and a constant light chain region (often referred to as CL or Cκ). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen (e.g., MSLN or CD3) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The antibodies described herein provide a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1):55-77 (2003):

TABLE 2

| | Kabat + | | | | | | |
|---|---|---|---|---|---|---|---|
| | Chothia | IMGT | Rabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the disclosure not only includes the enumerated antigen binding domains and antibodies herein, but those that compete for binding with the epitopes bound by the enumerated antigen binding domains.

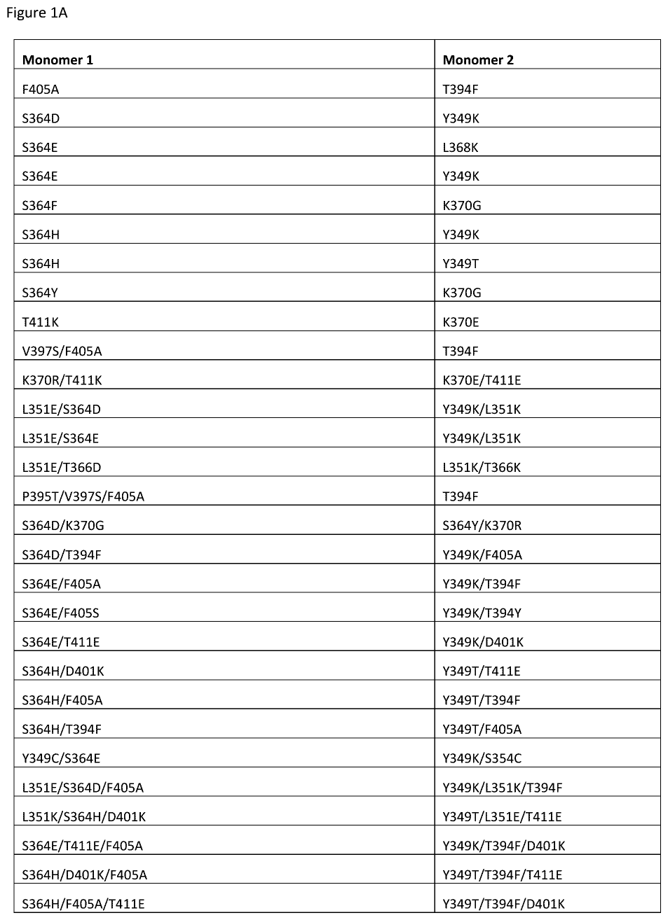

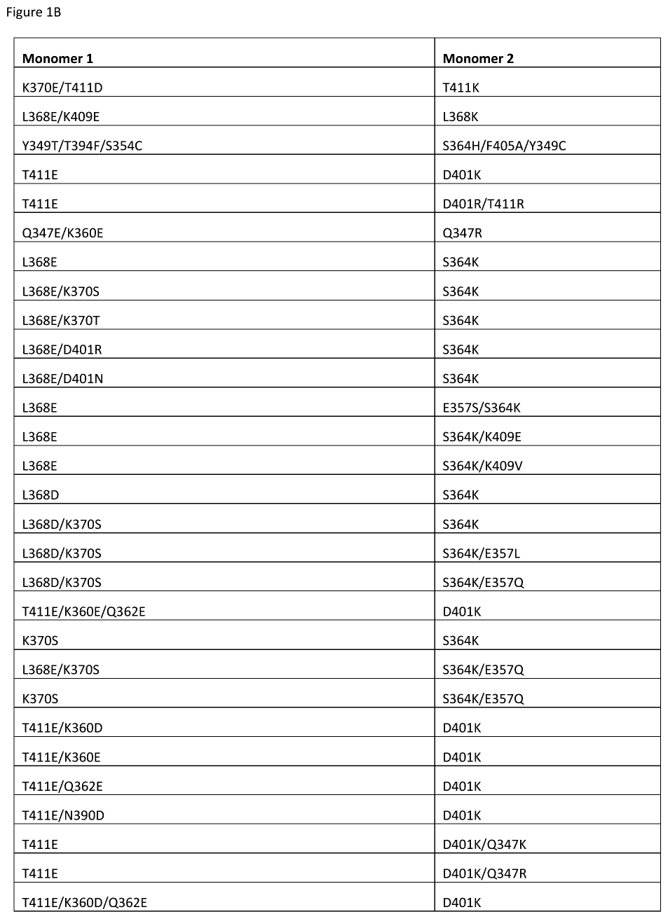

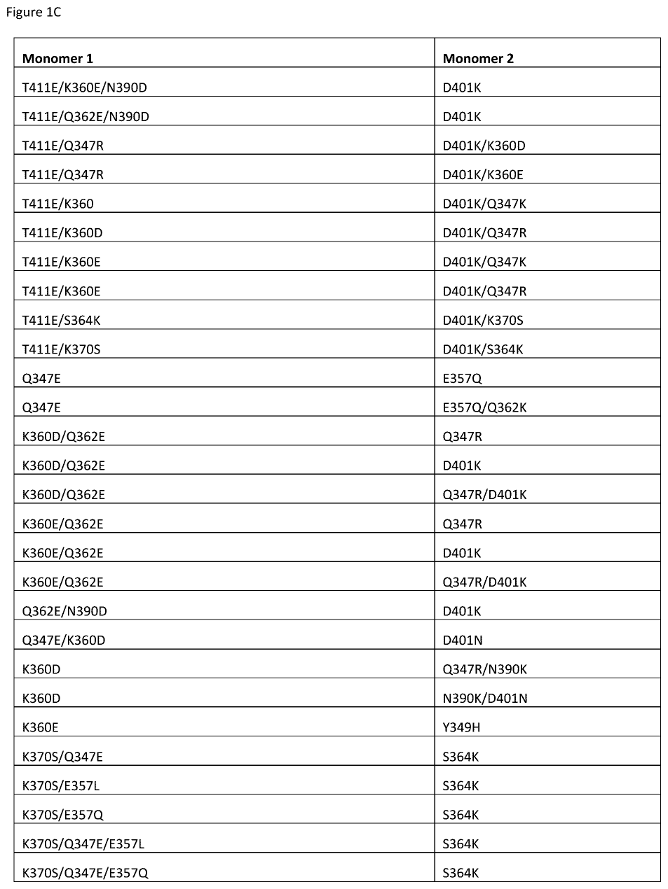

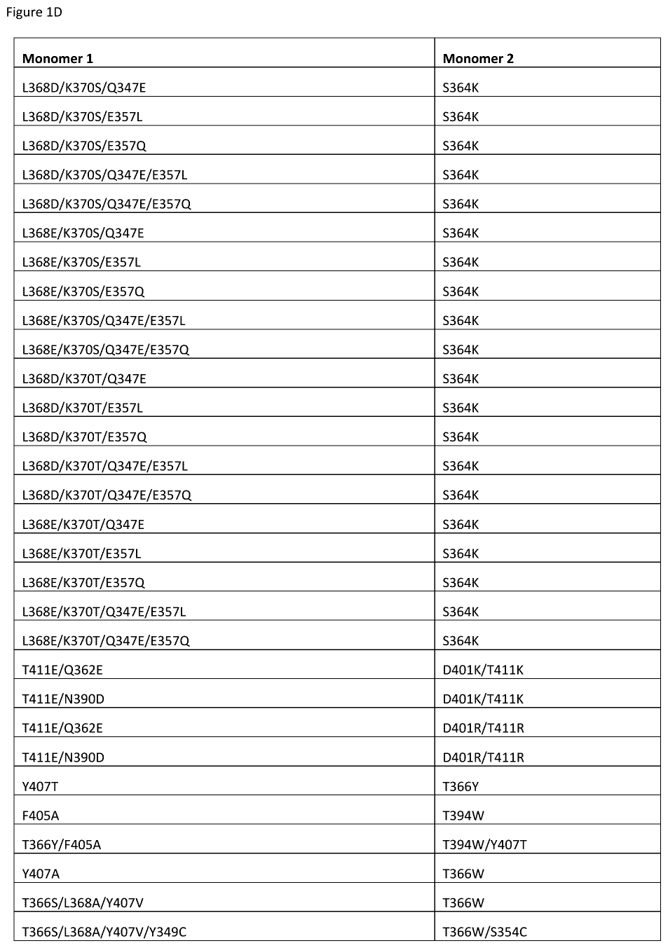

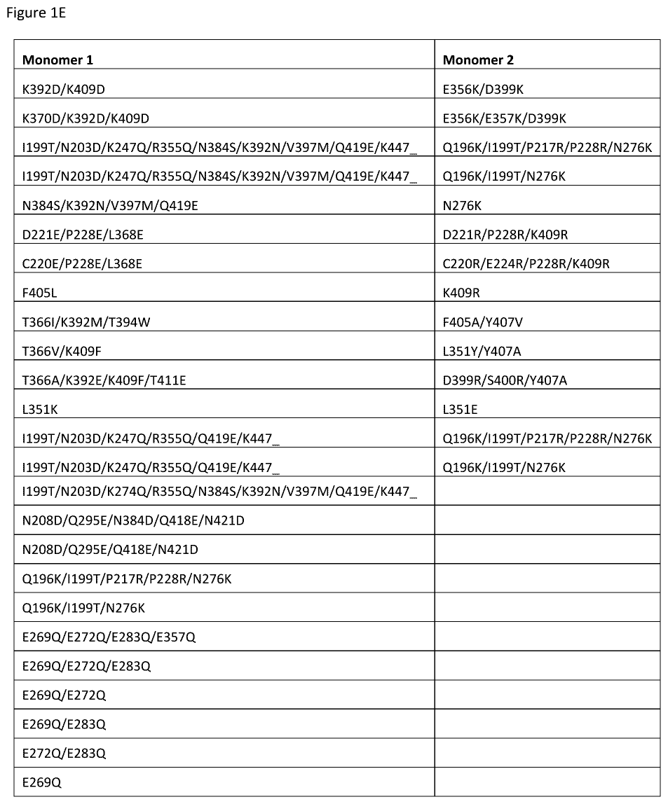

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used (e.g., from FIG. 1). In general, the C-terminus of the scFv domain is attached to the N-terminus of the hinge in the second monomer.

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (VHCDR1, VHCDR2 and VHCDR3 for the variable heavy domain and VLCDR1, VLCDR2 and VLCDR3 for the variable light domain) and four framework (FR) regions, arranged from amino-terminus to carboxy-terminus in the following order: FR1-CDR1-FR2-CDR2-FR3-CDR3-FR4. The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described in Table 2.

By “Fab” or “Fab region” as used herein is meant the polypeptide that comprises the VH, CH1, VL, and CL immunoglobulin domains, generally on two different polypeptide chains (e.g. VH-CH1 on one chain and VL-CL on the other). Fab may refer to this region in isolation, or this region in the context of a bispecific antibody described herein. In the context of a Fab, the Fab comprises an Fv region in addition to the CH1 and CL domains.

By “Fv” or “Fv fragment” or “Fv region” as used herein is meant a polypeptide that comprises the VL and VH domains of an ABD. Fv regions can be formatted as both Fabs (as discussed above, generally two different polypeptides that also include the constant regions as outlined above) and scFvs, where the VL and VH domains are combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (VH-linker-VL or VL-linker-VH). In the sequences depicted in the sequence listing and in the figures, the order of the VH and VL domain is indicated in the name, e.g. H.X_L. Y means N- to C-terminal is VH-linker-VL, and L. Y_H.X is VL-linker-VH.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ) or E233del designates a deletion of glutamic acid at position 233. Additionally, EDA233- or EDA233 #designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST® sequence alignment search tool. “Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference).

In general, variant Fc domains have at least about 80, 85, 90, 95, 97, 98 or 99 percent identity to the corresponding parental human IgG Fc domain (using the identity algorithms discussed below, with one embodiment utilizing the BLAST® sequence alignment search tool algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” herein is meant at least two covalently attached amino acids, which includes proteins, polypeptides, oligopeptides and peptides. In addition, polypeptides that make up the antibodies described herein may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or lgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is one that increases binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for antibody numbering.

By “target antigen” as used herein is meant the molecule that is bound specifically by the antigen binding domain comprising the variable regions of a given antibody.

By “strandedness” in the context of the monomers of the heterodimeric antibodies described herein is meant that, similar to the two strands of DNA that “match”, heterodimerization variants are incorporated into each monomer so as to preserve the ability to “match” to form heterodimers. For example, if some pI variants are engineered into monomer A (e.g. making the pI higher) then steric variants that are “charge pairs” that can be utilized as well do not interfere with the pI variants, e.g. the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the antibodies described herein is meant a cell that contains the exogeneous nucleic acids encoding the components of the bispecific antibody and is capable of expressing the bispecific antibody under suitable conditions. Suitable host cells are discussed below.

By “wild type or WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the BLAST® sequence alignment search tool algorithm, see blast.ncbi.nlm.nih.gov. When using any of the aforementioned algorithms, the default parameters (for Window length, gap penalty, etc) are used. In one embodiment, sequence identity is done using the BLAST® sequence alignment search tool algorithm, using default parameters

The antibodies described herein are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where K_{D} refers to a dissociation rate of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore™, SPR or BLI assay.

### IV. MSLN Binding Domains

In one aspect, provided herein are MSLN antigen binding domains (ABDs) and compositions that include such MSLN antigen binding domains (ABDs), including anti-MSLN antibodies. Subject antibodies that include such MSLN antigen binding domains (e.g., anti-MSLN×anti-CD3 bispecific antibodies) advantageously elicit a range of different immune responses. Such MSLN binding domains and related antibodies find use, for example, in the treatment of MSLN associated cancers.

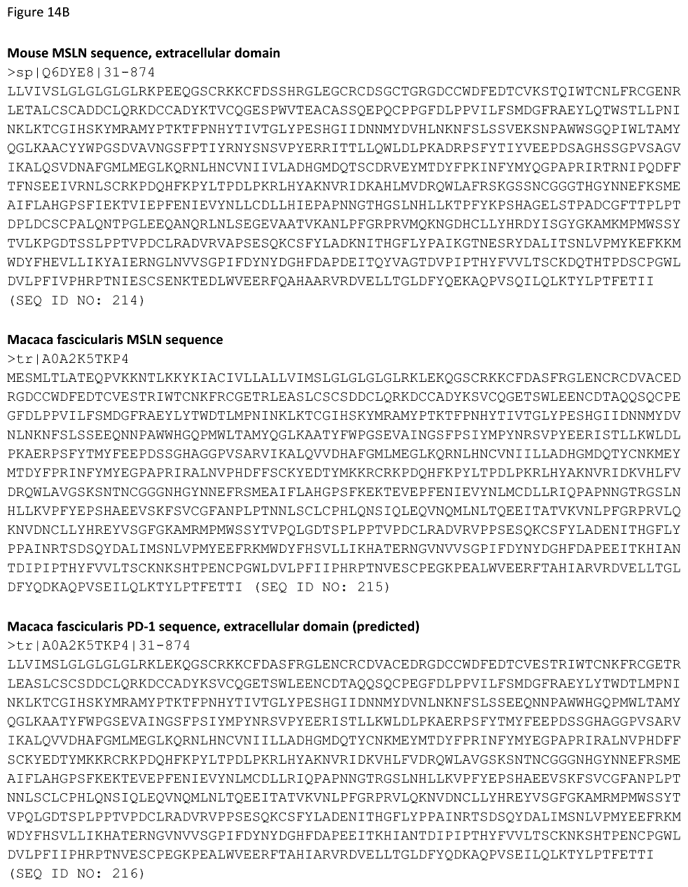

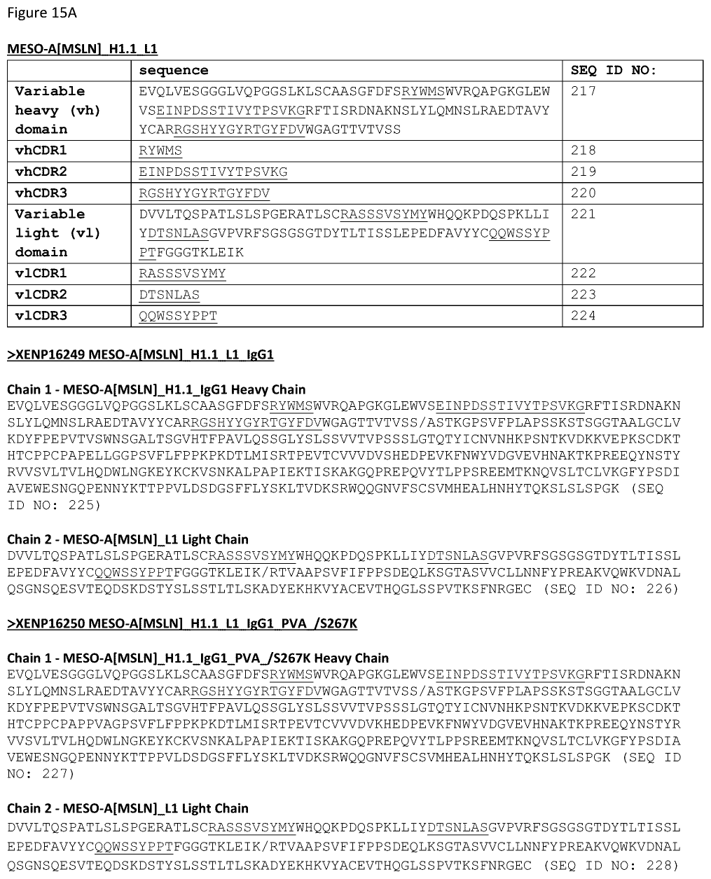

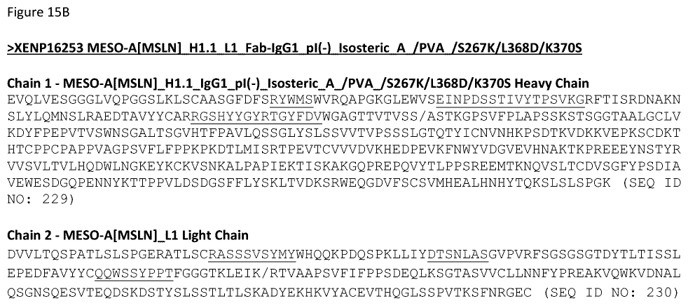

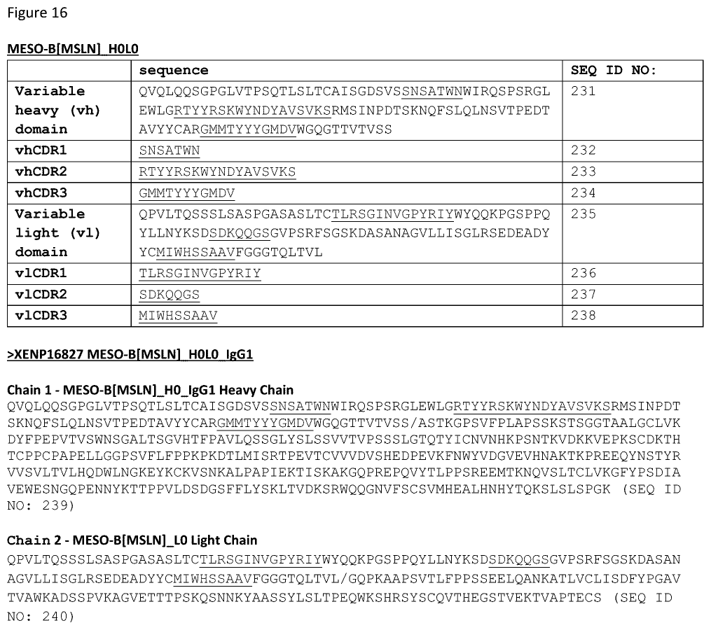

As will be appreciated by those in the art, suitable MSLN binding domains can comprise a set of 6 CDRs as depicted in the sequence listing and FIGS. 15-21, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIGS. 15-21 and the Sequence Listing (see Table 2). Suitable MSLN ABDs can also include the entire VH and VL sequences as depicted in these sequences and figures, used as scFvs or as Fab domains.

In one embodiment, the MSLN antigen binding domain includes the 6 CDRs (i.e., vhCDR1-3 and vlCDR1-3) of a MSLN ABD described herein, including the figures and sequence listing. In exemplary embodiments, the MSLN ABD is one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21). In addition to the parental CDR sets disclosed in the figures and sequence listing that form an ABD to MSLN, provided herein are variant MSLN ABDs having CDRs that include at least one modification of the MSLN ABD CDRs disclosed herein. In one embodiment, the MSLN ABD includes a set of 6 CDRs with 1, 2, 3, 4, 5, 6, 7, 8, 9, 10 amino acid modifications as compared to the 6 CDRs of a MSLN ABD described herein, including the figures and sequence listing. In exemplary embodiments, the MSLN ABD includes a set of 6 CDRs with 1, 2, 3, 4, 5, 6, 7, 8, 9, 10 amino acid modifications as compared to the 6 CDRs of one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In certain embodiments, the variant MSLN ABD is capable of binding MSLN antigen, as measured by at least one of a Biacore™, surface plasmon resonance (SPR) and/or BLI (biolayer interferometry, e.g., Octet® assay) assay, with the latter finding particular use in many embodiments. In particular embodiments, the MSLN ABD is capable of binding human MSLN antigen.

In one embodiment, the MSLN ABD includes 6 CDRs that are at least 90, 95, 97, 98 or 99% identical to the 6 CDRs of a MSLN ABD as described herein, including the figures and sequence listing. In exemplary embodiments, the MSLN ABD includes 6 CDRs that are at least 90, 95, 97, 98 or 99% identical to the 6 CDRs of one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21). In certain embodiments, the MSLN ABD is capable of binding to MSLN antigen, as measured by at least one of a Biacore™, surface plasmon resonance (SPR) and/or BLI (biolayer interferometry, e.g., Octet® assay) assay, with the latter finding particular use in many embodiments. In particular embodiments, the MSLN ABD is capable of binding human MSLN antigen (see FIG. 14A).

In another exemplary embodiment, the MSLN ABD include the variable heavy (VH) domain and variable light (VL) domain of any one of the MSLN ABDs described herein, including the figures and sequence listing (e.g., FIGS. 15-21). In exemplary embodiments, the MSLN ABD is one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In addition to the parental MSLN variable heavy and variable light domains disclosed herein, provided herein are MSLN ABDs that include a variable heavy domain and/or a variable light domain that are variants of a MSLN ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a MSLN ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21). In certain embodiments, the MSLN ABD is capable of binding to MSLN, as measured at least one of a Biacore™, surface plasmon resonance (SPR) and/or BLI (biolayer interferometry, e.g., Octet® assay) assay, with the latter finding particular use in many embodiments. In particular embodiments, the MSLN ABD is capable of binding human MSLN antigen.

In one embodiment, the variant VH and/or VL domain is at least 90, 95, 97, 98 or 99% identical to the VH and/or VL of a MSLN ABD as described herein, including the figures and sequence listing. In exemplary embodiments, the variant VH and/or VL domain is at least 90, 95, 97, 98 or 99% identical to the VH and/or VL of one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21). In certain embodiments, the MSLN ABD is capable of binding to the MSLN, as measured by at least one of a Biacore™, surface plasmon resonance (SPR) and/or BLI (biolayer interferometry, e.g., Octet® assay) assay, with the latter finding particular use in many embodiments. In particular embodiments, the MSLN ABD is capable of binding human MSLN antigen.

### V. Antibodies

In one aspect, provided herein are antibodies that bind to MSLN (e.g., anti-MSLN antibodies). In certain embodiments, the antibody binds to human MSLN (FIG. 14A). Subject anti-MSLN antibodies include monospecific MSLN antibodies, as well as multi-specific (e.g., bispecific) anti-MSLN antibodies. In certain embodiments, the anti-MSLN antibody has a format according to any one of the antibody formats depicted in FIGS. 22A, 22B, and 43A-43K.

In some embodiments, the subject compositions include a MSLN binding domain. In some embodiments, the composition includes an antibody having a MSLN binding domain. Antibodies provided herein include one, two, three, four, and five or more MSLN binding domains. In certain embodiments, the MSLN binding domain includes any one of the vhCDR1, vhCDR2, vhCDR3, vlCDR1, vlCDR2 and vlCDR3 sequences of a MSLN binding domain selected from those depicted in FIGS. 15-21. In some embodiments, the MSLN binding domain includes the underlined vhCDR1, vhCDR2, vhCDR3, vlCDR1, vlCDR2 and vlCDR3 sequences of a MSLN binding domain selected from those depicted in FIGS. 15-21. In some embodiments, the MSLN binding domain includes the variable heavy domain and variable light domain of a MSLN binding domain selected from those depicted in FIGS. 15-21. MSLN binding domains depicted in FIGS. 15-21 include: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In one aspect, provided herein are bispecific antibodies that bind to MSLN and CD3, in various formats as outlined below, and generally depicted in FIGS. 22A-22B, and 43A-43K. These bispecific, heterodimeric antibodies include a MSLN binding domain. In certain embodiments, the MSLN binding domain includes the VHCDR1, VHCDR2, VHCDR3, VLCDR1, VLCDR2 and VLCDR3 sequences of a MSLN binding domain selected from the group consisting of those depicted in FIGS. 15-21. In some embodiments, the MSLN binding domain includes the underlined VHCDR1, VHCDR2, VHCDR3, VLCDR1, VLCDR2 and VLCDR3 sequences of a MSLN binding domain selected from those depicted in FIGS. 15-21.

These bispecific heterodimeric antibodies bind MSLN and CD3. Such antibodies include a CD3 binding domain and at least one MSLN binding domain. Any suitable MSLN binding domain can be included in the anti-MSLN×anti-CD3 bispecific antibody. In some embodiments, the anti-MSLN×anti-CD3 bispecific antibody includes one, two, three, four or more MSLN binding domains, including but not limited to those depicted in FIGS. 15-21. In certain embodiments, the anti-MSLN×anti-CD3 antibody includes a MSLN binding domain that includes the VHCDR1, VHCDR2, VHCDR3, VLCDR1, VLCDR2 and VLCDR3 sequences of a MSLN binding domain selected from the group consisting of those depicted in FIGS. 15-21. In some embodiments, the anti-MSLN×anti-CD3 antibody includes a MSLN binding domain that includes the underlined VHCDR1, VHCDR2, VHCDR3, VLCDR1, VLCDR2 and VLCDR3 sequences of a MSLN binding domain selected from the group consisting of those depicted in FIGS. 15-21. In some embodiments, the anti-MSLN×anti-CD3 antibody includes a MSLN binding domain that includes the variable heavy domain and variable light domain of a MSLN binding domain selected from the group consisting of those depicted in FIGS. 15-21. In an exemplary embodiment, the anti-MSLN×anti-CD3 antibody includes an anti-MSLN MESO-C[MSLN]_H1L1 binding domain.

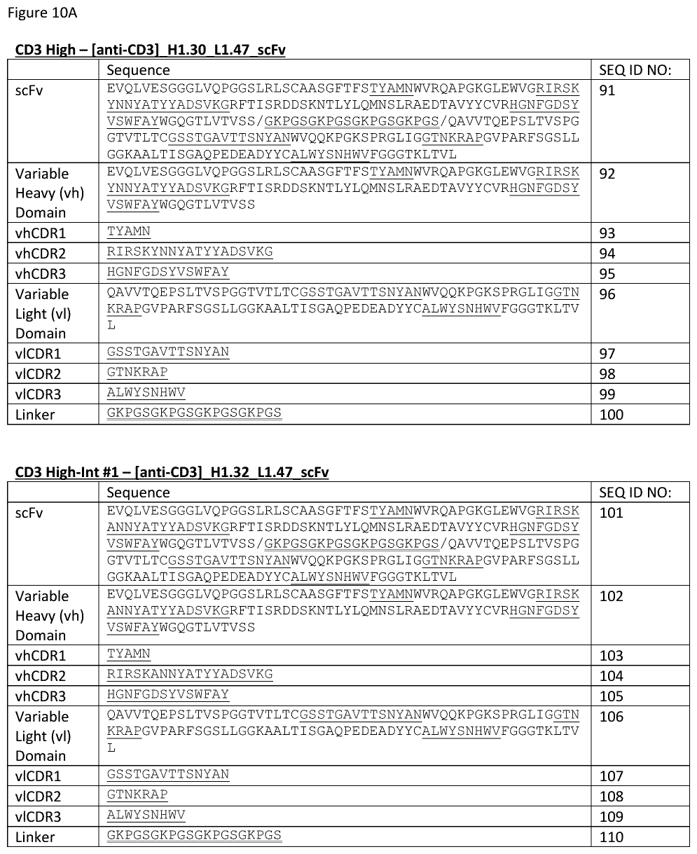

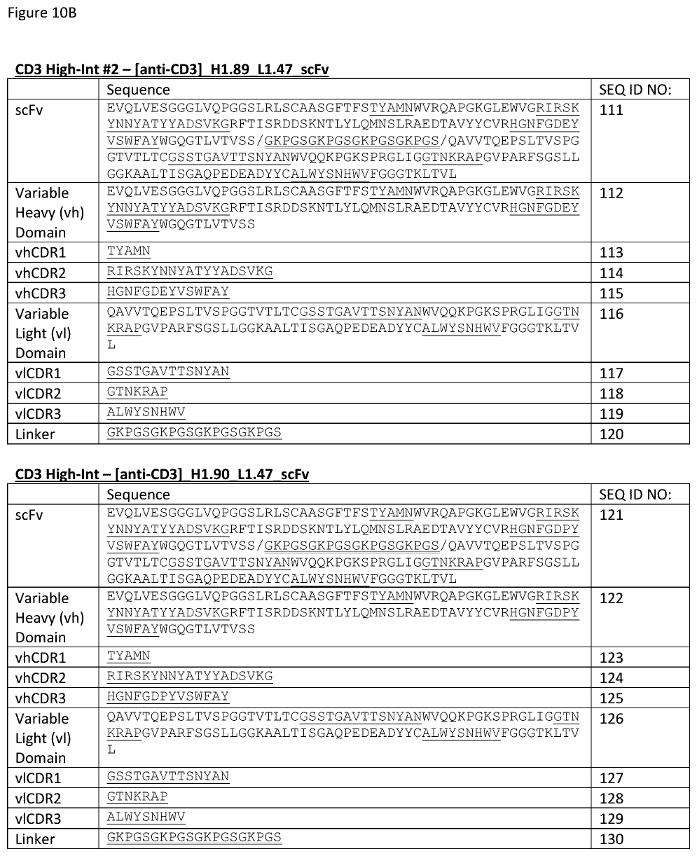

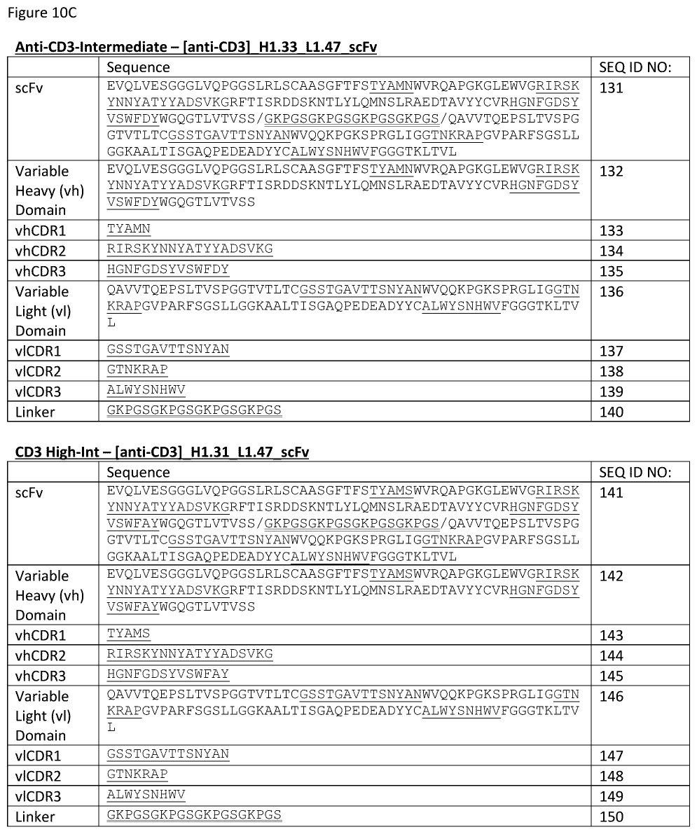

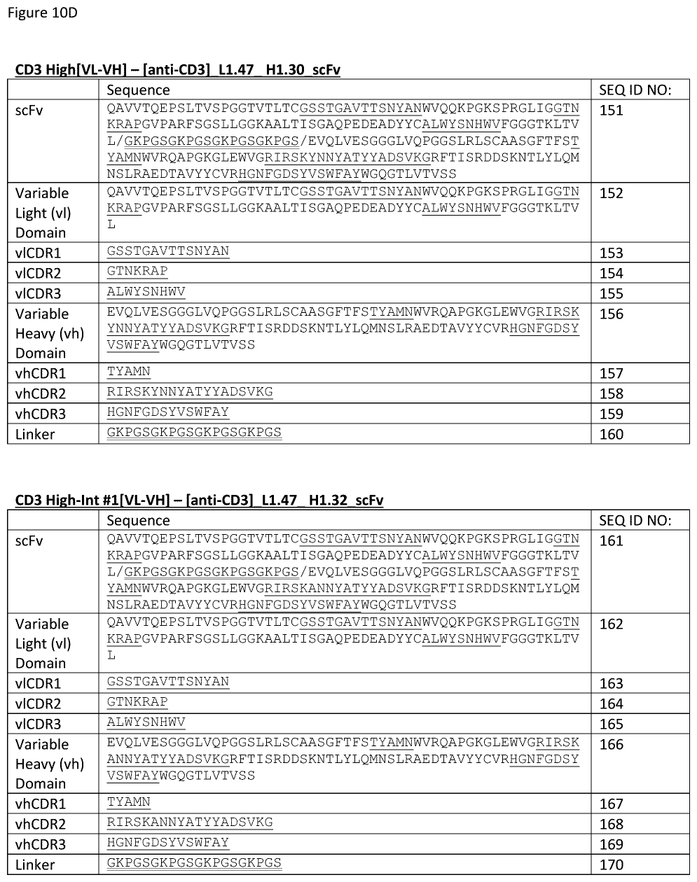

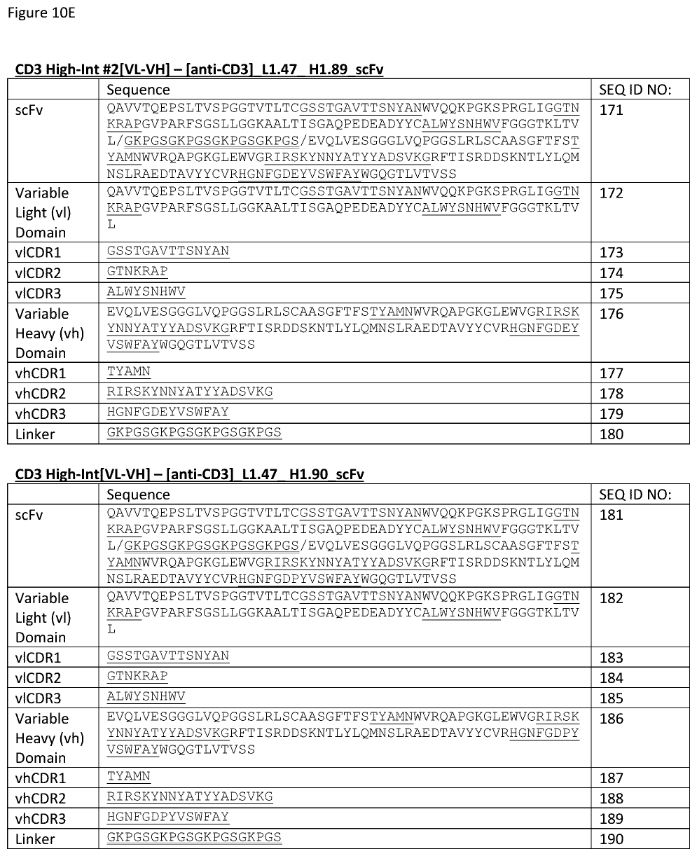

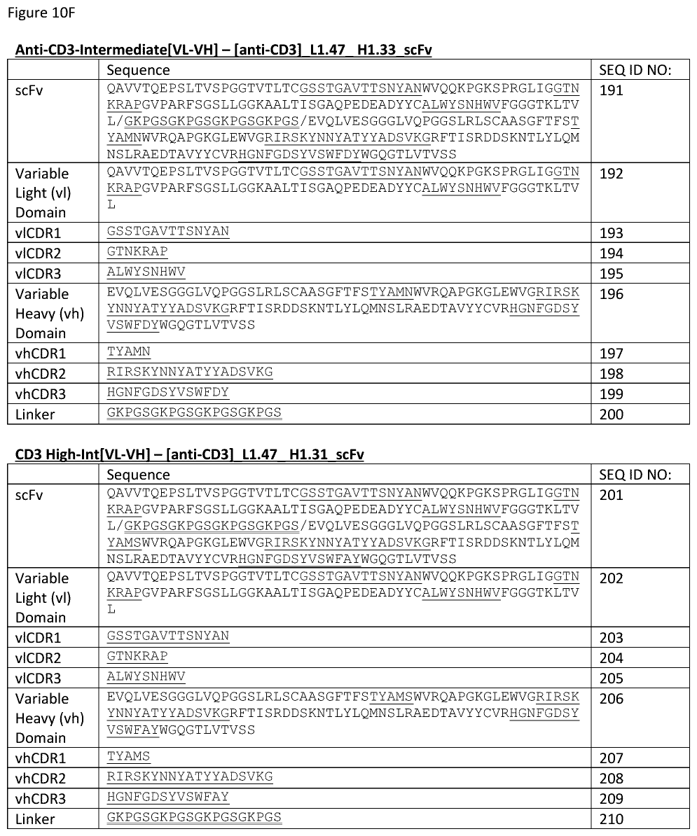

The anti-MSLN×anti-CD3 antibody provided herein can include any suitable CD3 binding domain. In certain embodiments, the anti-MSLN×anti-CD3 antibody includes a CD3 binding domain that includes the VHCDR1, VHCDR2, VHCDR3, VLCDR1, VLCDR2 and VLCDR3 sequences of a CD3 binding domain selected from the group consisting of those depicted in FIGS. 10A-F. In some embodiments, the anti-MSLN×anti-CD3 antibody includes a CD3 binding domain that includes the underlined VHCDR1, VHCDR2, VHCDR3, VLCDR1, VLCDR2 and VLCDR3 sequences of a CD3 binding domain selected from the group consisting of those depicted in FIGS. 10A-10F. In some embodiments, the anti-MSLN×anti-CD3 antibody includes a CD3 binding domain that includes the variable heavy domain and variable light domain of a CD3 binding domain selected from the group consisting of those depicted in FIG. 10A-10F. In some embodiments, the CD3 binding domain is selected from anti-CD3 H1.30_L1.47, anti-CD3 H1.32_L1.47; anti-CD3 H1.89_L1.48; anti-CD3 H1.90_L1.47; Anti-CD3 H1.33_L1.47; and anti-CD3 H1.31_L1.47. As outlined herein, these anti-CD3 antigen binding domains (CD3-ABDs) can be used in scFv formats in either orientation (e.g. from N- to C-terminal, VH-scFv linker-VL or VL-scFv linker-VH).

The antibodies provided herein include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

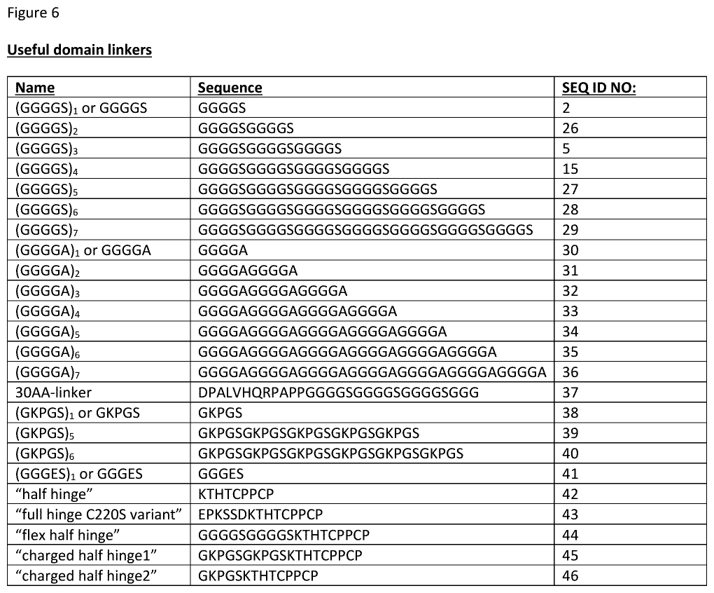

As shown herein, there are a number of suitable linkers (for use as either domain linkers or scFv linkers) that can be used to covalently attach the recited domains (e.g., scFvs, Fabs, Fc domains, etc.), including traditional peptide bonds, generated by recombinant techniques. Exemplary linkers to attach domains of the subject antibody to each other are depicted in FIG. 6. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO: 3), (GGGGS)n (SEQ ID NO: 618), and (GGGS)n (SEQ ID NO: 4), where n is an integer of at least one (and generally from 3 to 4), glycine-alanine polymers, alanine-serine polymers, and other flexible linkers, some of which are shown in FIG. 5 and FIG. 6. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example CK or CA. Linkers can be derived from immunoglobulin heavy chains of any isotype, including for example Cγ1, Cγ2, Cγ3, Cγ4, Cal, Ca2, C8, CE, and Cu. Linker sequences may also be derived from other proteins such as Ig-like proteins (e.g. TCR, FcR, KIR), hinge region-derived sequences, and other natural sequences from other proteins.

In some embodiments, the linker is a “domain linker”, used to link any two domains as outlined herein together. For example, in FIG. 22B, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n (SEQ ID NO: 3), (GGGGS)n (SEQ ID NO: 618), and (GGGS)n (SEQ ID NO: 4), where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used. Exemplary useful domain linkers are depicted in FIG. 6.

With particular reference to the domain linker used to attach the scFv domain to the Fc domain in the “2+1” format, there are several domain linkers that find particular use, including “full hinge C220S variant,” “flex half hinge,” “charged half hinge 1,” and “charged half hinge 2” as shown in FIG. 6.

In some embodiments, the linker is a “scFv linker”, used to covalently attach the VH and VL domains as discussed herein. In many cases, the scFv linker is a charged scFv linker, a number of which are shown in FIG. 5. Accordingly, in some embodiments, the antibodies described herein further provide charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen.

Charged domain linkers can also be used to increase the pI separation of the monomers of the antibodies described herein as well, and thus those included in FIG. 5 can be used in any embodiment herein where a linker is utilized.

In particular, the formats depicted in FIGS. 22A and 22B are antibodies, usually referred to as “heterodimeric antibodies”, meaning that the protein has at least two associated Fc sequences self-assembled into a heterodimeric Fc domain and at least two Fv regions, whether as Fabs or as scFvs.

The MSLN binding domains provided can be included in any useful antibody format including, for example, canonical immunoglobulin, as well as the 1+1 Fab-scFv-Fc and 2+1 Fab2-scFv-Fv formats provided herein. Other useful antibody formats include, but are not limited to, “mAb-Fv,” “mAb-scFv,” “central-Fv”, “one armed scFv-mAb,” “scFv-mAb,” “dual scFv,” and “trident” format antibodies, as disclosed in FIGS. 43A-43K.

In some embodiments, the subject antibody includes one or more of the MSLN ABDs provided herein. In some embodiments, the antibody includes one MSLN ABD. In other embodiments, the antibody includes two MSLN ABDs. In exemplary embodiments, the MSLN ABD includes the variable heavy domain and variable light domain of one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20,_MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In an exemplary embodiment, the antibody is a bispecific antibody that includes one or two MSLN ABDs, including any of the MSLN ABDs provided herein. Bispecific antibody that include such MSLN ABDs include, for example, 1+1 Fab-scFv-Fc and 2+1 Fab2-scFv-Fc bispecifics format antibodies. In exemplary embodiments, the MSLN ABD is one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20,_MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21). In exemplary embodiments, the MSLN binding domains is a Fab. In some embodiments, such bispecific antibodies are heterodimeric bispecific antibodies that include any of the heterodimerization skew variants, pI variants and/or ablation variants described herein.

### A. Chimeric and Humanized Antibodies

In certain embodiments, the antibodies described herein comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants described herein). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants described herein).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294: 151-162; Baca et al., 1997, J. Biol. Chem. 272(16): 10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### B. Heterodimeric Antibodies

In exemplary embodiments, the bispecific antibodies provided herein are heterodimeric bispecific antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in US Patent No. U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

### 1. Skew Variants

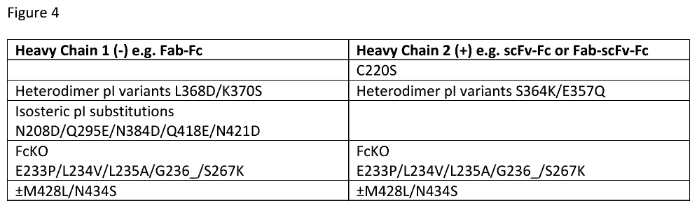

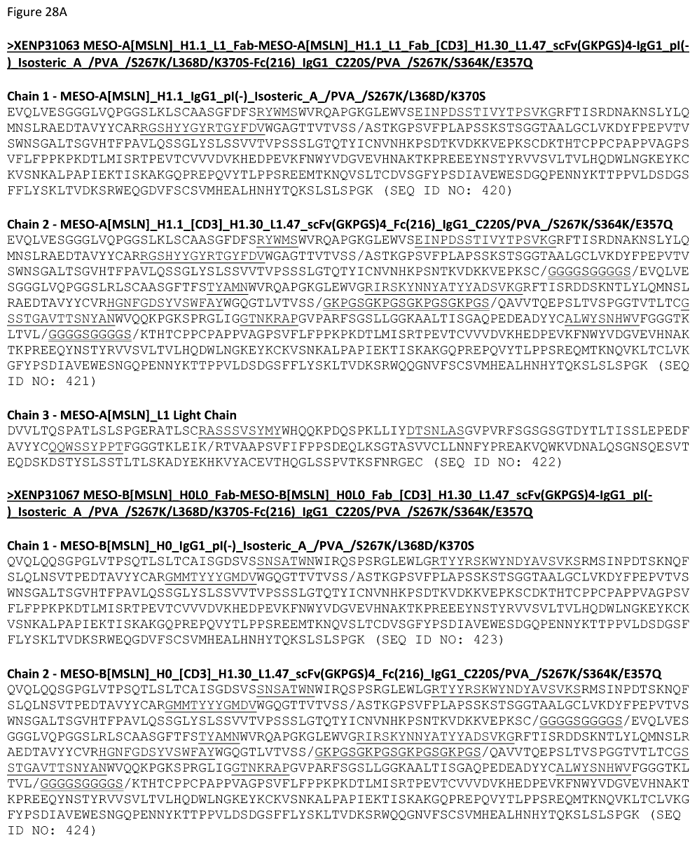

In some embodiments, the heterodimeric antibody includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 1A-1E and FIG. 4.

One mechanism is generally referred to in the art as “knobs and holes”, referring to amino acid engineering that creates steric influences to favor heterodimeric formation and disfavor homodimeric formation can also optionally be used; this is sometimes referred to as “knobs and holes”, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety. The Figures identify a number of “monomer A-monomer B” pairs that rely on “knobs and holes”. In addition, as described in Merchant et al., Nature Biotech. 16:677 (1998), these “knobs and hole” mutations can be combined with disulfide bonds to skew formation to heterodimerization.

An additional mechanism that finds use in the generation of heterodimers is sometimes referred to as “electrostatic steering” as described in Gunasekaran et al., J. Biol. Chem. 285(25): 19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R (e.g. these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIG. 4. In exemplary embodiments, the heterodimeric antibody includes a S364K/E357Q: L368D/K370S; L368D/K370S: S364K; L368E/K370S: S364K; T411T/E360E/Q362E: D401K; L368D/K370S: S364K/E357L; K370S: S364K/E357Q; or a T366S/L368A/Y407V: T366W (optionally including a bridging disulfide, T366S/L368A/Y407V/Y349C: T366W/S354C) “skew” variant amino acid substitution set. In an exemplary embodiment, the heterodimeric antibody includes a “S364K/E357Q: L368D/K370S” amino acid substitution set. In terms of nomenclature, the pair “S364K/E357Q: L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

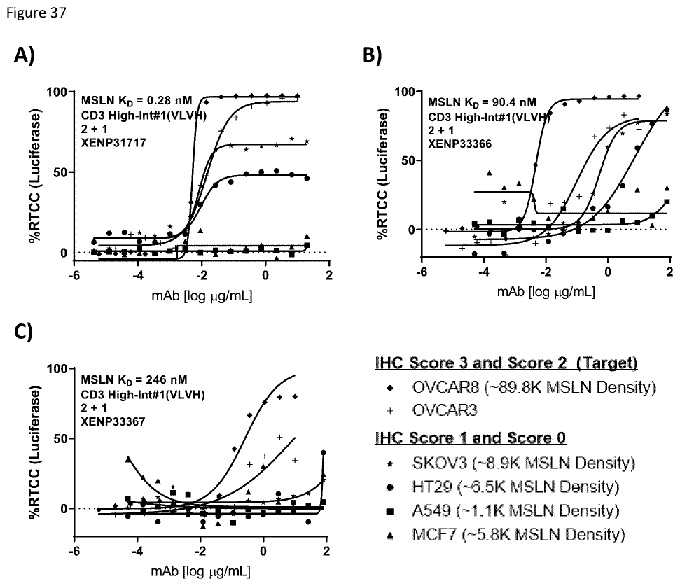

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotypic variants, FcRn variants, ablation variants, etc. into one or both of the first and second Fc domains of the heterodimeric antibody. Further, individual modifications can also independently and optionally be included or excluded from the subject the heterodimeric antibody.

Additional monomer A and monomer B variants that can be combined with other variants, optionally and independently in any amount, such as pI variants outlined herein or other steric variants that are shown in FIG. 37 of US 2012/0149876, the figure and legend and SEQ ID NOs of which are incorporated expressly by reference herein.

In some embodiments, the steric variants outlined herein can be optionally and independently incorporated with any pI variant (or other variants such as Fc variants, FcRn variants, etc.) into one or both monomers, and can be independently and optionally included or excluded from the proteins of the antibodies described herein.

A list of suitable skew variants is found in FIGS. 1A-1E, with FIG. 4 showing some pairs of particular utility in many embodiments. Of particular use in many embodiments are the pairs of sets including, but not limited to, S364K/E357Q: L368D/K370S; L368D/K370S: S364K; L368E/K370S: S364K; T411T/E360E/Q362E: D401K; L368D/K370S: S364K/E357L and K370S: S364K/E357Q. In terms of nomenclature, the pair “S364K/E357Q: L368D/K370S” means that one of the monomers has the double variant set S364K/E357Q and the other has the double variant set L368D/K370S.

### 2. pI (Isoelectric Point) Variants for Heterodimers

In some embodiments, the heterodimeric antibody includes purification variants that advantageously allow for the separation of heterodimeric antibody (e.g., anti-MSLN×anti-CD3 bispecific antibody) from homodimeric proteins.

There are several basic mechanisms that can lead to case of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format and the “2+1 Fab₂-scFv-Fc” format, also allows separation on the basis of size. As described above, it is also possible to “skew” the formation of heterodimers over homodimers using skew variants. Thus, a combination of heterodimerization skew variants and pI variants find particular use in the heterodimeric antibodies provided herein.

Additionally, as more fully outlined below, depending on the format of the heterodimeric antibody, pI variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc formats are useful with just charged scFv linkers and no additional pI adjustments, although the antibodies described herein do provide pI variants that are on one or both of the monomers, and/or charged domain linkers as well. In addition, additional amino acid engineering for alternative functionalities may also confer pI changes, such as Fc, FcRn and KO variants.

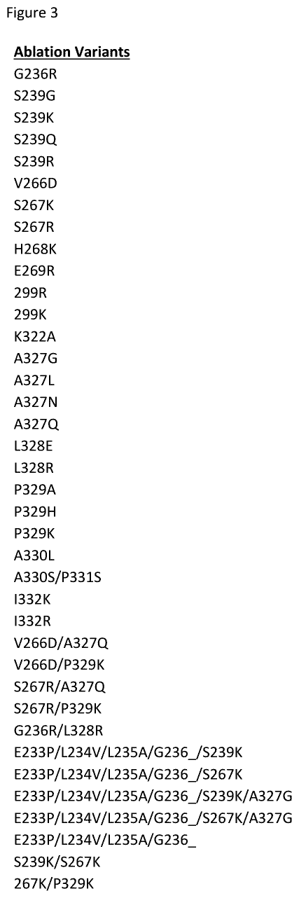

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine.). A number of these variants are shown in the FIGS. 3 and 4.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the antibodies described herein.

As will be appreciated by those in the art, the number of pI variants to be included on each or both monomer(s) to get good separation will depend in part on the starting pI of the components, for example in the 1+1 Fab-scFv-Fc and 2+1 Fab₂-scFv-Fc formats, the starting pI of the scFv and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the antibodies described herein. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively or in addition to isotypic substitutions, the possibility of immunogenicity resulting from the pI variants is significantly reduced by utilizing isosteric substitutions (e.g. Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

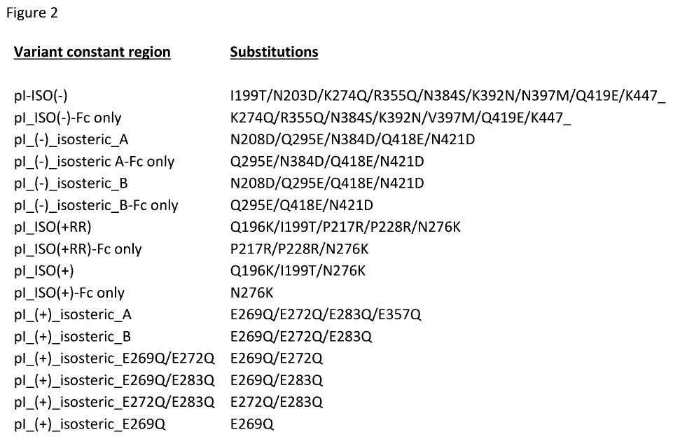

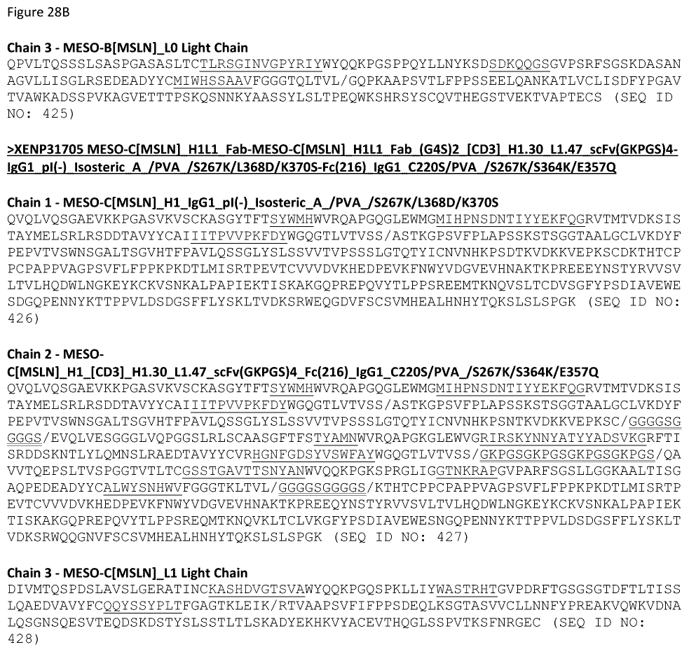

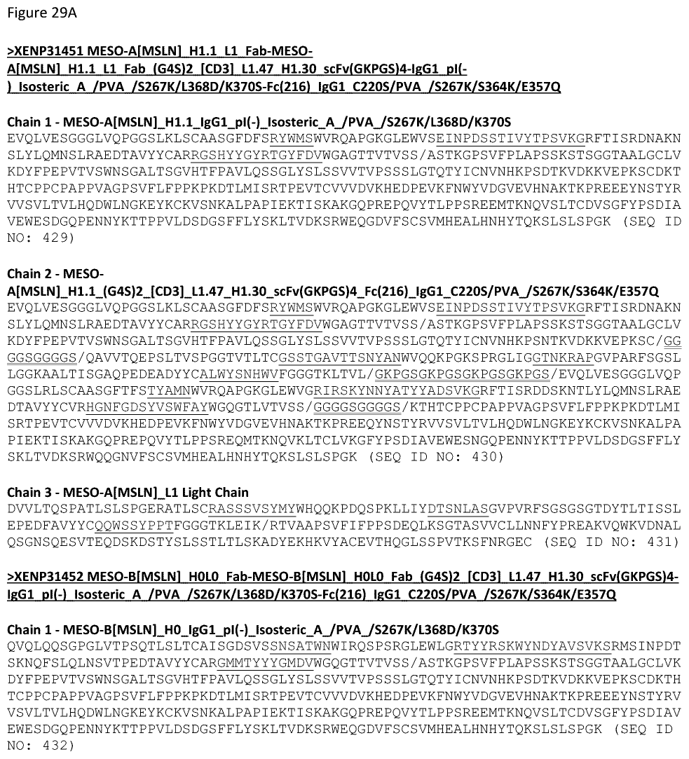

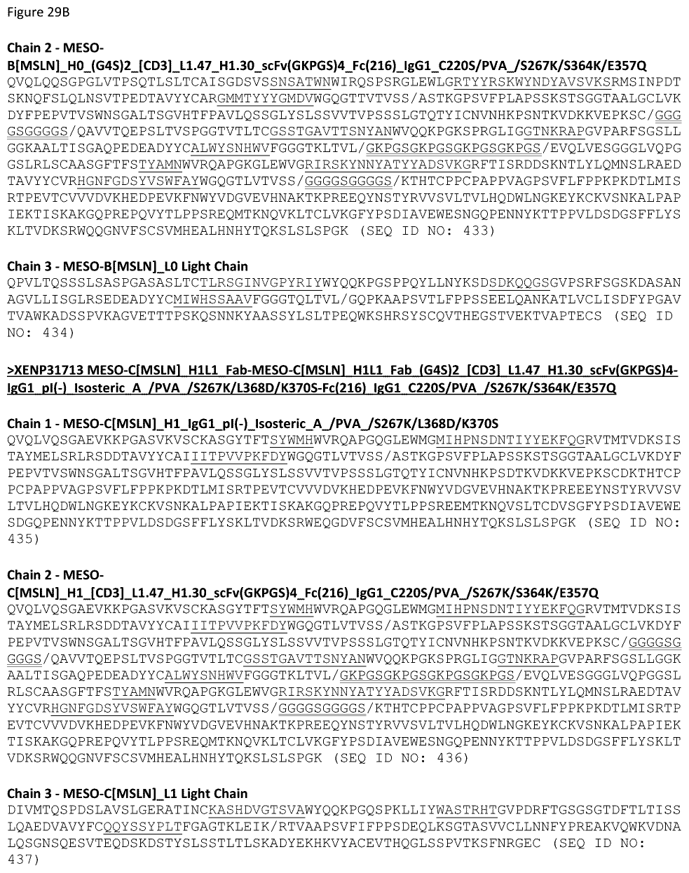

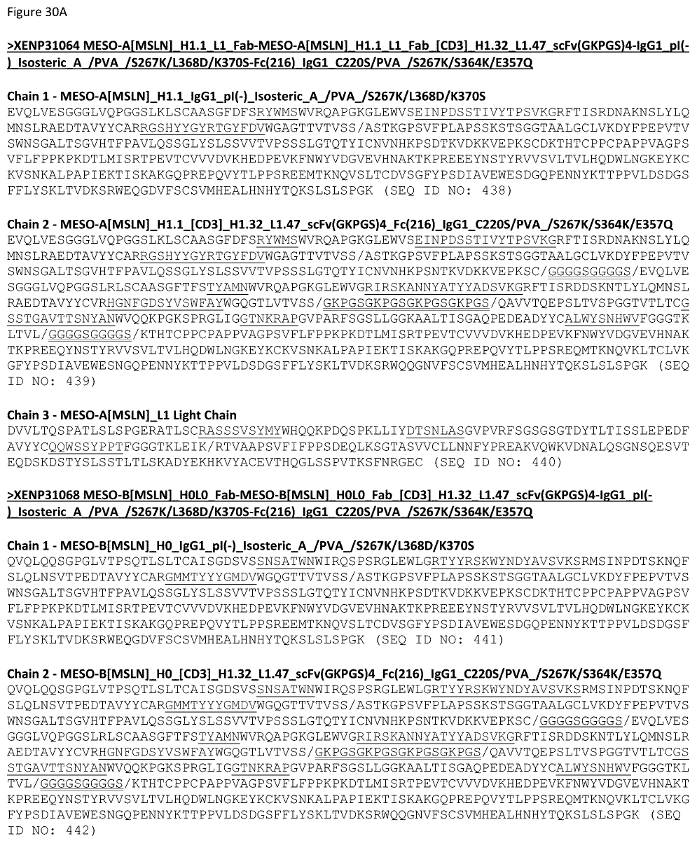

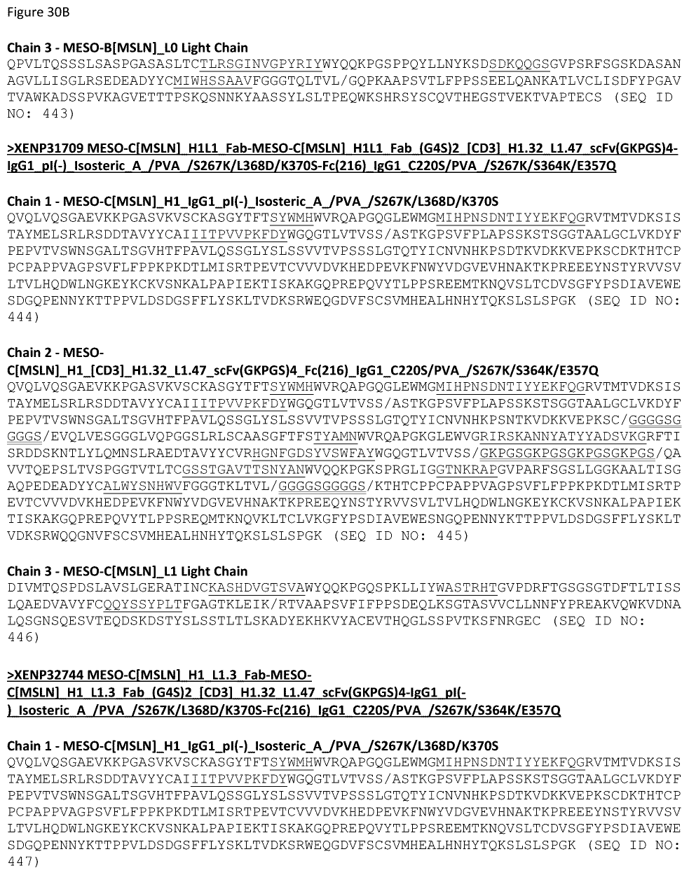

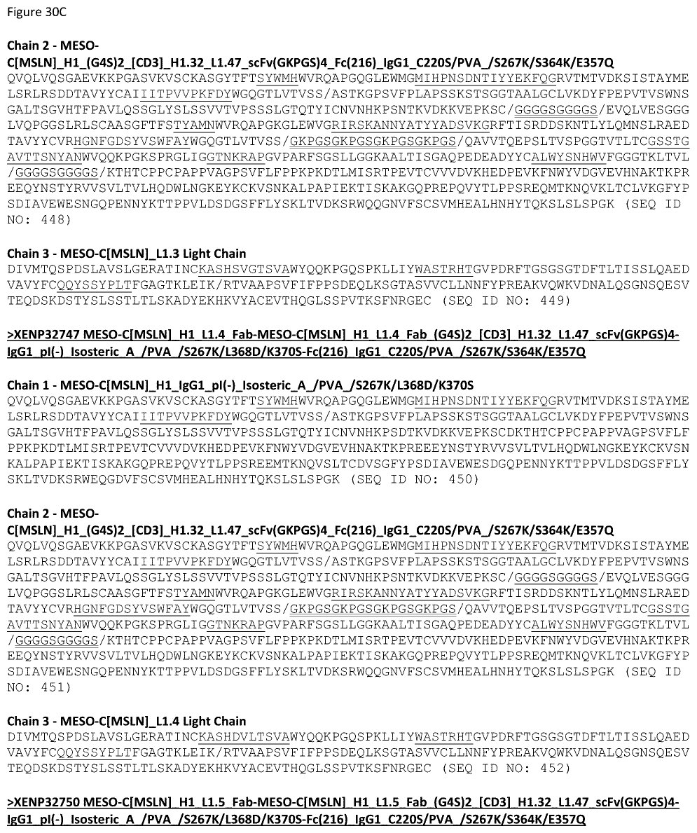

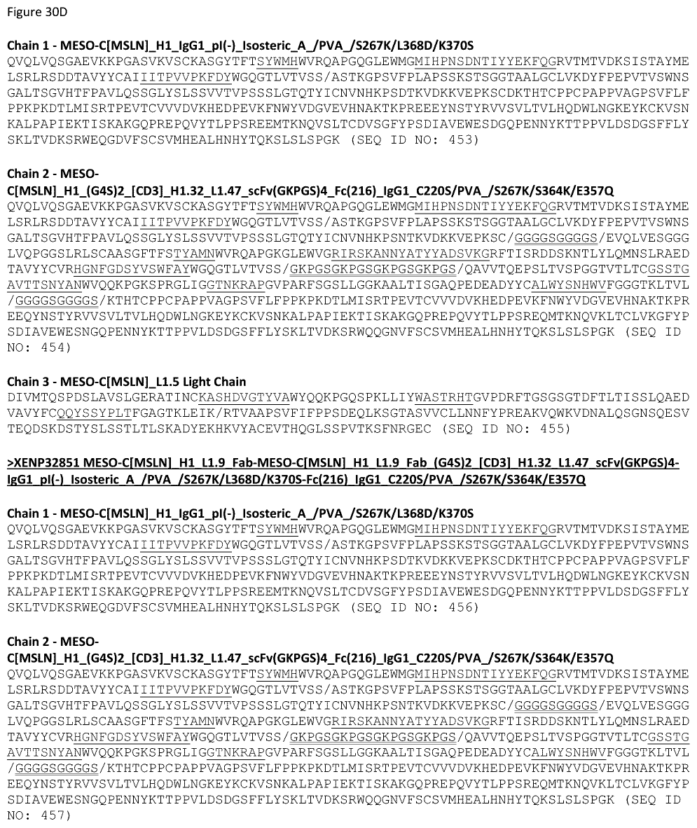

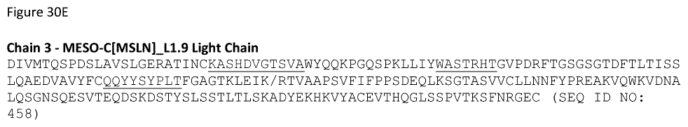

Exemplary combinations of pI variants are shown in FIGS. 4 and 5, and FIG. 30 of US Publ. App. No. 2016/0355608, all of which are herein incorporated by reference in its entirety and specifically for the disclosure of pI variants. Preferred combinations of pI variants are shown in FIGS. 1 and 2. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)+(SEQ ID NO: 1). However, as will be appreciated by those in the art, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 2 and the other monomer has a charged linker (either in the form of a charged scFv linker because that monomer comprises an scFv or a charged domain linker, as the format dictates, which can be selected from those depicted in FIG. 5).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., =may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Preferred combinations of pI variants are shown in FIG. 4. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, for example in the FIGS. 22A and 22B formats, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)+(SEQ ID NO: 1). However, as will be appreciated by those in the art, the first monomer includes a CH1 domain, including position 208. Accordingly, in constructs that do not include a CH1 domain (for example for antibodies that do not utilize a CH1 domain on one of the domains, for example in a dual scFv format or a “one armed” format such as those depicted in FIG. 43B, 43C, or 43D), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 4 and the other monomer has a charged linker (either in the form of a charged scFv linker because that monomer comprises an scFv or a charged domain linker, as the format dictates, which can be selected from those depicted in FIG. 5).

### 3. Isotypic Variants

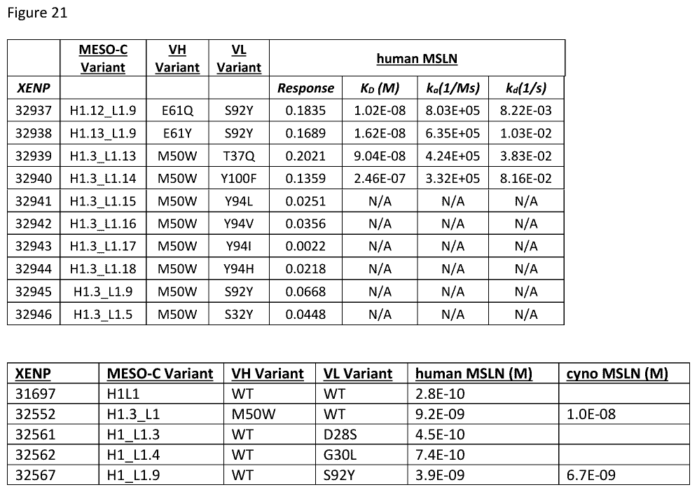

In addition, many embodiments of the antibodies described herein rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g. by changing a higher pI amino acid to a lower pI amino acid), or to allow accommodations in structure for stability, etc. as is more further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

### 4. Calculating pI

The pI of each monomer can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

5. pI Variants that Also Confer Better FcRn In Vivo Binding

In the case where the pI variant decreases the pI of the monomer, they can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall'Acqua et al. showed that Fc mutants with increased FcRn binding at pH 6 and pH 7.4 actually had reduced serum concentrations and the same half life as wild-type Fc (Dall'Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### C. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc, as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

### 1. FcγR Variants

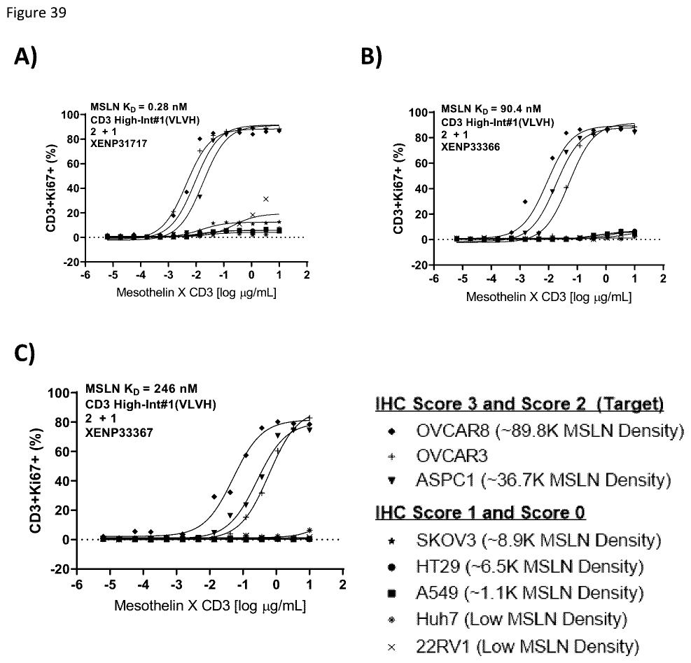

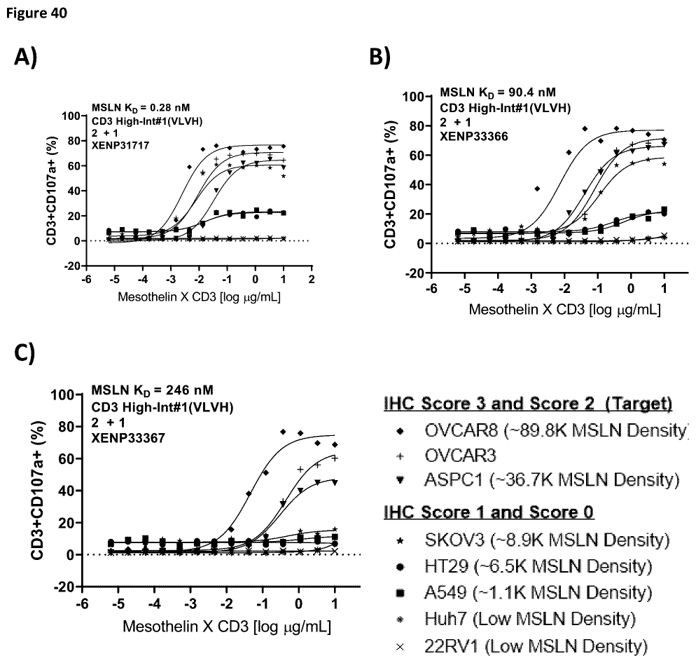

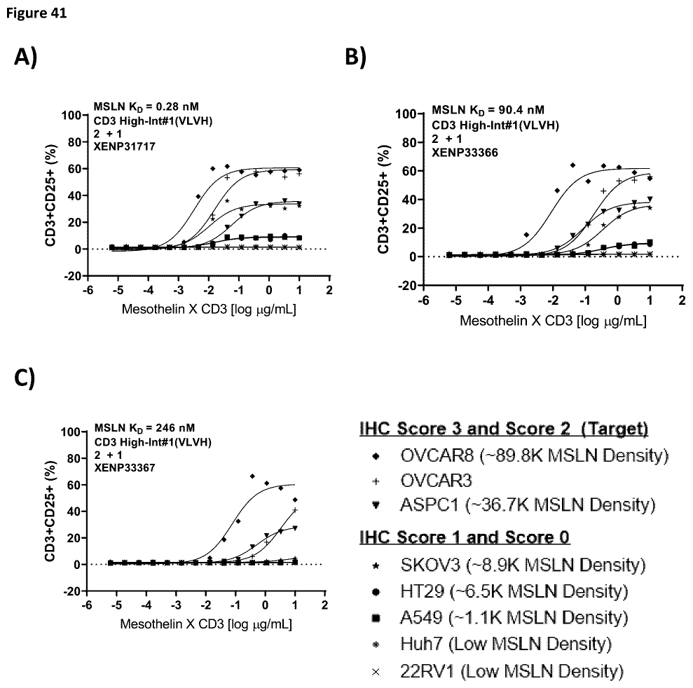

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the antibodies described herein include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D/332E/330L, 243A, 243L, 264A, 264V and 299T.

In addition, there are additional Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 2591, 428L/434S, 2591/308F, 436I/428L, 436I or V/434S, 436V/428L and 2591/308F/428L. Such modification may be included in one or both Fc domains of the subject antibody.

### 2. Ablation Variants

Similarly, another category of functional variants are “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g. FcγR1, FcγRIIa, FcγRIIb, FcγRIIIa, etc.) to avoid additional mechanisms of action. That is, for example, in many embodiments, particularly in the use of bispecific antibodies that bind CD3 monovalently it is generally desirable to ablate FcγRIIIa binding to eliminate or significantly reduce ADCC activity. wherein one of the Fc domains comprises one or more Fcγ receptor ablation variants. These ablation variants are depicted in FIG. 14, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### D. Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition”. In some embodiments, the heterodimeric antibodies provided herein include the combination of heterodimerizaition skew variants, isosteric pI substitutions and FcKO variants as depicted in FIG. 4. In addition, all of these variants can be combined into any of the heterodimerization formats.

In the case of pI variants, while embodiments finding particular use are shown in the Figures, other combinations can be generated, following the basic rule of altering the pI difference between two monomers to facilitate purification.

In addition, any of the heterodimerization variants, skew and pI, are also independently and optionally combined with Fc ablation variants, Fc variants, FcRn variants, as generally outlined herein.

Exemplary combination of variants that are included in some embodiments of the heterodimeric 1+1 Fab-scFv-Fc and 2+1 Fab₂-scFv-Fc format antibodies are included in FIG. 4. In certain embodiments, the antibody is a heterodimeric 1+1 Fab-scFv-Fc or 2+1 Fab₂-scFv-Fc format antibody as shown in FIGS. 15A and 15B.

### E. Anti-MSLN×Anti-CD3 Bispecific Antibodies

In another aspect, provided herein are anti-MSLN×anti-CD3 (also referred to herein as “αMSLN×αCD3”) bispecific antibodies. Such antibodies include at least one MSLN binding domain and at least one CD3 binding domain. In some embodiments, bispecific αMSLN×αCD3 provided herein immune responses selectively in tumor sites that express MSLN.

Note that unless specified herein, the order of the antigen list in the name does not confer structure; that is a MSLN×CD3 1+1 Fab-scFv-Fc antibody can have the scFv bind to MSLN or CD3, although in some cases, the order specifies structure as indicated.

As is more fully outlined herein, these combinations of ABDs can be in a variety of formats, as outlined below, generally in combinations where one ABD is in a Fab format and the other is in an scFv format. Exemplary formats that are used in the bispecific antibodies provided herein include the 1+1 Fab-scFv-Fc and 2+1 Fab2-scFv-Fv formats (see, e.g., FIGS. 22A and 22B). Other useful antibody formats include, but are not limited to, “mAb-Fv,” “mAb-scFv,” “central-Fv”, “one armed scFv-mAb,” “scFv-mAb,” “dual scFv,” and “trident” format antibodies, as disclosed in FIG. 43A-43K.

In addition, in general, one of the ABDs comprises a scFv as outlined herein, in an orientation from N- to C-terminus of VH-scFv linker-VL or VL-scFv linker-VH. One or both of the other ABDs, according to the format, generally is a Fab, comprising a VH domain on one protein chain (generally as a component of a heavy chain) and a VL on another protein chain (generally as a component of a light chain).

As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain). The scFv sequences contained in the sequence listing utilize a particular charged linker, but as outlined herein, uncharged or other charged linkers can be used, including those depicted in FIG. 5 and FIG. 6.

In addition, as discussed above, the numbering used in the Sequence Listing for the identification of the CDRs is Kabat, however, different numbering can be used, which will change the amino acid sequences of the CDRs as shown in Table 2.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85 or 90% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 85 or 90% identity to a human germline sequence selected from those listed in FIG. 1 of U.S. Pat. No. 7,657,380.

The anti-MSLN×anti-CD3 bispecific antibody can include any suitable CD3 ABD, including those described herein (see, e.g., FIGS. 10A-10F). In some embodiments, the CD3 ABD of the anti-MSLN×anti-CD3 bispecific antibody includes the variable heavy domain and variable light domain of a CD3 ABD provided herein, including those described in FIGS. 10A-10F and the sequence listing. In some embodiments, the CD3 ABD includes the variable heavy domain and variable light domain of one of the following CD3 ABDs: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F). In exemplary embodiments, the CD3 ABD is one of the following CD3 ABDs: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F) or a variant thereof.

The anti-MSLN×anti-CD3 bispecific antibody can include any suitable MSLN ABD, including those described herein (see, e.g., FIGS. 15-21). In some embodiments, the MSLN ABD of the anti-MSLN×anti-CD3 bispecific antibody includes the variable heavy domain and variable light domain of a MSLN ABD provided herein, including those described in FIGS. 15-21 and the sequence listing. In some embodiments, the MSLN ABD includes the variable heavy domain and variable light domain of one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In exemplary embodiments, the MSLN ABD is one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21) or variants thereof.

In some embodiments, the anti-MSLN×anti-CD3 bispecific antibody includes a MSLN binding domain variable heavy domain selected from any of FIGS. 15-18 and 20-21. In some embodiments, the MSLN binding domain includes a MSLN binding domain variable light domain selected from any of FIGS. 15-17 and 19-21. In some embodiments, the MSLN binding domain includes a combination of any of the MSLN binding domain variable heavy domains and variable light domains in FIGS. 15-21.

### F. Useful Formats of the Invention

As will be appreciated by those in the art and discussed more fully below, the heterodimeric bispecific antibodies provided herein can take on a wide variety of configurations, as are generally depicted in FIG. 1. Some figures depict “single ended” configurations, where there is one type of specificity on one “arm” of the molecule and a different specificity on the other “arm”. Other figures depict “dual ended” configurations, where there is at least one type of specificity at the “top” of the molecule and one or more different specificities at the “bottom” of the molecule. Thus, in some embodiments, the antibodies described herein are directed to novel immunoglobulin compositions that co-engage a different first and a second antigen.

As will be appreciated by those in the art, the heterodimeric formats of the antibodies described herein can have different valencies as well as be bispecific. That is, heterodimeric antibodies of the antibodies described herein can be bivalent and bispecific, wherein one target tumor antigen (e.g. CD3) is bound by one binding domain and the other target tumor antigen (e.g. MSLN) is bound by a second binding domain. The heterodimeric antibodies can also be trivalent and bispecific, wherein the first antigen is bound by two binding domains and the second antigen by a second binding domain. As is outlined herein, when CD3 is one of the target antigens, it is preferable that the CD3 is bound only monovalently, to reduce potential side effects.

The antibodies described herein utilize anti-CD3 antigen binding domains in combination with anti-MSLN binding domains. As will be appreciated by those in the art, any collection of anti-CD3 CDRs, anti-CD3 variable light and variable heavy domains, Fabs and scFvs as depicted in any of the Figures can be used. Similarly, any of the anti-MSLN antigen binding domains can be used, whether CDRs, variable light and variable heavy domains, Fabs and scFvs as depicted in any of the Figures (e.g., FIGS. 15-21) can be used, optionally and independently combined in any combination.

### 1. 1+1 Fab-scFv-Fc Format

One heterodimeric scaffold that finds particular use in the antibodies described herein is the “1+1 Fab-scFv-Fc” or “bottle-opener” format as shown in FIG. 15A with an exemplary combination of a CD3 binding domain and a tumor target antigen (MSLN) binding domain. In this embodiment, one heavy chain monomer of the antibody contains a single chain Fv (“scFv”, as defined below) and an Fc domain. The scFv includes a variable heavy domain (VH1) and a variable light domain (VL1), wherein the VH1 is attached to the VL1 using an scFv linker that can be charged (see, e.g., FIG. 5). The scFv is attached to the heavy chain using a domain linker (see, e.g., FIG. 6). The other heavy chain monomer is a “regular” heavy chain (VH-CH1-hinge-CH2-CH3). The 1+1 Fab-scFv-Fc also includes a light chain that interacts with the VH-CH1 to form a Fab. This structure is sometimes referred to herein as the “bottle-opener” format, due to a rough visual similarity to a bottle-opener. The two heavy chain monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below.

There are several distinct advantages to the present “1+1 Fab-scFv-Fc” format. As is known in the art, antibody analogs relying on two scFv constructs often have stability and aggregation problems, which can be alleviated in the antibodies described herein by the addition of a “regular” heavy and light chain pairing. In addition, as opposed to formats that rely on two heavy chains and two light chains, there is no issue with the incorrect pairing of heavy and light chains (e.g. heavy 1 pairing with light 2, etc.).

Many of the embodiments outlined herein rely in general on the 1+1 Fab-scFv-Fc or “bottle opener” format antibody that comprises a first monomer comprising an scFv, comprising a variable heavy and a variable light domain, covalently attached using an scFv linker (charged, in many but not all instances), where the scFv is covalently attached to the N-terminus of a first Fc domain usually through a domain linker The domain linker can be either charged or uncharged and exogenous or endogenous (e.g., all or part of the native hinge domain). Any suitable linker can be used to attach the scFv to the N-terminus of the first Fc domain. In some embodiments, the domain linker is chosen from the domain linkers in FIG. 6. The second monomer of the 1+1 Fab-scFv-Fc format or “bottle opener” format is a heavy chain, and the composition further comprises a light chain.

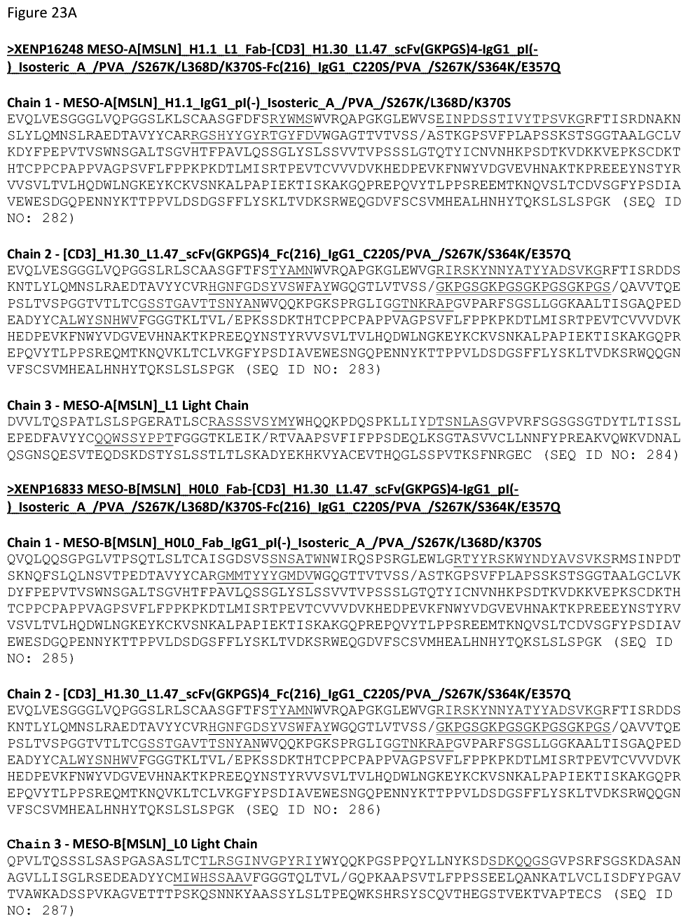

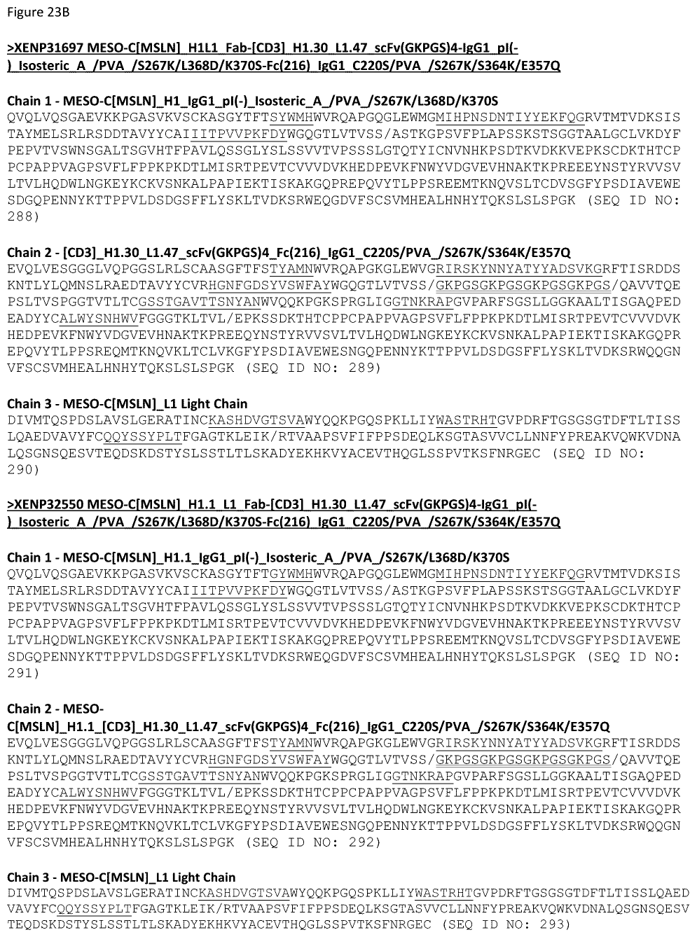

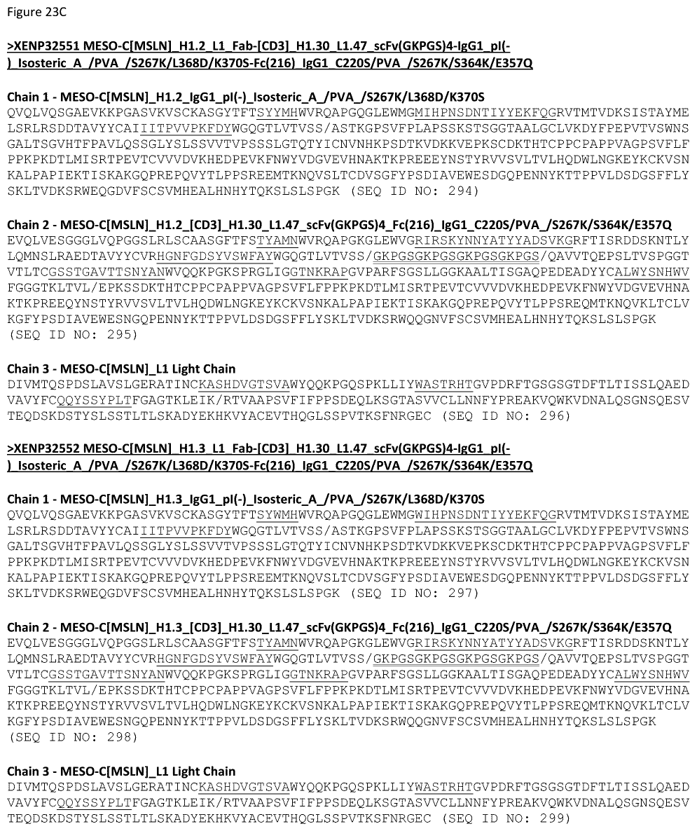

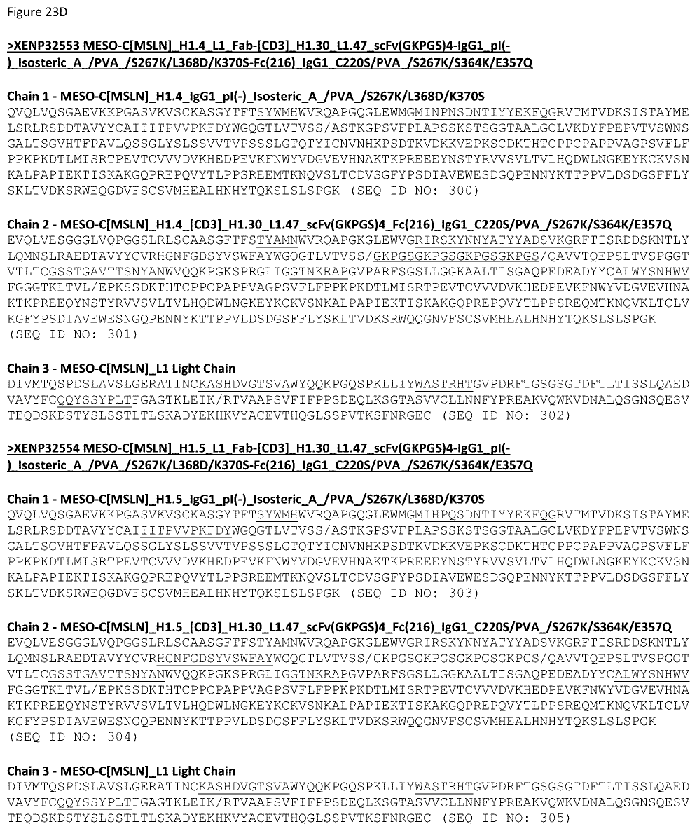

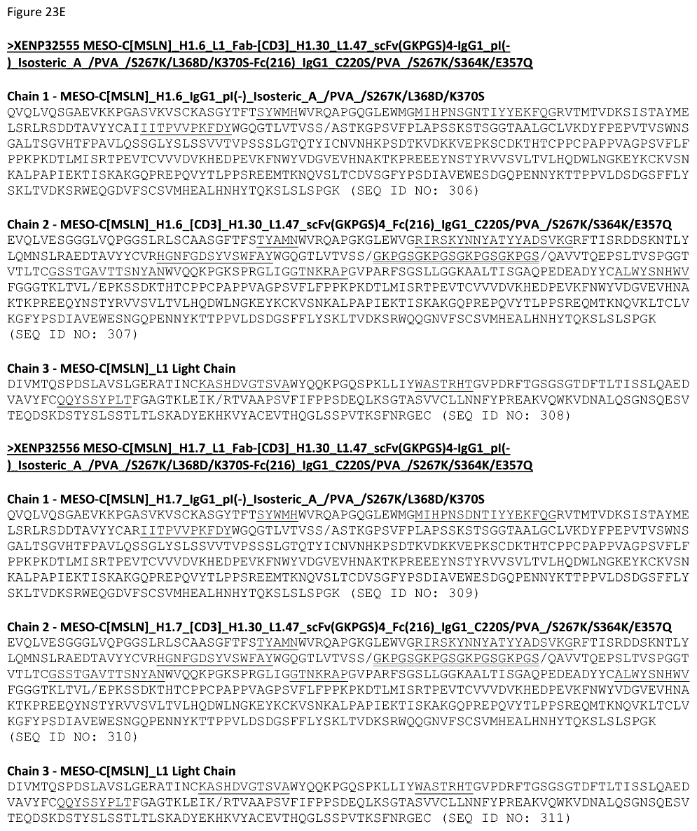

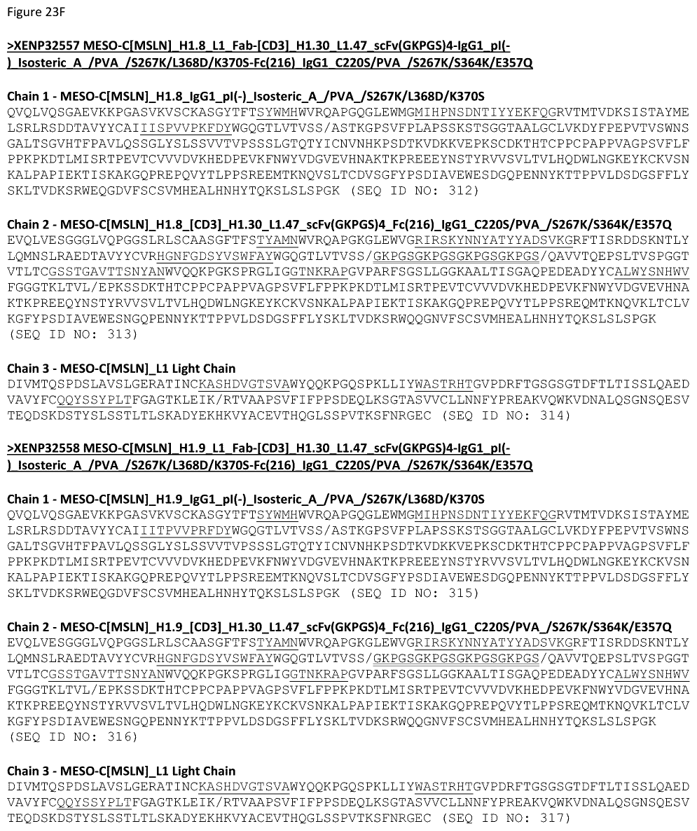

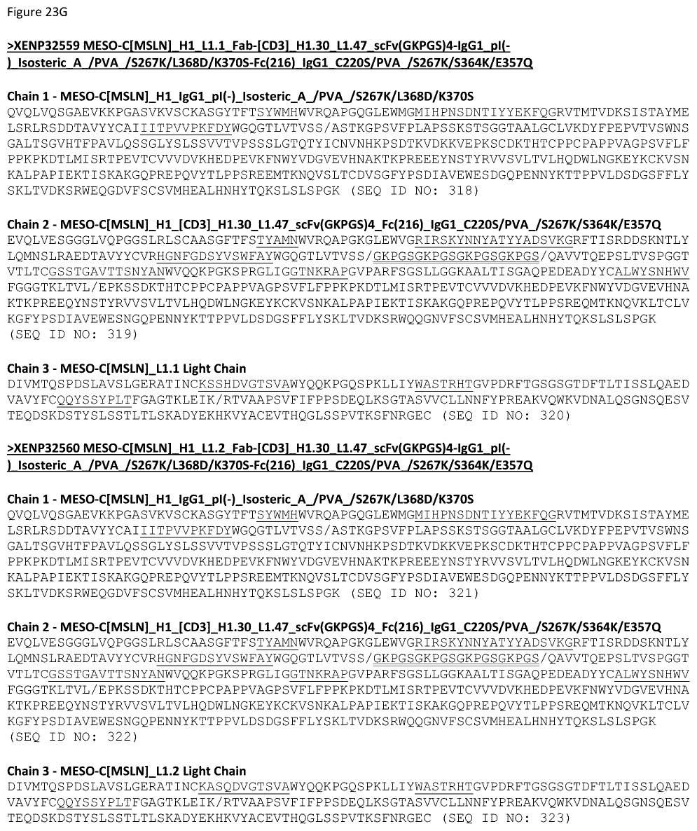

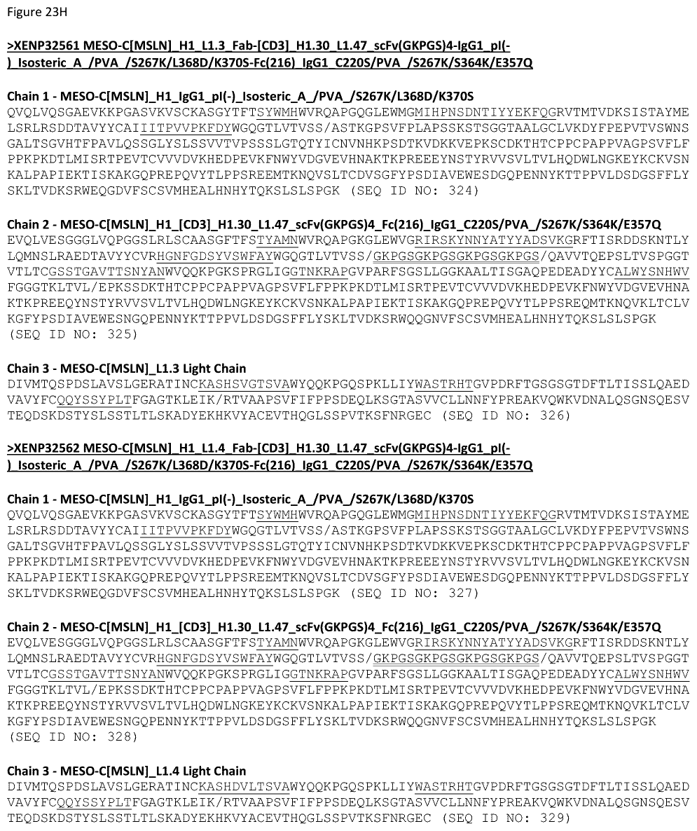

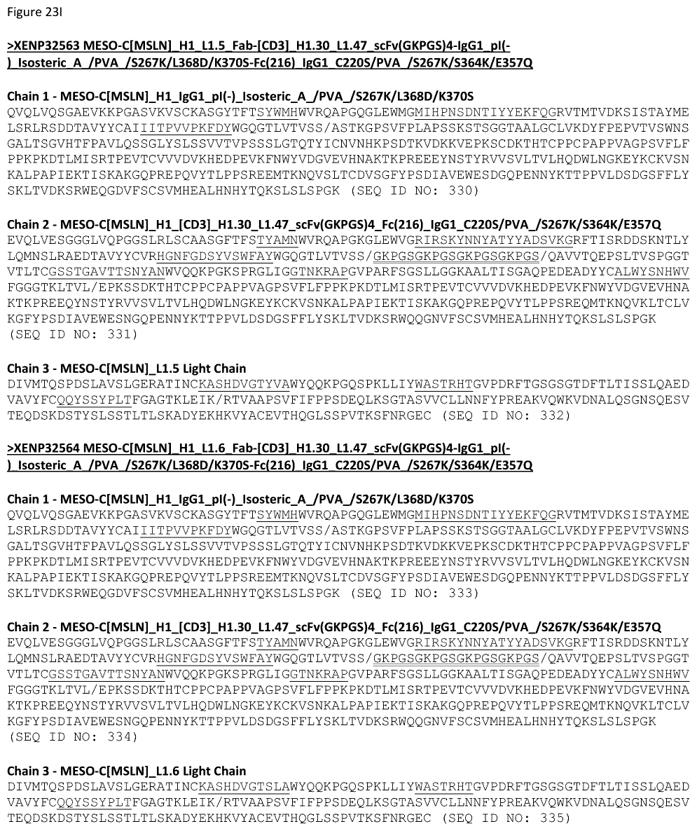

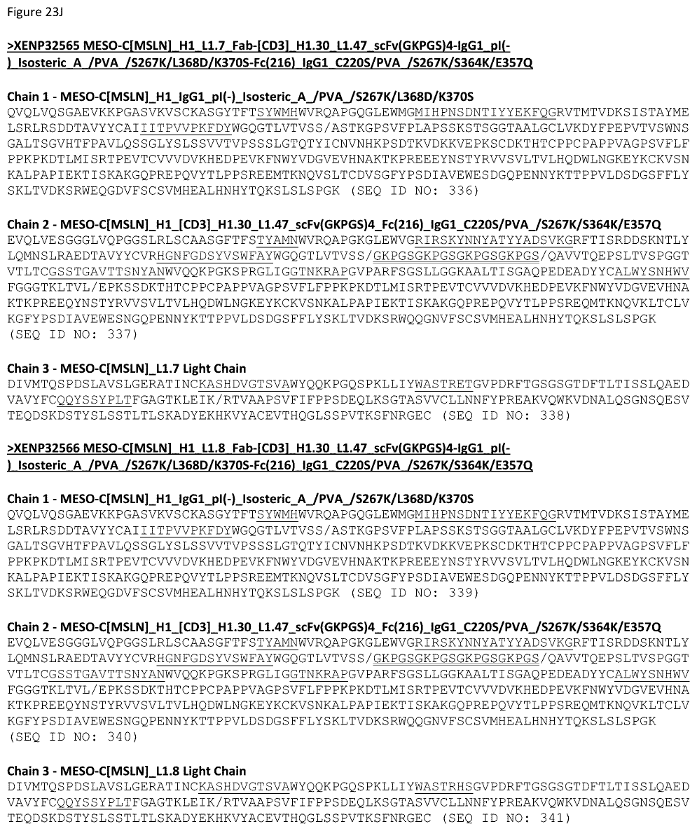

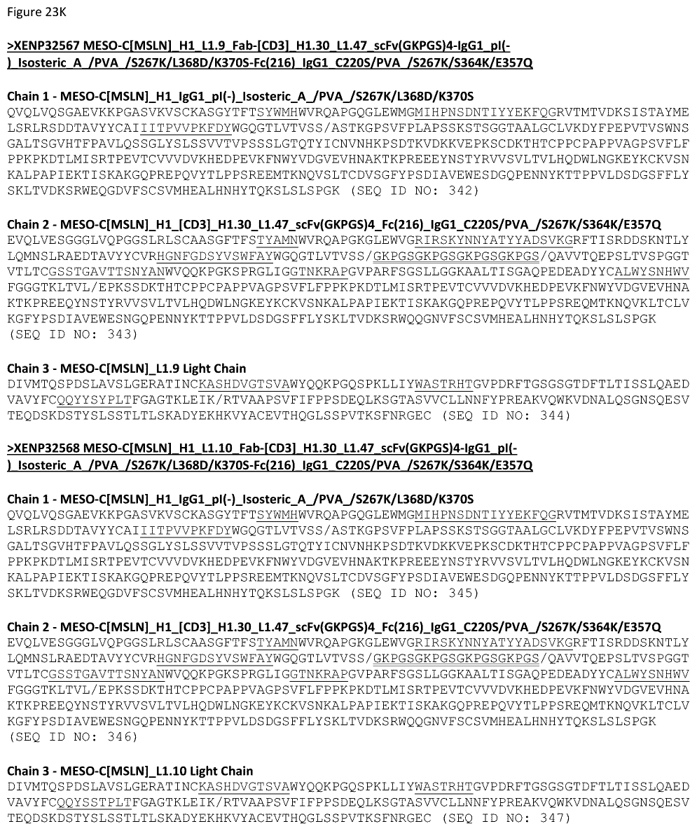

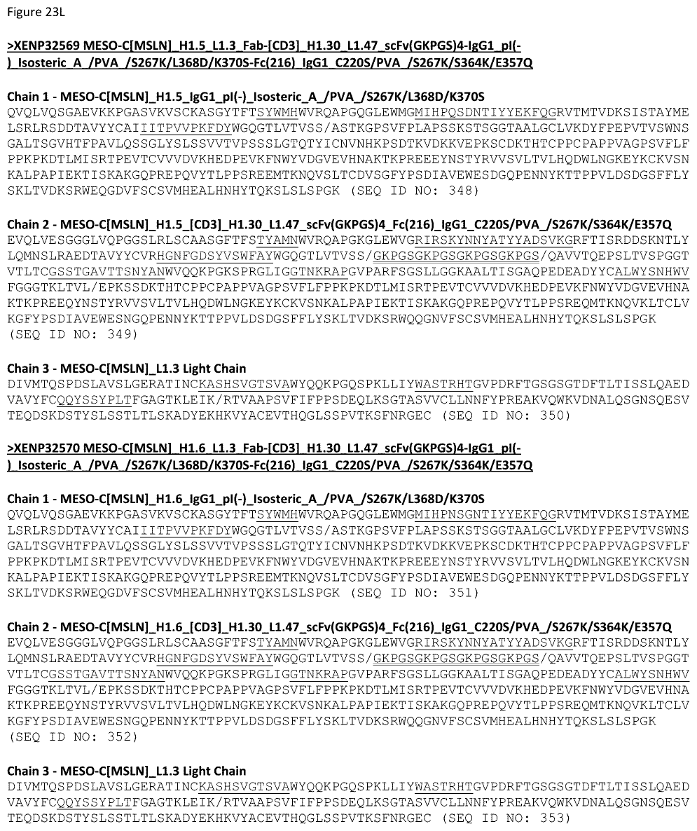

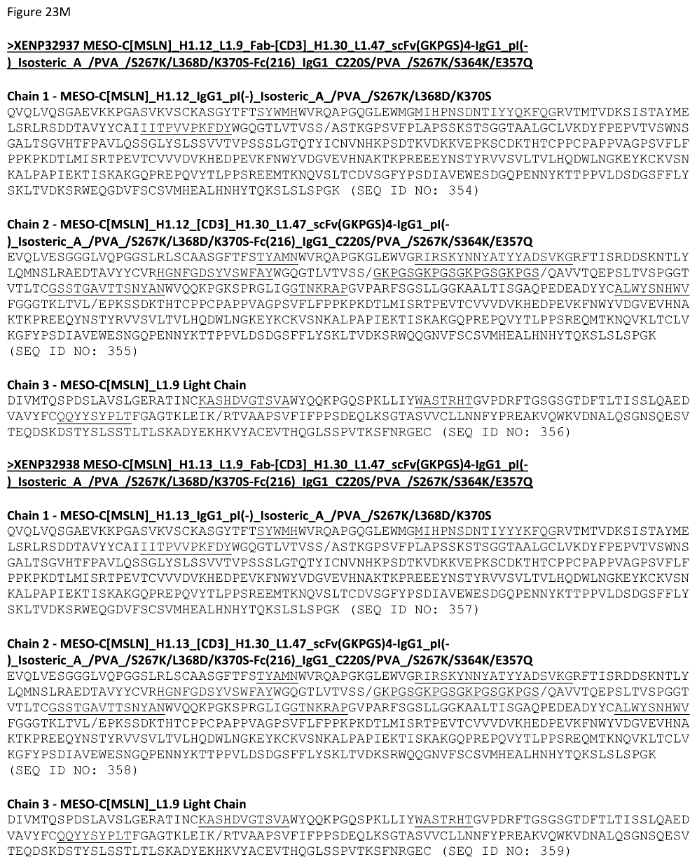

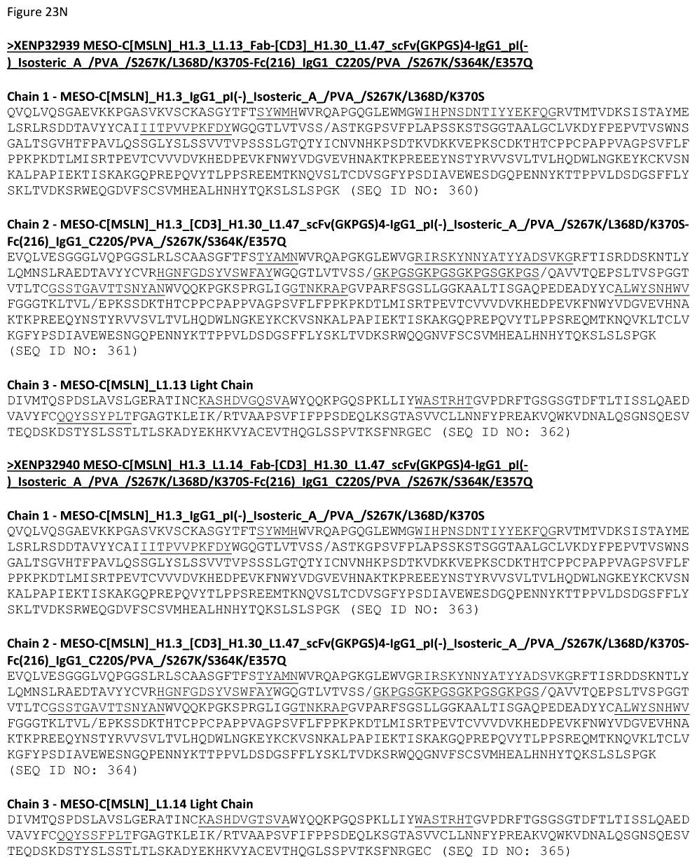

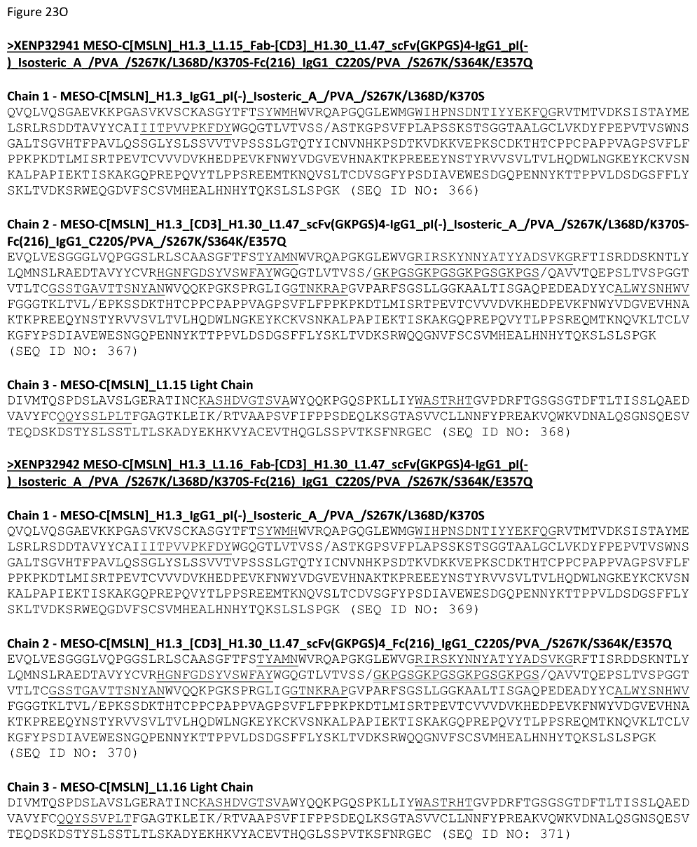

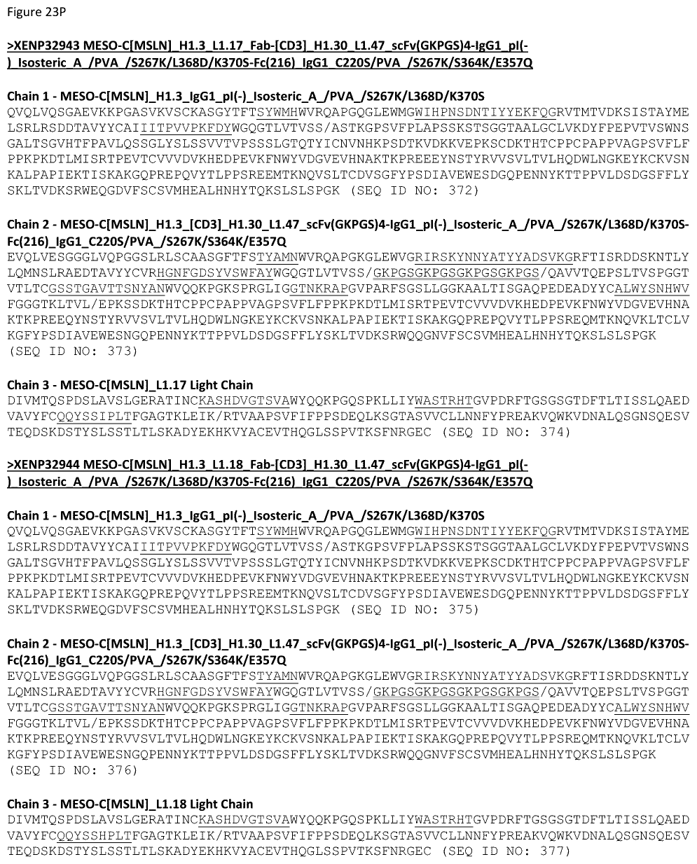

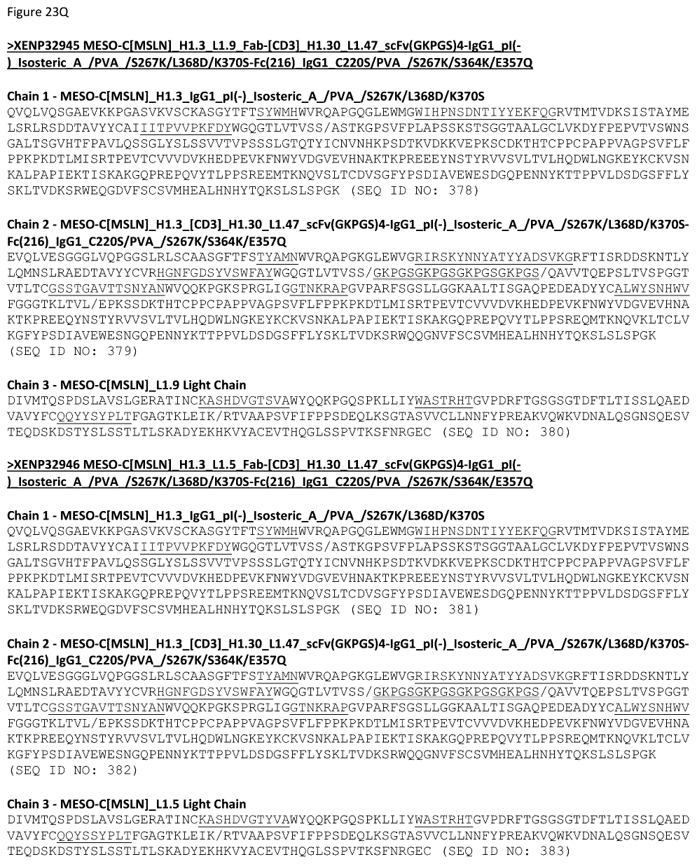

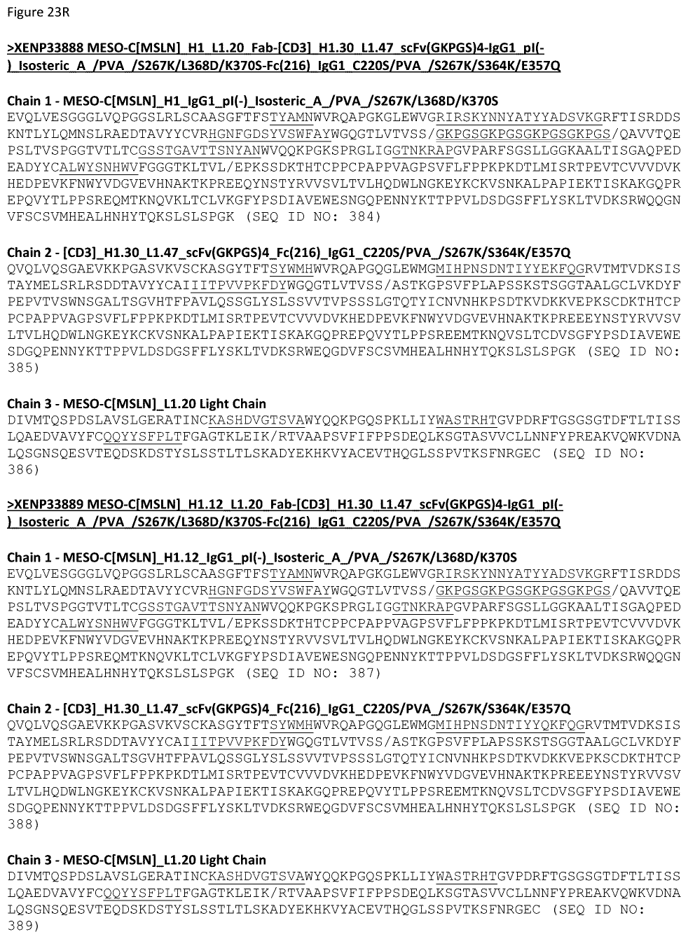

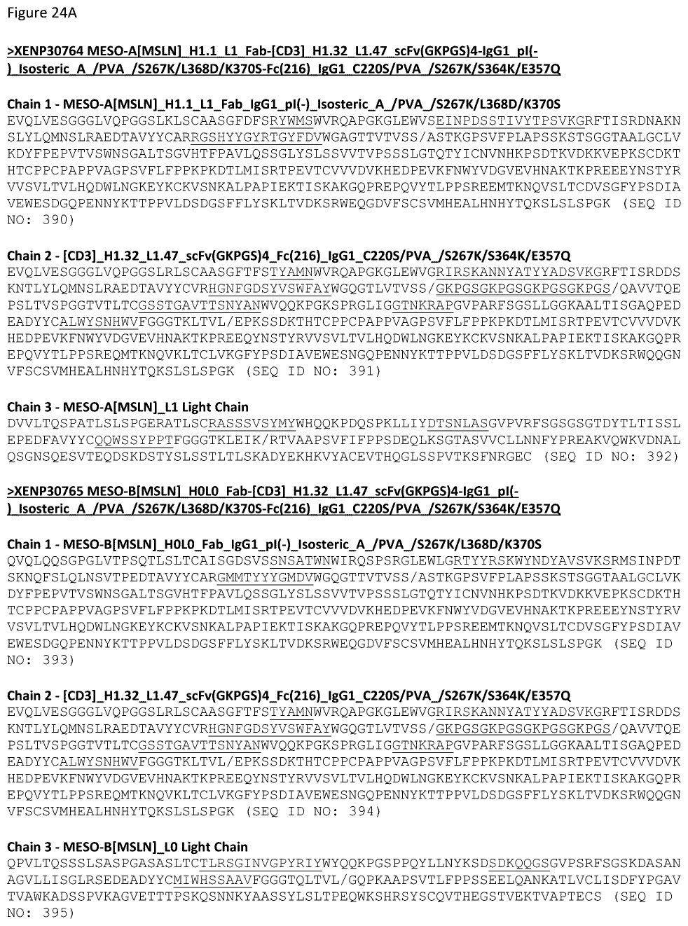

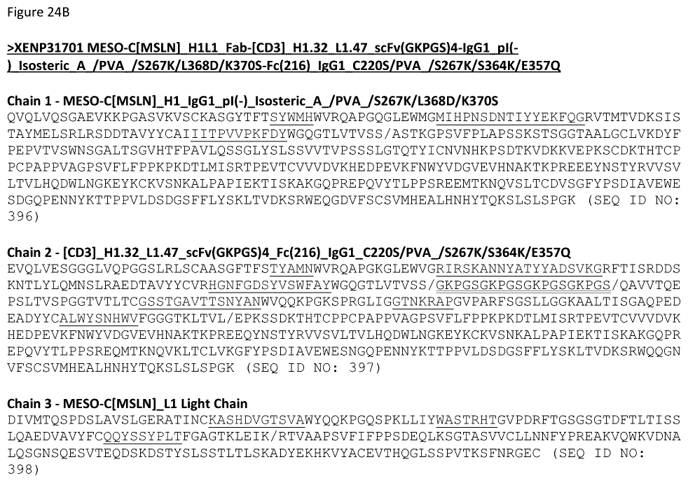

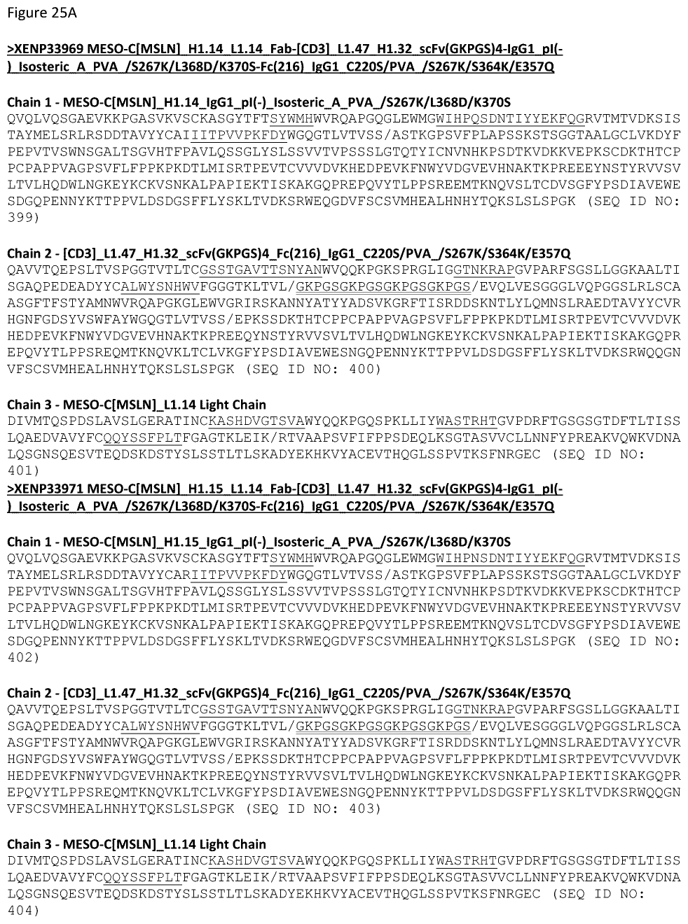

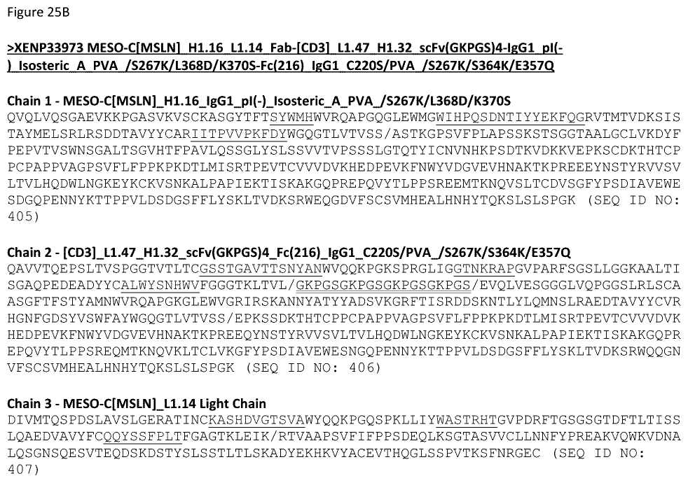

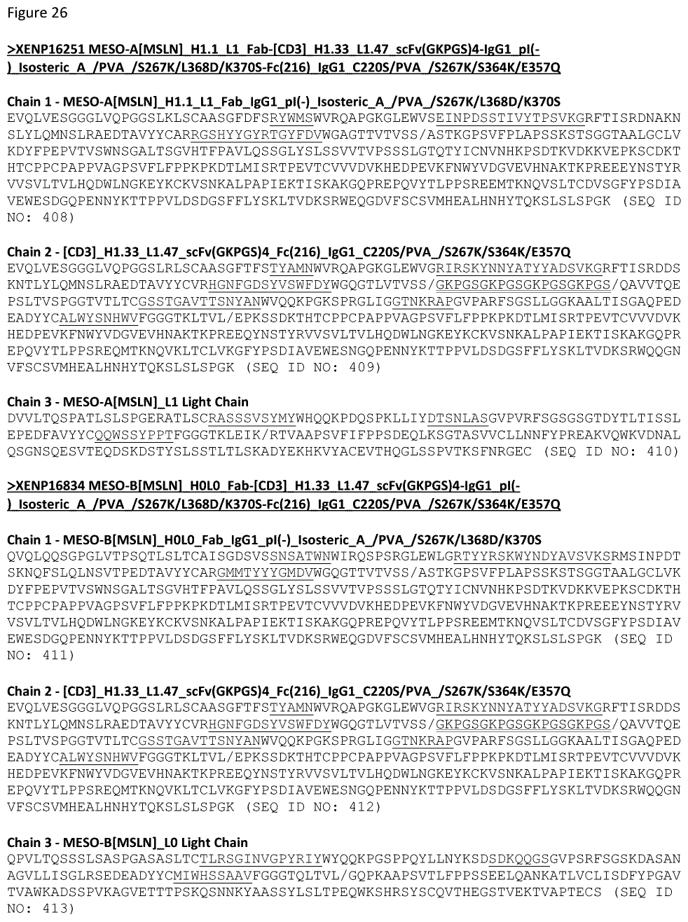

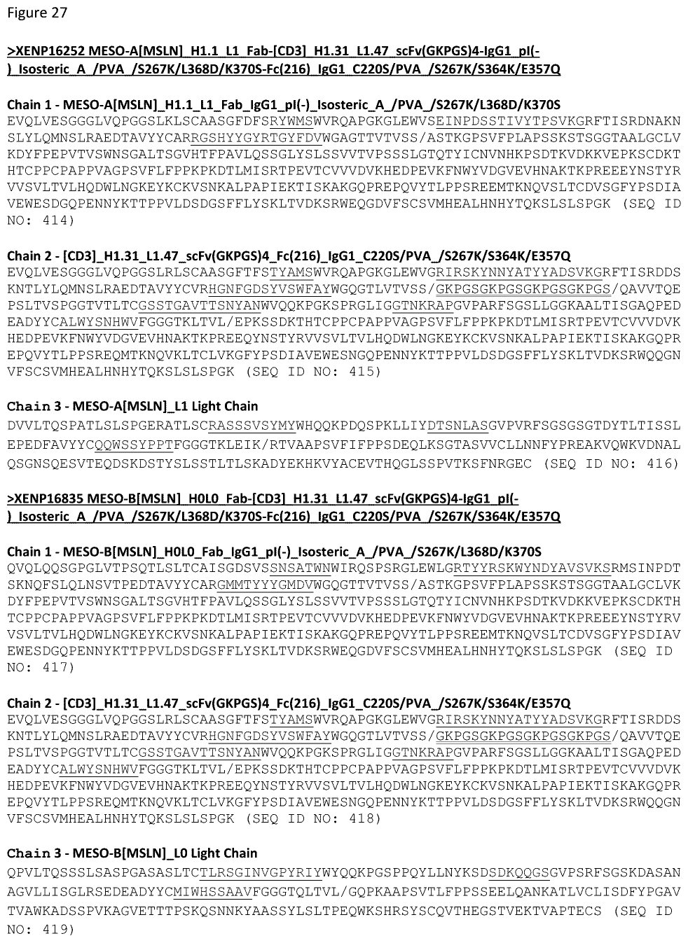

In general, in many preferred embodiments, the scFv is the domain that binds to the CD3, and the Fab forms a MSLN binding domain. An exemplary anti-MSLN×anti-CD3 bispecific antibody in the 1+1 Fab-scFv-Fc format is depicted in FIG. 22A. Exemplary anti-MSLN×anti-CD3 bispecific antibody in the 1+1 Fab-scFv-Fc format is depicted in FIGS. 23A-23R, FIGS. 24A-24B, FIGS. 25A-25B, FIG. 26, and FIG. 27.

In addition, the Fc domains of the antibodies described herein generally include skew variants (e.g. a set of amino acid substitutions as shown in FIGS. 1 and 4, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C), optionally ablation variants (including those shown in FIG. 3), optionally charged scFv linkers (including those shown in FIG. 5) and the heavy chain comprises pI variants (including those shown in FIG. 4).

In certain embodiments, the 1+1 Fab-scFv-Fc scaffold format includes a first monomer that includes a scFv-domain linker-CH2-CH3 monomer, a second monomer that includes a first variable heavy domain-CH1-hinge-CH2-CH3 monomer and a third monomer that includes a first variable light domain. In some embodiments, the CH2-CH3 of the first monomer is a first variant Fc domain and the CH2-CH3 of the second monomer is a second variant Fc domain. In some embodiments, the scFv includes a scFv variable heavy domain and a scFv variable light domain that form a CD3 binding moiety. In certain embodiments, the scFv variable heavy domain and scFv variable light domain are covalently attached using an scFv linker (charged, in many but not all instances. Sec, e.g., FIG. 5). In some embodiments, the first variable heavy domain and first variable light domain form a MSLN binding domain. In some embodiments, the 1+1 Fab-scFv-Fc format includes skew variants, pI variants, and ablation variants. Accordingly, some embodiments include 1+1 Fab-scFv-Fc formats that comprise: a) a first monomer (the “scFv monomer”) that comprises a charged scFv linker (with the +H sequence of FIG. 5 being preferred in some embodiments), the skew variants S364K/E357Q, the ablation variants E233P/L234V/L235A/G236del/S267K, and an scFv that binds to CD3 as outlined herein; b) a second monomer (the “Fab monomer”) that comprises the skew variants L368D/K370S, the pI variants N208D/Q295E/N384D/Q418E/N421D, the ablation variants E233P/L234V/L235A/G236del/S267K, and a variable heavy domain; and c) a light chain that includes a variable light domain light domain (V_{L}) and a constant light domain (CL), wherein numbering is according to EU numbering. The variable heavy domain and variable light domain make up a MSLN binding moiety. CD3 binding domain sequences finding particular use in these embodiments include, but are not limited to, H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 as well as those depicted in FIGS. 10A-10F. In some embodiments, the MSLN binding domains include the variable heavy and variable light domain of any one of the MSLN binding domains disclosed in FIGS. 15-21. MSLN binding domain sequences that are of particular use in these embodiments include, but are not limited to: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

Particularly useful MSLN and CD3 sequence combinations for use with the 1+1 Fab₂-scFv-Fc format antibody include, for example, MESO-A[MSLN]_H1.1_L1×CD3 H1.30 L1.47, MESO-B[MSLN]_H0L0×CD3 H1.30 L1.47, MESO-C[MSLN]_H1L1_×CD3 H1.30 L1.47, MESO-C[MSLN]_H1.1_L1×CD3 H1.30 L1.47, MESO-C[MSLN]_H1.2_L1×CD3 H1.30 L1.47, MESO-C[MSLN]_H1.3_L1_×CD3 H1.30 L1.47, MESO-C[MSLN]_H1.4_L1×CD3 H1.30 L1.47, MESO-C[MSLN]_H1.5_L1×CD3 H1.30 L1.47, MESO-C[MSLN]_H1.6_L1×CD3 H1.30 L1.47, MESO-C[MSLN]_H1.7_L1×CD3 H1.30 L1.47, MESO-C[MSLN]_H1.8_L1×CD3 H1.30 L1.47, MESO-C[MSLN]_H1.9_L1×CD3 H1.30 L1.47, MESO-C[MSLN]_H1_L1.1×CD3 H1.30 L1.47, MESO-C[MSLN]_H1_L1.2×CD3 H1.30 L1.47, MESO-C[MSLN]_H1_L1.3×CD3 H1.30 L1.47, MESO-C[MSLN]_H1_L1.4_×CD3 H.130 L1.47, MESO-C[MSLN]_H1_L1.5×CD3 H1.30 L1.47, MESO-C[MSLN]_H1_L1.6_×CD3 H1.30 L1.47, MESO-C[MSLN]_H1_L1.7×CD3 H1.30 L1.47, MESO-C[MSLN]_H1_L1.8×CD3 H1.30 L1.47, MESO-C[MSLN]_H1_L1.9×CD3 H1.30 L1.47, MESO-C[MSLN]_H1_L1.10×CD3 H1.30 L1.47, MESO-C[MSLN]_H1.5_L1.3×CD3 H1.30 L1.47, MESO-C[MSLN]_H1.6_L1.3×CD3 H.130 L1.47, MESO-C[MSLN]_H1.12_L1.9×CD3 H1.30 L1.47, MESO-C[MSLN]_H1.13_L1.9×CD3 H1.30 L1.47, MESO-C[MSLN]_H1.3_L1.13×CD3 H1.30 L1.47, MESO-C[MSLN]_H1.3_L1.14×CD3 H1.30 L1.47, MESO-C[MSLN]_H1.3_L1.15×CD3 H1.30 L1.47, MESO-C[MSLN]_H1.3_L1.16×CD3 H1.30 L1.47, MESO-C[MSLN]_H1.3_L1.17×CD3 H1.30 L1.47, MESO-C[MSLN]_H1.3_L1.18×CD3 H1.30 L1.47, MESO-C[MSLN]_H1.3_L1.9×CD3 H1.30 L1.47, MESO-C[MSLN]_H1.3_L1.5×CD3 H1.30 L1.47, MESO-C[MSLN]_H1_L1.20×CD3 H1.30 L1.47, MESO-C[MSLN]_H1.12_L1.20×CD3 H1.30 L1.47, MESO-A[MSLN]_H1.1_L1×CD3 H1.32 L1.47, MESO-B[MSLN]_H0L0×CD3 H1.32 L1.47, MESO-C[MSLN]_HIL1×CD3 H1.32 L1.47, MESO-C[MSLN]_H1.14_L1.14×CD3 L1.47 H1.32, MESO-C[MSLN]_H1.15_L1.14×CD3 L1.47 H1.32, MESO-C[MSLN]_H1.16_L1.14×CD3 L1.47 H1.32, MESO-A[MSLN]_H1.1_L1×CD3 H1.33 L1.47, MESO-B[MSLN]_H0L0×CD3 H1.33 L1.47, MESO-A[MSLN]_H1.1_L1×CD3 H1.31 L1.47, and MESO-B[MSLN]_H0L0×CD3 H1.31 L1.47 (FIGS. 23A-27).

Exemplary variable heavy and light domains of the scFv that binds to CD3 are included in FIG. 10A-10F. Exemplary variable heavy and light domains of the Fv that binds to MSLN are included in FIGS. 15-21. In some embodiments, the MSLN binding domains include the variable heavy and variable light domain of any one of the MSLN binding domains disclosed in FIGS. 15-21. In an exemplary embodiment, the MSLN binding domain of the 1+1 Fab-scFv-Fc MSLN×CD3 bispecific antibody includes the VH and VL of one of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-MESO-C[MSLN]_H1.12_L1.21, MESO-C[MSLN]_H1.12_L1.22, and MESO-C[MSLN]_H1.12_L1.9 (FIGS. 15-21). In one embodiment, the CD3 binding domain of the 1+1 Fab-scFv-Fc MSLN×CD3 bispecific antibody includes the V_{H} and V_{L} of one of the following CD3 binding domains: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F). Particularly useful MSLN and CD3 combinations for use in the 1+1 Fab-scFv-Fc MSLN×CD3 bispecific antibody format are disclosed in FIGS. 23A-27 and include: MESO-A[MSLN]_H1.1_L1×CD3 H1.30 L1.47, MESO-B[MSLN]_H0L0×CD3 H1.30 L1.47, MESO-C[MSLN]_H1L1_×CD3 H1.30 L1.47, MESO-C[MSLN]_H1.1_L1×CD3 H1.30 L1.47, MESO-C[MSLN]_H1.2_L1×CD3 H1.30 L1.47, MESO-C[MSLN]_H1.3_L1_×CD3 H1.30 L1.47, MESO-C[MSLN]_H1.4_L1×CD3 H1.30 L1.47, MESO-C[MSLN]_H1.5_L1×CD3 H1.30 L1.47, MESO-C[MSLN]_H1.6_L1×CD3 H1.30 L1.47, MESO-C[MSLN]_H1.7_L1×CD3 H1.30 L1.47, MESO-C[MSLN]_H1.8_L1×CD3 H1.30 L1.47, MESO-C[MSLN]_H1.9_L1×CD3 H1.30 L1.47, MESO-C[MSLN]_H1_L1.1×CD3 H1.30 L1.47, MESO-C[MSLN]_H1_L1.2×CD3 H1.30 L1.47, MESO-C[MSLN]_H1_L1.3×CD3 H1.30 L1.47, MESO-C[MSLN]_H1_L1.4_×CD3 H.130 L1.47, MESO-C[MSLN]_H1_L1.5×CD3 H1.30 L1.47, MESO-C[MSLN]_H1_L1.6_×CD3 H1.30 L1.47, MESO-C[MSLN]_H1_L1.7×CD3 H1.30 L1.47, MESO-C[MSLN]_H1_L1.8×CD3 H1.30 L1.47, MESO-C[MSLN]_H1_L1.9×CD3 H1.30 L1.47, MESO-C[MSLN]_H1_L1.10×CD3 H1.30 L1.47, MESO-C[MSLN]_H1.5_L1.3×CD3 H1.30 L1.47, MESO-C[MSLN]_H1.6_L1.3×CD3 H.130 L1.47, MESO-C[MSLN]_H1.12_L1.9×CD3 H1.30 L1.47, MESO-C[MSLN]_H1.13_L1.9×CD3 H1.30 L1.47, MESO-C[MSLN]_H1.3_L1.13×CD3 H1.30 L1.47, MESO-C[MSLN]_H1.3_L1.14×CD3 H1.30 L1.47, MESO-C[MSLN]_H1.3_L1.15×CD3 H1.30 L1.47, MESO-C[MSLN]_H1.3_L1.16×CD3 H1.30 L1.47, MESO-C[MSLN]_H1.3_L1.17×CD3 H1.30 L1.47, MESO-C[MSLN]_H1.3_L1.18×CD3 H1.30 L1.47, MESO-C[MSLN]_H1.3_L1.9×CD3 H1.30 L1.47, MESO-C[MSLN]_H1.3_L1.5×CD3 H1.30 L1.47, MESO-C[MSLN]_H1_L1.20×CD3 H1.30 L1.47, MESO-C[MSLN]_H1.12_L1.20×CD3 H1.30 L1.47, MESO-A[MSLN]_H1.1_L1×CD3 H1.32 L1.47, MESO-B[MSLN]_H0L0×CD3 H1.32 L1.47, MESO-C[MSLN]_H1L1×CD3 H1.32 L1.47, MESO-C[MSLN]_H1.14_L1.14×CD3 L1.47 H1.32, MESO-C[MSLN]_H1.15_L1.14×CD3 L1.47 H1.32, MESO-C[MSLN]_H1.16_L1.14×CD3 L1.47 H1.32, MESO-A[MSLN]_H1.1_L1×CD3 H1.33 L1.47, MESO-B[MSLN]_H0L0×CD3 H1.33 L1.47, MESO-A[MSLN]_H1.1_L1×CD3 H1.31 L1.47, and MESO-B[MSLN]_H0L0×CD3 H1.31 L1.47.

In some embodiments, the 1+1 Fab-scFv-Fc format includes skew variants, pI variants, ablation variants and FcRn variants. Accordingly, some embodiments include 1+1 Fab-scFv-Fc formats that comprise: a) a first monomer (the “scFv monomer”) that comprises a charged scFv linker (with the +H sequence of FIG. 6 being preferred in some embodiments), the skew variants S364K/E357Q, the ablation variants E233P/L234V/L235A/G236del/S267K, the FcRn variants M428L/N434S and an scFv that binds to CD3 as outlined herein; b) a second monomer (the “Fab monomer”) that comprises the skew variants L368D/K370S, the pI variants N208D/Q295E/N384D/Q418E/N421D, the ablation variants E233P/L234V/L235A/G236del/S267K, the FcRn variants M428L/N434S, and a variable heavy domain; and c) a light chain that includes a variable light domain (V_{L}) and a constant light domain (CL), wherein numbering is according to EU numbering. The variable heavy domain and variable light domain make up a MSLN binding domain. CD3 binding domain sequences finding particular use in these embodiments include, but are not limited to, H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 as well as those depicted in FIGS. 10A-10F. MSLN binding domain sequences that are of particular use in these embodiments include, but are not limited to: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

Particularly useful MSLN and CD3 sequence combinations for use with the 1+1 Fab₂-scFv-Fc format antibody include, for example, are disclosed in FIGS. 23-27 and include: MESO-A[MSLN]_H1.1_L1×CD3 H1.30 L1.47, MESO-B[MSLN]_H0L0×CD3 H1.30 L1.47, MESO-C[MSLN]_H1L1_×CD3 H1.30 L1.47, MESO-C[MSLN]_H1.1_L1×CD3 H1.30 L1.47, MESO-C[MSLN]_H1.2_L1×CD3 H1.30 L1.47, MESO-C[MSLN]_H1.3_L1_×CD3 H1.30 L1.47, MESO-C[MSLN]_H1.4_L1×CD3 H1.30 L1.47, MESO-C[MSLN]_H1.5_L1×CD3 H1.30 L1.47, MESO-C[MSLN]_H1.6_L1×CD3 H1.30 L1.47, MESO-C[MSLN]_H1.7_L1×CD3 H1.30 L1.47, MESO-C[MSLN]_H1.8_L1×CD3 H1.30 L1.47, MESO-C[MSLN]_H1.9_L1×CD3 H1.30 L1.47, MESO-C[MSLN]_H1_L1.1×CD3 H1.30 L1.47, MESO-C[MSLN]_H1_L1.2×CD3 H1.30 L1.47, MESO-C[MSLN]_H1_L1.3×CD3 H1.30 L1.47, MESO-C[MSLN]_H1_L1.4_×CD3 H.130 L1.47, MESO-C[MSLN]_H1_L1.5×CD3 H1.30 L1.47, MESO-C[MSLN]_H1_L1.6_×CD3 H1.30 L1.47, MESO-C[MSLN]_H1_L1.7×CD3 H1.30 L1.47, MESO-C[MSLN]_H1_L1.8×CD3 H1.30 L1.47, MESO-C[MSLN]_H1_L1.9×CD3 H1.30 L1.47, MESO-C[MSLN]_H1_L1.10×CD3 H1.30 L1.47, MESO-C[MSLN]_H1.5_L1.3×CD3 H1.30 L1.47, MESO-C[MSLN]_H1.6_L1.3×CD3 H.130 L1.47, MESO-C[MSLN]_H1.12_L1.9×CD3 H1.30 L1.47, MESO-C[MSLN]_H1.13_L1.9×CD3 H1.30 L1.47, MESO-C[MSLN]_H1.3_L1.13×CD3 H1.30 L1.47, MESO-C[MSLN]_H1.3_L1.14×CD3 H1.30 L1.47, MESO-C[MSLN]_H1.3_L1.15×CD3 H1.30 L1.47, MESO-C[MSLN]_H1.3_L1.16×CD3 H1.30 L1.47, MESO-C[MSLN]_H1.3_L1.17×CD3 H1.30 L1.47, MESO-C[MSLN]_H1.3_L1.18×CD3 H1.30 L1.47, MESO-C[MSLN]_H1.3_L1.9×CD3 H1.30 L1.47, MESO-C[MSLN]_H1.3_L1.5×CD3 H1.30 L1.47, MESO-C[MSLN]_H1_L1.20×CD3 H1.30 L1.47, MESO-C[MSLN]_H1.12_L1.20×CD3 H1.30 L1.47, MESO-A[MSLN]_H1.1_L1×CD3 H1.32 L1.47, MESO-B[MSLN]_H0L0×CD3 H1.32 L1.47, MESO-C[MSLN]_H1L1×CD3 H1.32 L1.47, MESO-C[MSLN]_H1.14_L1.14×CD3 L1.47 H1.32, MESO-C[MSLN]_H1.15_L1.14×CD3 L1.47 H1.32, MESO-C[MSLN]_H1.16_L1.14×CD3 L1.47 H1.32, MESO-A[MSLN]_H1.1_L1×CD3 H1.33 L1.47, MESO-B[MSLN]_H0L0×CD3 H1.33 L1.47, MESO-A[MSLN]_H1.1_L1×CD3 H1.31 L1.47, and MESO-B[MSLN]_H0L0×CD3 H1.31 L1.47.

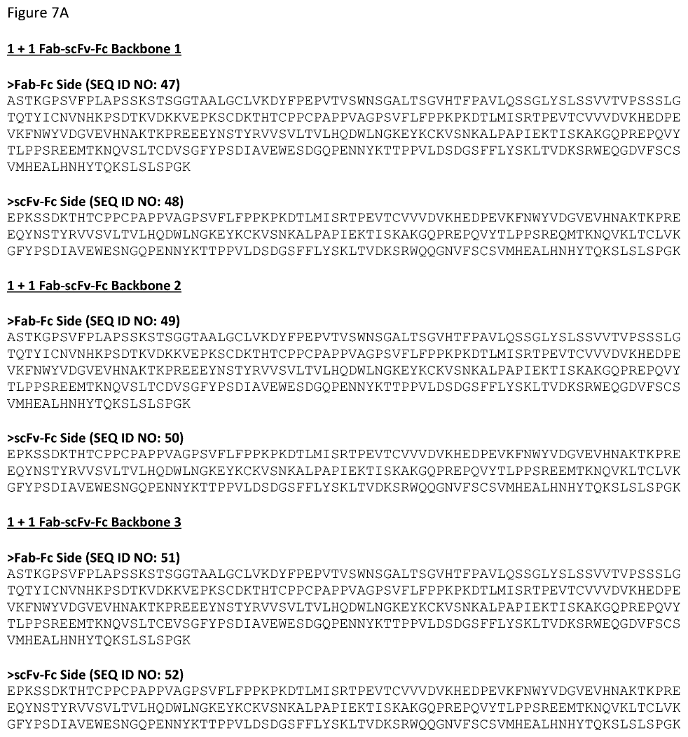

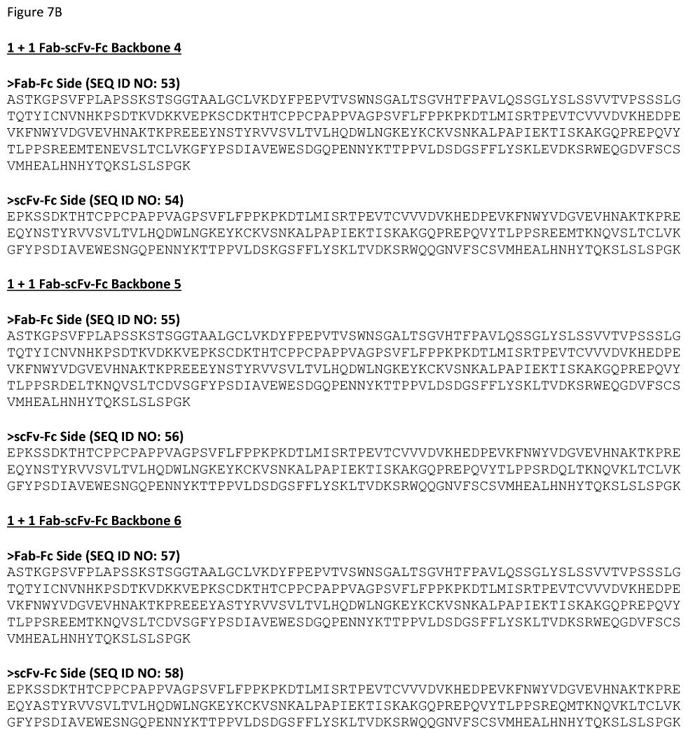

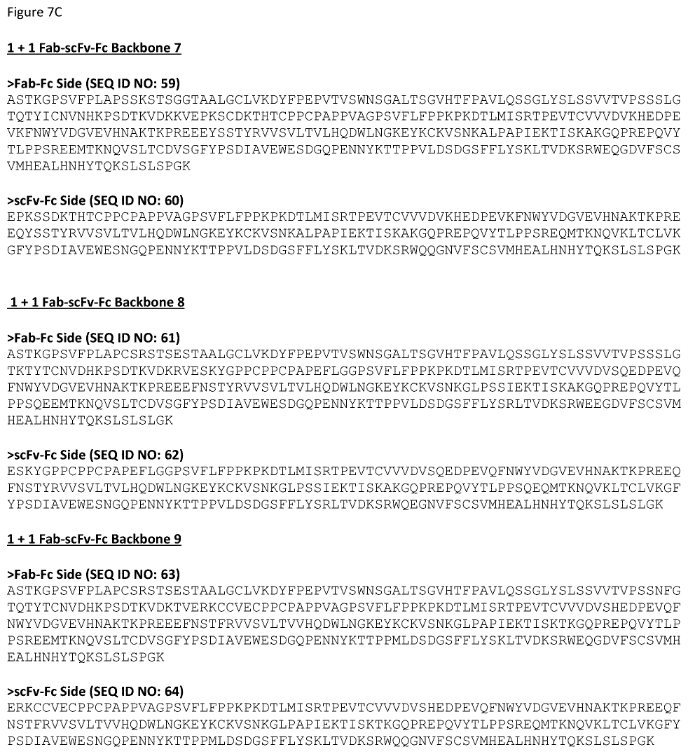

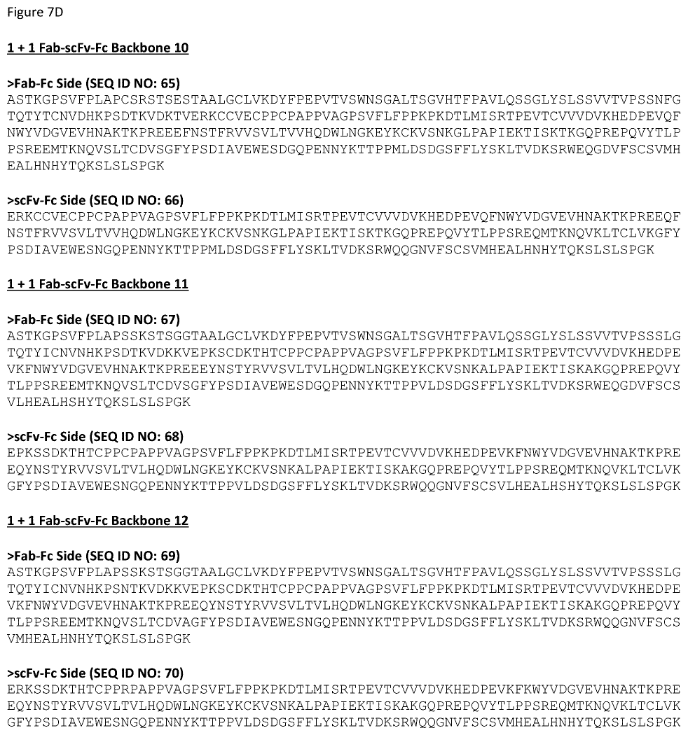

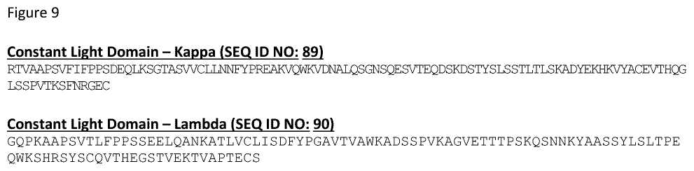

FIGS. 7A-7D show some exemplary Fc domain sequences that are useful in the 1+1 Fab-scFv-Fc format antibodies. The “monomer 1” sequences depicted in FIGS. 7A-7D typically refer to the Fc domain of the “Fab-Fc heavy chain” and the “monomer 2” sequences refer to the Fc domain of the “scFv-Fc heavy chain.” Further, FIG. 9 provides useful CL sequences that can be used with this format.

In some embodiments, any of the VH and VL sequences depicted herein (including all VH and VL sequences depicted in the Figures and Sequence Listings, including those directed to MSLN) can be added to the bottle opener backbone formats of FIG. 7A-7D as the “Fab side”, using any of the anti-CD3 scFv sequences shown in the Figures and Sequence Listings.

For bottle opener backbone 1 from FIG. 7A, (optionally including the 428L/434S variants), CD3 binding domain sequences finding particular use in these embodiments include, but are not limited to, CD3 binding domain anti-CD3 H1.30_L1.47, anti-CD3 H1.32_L1.47, anti-CD3 H1.89_L1.47, anti-CD3 H1.90_L1.47, anti-CD3 H1.33_L1.47 and anti-CD3 H1.31_L1.47, as well as those depicted in FIG. 10A-10F, attached as the scFv side of the backbones shown in FIGS. 7A-7D.

Particularly useful MSLN and CD3 sequence combinations for use (optionally including the 428L/434S variants), are disclosed in FIGS. 23-27.

In some embodiments, the 1+1 Fab-scFv-Fc format includes sequences selected from: XENP16249, XENP16250, XENP16253, XENP16827, XENP31693.

In some embodiments, the 1+1 Fab-scFv-Fc format comprises sequences selected from: XENP16248, XENP16833, XENP31697, XENP32550, XENP32551, XENP32552, XENP32553, XENP32554, XENP32555, XENP32556, XENP32557, XENP32558, XENP32559, XENP32560, XENP32561, XENP32562, XENP32563, XENP32564, XENP32565, XENP32566, XENP32567, XENP32568, XENP32569, XENP32570, XENP32937, XENP32938, XENP32939, XENP32940, XENP32941, XENP32942, XENP32943, XENP32944, XENP32945, XENP32946, XENP33888, XENP33889, XENP30764, XENP30765, XENP31701, XENP33969, XENP33971, XENP33973, XENP16251, XENP16834, XENP16252, and XENP16835 (FIGS. 23-27).

In some embodiments, the 1+1 Fab-scFv-Fc format comprises a variable heavy chain MSLN sequence comprising one or more of the following mutations: S31G, W32Y, M50W, H52N, N53Q, D55G, 194R, T97S, E61Q, E61Y, and/or K100bR.

In some embodiments, the 1+1 Fab-scFv-Fc format comprises a variable light chain MSLN sequence comprising one or more of the following mutations: A25S, H27Q, D28S, G30L, S32Y, V33L, T37Q, H55E, T56S, S92Y, Y94T, Y94L, Y94V, Y94I, Y94H, D28S and Y100F.

In some embodiments, the 1+1 Fab-scFv-Fc format includes a MSLN binding domain that includes a MSLN binding domain variable heavy domain selected from any of FIGS. 15-18 and 20-21. In some embodiments, the 1+1 Fab-scFv-Fc format includes a MSLN binding domain that includes a MSLN binding domain variable light domain selected from any of FIGS. 15-17 and 19-21. In some embodiments, the 1+1 Fab-scFv-Fc format includes a MSLN binding domain that includes a combination of any of the MSLN binding domain variable heavy domains and variable light domains in FIGS. 15-21.

2. mAb-Fv

One heterodimeric scaffold that finds particular use in the antibodies described herein is the mAb-Fv format. In this embodiment, the format relies on the use of a C-terminal attachment of an “extra” variable heavy domain to one monomer and the C-terminal attachment of an “extra” variable light domain to the other monomer, thus forming a third antigen binding domain, wherein the Fab portions of the two monomers bind a MSLN and the “extra” scFv domain binds CD3.

In this embodiment, the first monomer comprises a first heavy chain, comprising a first variable heavy domain and a first constant heavy domain comprising a first Fc domain, with a first variable light domain covalently attached to the C-terminus of the first Fc domain using a domain linker (VH1-CH1-hinge-CH2-CH3-[optional linker]-VL2). The second monomer comprises a second variable heavy domain of the second constant heavy domain comprising a second Fc domain, and a third variable heavy domain covalently attached to the C-terminus of the second Fc domain using a domain linker (vh1-CH1-hinge-CH2-CH3-[optional linker]-VH2. The two C-terminally attached variable domains make up a Fv that binds CD3 (as it is less preferred to have bivalent CD3 binding). This embodiment further utilizes a common light chain comprising a variable light domain and a constant light domain that associates with the heavy chains to form two identical Fabs that bind a MSLN. As for many of the embodiments herein, these constructs include skew variants, pI variants, ablation variants, additional Fc variants, etc. as desired and described herein.

The antibodies described herein provide mAb-Fv formats where the CD3 binding domain sequences are as shown in FIG. 10A-10F. The antibodies described herein provide mAb-Fv formats wherein the MSLN binding domain sequences are as shown in FIGS. 15-21.

In addition, the Fc domains of the mAb-Fv format comprise skew variants (e.g. a set of amino acid substitutions as shown in FIGS. 1 and 4, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q, T366S/L368A/Y407V: T366W and T366S/L368A/Y407V/Y349C: T366W/S354C), optionally ablation variants (including those shown in FIG. 3), optionally charged scFv linkers (including those shown in FIG. 5) and the heavy chain comprises pI variants (including those shown in FIG. 2).

In some embodiments, the mAb-Fv format includes skew variants, pI variants, and ablation variants. Accordingly, some embodiments include mAb-Fv formats that comprise: a) a first monomer that comprises the skew variants S364K/E357Q, the ablation variants E233P/L234V/L235A/G236del/S267K, and a first variable heavy domain that, with the first variable light domain of the light chain, makes up an Fv that binds to MSLN, and a second variable heavy domain; b) a second monomer that comprises the skew variants L368D/K370S, the pI variants N208D/Q295E/N384D/Q418E/N421D, the ablation variants E233P/L234V/L235A/G236del/S267K, and a first variable heavy domain that, with the first variable light domain, makes up the Fv that binds to MSLN as outlined herein, and a second variable light chain, that together with the second variable heavy domain forms an Fv (ABD) that binds to CD3; and c) a light chain comprising a first variable light domain and a constant light domain.

In some embodiments, the mAb-Fv format includes skew variants, pI variants, ablation variants and FcRn variants. Accordingly, some embodiments include mAb-Fv formats that comprise: a) a first monomer that comprises the skew variants S364K/E357Q, the ablation variants E233P/L234V/L235A/G236del/S267K, the FcRn variants M428L/N434S and a first variable heavy domain that, with the first variable light domain of the light chain, makes up an Fv that binds to MSLN, and a second variable heavy domain; b) a second monomer that comprises the skew variants L368D/K370S, the pI variants N208D/Q295E/N384D/Q418E/N421D, the ablation variants E233P/L234V/L235A/G236del/S267K, the FcRn variants M428L/N434S and a first variable heavy domain that, with the first variable light domain, makes up the Fv that binds to MSLN as outlined herein, and a second variable light chain, that together with the second variable heavy domain of the first monomer forms an Fv (ABD) that binds CD3; and c) a light chain comprising a first variable light domain and a constant light domain.

In some embodiments, the MSLN binding domains include the variable heavy and variable light domain of any one of the MSLN binding domains disclosed in FIGS. 15-21.

3. mAb-scFv

One heterodimeric scaffold that finds particular use in the antibodies described herein is the mAb-scFv format. In this embodiment, the format relies on the use of a C-terminal attachment of a scFv to one of the monomers, thus forming a third antigen binding domain, wherein the Fab portions of the two monomers bind MSLN and the “extra” scFv domain binds CD3. Thus, the first monomer comprises a first heavy chain (comprising a variable heavy domain and a constant domain), with a C-terminally covalently attached scFv comprising a scFv variable light domain, an scFv linker and a scFv variable heavy domain in either orientation (VH1-CH1-hinge-CH2-CH3-[optional linker]-VH2-scFv linker-VL2 or VH1-CH1-hinge-CH2-CH3-[optional linker]-VL2-scFv linker-VH2). This embodiment further utilizes a common light chain comprising a variable light domain and a constant light domain that associates with the heavy chains to form two identical Fabs that bind MSLN. As for many of the embodiments herein, these constructs include skew variants, pI variants, ablation variants, additional Fc variants, etc. as desired and described herein.

The antibodies described herein provide mAb-scFv formats where the CD binding domain sequences are as shown in FIG. 10A-10F and the MSLN binding domain sequences are as shown in FIGS. 15-21.

In addition, the Fc domains of the mAb-scFv format comprise skew variants (e.g. a set of amino acid substitutions as shown in FIGS. 1 and 4, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q, T366S/L368A/Y407V: T366W and T366S/L368A/Y407V/Y349C: T366W/S354C), optionally ablation variants (including those shown in FIG. 3), optionally charged scFv linkers (including those shown in FIG. 5) and the heavy chain comprises pI variants (including those shown in FIG. 2).

In some embodiments, the mAb-scFv format includes skew variants, pI variants, and ablation variants. Accordingly, some embodiments include mAb-scFv formats that comprise: a) a first monomer that comprises the skew variants S364K/E357Q, the ablation variants E233P/L234V/L235A/G236del/S267K, and a variable heavy domain that, with the variable light domain of the common light chain, makes up an Fv that binds to MSLN as outlined herein, and a scFv domain that binds to CD3; b) a second monomer that comprises the skew variants L368D/K370S, the pI variants N208D/Q295E/N384D/Q418E/N421D, the ablation variants E233P/L234V/L235A/G236del/S267K, and a variable heavy domain that, with the variable light domain of the common light chain, makes up an Fv that binds to MSLN as outlined herein; and c) a common light chain comprising a variable light domain and a constant light domain.

In some embodiments, the mAb-scFv format includes skew variants, pI variants, ablation variants and FcRn variants. Accordingly, some embodiments include mAb-scFv formats that comprise: a) a first monomer that comprises the skew variants S364K/E357Q, the ablation variants E233P/L234V/L235A/G236del/S267K, the FcRn variants M428L/N434S and a variable heavy domain that, with the variable light domain of the common light chain, makes up an Fv that binds to MSLN as outlined herein, and a scFv domain that binds to CD3; b) a second monomer that comprises the skew variants L368D/K370S, the pI variants N208D/Q295E/N384D/Q418E/N421D, the ablation variants E233P/L234V/L235A/G236del/S267K, the FcRn variants M428L/N434S and a variable heavy domain that, with the variable light domain of the common light chain, makes up an Fv that binds to MSLN as outlined herein; and c) a common light chain comprising a variable light domain and a constant light domain.

In some embodiments, the MSLN binding domains include the variable heavy and variable light domain of any one of the MSLN binding domains disclosed in FIGS. 15-21.

### 4. 2+1 Fab₂-scFv-Fc Format

One heterodimeric scaffold that finds particular use in the antibodies described herein is the “2+1 Fab₂-scFv-Fc” format (also referred to in previous related filings as “Central-scFv format”) shown in FIG. 22B with an exemplary combination of a CD3 binding domain and two tumor target antigen (MSLN) binding domains. In this embodiment, the format relies on the use of an inserted scFv domain thus forming a third antigen binding domain, wherein the Fab portions of the two monomers bind MSLN and the “extra” scFv domain binds CD3. The scFv domain is inserted between the Fc domain and the CH1-Fv region of one of the monomers, thus providing a third antigen binding domain. As described, MSLN×CD3 bispecific antibodies having the 2+1 Fab2-scFv-Fc format are potent in inducing redirected T cell cytotoxicity in cellular environments that express low levels of MSLN. Moreover, as shown in the examples, MSLN×CD3 bispecific antibodies having the 2+1 Fab2-scFv-Fc format allow for the “fine tuning” of immune responses as such antibodies exhibit a wide variety of different properties, depending on the MSLN and/or CD3 binding domains used. For example, such antibodies exhibit differences in selectivity for cells with different MSLN expression, potencies for MSLN expressing cells, ability to elicit cytokine release, and sensitivity to soluble MSLN. These MSLN antibodies find use, for example, in the treatment of MSLN associated cancers.

In this embodiment, one monomer comprises a first heavy chain comprising a first variable heavy domain, a CH1 domain (and optional hinge) and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using optional domain linkers (VH1-CH1-[optional linker]-VH2-scFv linker-VL2-[optional linker including the hinge]-CH2-CH3, or the opposite orientation for the scFv, VH1-CH1-[optional linker]-VL2-scFv linker-VH2-[optional linker including the hinge]-CH2-CH3). The optional linkers can be any suitable peptide linkers, including, for example, the domain linkers included in FIG. 6. In some embodiments, the optional linker is a hinge or a fragment thereof. The other monomer is a standard Fab side (i.e., VH1-CH1-hinge-CH2-CH3). This embodiment further utilizes a common light chain comprising a variable light domain and a constant light domain, that associates with the heavy chains to form two identical Fabs that bind MSLN. As for many of the embodiments herein, these constructs include skew variants, pI variants, ablation variants, additional Fc variants, etc. as desired and described herein.

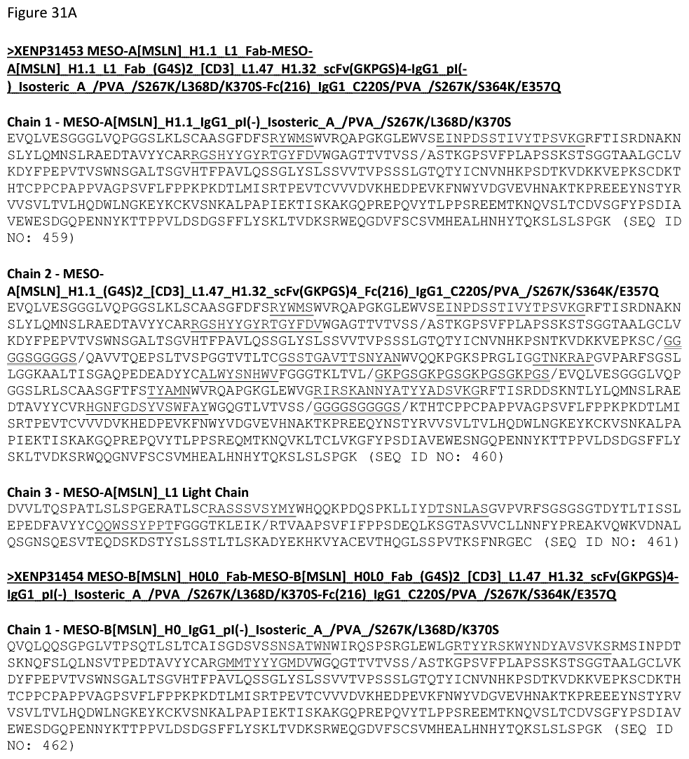

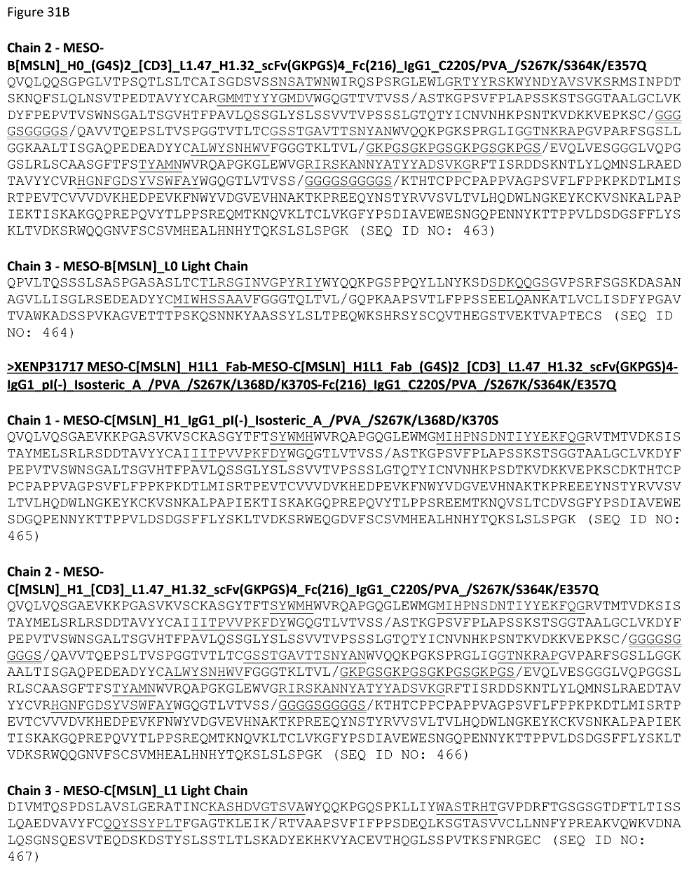

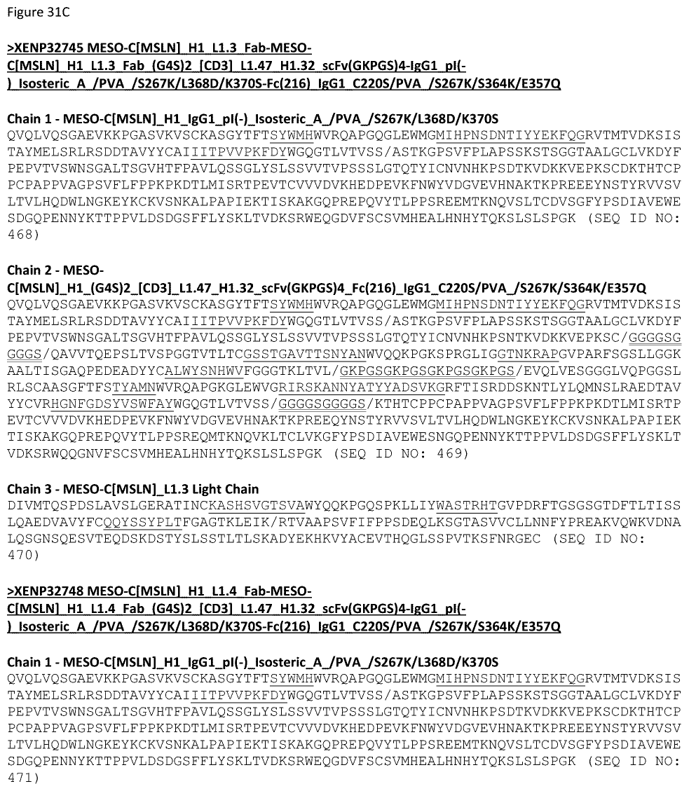

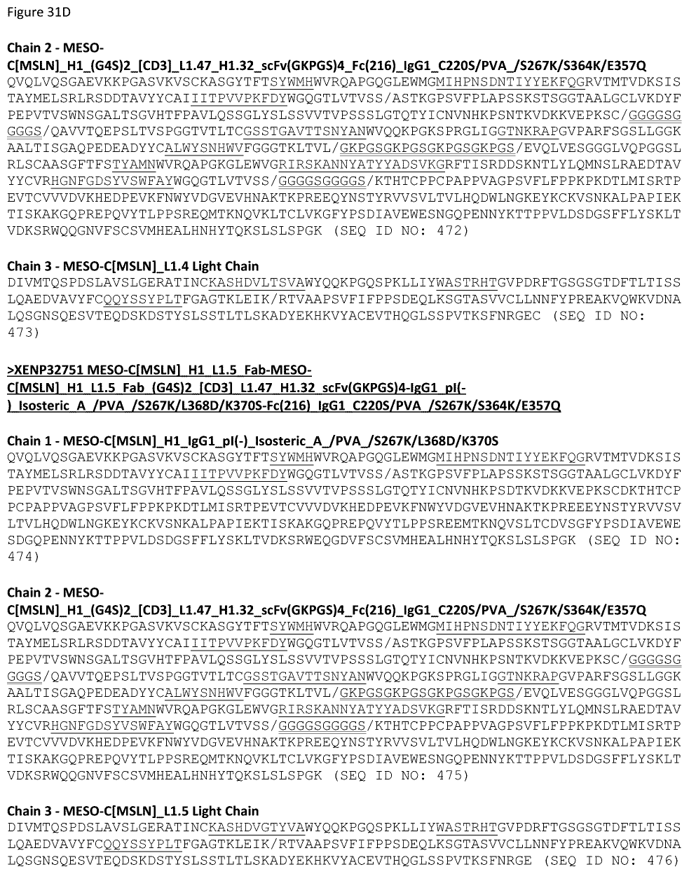

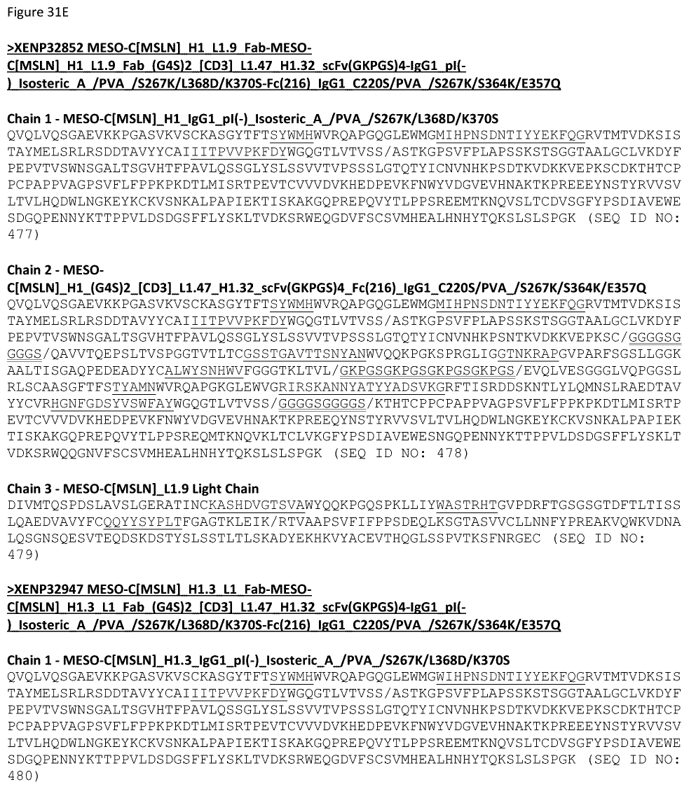

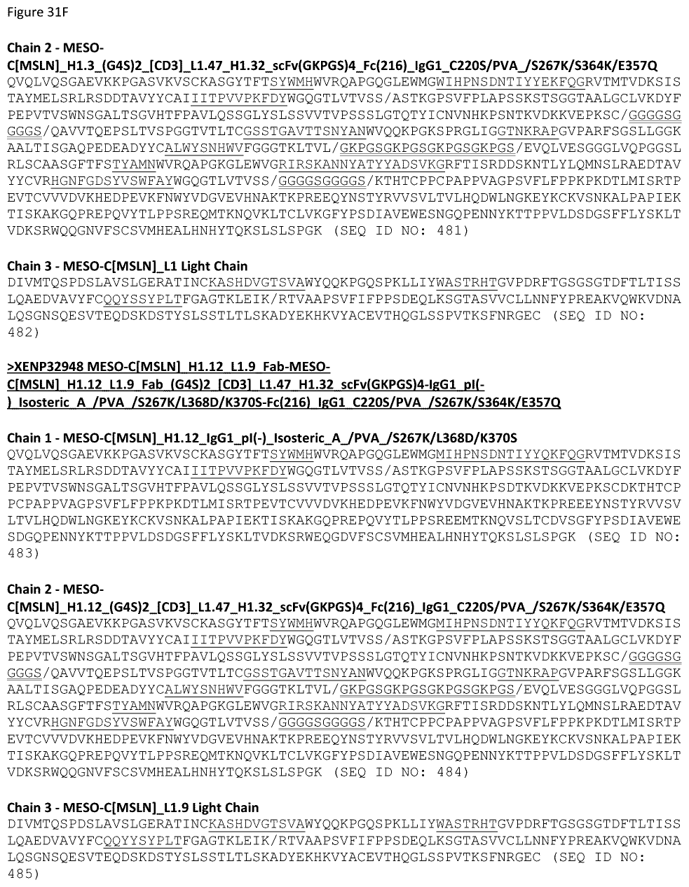

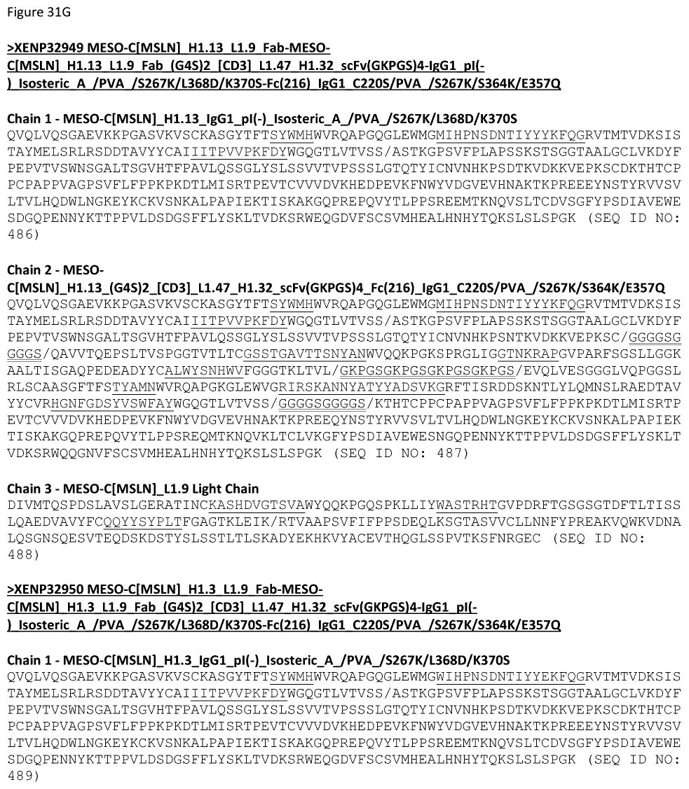

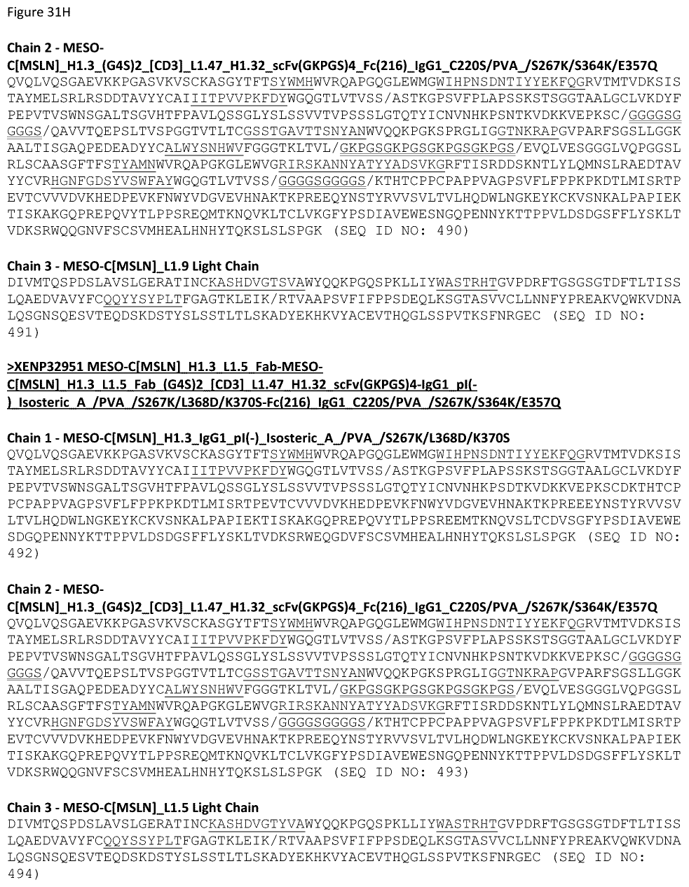

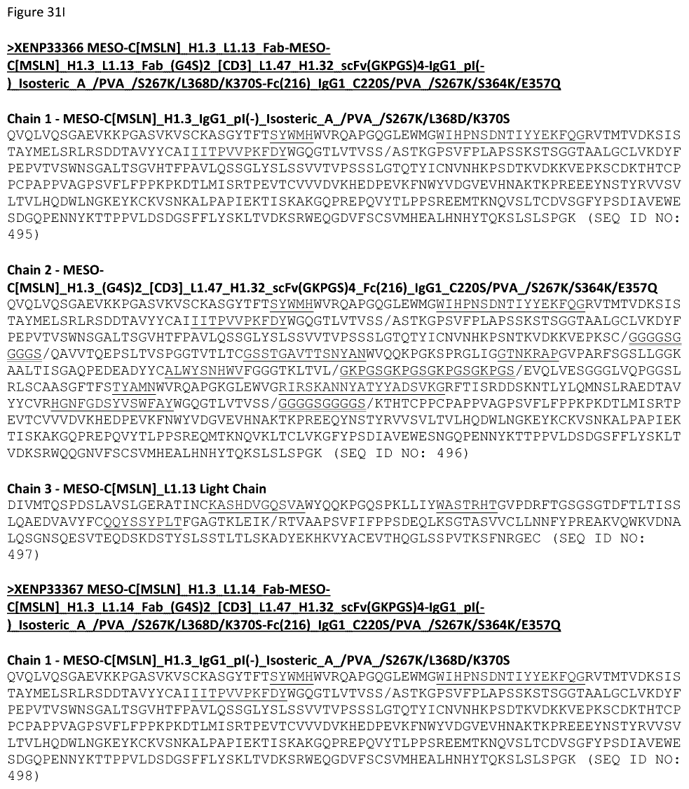

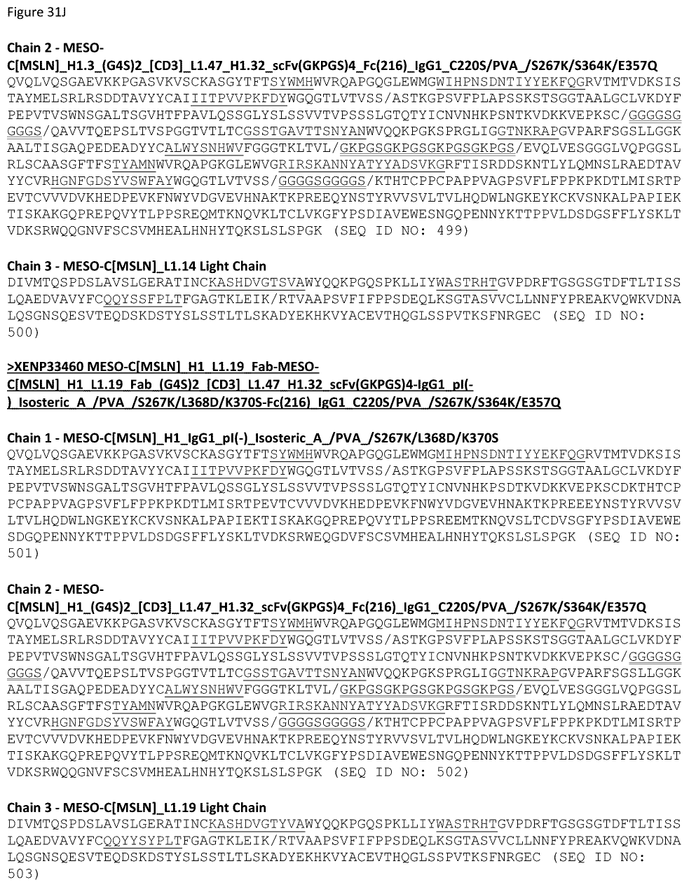

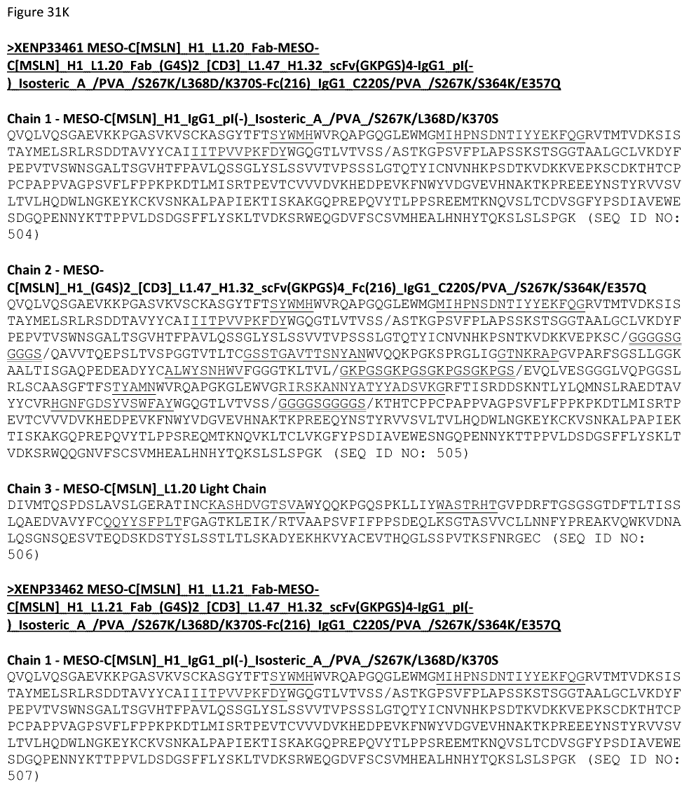

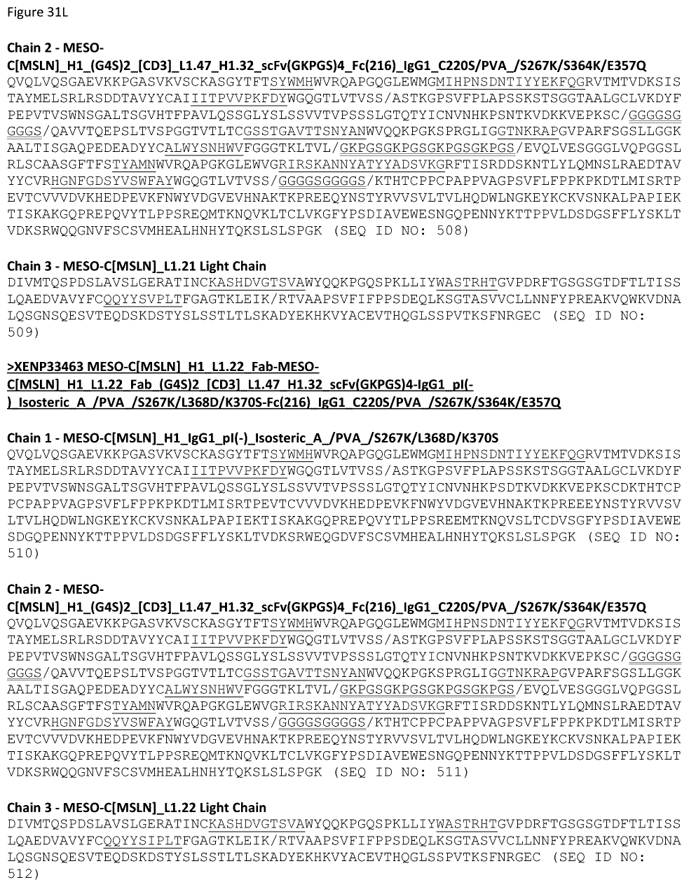

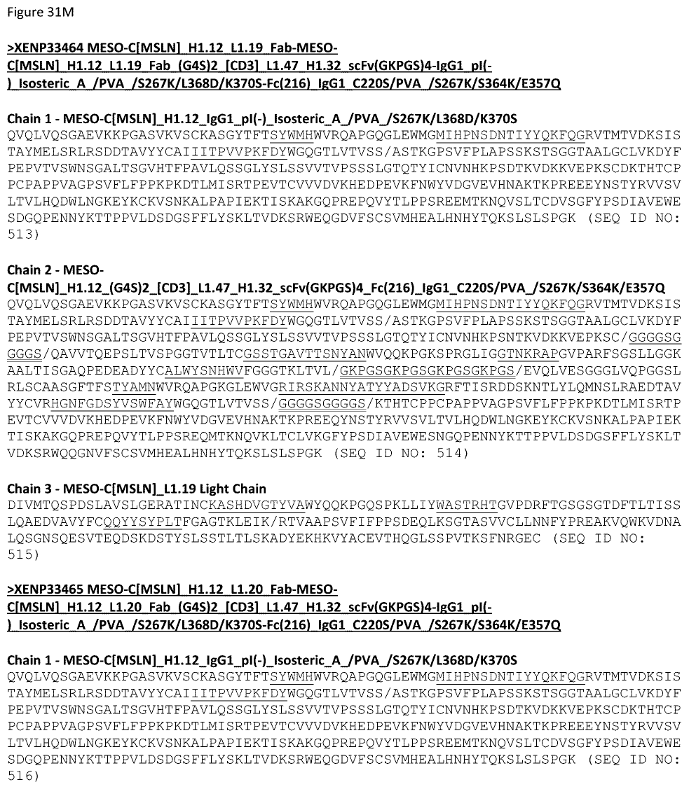

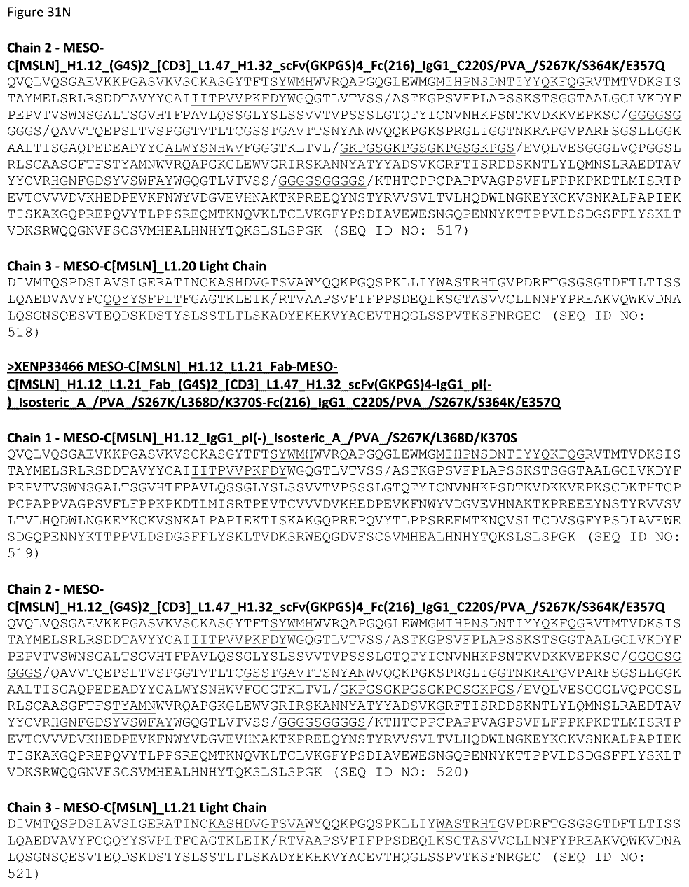

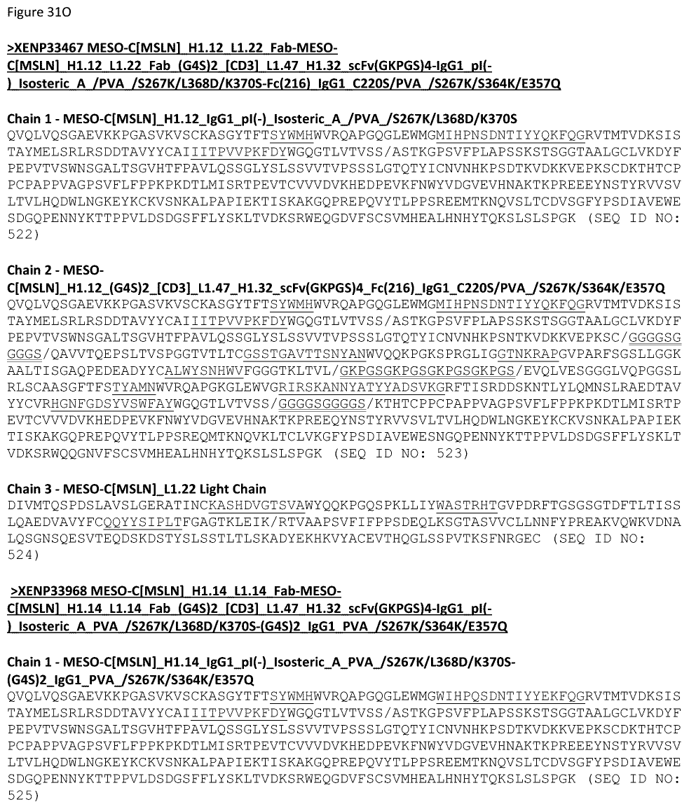

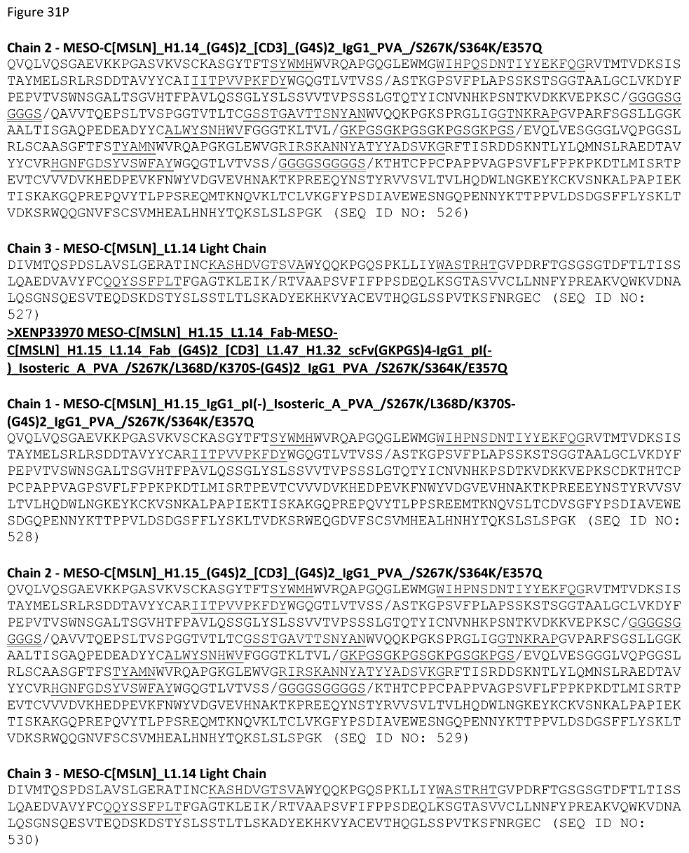

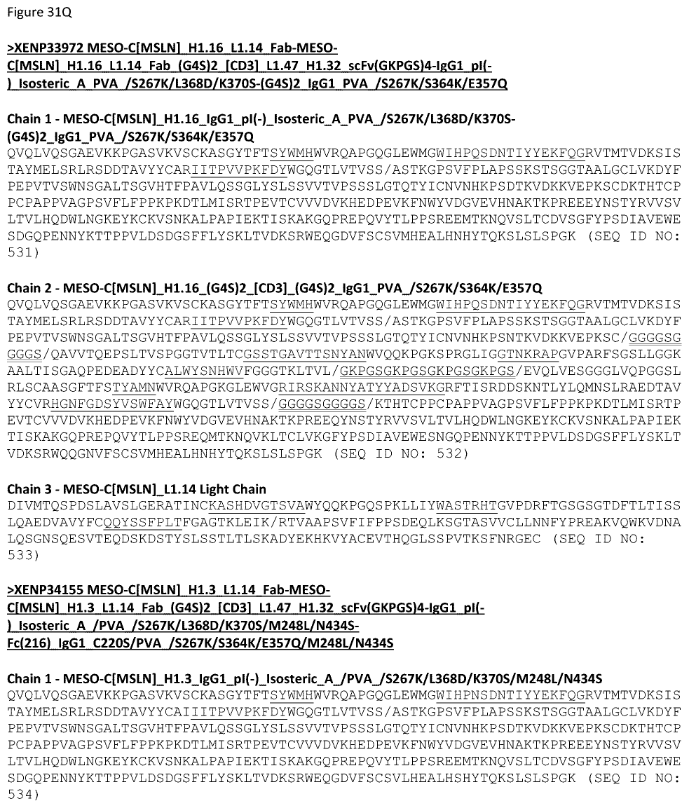

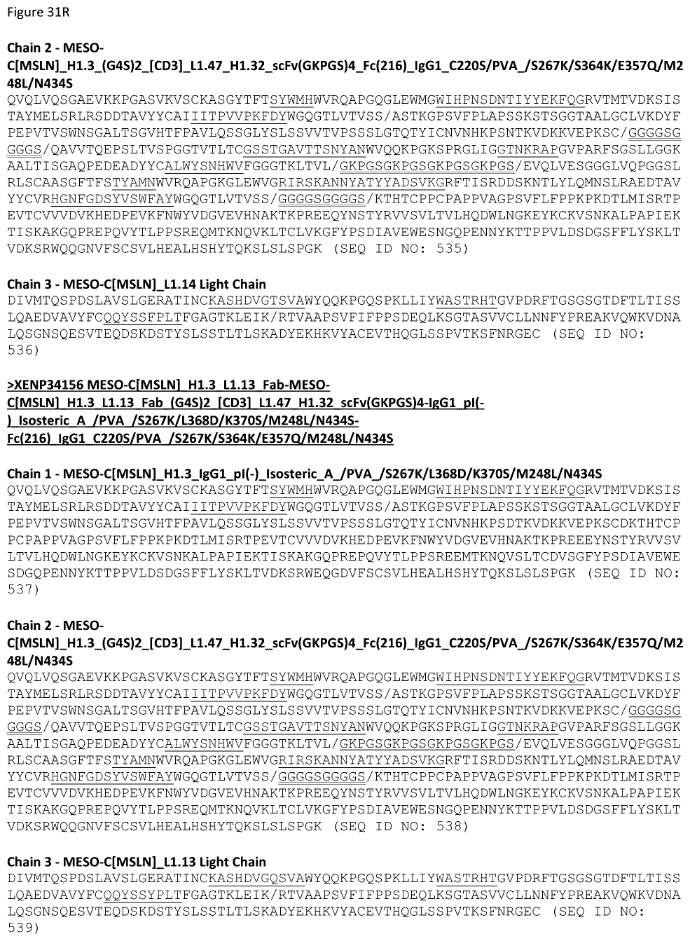

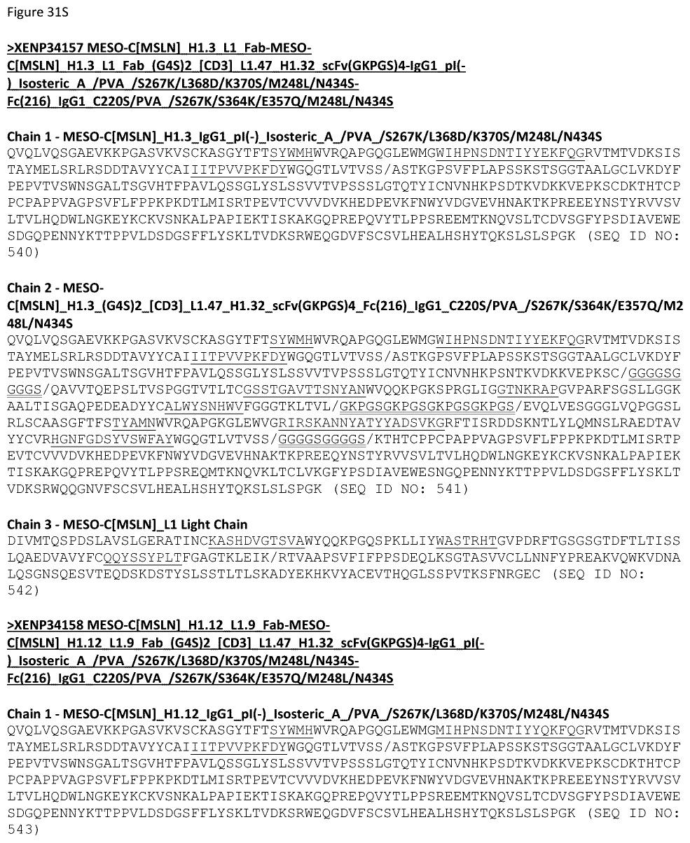

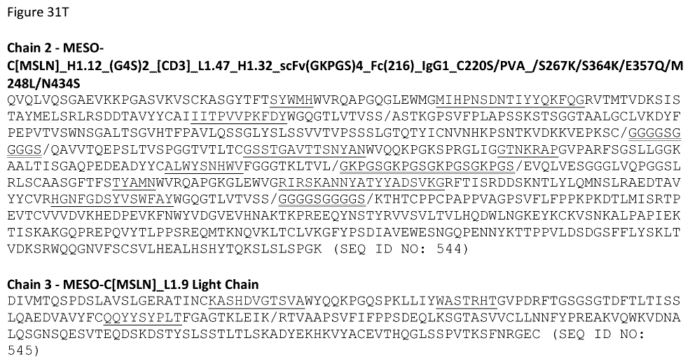

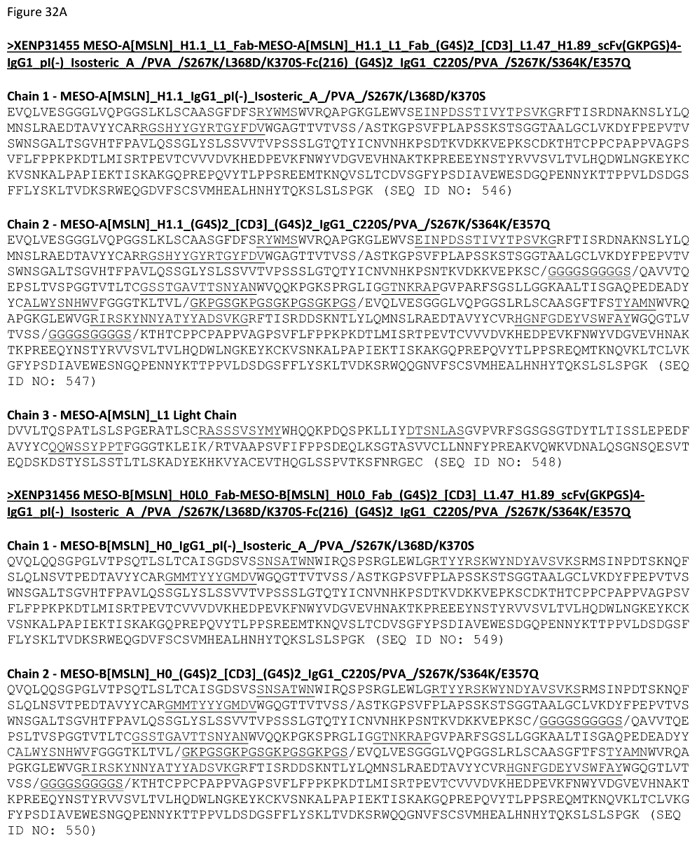

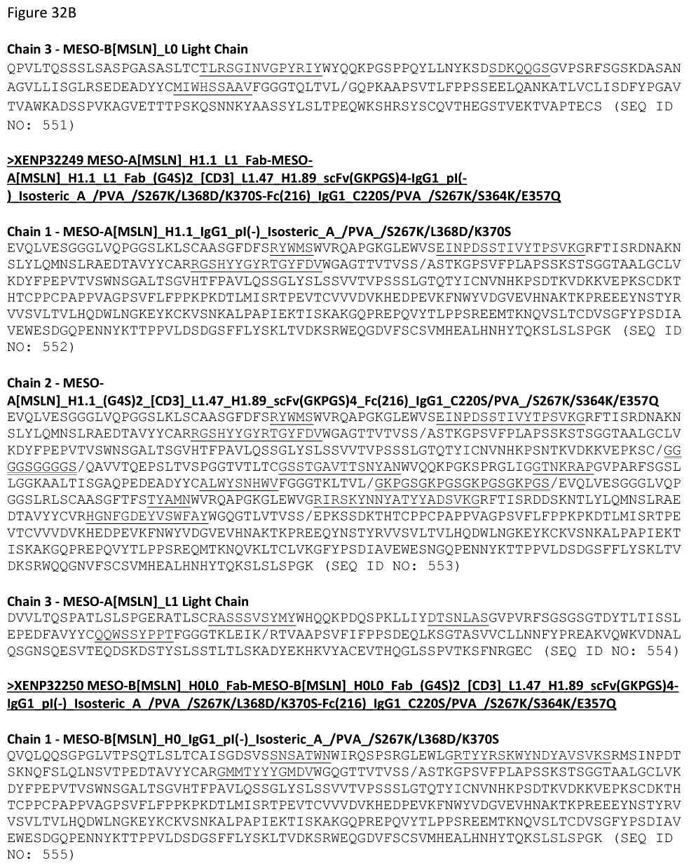

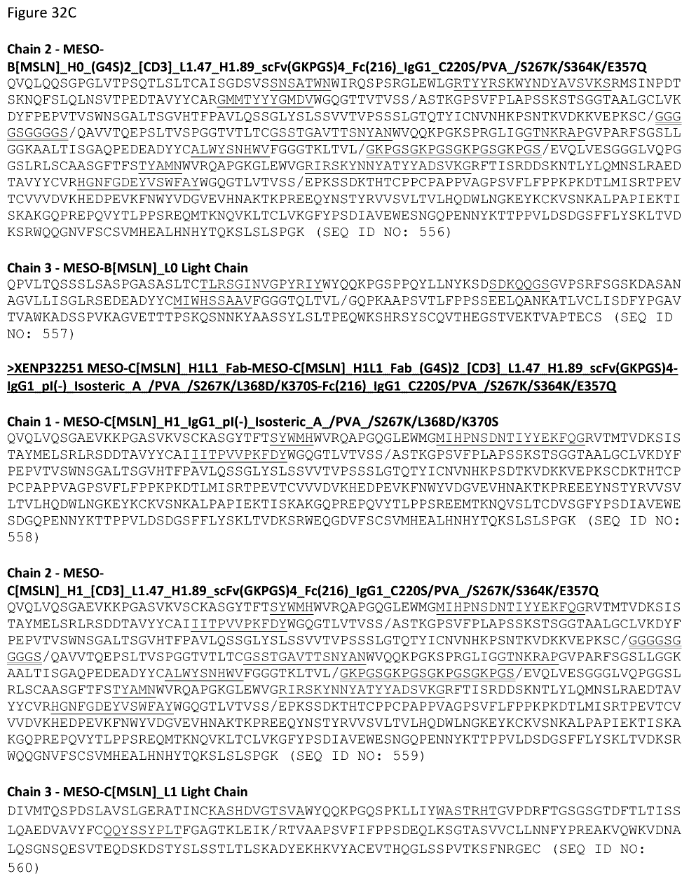

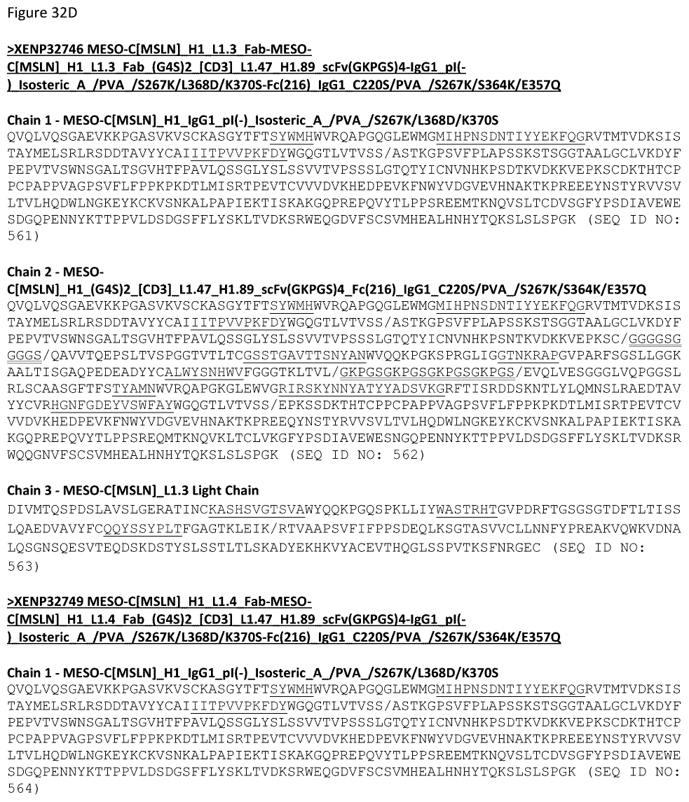

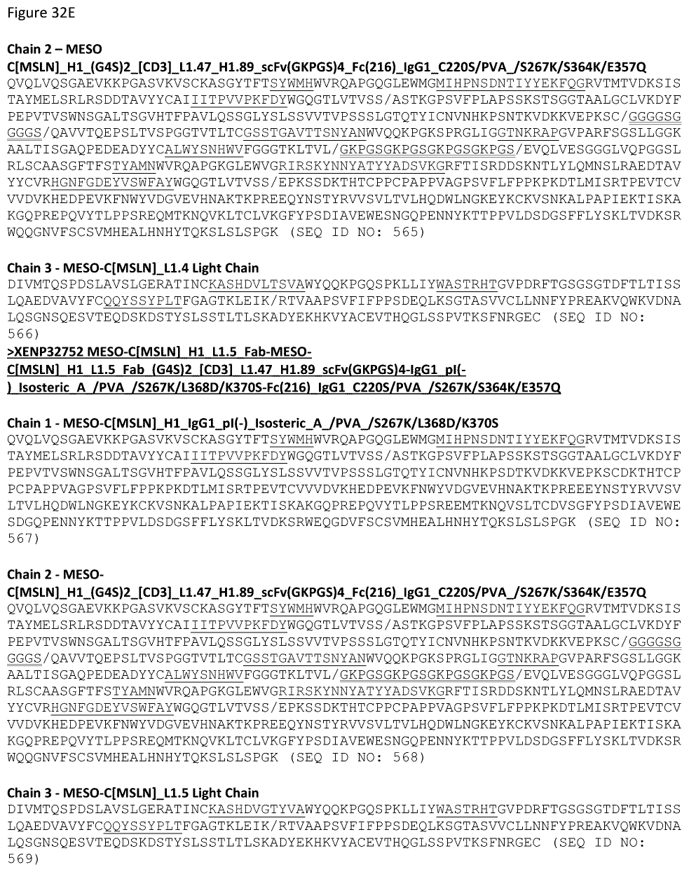

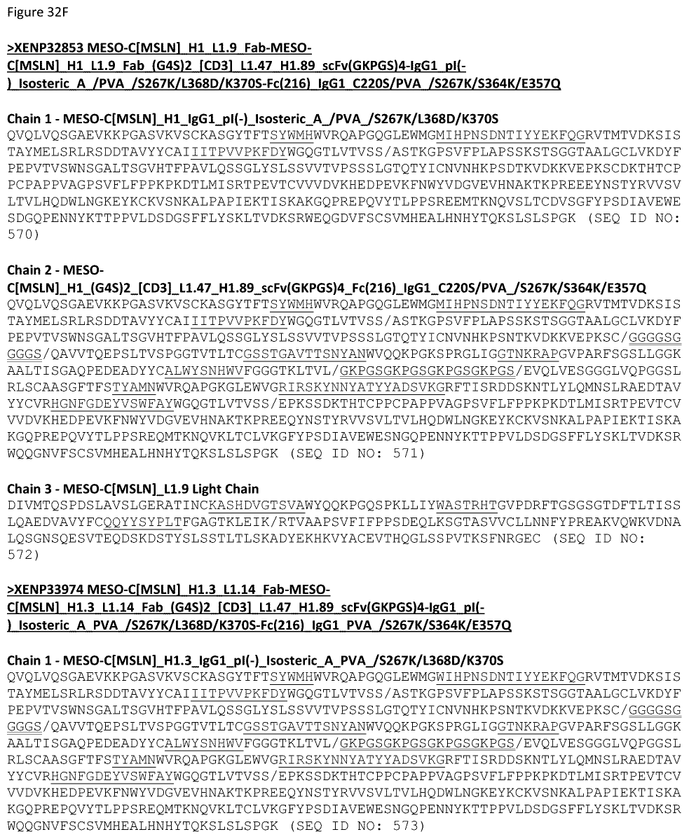

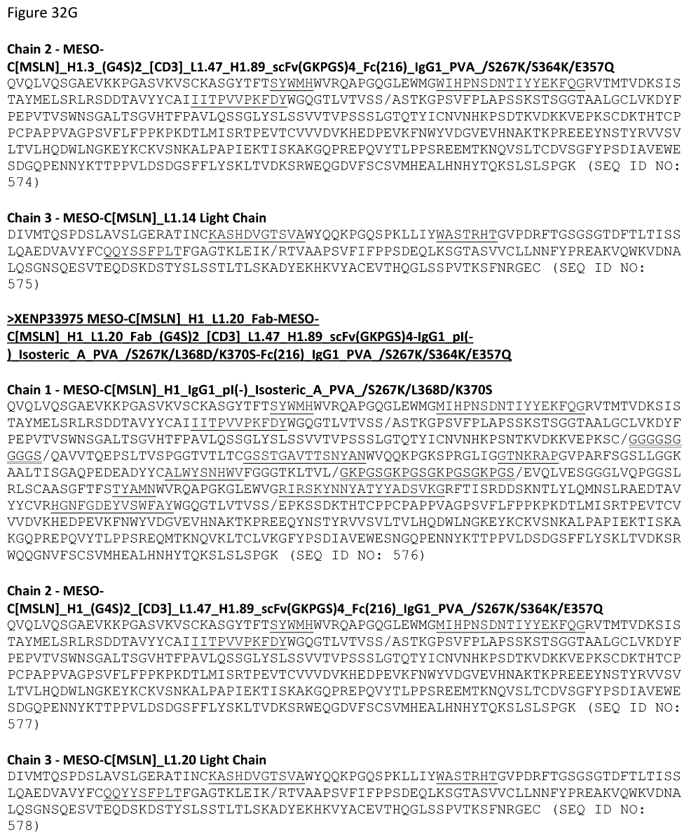

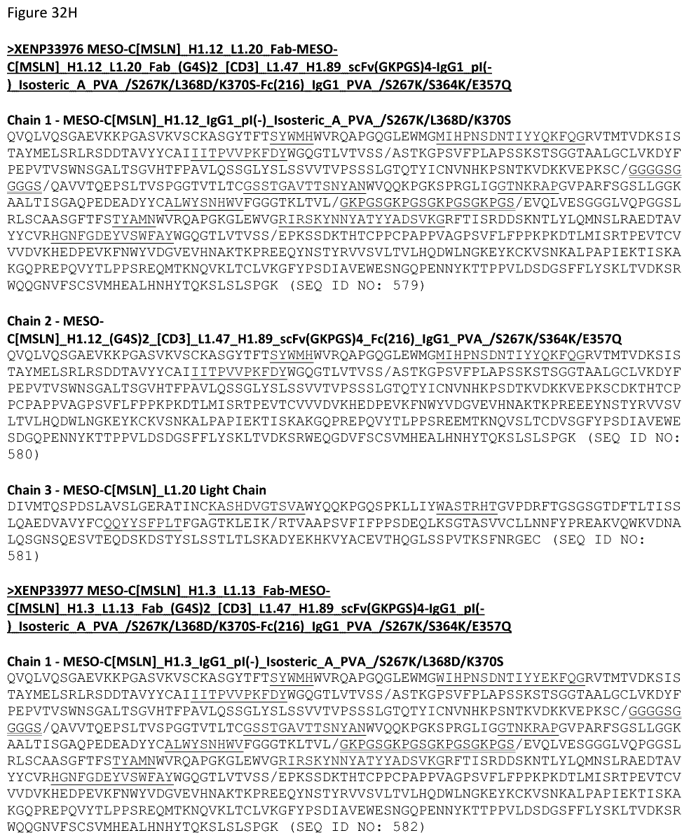

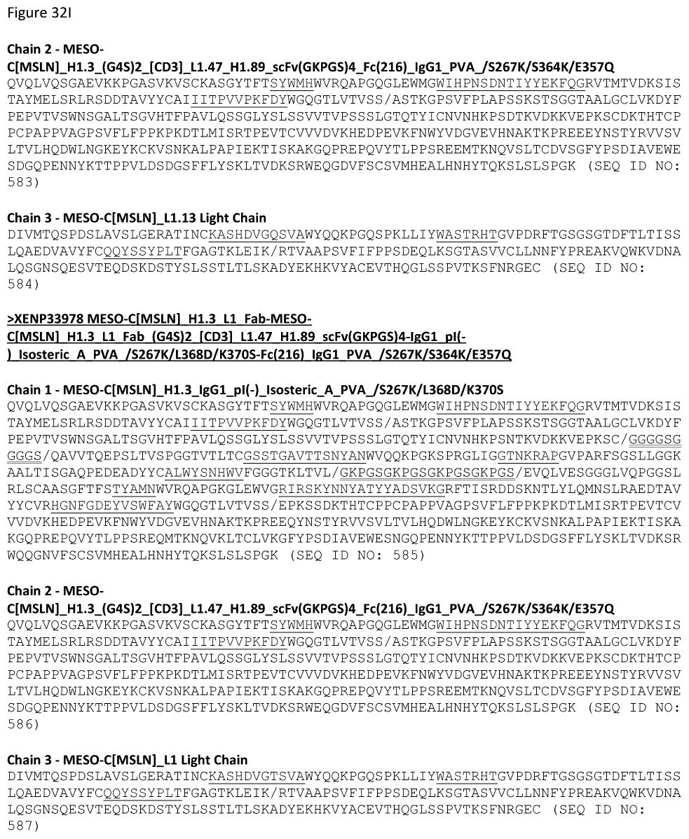

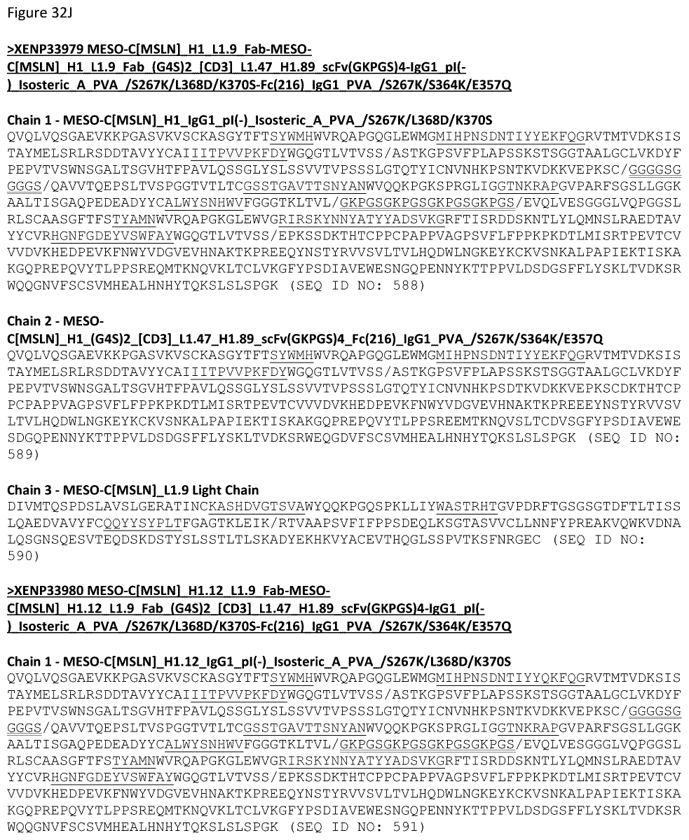

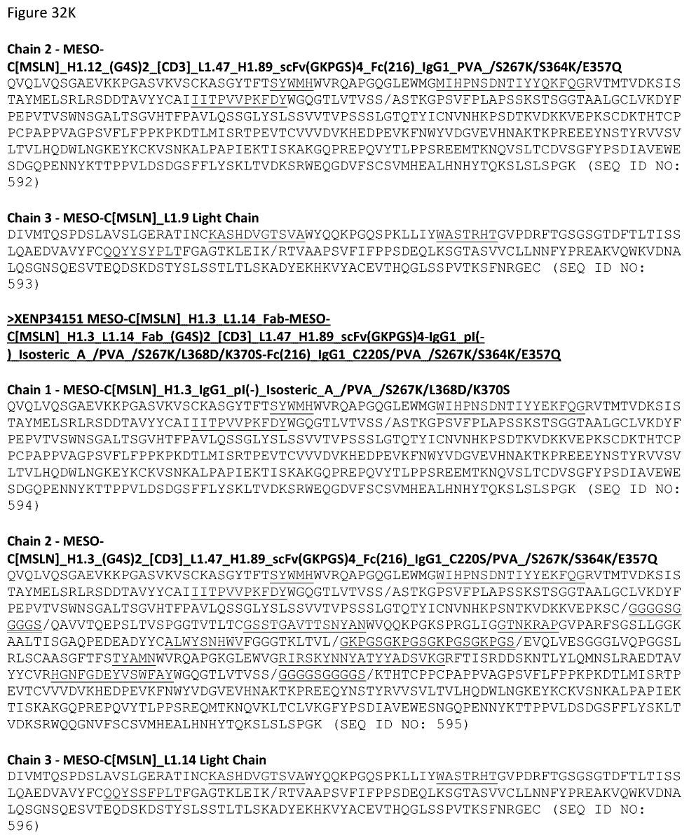

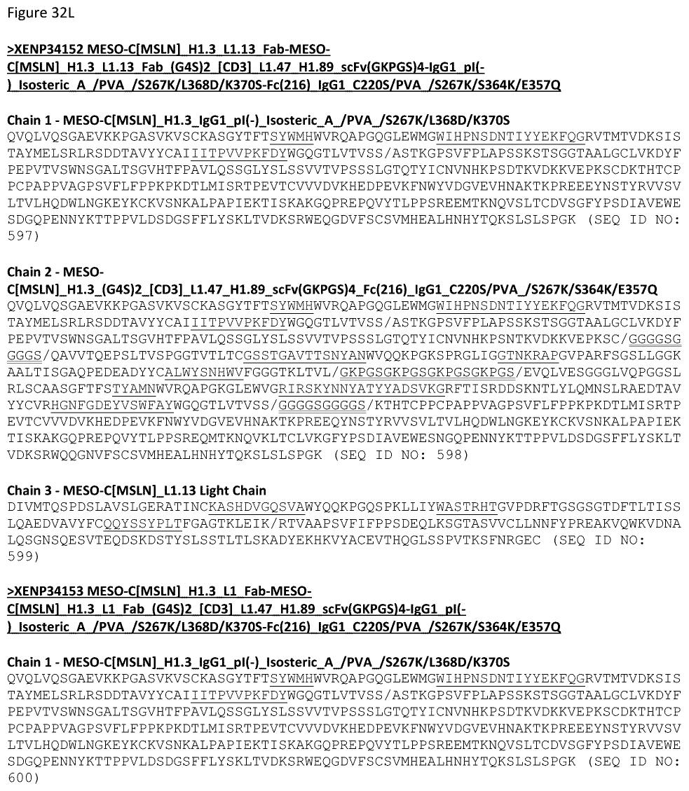

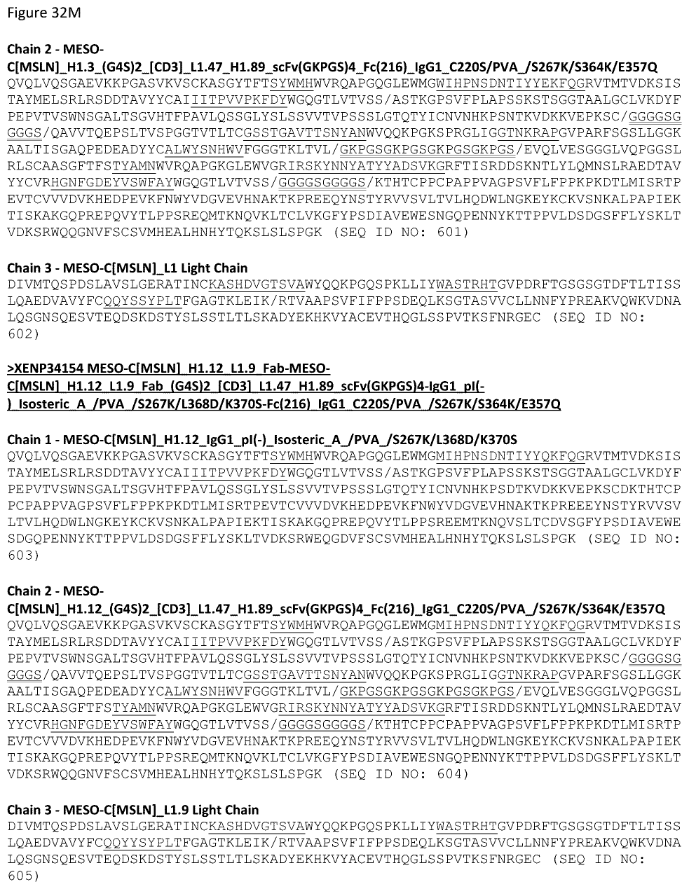

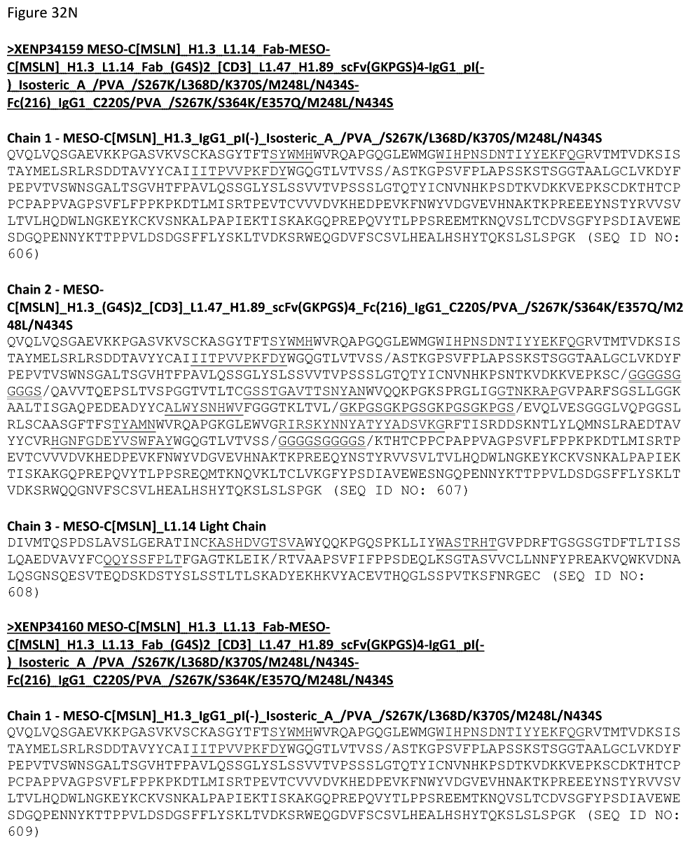

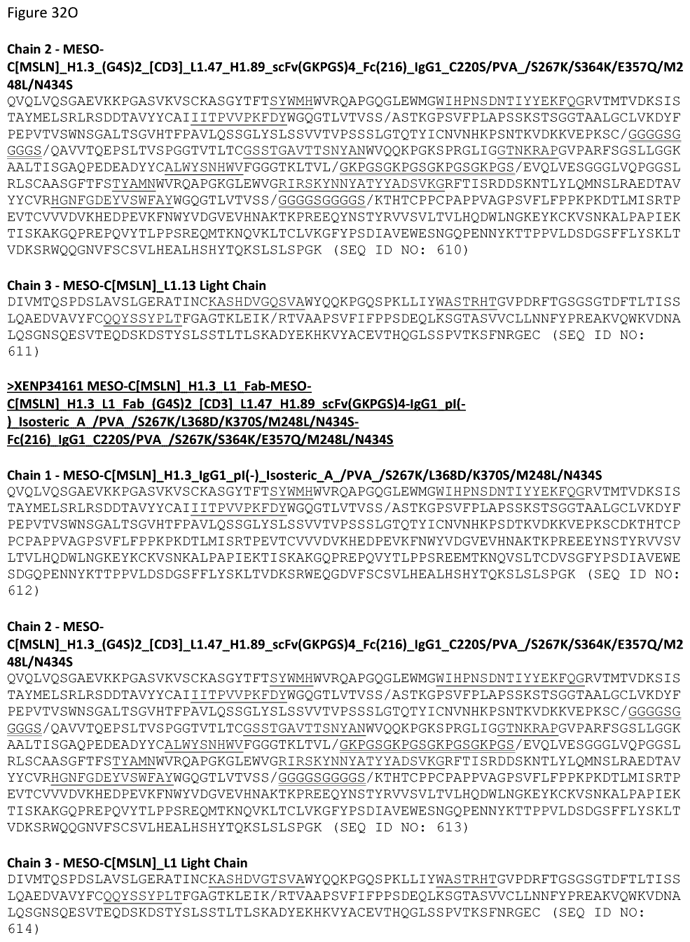

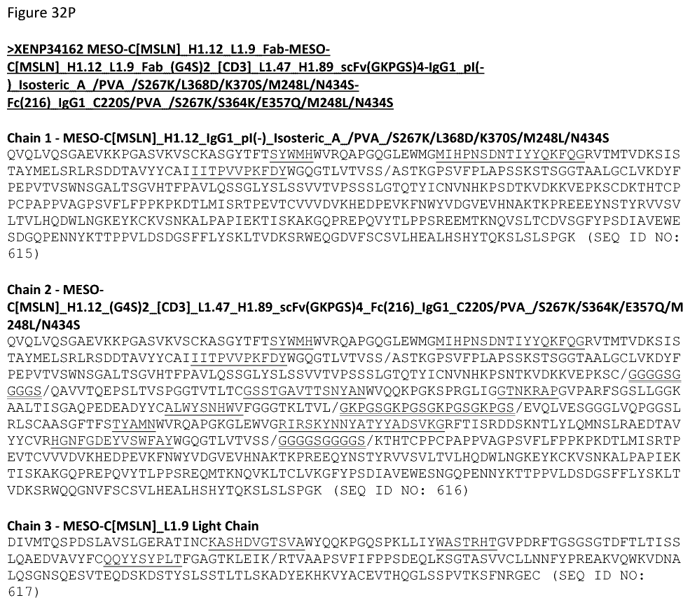

In one embodiment, the 2+1 Fab₂-scFv-Fc format antibody includes an scFv with the VH and VL of a CD3 binding domain sequence depicted in FIG. 10A-10F or the Sequence Listing. In one embodiment, the 2+1 Fab₂-scFv-Fc format antibody includes two Fabs having the VH and VL of a MSLN binding domain as shown in FIGS. 15-21 and the Sequence Listing. In an exemplary embodiment, the MSLN binding domain of the 2+1 Fab₂-scFv-Fc MSLN×CD3 bispecific antibody includes the VH and VL of one of the following MSLN binding domains: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21). In one embodiment, the CD3 binding domain of the 2+1 Fab2-scFv-Fc format antibody includes the VH and VL of one of the following CD3 binding domains: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F). Particularly useful MSLN and CD3 combinations for use in the 2+1 Fab2-scFv-Fc format antibody format are disclosed in FIGS. 28-32 and include: MESO-A[MSLN]_H1.1_L1×CD3 H1.30 L1.47, MESO-B[MSLN]_H0L0×CD3 H1.30 L1.47, MESO-C[MSLN]_H1L1×CD3 H1.30 L1.47, MESO-A[MSLN]_H1.1_L1×CD3 L1.47 H1.30, MESO-B[MSLN]_H0L0×CD3 L1.47 H1.30, MESO-C[MSLN]_H1L1×CD3 L1.47 H1.30, MESO-A[MSLN]_H1.1_L1×CD3 H1.32 L1.47, MESO-B[MSLN]_H0L0×CD3 H1.32 L1.47, MESO-C[MSLN]_H1L1×CD3 H1.32 L1.47, MESO-C[MSLN]_H1 L1.3×CD3 H1.32 L1.47, MESO-C[MSLN]_H1L1.4×CD3 H1.32 L1.47, MESO-C[MSLN]_H1 L1.5×CD3 H1.32 L1.47, MESO-C[MSLN]_H1 L1.9×CD3 H1.32 L1.47, MESO-A[MSLN]_H1.1 L1×CD3 L1.47 H1.32, MESO-B[MSLN]_H0L0×CD3 L1.47 H1.32, MESO-C[MSLN]_H1L1×CD3 L1.47 H1.32, MESO-C[MSLN]_H1 L1.3×CD3 L1.47 H1.32, MESO-C[MSLN]_H1 L1.4×CD3 L1.47 H1.32, MESO-C[MSLN]_H1 L1.5×CD3 L1.47 H1.32, MESO-C[MSLN]_H1 L1.9×CD3 L1.47 H1.32, MESO-C[MSLN]_H1.3 L1×CD3 L1.47 H1.32, MESO-C[MSLN]_H1.12 L1.9×CD3 L1.47 H1.32, MESO-C[MSLN]_H1.13 L1.9×CD3 L1.47 H1.32, MESO-C[MSLN]_H1.3 L1.9×CD3 L1.47 H1.32, MESO-C[MSLN]_H1.3 L1.5×CD3 L1.47 H1.32, MESO-C[MSLN]_H1.3 L1.13×CD3 L1.47 H1.32, MESO-C[MSLN]_H1.3 L1.14×CD3 L1.47 H1.32, MESO-C[MSLN]_H1 L1.19×CD3 L1.47 H1.32, MESO-C[MSLN]_H1 L1.20×CD3 L1.47 H1.32, MESO-C[MSLN]_H1 L1.21×CD3 L1.47 H1.32, MESO-C[MSLN]_H1 L1.22×CD3 L1.47 H1.32, MESO-C[MSLN]_H1.12 L1.19×CD3 L1.47 H1.32, MESO-C[MSLN]_H1.12 L1.20×CD3 L1.47 H1.32, MESO-C[MSLN]_H1.12 L1.21×CD3 L1.47 H1.32, MESO-C[MSLN]_H1.12 L1.22×CD3 L1.47 H1.32, MESO-C[MSLN]_H1.14 L1.14×CD3 L1.47 H1.32, MESO-C[MSLN]_H1.15 L1.14×CD3 L1.47 H1.32, MESO-C[MSLN]_H1.12 L1.19×CD3 L1.47 H1.32, MESO-C[MSLN]_H1.3 L1.14×CD3 L1.47 H1.32, MESO-C[MSLN]_H1.3 L1.13×CD3 L1.47 H1.32, MESO-C[MSLN]_H1.3 L1×CD3 L1.47 H1.32, MESO-C[MSLN]_H1.12 L1.9×CD3 L1.47 H1.32, MESO-A[MSLN]_H1.1 L1×CD3 L1.47 H1.89, MESO-B[MSLN]_H0L0×CD3 L1.47 H1.89, MESO-C[MSLN]_H1L1×CD3 L1.47 H1.89, MESO-C[MSLN]_H1 L1.3×CD3 L1.47 H1.89, MESO-C[MSLN]_H1 L1.4×CD3 L1.47 H1.89, MESO-C[MSLN]_H1 L1.5×CD3 L1.47 H1.89, MESO-C[MSLN]_H1 L1.9×CD3 L1.47 H1.89, MESO-C[MSLN]_H1.3 L1.14×CD3 L1.47 H1.89, MESO-C[MSLN]_H1 L1.20×CD3 L1.47 H1.89, MESO-C[MSLN]_H1.12 L1.20×CD3 L1.47 H1.89, MESO-C[MSLN]_H1.3 L1.13×CD3 L1.47 H1.89, MESO-C[MSLN]_H1.3 L1×CD3 L1.47 H1.89, MESO-C[MSLN]_H1 L1.9×CD3 L1.47 H1.89, MESO-C[MSLN]_H1.12 L1.9×CD3 L1.47 H1.89, MESO-C[MSLN]_H1.3 L1.14×CD3 L1.47 H1.89, MESO-C[MSLN]_H1.3 L1.13×CD3 L1.47 H1.89, MESO-C[MSLN]_H1.3 L1×CD3 L1.47 H1.89, MESO-C[MSLN]_H1.12 L1.9×CD3 L1.47 H1.89, MESO-C[MSLN]_H1.3 L1.14×CD3 L1.47 H1.89, MESO-C[MSLN]_H1.3 L1.13×CD3 L1.47 H1.89, MESO-C[MSLN]_H1.3 L1×CD3 L1.47 H1.89, and MESO-C[MSLN]_H1.12 L1.9×CD3 L1.47 H1.89.

In addition, the Fc domains of the 2+1 Fab₂-scFv-Fc format comprise skew variants (e.g. a set of amino acid substitutions as shown in FIGS. 1 and 4, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q, T366S/L368A/Y407V: T366W and T366S/L368A/Y407V/Y349C: T366W/S354C), optionally ablation variants (including those shown in FIG. 3), optionally charged scFv linkers (including those shown in FIG. 5) and the heavy chain comprises pI variants (including those shown in FIG. 2).

In some embodiments, the 2+1 Fab₂-scFv-Fc format antibody includes skew variants, pI variants, and ablation variants. Accordingly, some embodiments include 2+1 Fab₂-scFv-Fc formats that comprise: a) a first monomer (the Fab-scFv-Fc side) that comprises the skew variants S364K/E357Q, the ablation variants E233P/L234V/L235A/G236del/S267K, and a variable heavy domain that, with the variable light domain of the common light chain, makes up an Fv that binds to MSLN as outlined herein, and an scFv domain that binds to CD3; b) a second monomer (the Fab-Fc side) that comprises the skew variants L368D/K370S, the pI variants N208D/Q295E/N384D/Q418E/N421D, the ablation variants E233P/L234V/L235A/G236del/S267K, and a variable heavy domain that, with variable light domain of the common light chain, makes up an Fv that binds to MSLN as outlined herein; and c) a common light chain comprising the variable light domain and a constant light domain, where numbering is according to EU numbering. In some embodiments, the common light chain and variable heavy domains on each monomer for MSLN binding domains. CD3 binding domain sequences finding particular use in these embodiments include, but are not limited to, H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 as well as those depicted in FIGS. 10A-10F. MSLN binding domain sequences that are of particular use in these embodiments include, but are not limited to MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In some embodiments, the 2+1 Fab₂-scFv-Fc format antibody includes skew variants, pI variants, ablation variants and FcRn variants. Accordingly, some embodiments include 2+1 Fab₂-scFv-Fc formats that comprise: a) a first monomer (the Fab-scFv-Fc side) that comprises the skew variants S364K/E357Q, the ablation variants E233P/L234V/L235A/G236del/S267K, the FcRn variants M428L/N434S and a variable heavy domain that, with the variable light domain of the common light chain, makes up an Fv that binds to MSLN as outlined herein, and an scFv domain that binds to CD3; b) a second monomer (the Fab-Fc side) that comprises the skew variants L368D/K370S, the pI variants N208D/Q295E/N384D/Q418E/N421D, the ablation variants E233P/L234V/L235A/G236del/S267K, the FcRn variants M428L/N434S and a variable heavy domain that, with variable light domain of the common light chain, makes up an Fv that binds to MSLN as outlined herein; and c) a common light chain comprising a variable light domain and a constant light domain, where numbering is according to EU numbering. In some embodiments, the common light chain and variable heavy domains on each monomer for MSLN binding domains. CD3 binding domain sequences finding particular use in these embodiments include, but are not limited to, H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 as well as those depicted in FIGS. 10A-10F. In some embodiments, the MSLN binding domains include the variable heavy and variable light domain of any one of the MSLN binding domains disclosed in FIGS. 15-21. MSLN binding domain sequences that are of particular use in these embodiments include but are not limited to: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

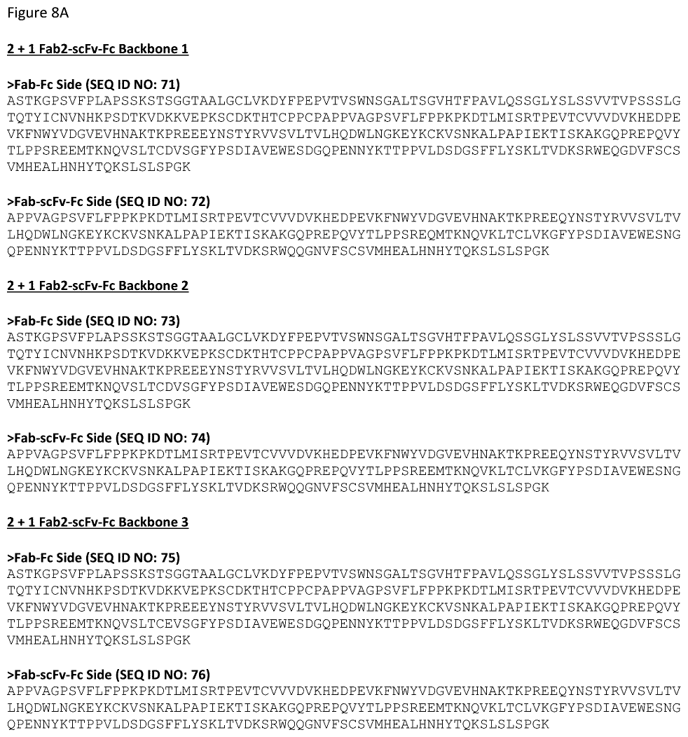

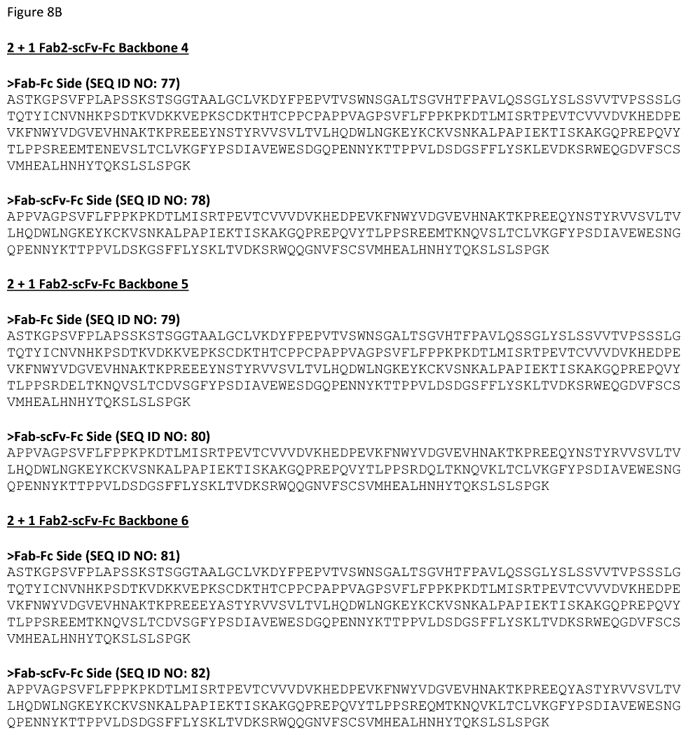

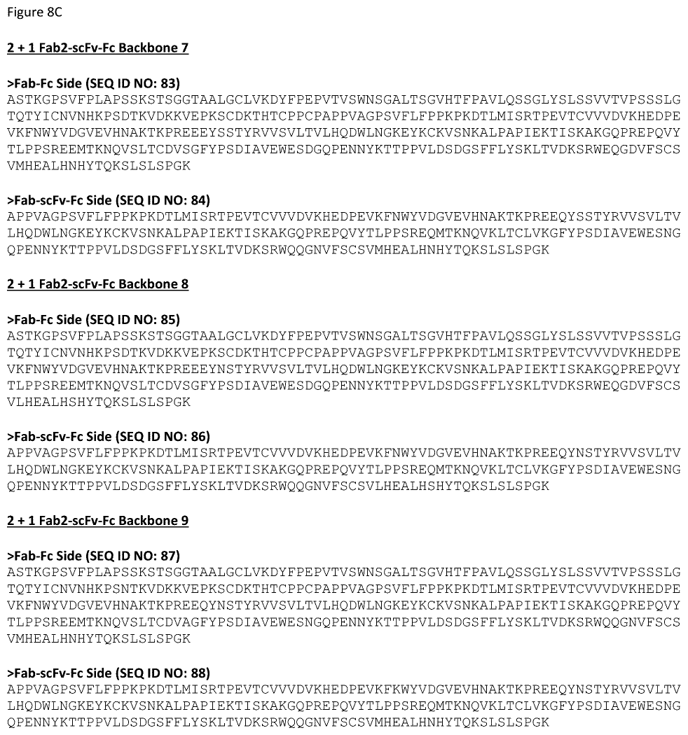

FIGS. 8A-8C shows some exemplary Fc domain sequences that are useful with the 2+1 Fab₂-scFv-Fc format. The “monomer 1” sequences depicted in FIGS. 8A-8C typically refer to the Fc domain of the “Fab-Fc heavy chain” and the “monomer 2” sequences refer to the Fc domain of the “Fab-scFv-Fc heavy chain.” Further, FIG. 9 provides useful CL sequences that can be used with this format.

In some embodiments, the 2+1 Fab₂-scFv-Fc format includes sequences selected from: XENP16249, XENP16250, XENP16253, XENP16827, XENP31693.

In some embodiments, the 2+1 Fab₂-scFv-Fc format comprises sequences selected from: XENP31063, XENP31067, XENP31705, XENP31451, XENP31452, XENP31713, XENP31064, XENP31068, XENP31709, ENP32744, XENP32747, XENP32750, XENP32851, XENP31453, XENP31454, XENP31717, XENP32745, XENP32748, XENP32751, XENP32852, XENP32947, XENP32948, XENP32949, XENP32950, XENP32951, XENP33366, XENP33367, XENP33460, XENP33461, XENP33462, XENP33463, XENP33464, XENP33465, XENP33466, XENP33467, XENP33968, XENP33970, XENP33972, XENP34155, XENP34156, XENP34157, XENP34158, XENP31455, XENP31456, XENP32249, XENP32250, XENP32251, XENP32746, XENP32749, XENP32752, XENP32853, XENP33974, XENP33975, XENP33976, XENP33977, XENP33978, XENP33979, XENP33980, XENP34151, XENP34152, XENP34153, XENP34154, XENP34159, XENP34160, XENP34161, and XENP34162 (FIGS. 28-32).

In some embodiments, the 2+1 Fab₂-scFv-Fc format comprises a variable heavy chain MSLN sequence comprising one or more of the following mutations: S31G, W32Y, M50W, H52N, N53Q, D55G, 194R, T97S, E61Q, E61Y, and/or K100bR.

In some embodiments, the 2+1 Fab2-scFv-Fc format comprises a variable light chain MSLN sequence comprising one or more of the following mutations: A25S, H27Q, D28S, G30L, S32Y, V33L, T37Q, H55E, T56S, S92Y, Y94T, Y94L, Y94V, Y94I, Y94H, D28S and Y100F.

In some embodiments, the 2+1 Fab₂-scFv-Fc format includes a MSLN binding domain that includes a MSLN binding domain variable heavy domain selected from any of FIGS. 15-18 and 20-21. In some embodiments, the 2+1 Fab₂-scFv-Fc format includes a MSLN binding domain that includes a MSLN binding domain variable light domain selected from any of FIGS. 15-17 and 19-21. In some embodiments, the 2+1 Fab₂-scFv-Fc format includes a MSLN binding domain that includes a combination of any of the MSLN binding domain variable heavy domains and variable light domains in FIGS. 15-21.

### 5. Central-Fv

One heterodimeric scaffold that finds particular use in the antibodies described herein is the Central-Fv format. In this embodiment, the format relies on the use of an inserted Fv domain (i.e., the central Fv domain) thus forming an “extra” third antigen binding domain, wherein the Fab portions of the two monomers bind a MSLN and the “extra” central Fv domain binds CD3. The “extra” central Fv domain is inserted between the Fc domain and the CH1-Fv region of the monomers, thus providing a third antigen binding domain (i.e., the “extra” central Fv domain), wherein each monomer contains a component of the “extra” central Fv domain (i.e., one monomer comprises the variable heavy domain and the other a variable light domain of the “extra” central Fv domain).

In this embodiment, one monomer comprises a first heavy chain comprising a first variable heavy domain, a CH1 domain, and Fc domain and an additional variable light domain. The light domain is covalently attached between the C-terminus of the CH1 domain of the heavy constant domain and the N-terminus of the first Fc domain using domain linkers (VH1-CH1-[optional linker]-VL2-hinge-CH2-CH3). The other monomer comprises a first heavy chain comprising a first variable heavy domain, a CH1 domain and Fc domain and an additional variable heavy domain (VH1-CH1-[optional linker]-VH2-hinge-CH2-CH3). The light domain is covalently attached between the C-terminus of the CH1 domain of the heavy constant domain and the N-terminus of the first Fc domain using domain linkers.

This embodiment further utilizes a common light chain comprising a variable light domain and a constant light domain, that associates with the heavy chains to form two identical Fabs that each bind a MSLN. As for many of the embodiments herein, these constructs include skew variants, pI variants, ablation variants, additional Fc variants, etc. as desired and described herein.

The antibodies described herein provide central-Fv formats where the CD3 binding domain sequences are as shown in 10A-10F and the MSLN binding domain sequences are as shown in FIGS. 15-21.

In some embodiments, the MSLN binding domains include the variable heavy and variable light domain of any one of the MSLN binding domains disclosed in FIGS. 15-21.

### 6. One Armed Central-scFv

One heterodimeric scaffold that finds particular use in the antibodies described herein is the one armed central-scFv format. In this embodiment, one monomer comprises just an Fc domain, while the other monomer includes a Fab domain (a first antigen binding domain), a scFv domain (a second antigen binding domain) and an Fc domain, where the scFv domain is inserted between the Fab domain and the Fc domain. In this format, the Fab portion binds one receptor target and the scFv binds another. In this format, either the Fab portion binds a MSLN and the scFv binds CD3 or vice versa.

In this embodiment, one monomer comprises a first heavy chain comprising a first variable heavy domain, a CH1 domain and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain comprising a variable light domain and a constant light domain that associates with the heavy chain to form a Fab.

As for many of the embodiments herein, these constructs include skew variants, pI variants, ablation variants, additional Fc variants, etc. as desired and described herein.

The antibodies described herein provide central-Fv formats where the CD3 binding domain sequences are as shown in FIG. 10A-10F and the MSLN binding domain sequences are as shown in FIGS. 15-21.

In addition, the Fc domains of the one armed central-scFv format generally include skew variants (e.g. a set of amino acid substitutions as shown in FIGS. 1 and 4, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q, T366S/L368A/Y407V: T366W and T366S/L368A/Y407V/Y349C: T366W/S354C), optionally ablation variants (including those shown in FIG. 3), optionally charged scFv linkers (including those shown in FIG. 5) and the heavy chain comprises pI variants (including those shown in FIG. 2).

In some embodiments, the one armed central-scFv format includes skew variants, pI variants, and ablation variants. Accordingly, some embodiments of the one armed central-scFv formats comprise: a) a first monomer that comprises the skew variants S364K/E357Q, the ablation variants E233P/L234V/L235A/G236del/S267K, and a variable heavy domain that, with the variable light domain of the light chain, makes up an Fv that binds to MSLN as outlined herein, and a scFv domain that binds to CD3; b) a second monomer that includes an Fc domain having the skew variants L368D/K370S, the pI variants N208D/Q295E/N384D/Q418E/N421D, the ablation variants E233P/L234V/L235A/G236del/S267K; and c) a light chain comprising a variable light domain and a constant light domain.

In some embodiments, the one armed central-scFv format includes skew variants, pI variants, ablation variants and FcRn variants. Accordingly, some embodiments of the one armed central-scFv formats comprise: a) a first monomer that comprises the skew variants S364K/E357Q, the ablation variants E233P/L234V/L235A/G236del/S267K, the FcRn variants M428L/N434S and a variable heavy domain that, with the variable light domain of the light chain, makes up an Fv that binds to MSLN as outlined herein, and a scFv domain that binds to CD3; b) a second monomer that includes an Fc domain having the skew variants L368D/K370S, the pI variants N208D/Q295E/N384D/Q418E/N421D, the ablation variants E233P/L234V/L235A/G236del/S267K, and the FcRn variants M428L/N434S; and c) a light chain comprising a variable light domain and a constant light domain.

In some embodiments, the MSLN binding domains include the variable heavy and variable light domain of any one of the MSLN binding domains disclosed in FIGS. 15-21.

7. One Armed scFv-mAb

One heterodimeric scaffold that finds particular use in the antibodies described herein is the one armed scFv-mAb format. In this embodiment, one monomer comprises just an Fc domain, while the other monomer uses a scFv domain attached at the N-terminus of the heavy chain, generally through the use of a linker: VH-scFv linker-VL-[optional domain linker]-CH1-hinge-CH2-CH3 or (in the opposite orientation) VL-scFv linker-VH-[optional domain linker]-CH1-hinge-CH2-CH3. In this format, the Fab portions each bind MSLN and the scFv binds CD3. This embodiment further utilizes a light chain comprising a variable light domain and a constant light domain, that associates with the heavy chain to form a Fab. As for many of the embodiments herein, these constructs include skew variants, pI variants, ablation variants, additional Fc variants, etc. as desired and described herein.

The antibodies described herein provide one armed scFv-mAb formats where the CD3 binding domain sequences are as shown in 10A-10F and wherein the MSLN binding domain sequences are as shown in FIGS. 15-21.

In addition, the Fc domains of the one armed scFv-mAb format generally include skew variants (e.g. a set of amino acid substitutions as shown in FIGS. 1 and 4, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q, T366S/L368A/Y407V: T366W and T366S/L368A/Y407V/Y349C: T366W/S354C), optionally ablation variants (including those shown in FIG. 3), optionally charged scFv linkers (including those shown in FIG. 5) and the heavy chain comprises pI variants (including those shown in FIG. 2).

In some embodiments, the one armed scFv-mAb format includes skew variants, pI variants, and ablation variants. Accordingly, some embodiments of the one armed scFv-mAb formats comprise: a) a first monomer that comprises the skew variants S364K/E357Q, the ablation variants E233P/L234V/L235A/G236del/S267K, and a variable heavy domain that, with the variable light domain of the light chain, makes up an Fv that binds to MSLN as outlined herein, and a scFv domain that binds to CD3; b) a second monomer that includes an Fc domain having the skew variants L368D/K370S, the pI variants N208D/Q295E/N384D/Q418E/N421D, the ablation variants E233P/L234V/L235A/G236del/S267K; and c) a light chain comprising a variable light domain and a constant light domain.

In some embodiments, the one armed scFv-mAb format includes skew variants, pI variants, ablation variants and FcRn variants. Accordingly, some embodiments one armed scFv-mAb formats comprise: a) a first monomer that comprises the skew variants S364K/E357Q, the ablation variants E233P/L234V/L235A/G236del/S267K, the FcRn variants M428L/N434S and a variable heavy domain that, with the variable light domain of the light chain, makes up an Fv that binds to MSLN as outlined herein, and a scFv domain that binds to CD3; b) a second monomer that includes an Fc domain having the skew variants L368D/K370S, the pI variants N208D/Q295E/N384D/Q418E/N421D, the ablation variants E233P/L234V/L235A/G236del/S267K, and the FcRn variants M428L/N434S; and c) a light chain comprising a variable light domain and a constant light domain.

In some embodiments, the MSLN binding domains include the variable heavy and variable light domain of any one of the MSLN binding domains disclosed in FIGS. 15-21.

8. scFv-mAb

One heterodimeric scaffold that finds particular use in the antibodies described herein is the mAb-scFv format. In this embodiment, the format relies on the use of a N-terminal attachment of a scFv to one of the monomers, thus forming a third antigen binding domain, wherein the Fab portions of the two monomers bind MSLN and the “extra” scFv domain binds CD3.

In this embodiment, the first monomer comprises a first heavy chain (comprising a variable heavy domain and a constant domain), with a N-terminally covalently attached scFv comprising a scFv variable light domain, an scFv linker and a scFv variable heavy domain in either orientation ((VH1-scFv linker-VL1-[optional domain linker]-VH2-CH1-hinge-CH2-CH3) or (with the scFv in the opposite orientation) ((VL1-scFv linker-VH1-[optional domain linker]-VH2-CH1-hinge-CH2-CH3)). This embodiment further utilizes a common light chain comprising a variable light domain and a constant light domain that associates with the heavy chains to form two identical Fabs that bind MSLN. As for many of the embodiments herein, these constructs include skew variants, pI variants, ablation variants, additional Fc variants, etc. as desired and described herein.

The antibodies described herein provide scFv-mAb formats where the CD3 binding domain sequences are as shown in 10A-10F and wherein the MSLN binding domain sequences are as shown in FIGS. 15-21.

In addition, the Fc domains of the scFv-mAb format generally include skew variants (e.g. a set of amino acid substitutions as shown in FIGS. 1 and 4, with particularly useful skew variants being selected from the group consisting of S364K/E357Q: L368D/K370S; L368D/K370S: S364K; L368E/K370S: S364K; T411T/E360E/Q362E: D401K; L368D/K370S: S364K/E357L, K370S: S364K/E357Q, T366S/L368A/Y407V: T366W and T366S/L368A/Y407V/Y349C: T366W/S354C), optionally ablation variants (including those shown in FIG. 3), optionally charged scFv linkers (including those shown in FIG. 5) and the heavy chain comprises pI variants (including those shown in FIG. 2).

In some embodiments, the scFv-mAb format includes skew variants, pI variants, and ablation variants. Accordingly, some embodiments include scFv-mAb formats that comprise: a) a first monomer that comprises the skew variants S364K/E357Q, the ablation variants E233P/L234V/L235A/G236del/S267K, and a variable heavy domain that, with the variable light domain of the common light chain, makes up an Fv that binds to MSLN as outlined herein, and a scFv domain that binds to CD3; b) a second monomer that comprises the skew variants L368D/K370S, the pI variants N208D/Q295E/N384D/Q418E/N421D, the ablation variants E233P/L234V/L235A/G236del/S267K, and a variable heavy domain that, with the variable light domain of the common light chain, makes up an Fv that binds to MSLN as outlined herein; and c) a common light chain comprising a variable light domain and a constant light domain.

In some embodiments, the scFv-mAb format includes skew variants, pI variants, ablation variants and FcRn variants. Accordingly, some embodiments include scFv-mAb formats that comprise: a) a first monomer that comprises the skew variants S364K/E357Q, the ablation variants E233P/L234V/L235A/G236del/S267K, the FcRn variants M428L/N434S and a variable heavy domain that, with the variable light domain of the common light chain, makes up an Fv that binds to MSLN as outlined herein, and a scFv domain that binds to CD3; b) a second monomer that comprises the skew variants L368D/K370S, the pI variants N208D/Q295E/N384D/Q418E/N421D, the ablation variants E233P/L234V/L235A/G236del/S267K, the FcRn variants M428L/N434S and a variable heavy domain that, with the variable light domain of the common light chain, makes up an Fv that binds to MSLN as outlined herein; and c) a common light chain comprising a variable light domain and a constant light domain.

In some embodiments, the MSLN binding domains include the variable heavy and variable light domain of any one of the MSLN binding domains disclosed in FIGS. 15-21.

9. Dual scFv Formats

The antibodies described herein also provide dual scFv formats as are known in the art. In this embodiment, the MSLN×CD3 heterodimeric bispecific antibody is made up of two scFv-Fc monomers (both in either (VH-scFv linker-VL-[optional domain linker]-CH2-CH3) format or (VL-scFv linker-VH-[optional domain linker]-CH2-CH3) format, or with one monomer in one orientation and the other in the other orientation.

The antibodies described herein provide dual scFv formats where the CD3 binding domain sequences are as shown in FIG. 10A-10F and wherein the MSLN binding domain sequences are as shown in FIGS. 15-21. In some embodiments, the dual scFv format includes skew variants, pI variants, and ablation variants. Accordingly, some embodiments include dual scFv formats that comprise: a) a first monomer that comprises the skew variants S364K/E357Q, the ablation variants E233P/L234V/L235A/G236del/S267K, and a first scFv that binds either CD3 or MSLN; and b) a second monomer that comprises the skew variants L368D/K370S, the pI variants N208D/Q295E/N384D/Q418E/N421D, the ablation variants E233P/L234V/L235A/G236del/S267K, and a second scFv that binds either CD3 or MSLN. In some embodiments, the dual scFv format includes skew variants, pI variants, ablation variants and FcRn variants. In some embodiments, the dual scFv format includes skew variants, pI variants, and ablation variants. Accordingly, some embodiments include dual scFv formats that comprise: a) a first monomer that comprises the skew variants S364K/E357Q, the ablation variants E233P/L234V/L235A/G236del/S267K, the FcRn variants M428L/N434S and a first scFv that binds either CD3 or MSLN; and b) a second monomer that comprises the skew variants L368D/K370S, the pI variants N208D/Q295E/N384D/Q418E/N421D, the ablation variants E233P/L234V/L235A/G236del/S267K, the FcRn variants M428L/N434S and a second scFv that binds either CD3 or MSLN.

In some embodiments, the MSLN binding domains include the variable heavy and variable light domain of any one of the MSLN binding domains disclosed in FIGS. 15-21.

### 10. Non-Heterodimeric Bispecific Antibodies

As will be appreciated by those in the art, the MSLN and CD3 Fv sequences outlined herein can also be used in both monospecific antibodies (e.g., “traditional monoclonal antibodies”) or non-heterodimeric bispecific formats.

CD3 binding domain sequences finding particular use include, but are not limited to H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

MSLN binding domain sequences that are of particular use include, but are not limited to: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In some embodiments, the MSLN binding domains include the variable heavy and variable light domain of any one of the MSLN binding domains disclosed in FIGS. 15-21.

Suitable non-heterodimeric bispecific formats are known in the art, and include a number of different formats as generally depicted in Spiess et al., Molecular Immunology (67):95-106 (2015) and Kontermann, mAbs 4:2, 182-197 (2012), both of which are expressly incorporated by reference and in particular for the figures, legends and citations to the formats therein.

### 11. Trident Format

In some embodiments, the bispecific antibodies described herein are in the “Trident” format as generally described in WO2015/184203, hereby expressly incorporated by reference in its entirety and in particular for the Figures, Legends, definitions and sequences of “Heterodimer-Promoting Domains” or “HPDs”, including “K-coil” and “E-coil” sequences. Tridents rely on using two different HPDs that associate to form a heterodimeric structure as a component of the structure, see FIG. 1K. In this embodiment, the Trident format include a “traditional” heavy and light chain (e.g., VH1-CH1-hinge-CH2-CH3 and VL1-CL), a third chain comprising a first “diabody-type binding domain” or “DART®”, VH2-(linker)-VL3-HPD1 and a fourth chain comprising a second DART®, VH3-(linker)-(linker)-VL2-HPD2. The VH1 and VLI form a first ABD, the VH2 and VL2 form a second ABD, and the VH3 and VL3 form a third ABD. In some cases, as is shown in FIG. 1K, the second and third ABDs bind the same antigen, in this instance generally MSLN, e.g., bivalently, with the first ABD binding a CD3 monovalently.

In some embodiments, the MSLN binding domains include the variable heavy and variable light domain of any one of the MSLN binding domains disclosed in FIGS. 15-21.

### 12. Monospecific, Monoclonal Antibodies

As will be appreciated by those in the art, the novel Fv sequences outlined herein can also be used in both monospecific antibodies (e.g., “traditional monoclonal antibodies”) or non-heterodimeric bispecific formats. Accordingly, in some embodiments, the antibodies described herein provide monoclonal (monospecific) antibodies comprising the 6 CDRs and/or the vh and vl sequences from the figures, generally with IgG1, IgG2, IgG3 or IgG4 constant regions, with IgG1, IgG2 and IgG4 (including IgG4 constant regions comprising a S228P amino acid substitution) finding particular use in some embodiments. That is, any sequence herein with a “H_L” designation can be linked to the constant region of a human IgG1 antibody.

In some embodiments, the monospecific antibody is a MSLN monospecific antibody. In certain embodiments, the monospecific anti-MSLN antibody includes the 6 CDRs of any of the anti-MSLN antibodies selected from: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20,_MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21).

In some embodiments, the MSLN binding domains include the variable heavy and variable light domain of any one of the MSLN binding domains disclosed in FIGS. 15-21.

### G. Antigen Binding Domains

As discussed herein, the subject heterodimeric antibodies include two antigen binding domains (ABDs), each of which bind to MSLN or CD3. As outlined herein, these heterodimeric antibodies can be bispecific and bivalent (each antigen is bound by a single ABD, for example, in the format depicted in FIG. 15A), or bispecific and trivalent (one antigen is bound by a single ABD and the other is bound by two ABDs, for example as depicted in FIG. 15B).

In addition, in general, one of the ABDs comprises a scFv as outlined herein, in an orientation from N- to C-terminus of VH-scFv linker-VL or VL-scFv linker-VH. One or both of the other ABDs, according to the format, generally is a Fab, comprising a VH domain on one protein chain (generally as a component of a heavy chain) and a VL on another protein chain (generally as a component of a light chain).

The disclosure provides a number of ABDs that bind to a number of different checkpoint proteins, as outlined below. As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain). The scFv sequences contained in the sequence listing utilize a particular charged linker, but as outlined herein, uncharged or other charged linkers can be used, including those depicted in FIG. 7.

In addition, as discussed above, the numbering used in the Sequence Listing for the identification of the CDRs is Kabat, however, different numbering can be used, which will change the amino acid sequences of the CDRs as shown in Table 2.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85 or 90% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g. from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g. there may be one change in VLCDR1, two in VHCDR2, none in VHCDR3, etc.)), as well as having framework region changes, as long as the framework regions retain at least 80, 85 or 90% identity to a human germline sequence selected from those listed in FIG. 1 of U.S. Pat. No. 7,657,380.

### 1. MSLN Antigen Binding Domains

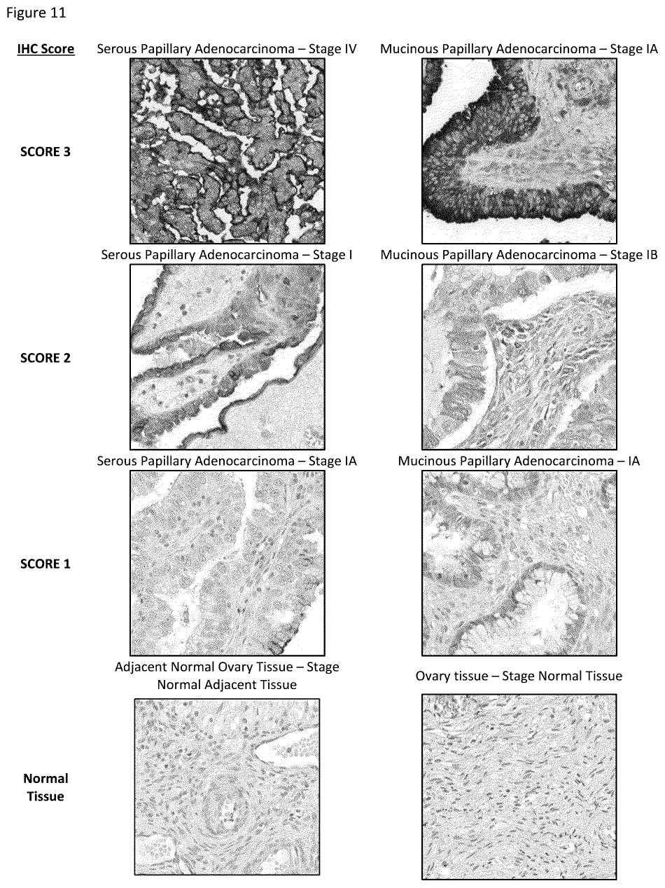

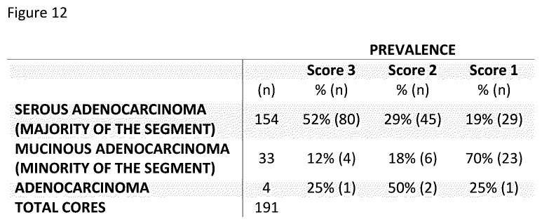

In some embodiments, one of the ABDs binds MSLN. Suitable sets of 6 CDRs and/or VH and VL domains are depicted in FIGS. 12A-12B, 13, 14, 15, and 16A-16B. In some embodiments, the heterodimeric antibody is a 1+1 Fab-scFv-Fc or 2+1 Fab2-scFv-Fv format antibody (scc, e.g., FIGS. 22A and 22B).

In one embodiment, the MSLN antigen binding domain includes the 6 CDRs (i.e., vhCDR1-3 and vlCDR1-3) of a MSLN ABD described herein, including the figures and sequence listing. In exemplary embodiments, the MSLN ABD is one of the following MSLN ABDs: MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.3_L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.5, MESO-C[MSLN]_H1_L1.6, MESO-C[MSLN]_H1_L1.7, MESO-C[MSLN]_H1_L1.8, MESO-C[MSLN]_H1_L1.9, MESO-C[MSLN]_H1_L1.10, MESO-C[MSLN]_H1.5_L1.3, MESO-C[MSLN]_H1.6_L1.3, MESO-C[MSLN]_H1.12_L1.9, MESO-C[MSLN]_H1.13_L1.9, MESO-C[MSLN]_H1.3_L1.13, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, MESO-C[MSLN]_H1.12_L1.22, and MESO-C[MSLN]_H1.12_L1.9 (FIGS. 15-21). As will be appreciated by those in the art, suitable MSLN binding domains can comprise a set of 6 CDRs as depicted in the Figures, either as they are underlined or, in the case where a different numbering scheme is used as described herein and as shown in Table 2, as the CDRs that are identified using other alignments within the VH and VL sequences of those depicted in FIGS. 15-21. Suitable ABDs can also include the entire VH and VL sequences as depicted in these sequences and Figures, used as scFvs or as Fabs. In many of the embodiments herein that contain an Fv to MSLN, it is the Fab monomer that binds MSLN.

In addition to the parental CDR sets disclosed in the figures and sequence listing that form an ABD to MSLN, the disclosure provides variant CDR sets. In one embodiment, a set of 6 CDRs can have 1, 2, 3, 4 or 5 amino acid changes from the parental CDRs, as long as the MSLN ABD is still able to bind to the target antigen, as measured by at least one of a Biacore™, surface plasmon resonance (SPR) and/or BLI (biolayer interferometry, e.g. Octet® assay) assay, with the latter finding particular use in many embodiments.

In addition to the parental variable heavy and variable light domains disclosed herein that form an ABD to MSLN, the disclosure provides variant VH and VL domains. In one embodiment, the variant VH and VL domains each can have from 1, 2, 3, 4, 5, 6, 7, 8, 9 or 10 amino acid changes from the parental VH and VL domain, as long as the ABD is still able to bind to the target antigen, as measured at least one of a Biacore™, surface plasmon resonance (SPR) and/or BLI (biolayer interferometry, e.g. Octet® assay) assay, with the latter finding particular use in many embodiments. In another embodiment, the variant VH and VL are at least 90, 95, 97, 98 or 99% identical to the respective parental VH or VL, as long as the ABD is still able to bind to the target antigen, as measured by at least one of a Biacore™, surface plasmon resonance (SPR) and/or BLI (biolayer interferometry, e.g. Octet® assay) assay, with the latter finding particular use in many embodiments.

Some useful embodiments include: XENP16249, XENP16250, XENP16253, XENP16827, XENP31693.

In some embodiments, the present invention comprises sequences selected from: XENP31701, XENP32550, XENP32551, XENP32552, XENP32553, XENP32554, XENP32555, XENP32556, XENP32557, XENP32558, XENP32559, XENP32560, XENP32561, XENP32562, XENP32563, XENP32564, XENP32565, XENP32566, XENP32567, XENP32568, XENP32569, XENP32570, XENP32937, XENP32938, XENP32939, XENP32940, XENP32941, XENP32942, XENP32942, XENP32943, XENP32944, XENP32945, and XENP32946.

In some embodiments, present invention comprises a variable heavy chain MSLN sequence comprising one or more of the following mutations: S31G, W32Y, M50W, H52N, N53Q, D55G, 194R, T97S, E61Q, E61Y, and/or K100bR.

In some embodiments, the present invention comprises a variable light chain MSLN sequence comprising one or more of the following mutations: A25S, H27Q, D28S, G30L, S32Y, V33L, T37Q, H55E, T56S, S92Y, Y94T, Y94L, Y94V, Y94I, Y94H, D28S and Y100F.

In some embodiments, the MSLN binding domain includes a MSLN binding domain variable heavy domain selected from any of FIGS. 15-18 and 20-21. In some embodiments, the MSLN binding domain includes a MSLN binding domain variable light domain selected from any of FIGS. 15-17 and 19-21. In some embodiments, the MSLN binding domain includes a combination of any of the MSLN binding domain variable heavy domains and variable light domains in FIGS. 15-21.

In some embodiments, the MSLN binding domains include the variable heavy and variable light domain of any one of the MSLN binding domains disclosed in FIGS. 15-21.

### 2. CD3 Antigen Binding Domains

In some embodiments, one of the ABDs binds CD3. Suitable sets of 6 CDRs and/or VH and VL domains, as well as scFv sequences, are depicted in FIGS. 10A-10F and the Sequence Listing. CD3 binding domain sequences that are of particular use include, but are not limited to, anti-CD3 H1.30_L1.47, anti-CD3 H1.32, anti-CD3 L1.47, anti-CD3 H1.89_L1.47, anti-CD3 H1.90_L1.47, anti-CD3 H1.33_L1.47, anti-CD3 H1.31_L1.47, anti-CD3 L1.47_H1.30, anti-CD3 L1.47_H1.30, anti-CD3 L1.47_H1.32, anti-CD3 L1.47_H1.89, anti-CD3 L1.47_H1.90, anti-CD3 L1.47_H1.33, and anti-CD3 L1.47_H1.31 as depicted in FIGS. 10A-10F.

As will be appreciated by those in the art, suitable CD3 binding domains can comprise a set of 6 CDRs as depicted in FIGS. 10A-10F, either as they are underlined or, in the case where a different numbering scheme is used as described herein and as shown in Table 2, as the CDRs that are identified using other alignments within the VH and VL sequences of those depicted in FIGS. 10A-10F. Suitable ABDs can also include the entire VH and VL sequences as depicted in these sequences and Figures, used as scFvs or as Fabs. In many of the embodiments herein that contain an Fv to CD3, it is the scFv monomer that binds CD3.

In addition to the parental CDR sets disclosed in the figures and sequence listing that form an ABD to CD3, the disclosure provides variant CDR sets. In one embodiment, a set of 6 CDRs can have 1, 2, 3, 4 or 5 amino acid changes from the parental CDRs, as long as the CD3 ABD is still able to bind to the target antigen, as measured by at least one of a Biacore™, surface plasmon resonance (SPR) and/or BLI (biolayer interferometry, e.g. Octet® assay) assay, with the latter finding particular use in many embodiments.

In addition to the parental variable heavy and variable light domains disclosed herein that form an ABD to CD3, the disclosure provides variant VH and VL domains. In one embodiment, the variant VH and VL domains each can have from 1, 2, 3, 4, 5, 6, 7, 8, 9 or 10 amino acid changes from the parental VH and VL domain, as long as the ABD is still able to bind to the target antigen, as measured at least one of a Biacore™, surface plasmon resonance (SPR) and/or BLI (biolayer interferometry, e.g. Octet® assay) assay, with the latter finding particular use in many embodiments. In another embodiment, the variant VH and VL are at least 90, 95, 97, 98 or 99% identical to the respective parental VH or VL, as long as the ABD is still able to bind to the target antigen, as measured by at least one of a Biacore™, surface plasmon resonance (SPR) and/or BLI (biolayer interferometry, e.g. Octet® assay) assay, with the latter finding particular use in many embodiments.

### H. Useful Embodiments

Useful embodiments include 1+1 Fab-scFv-Fc formats that comprise: a) a first monomer (the “scFv monomer”) that comprises a charged scFv linker (with the +H sequence of FIG. 5 being preferred in some embodiments), the skew variants S364K/E357Q, the ablation variants E233P/L234V/L235A/G236del/S267K, and an scFv that binds to CD3 as outlined herein; b) a second monomer (the “Fab monomer”) that comprises the skew variants L368D/K370S, the pI variants N208D/Q295E/N384D/Q418E/N421D, the ablation variants E233P/L234V/L235A/G236del/S267K, and a variable heavy domain; and c) a light chain that includes a variable light domain light domain (V_{L}) and a constant light domain (CL), wherein numbering is according to EU numbering. In some embodiments, the variable heavy domain and variable light domain make up a MSLN binding moiety.

Other useful embodiments include 2+1 Fab₂-scFv-Fc formats that comprise: a) a first monomer (the Fab-scFv-Fc side) that comprises the skew variants S364K/E357Q, the ablation variants E233P/L234V/L235A/G236del/S267K, and a variable heavy domain that, with the variable light domain of the common light chain, makes up an Fv that binds to MSLN as outlined herein, and an scFv domain that binds to CD3; b) a second monomer (the Fab-Fc side) that comprises the skew variants L368D/K370S, the pI variants N208D/Q295E/N384D/Q418E/N421D, the ablation variants E233P/L234V/L235A/G236del/S267K, and a variable heavy domain that, with variable light domain of the common light chain, makes up an Fv that binds to MSLN as outlined herein; and c) a common light chain comprising the variable light domain and a constant light domain, where numbering is according to EU numbering. In some embodiments, the common light chain and variable heavy domains on each monomer form MSLN binding domains.

Some useful embodiments include: XENP16248, XENP16833, XENP31697, XENP32550, XENP32551, XENP32552, XENP32553, XENP32554, XENP32555, XENP32556, XENP32557, XENP32558, XENP32559, XENP32560, XENP32561, XENP32562, XENP32563, XENP32564, XENP32565, XENP32566, XENP32567, XENP32568, XENP32569, XENP32570, XENP32937, XENP32938, XENP32939, XENP32940, XENP32941, XENP32942, XENP32943, XENP32944, XENP32945, XENP32946, XENP33888, XENP33889, XENP30764, XENP30765, XENP31701, XENP33969, XENP33971, XENP33973, XENP16251, XENP16834, XENP16252, and XENP16835.

Other useful embodiments include: XENP31063, XENP31067, XENP31705, XENP31451, XENP31452, XENP31713, XENP31064, XENP31068, XENP31709, ENP32744, XENP32747, XENP32750, XENP32851, XENP31453, XENP31454, XENP31717, XENP32745, XENP32748, XENP32751, XENP32852, XENP32947, XENP32948, XENP32949, XENP32950, XENP32951, XENP33366, XENP33367, XENP33460, XENP33461, XENP33462, XENP33463, XENP33464, XENP33465, XENP33466, XENP33467, XENP33968, XENP33970, XENP33972, XENP34155, XENP34156, XENP34157, XENP34158, XENP31455, XENP31456, XENP32249, XENP32250, XENP32251, XENP32746, XENP32749, XENP32752, XENP32853, XENP33974, XENP33975, XENP33976, XENP33977, XENP33978, XENP33979, XENP33980, XENP34151, XENP34152, XENP34153, XENP34154, XENP34159, XENP34160, XENP34161, and XENP34162.

Some useful embodiments include: XENP16249, XENP16250, XENP16253, XENP16827, XENP31693.

In some embodiments, the present invention comprises sequences selected from: XENP31701, XENP32550, XENP32551, XENP32552, XENP32553, XENP32554, XENP32555, XENP32556, XENP32557, XENP32558, XENP32559, XENP32560, XENP32561, XENP32562, XENP32563, XENP32564, XENP32565, XENP32566, XENP32567, XENP32568, XENP32569, XENP32570, XENP32937, XENP32938, XENP32939, XENP32940, XENP32941, XENP32942, XENP32942, XENP32943, XENP32944, XENP32945, and XENP32946.

In some embodiments, present invention comprises a variable heavy chain MSLN sequence comprising one or more of the following mutations: S31G, W32Y, M50W, H52N, N53Q, D55G, 194R, T97S, E61Q, E61Y, and/or K100bR.

In some embodiments, the present invention comprises a variable light chain MSLN sequence comprising one or more of the following mutations: A25S, H27Q, D28S, G30L, S32Y, V33L, T37Q, H55E, T56S, S92Y, Y94T, Y94L, Y94V, Y94I, Y94H, D28S and Y100F.

In some embodiments, the present invention comprises a MSLN binding domain that includes a MSLN binding domain variable heavy domain selected from any of FIGS. 15-18 and 20-21. In some embodiments, the present invention comprises a MSLN binding domain that includes a MSLN binding domain variable light domain selected from any of FIGS. 15-17 and 19-21. In some embodiments, the present invention comprises a MSLN binding domain that includes a combination of any of the MSLN binding domain variable heavy domains and variable light domains in FIGS. 15-21.

In some embodiments, the MSLN binding domains include the variable heavy and variable light domain of any one of the MSLN binding domains disclosed in FIGS. 15-21.

### VI. Nucleic Acids of the Invention

The disclosure further provides nucleic acid compositions encoding the anti-MSLN antibodies provided herein, including, but not limited to, anti-MSLN×anti-CD3 bispecific antibodies and MSLN monospecific antibodies.

As will be appreciated by those in the art, the nucleic acid compositions will depend on the format and scaffold of the heterodimeric protein. Thus, for example, when the format requires three amino acid sequences, such as for the 1+1 Fab-scFv-Fc format (e.g. a first amino acid monomer comprising an Fc domain and a scFv, a second amino acid monomer comprising a heavy chain and a light chain), three nucleic acid sequences can be incorporated into one or more expression vectors for expression. Similarly, some formats (e.g. dual scFv formats such as disclosed in FIG. 1) only two nucleic acids are needed; again, they can be put into one or two expression vectors.

As is known in the art, the nucleic acids encoding the components of the antibodies described herein can be incorporated into expression vectors as is known in the art, and depending on the host cells used to produce the heterodimeric antibodies described herein. Generally the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the antibodies described herein are then transformed into any number of different types of host cells as is well known in the art, including mammalian, bacterial, yeast, insect and/or fungal cells, with mammalian cells (e.g. CHO cells), finding use in many embodiments.

In some embodiments, nucleic acids encoding each monomer and the optional nucleic acid encoding a light chain, as applicable depending on the format, are each contained within a single expression vector, generally under different or the same promoter controls. In embodiments of particular use in the antibodies described herein, each of these two or three nucleic acids are contained on a different expression vector. As shown herein and in 62/025,931, hereby incorporated by reference, different vector ratios can be used to drive heterodimer formation. That is, surprisingly, while the proteins comprise first monomer:second monomer:light chains (in the case of many of the embodiments herein that have three polypeptides comprising the heterodimeric antibody) in a 1:1:2 ratio, these are not the ratios that give the best results.

The heterodimeric antibodies described herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” and “2+1” heterodimers (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### VII. Biological and Biochemical Functionality of the Heterodimeric Bispecific Antibodies

Generally the bispecific MSLN×CD3 antibodies described herein are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### VIII. Treatments

Once made, the compositions of the antibodies described herein find use in a number of applications. MSLN is highly expressed in renal cell carcinoma, accordingly, the heterodimeric compositions of the antibodies described herein find use in the treatment of such MSLN positive cancers.

### IX. Antibody Compositions for In Vivo Administration

Formulations of the antibodies used in accordance with the antibodies described herein are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ nonionic surfactant, PLURONICS™ poloxamers or polyethylene glycol (PEG).

### X. Administrative Modalities

The antibodies and chemotherapeutic agents described herein are administered to a subject, in accord with known methods, such as intravenous administration as a bolus or by continuous infusion over a period of time.

### XI. Treatment Modalities

In the methods described herein, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the disclosure includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the disclosure are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies described herein depend on the disease or condition to be treated and may be determined by the persons skilled in the art.

An exemplary, non-limiting range for a therapeutically effective amount of an bispecific antibody used in the antibodies described herein is about 0.1-100 mg/kg.

All cited references are herein expressly incorporated by reference in their entirety.

Whereas particular embodiments of the disclosure have been described above for purposes of illustration, it will be appreciated by those skilled in the art that numerous variations of the details may be made without departing from the invention as described in the appended claims.

### EXAMPLES

Examples are provided below to illustrate the antibodies described herein. These examples are not meant to constrain the antibodies described herein to any particular application or theory of operation. For all constant region positions discussed in the antibodies described herein, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publications 2015/0307629, 2014/0288275 and WO2014/145806, all of which are expressly incorporated by reference in their entirety and particularly for the techniques outlined therein.

### Example 1: Identifying Surrogates for MSLN-Expressing Tumors

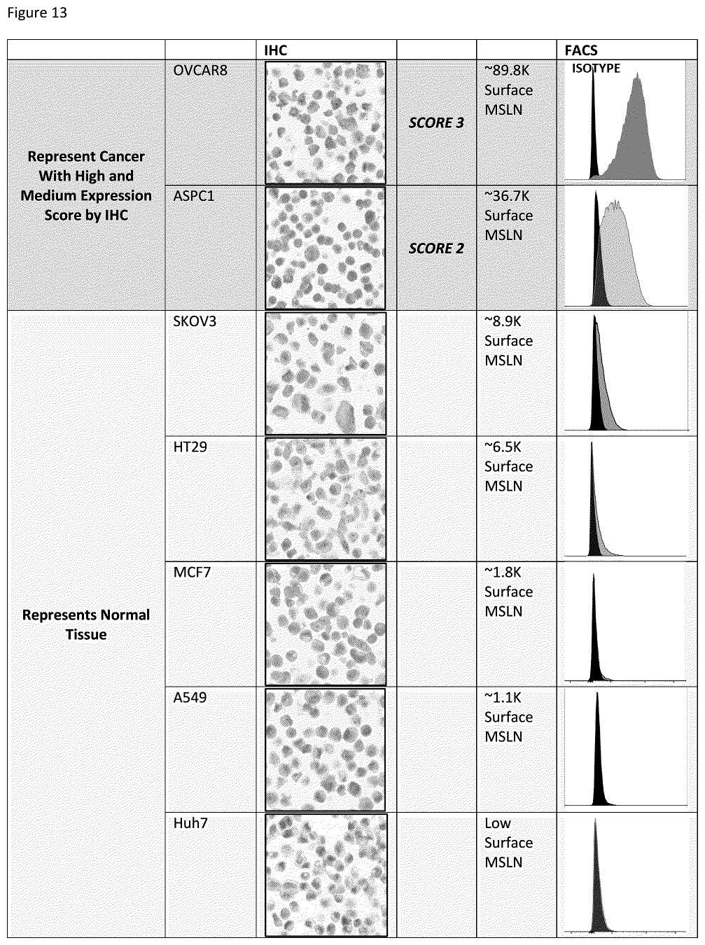

To ensure cell line with biologically valid MSLN antigen densities were used to inform the development of the novel anti-MSLN×anti-CD3 bispecific antibodies of the invention, the expression of MSLN on cancer cell lines was correlated to 191 biopsy cores of serous and mucinous ovarian cancer tissues by IHC to identify surrogate cell lines to use in research and development of the novel bispecific antibodies of the invention. Illustrative IHC of biopsy cores are depicted in FIG. 11, and samples were qualitatively scored in-house on a scale of 0-3 with 0 representing little to no MSLN expression and 3 representing high MSLN expression (herein referred to as IHC score; breakdown of score for each core is depicted in FIG. 12). Based on the results, it was determined that the novel bispecific antibodies of the invention should target score 3 and score 2 cell lines as they represent 80% of the patient segment and should not target score 1 cell lines as they resembles normal tissue. Next, endogenous MSLN expression on cancer cell lines were investigated by IHC and flow cytometry, data for which are depicted in FIG. 13. MSLN-transfected A549 cells were found to stain as intensely as ovarian cancer tumors expressing high amounts of MSLN (data not shown). OVCAR-8 cancer cells and ASPC-1 cells represent high and medium MSLN-expressing tumors (score 3 or 2 by IHC). Finally, SKOV3, HT29, MCF7, Huh7, and A549 (parental) cancer cells were found to have MSLN-expression level correlating to non-cancer adjacent tissues and therefore identified as appropriate surrogates of normal tissues.

### Example 2: Binding Domains

### 2A: CD3 Binding Domains

Sequences for CD3 binding domains having different CD3 binding affinities are depicted in FIG. 10.

### 2B: MSLN Binding Domains

The variable regions of anti-human MSLN binding domains designated MSLN-A, MSLN-B, and MSLN-C are depicted in FIGS. 15-17.

### 2B(a): Tuning MSLN-C Binding Affinity for MSLN

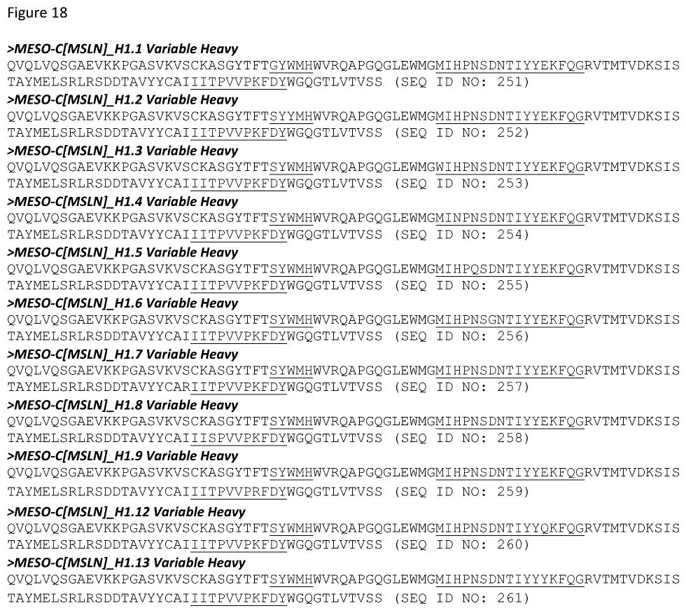

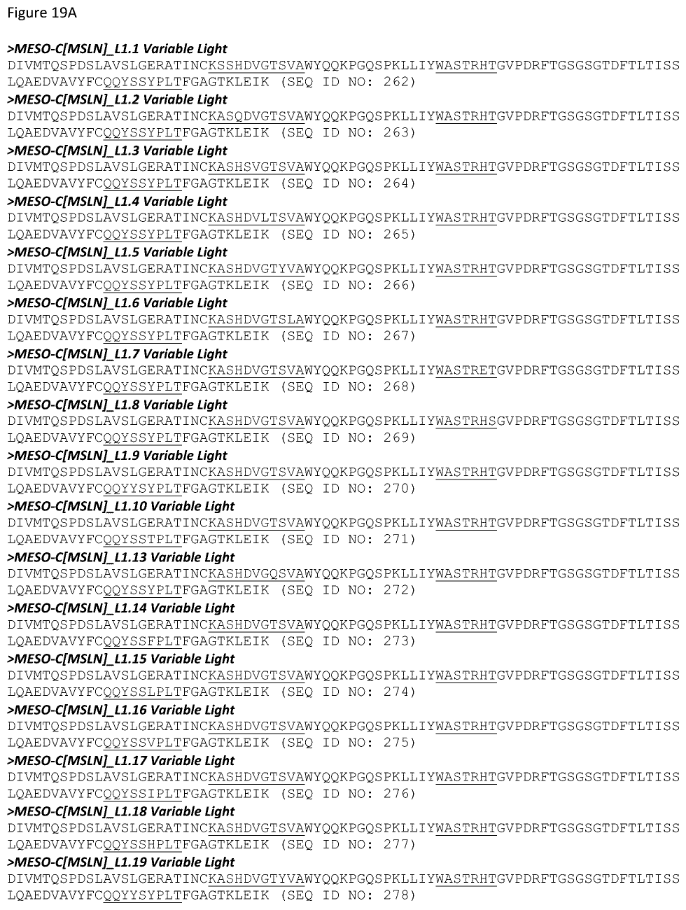

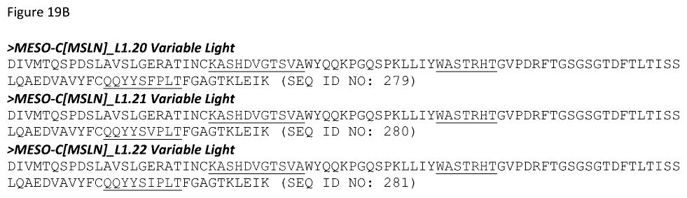

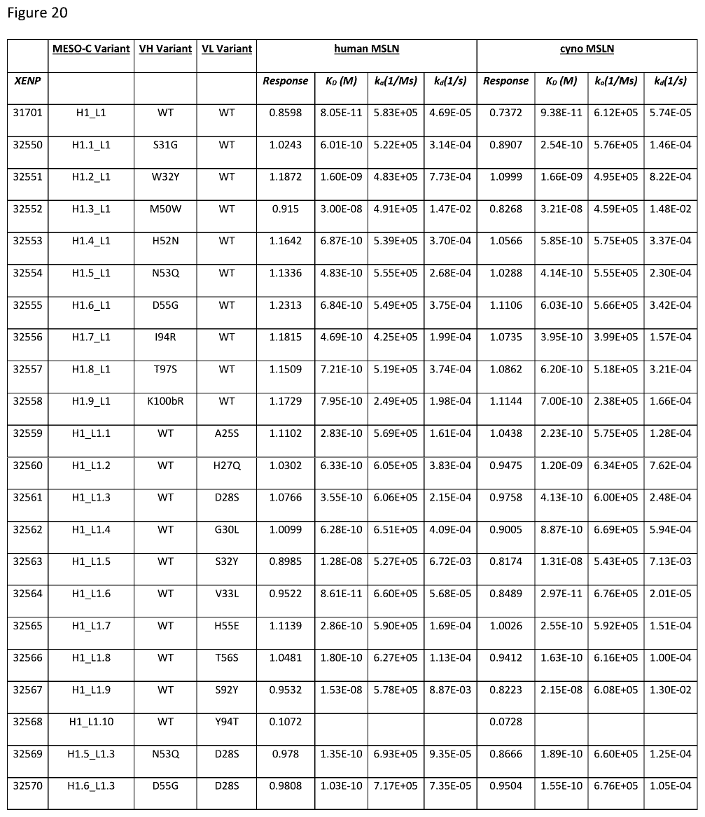

Variants of MESO-C were engineered by introducing point substitutions into the variable heavy region (VH) and the variable light region (VL) to generate 11 VH variants and 16 VL variants (sequences for which are depicted in FIG. 18 as H1.1-H1.13 and in FIG. 19 as L1.1-L1.18). An additional 4 VL variants were generated by combining useful point substitutions (sequences for which are depicted in FIG. 19 as L1.19-L1.22). WT and variant variable heavy regions were paired with WT and variant variable light regions to generate variant variable regions (Fvs) which were produced in the context of anti-MSLN×anti-CD3 bispecific antibodies. The bispecific antibodies were screened for MSLN binding affinity using Octet®, a BioLayer Interferometry (BLI)-based method. Experimental steps for Octet® generally include the following: Immobilization (capture of ligand to a biosensor); Association (dipping of ligand-coated biosensors into wells containing the analyte); and Dissociation (returning of biosensors to well containing buffer). In an initial screening, anti-human Fc sensors were used to capture the bispecific mAbs and dipped into human MSLN antigen (one concentration to screen large library). The resulting dissociation constant of illustrative variants (K_{D}), association rate (kₐ), dissociation rate (k_{d}), as well as sensorgram response are depicted in FIG. 20. In further screening of favorite variants identified in the initial screen (and combination variants), bispecific mAbs were dipped into multiple concentrations of human MSLN antigen, data for which are depicted in FIG. 21 (from various screens).

2C: Engineering Anti-MSLN×Anti-CD3 Bispecific Antibodies (bsAbs)

A number of formats for αMSLN×αCD3 bispecific antibodies (bsAbs) were conceived, illustrative formats for which are outlined below and in FIG. 22.

One such format is the 1+1 Fab-scFv-Fc format which comprises a single-chain Fv (“scFv”) covalently attached to a first heterodimeric Fc domain, a heavy chain variable region (VH) covalently attached to a complementary second heterodimeric Fc domain, and a light chain (LC) transfected separately so that a Fab domain is formed with the variable heavy domain.

Another format is the 2+1 Fab₂-scFv-Fc format which comprises a VH domain covalently attached to a CH1 domain covalently attached to an scFv covalently attached to a first heterodimeric Fc domain (VH-CH1-scFv-Fc), a VH domain covalently attached to a complementary second heterodimeric Fc domain, and a LC transfected separately so that Fab domains are formed with the VH domains.

DNA encoding chains of the αMSLN×αCD3 bsAbs were generated by standard gene synthesis followed by isothermal cloning (Gibson Assembly® molecular cloning method) or subcloning into a pTT5 expression vector containing fusion partners (e.g. domain linkers as depicted in FIG. 6 and/or backbones as depicted in FIGS. 7-9). DNA was transfected into HEK293E cells for expression. Sequences for illustrative αMLSN×αCD3 bsAbs (based on binding domains as described in Example 2B) in the 1+1 Fab-scFv-Fc format and in the 2+1 Fab₂-scFv-Fc format are depicted respectively in FIGS. 23-32. A number of formats for αMSLN×αCD3 bispecific antibodies (bsAbs) were conceived, illustrative formats for which are outlined below and in FIG. 19.

One such format is the 1+1 Fab-scFv-Fc format which comprises a single-chain Fv (“scFv”) covalently attached to a first heterodimeric Fc domain, a heavy chain variable region (VH) covalently attached to a complementary second heterodimeric Fc domain, and a light chain (LC) transfected separately so that a Fab domain is formed with the variable heavy domain.

Another format is the 2+1 Fab₂-scFv-Fc format which comprises a VH domain covalently attached to a CH1 domain covalently attached to an scFv covalently attached to a first heterodimeric Fc domain (VH-CH1-scFv-Fc), a VH domain covalently attached to a complementary second heterodimeric Fc domain, and a LC transfected separately so that Fab domains are formed with the VH domains.

DNA encoding chains of the αMSLN×αCD3 bsAbs were generated by standard gene synthesis followed by isothermal cloning (Gibson Assembly® molecular cloning method) or subcloning into a pTT5 expression vector containing fusion partners (e.g. domain linkers as depicted in FIG. 6 and/or backbones as depicted in FIGS. 7-9). DNA was transfected into HEK293E cells for expression. Sequences for illustrative αMLSN×αCD3 bsAbs (based on binding domains as described in Example 2B) in the 1+1 Fab-scFv-Fc format and in the 2+1 Fab₂-scFv-Fc format are depicted respectively in FIGS. 20-29.

### Example 3: Tuning Anti-MSLN×Anti-CD3 bsAbs to Enhance Selectivity and Therapeutic Index

### 3A: Higher Valency MSLN Binding Promotes T Cell Activity

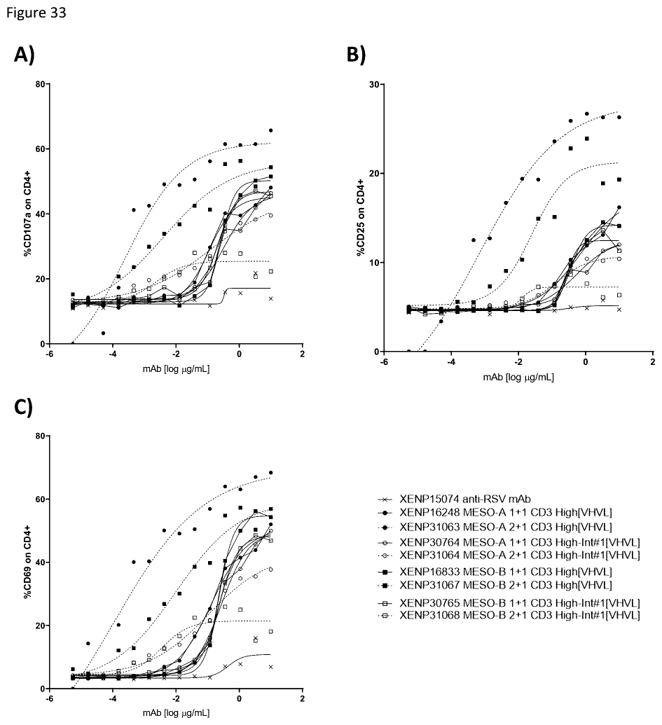

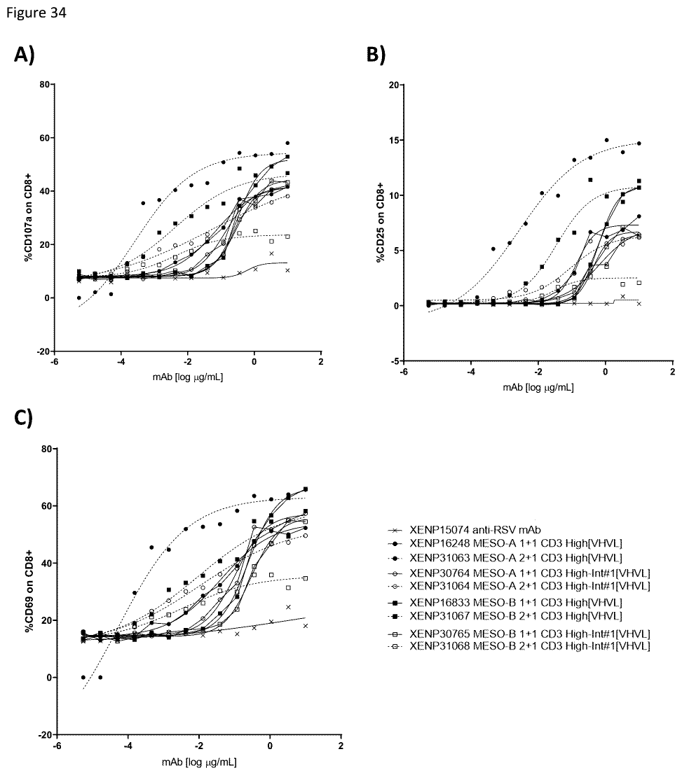

In initial investigations, the activity of anti-MSLN×anti-CD3 bsAbs in the 1+1 Fab-scFv-Fc format were compared to anti-MSLN×anti-CD3 bsAbs in the 2+1 Fab2-scFv-Fc format. Test articles were incubated with 10,000 Kuramochi cells and 100,000 human T cells. 5 μL anti-human CD107a-APC were added. Cells were incubated for 24 hours at 37° C., and analyzed by flow cytometry, data for which are presented in FIGS. 33-34. Collectively, the data show that anti-MSLN×anti-CD3 bsAbs in the 2+1 Fab2-scFv-Fc format promoted activity of CD8+ and CD4⁺ T cells (as indicated by degranulation with CD107a, CD25 expression, and CD69 expression) more potently than corresponding 1+1 Fab-scFv-Fc bsAbs.

### 3B: Tuning MSLN Binding Affinity

### 3B(a): Reducing MSLN Binding Affinity Promotes Avid Binding in the 2+1 Format

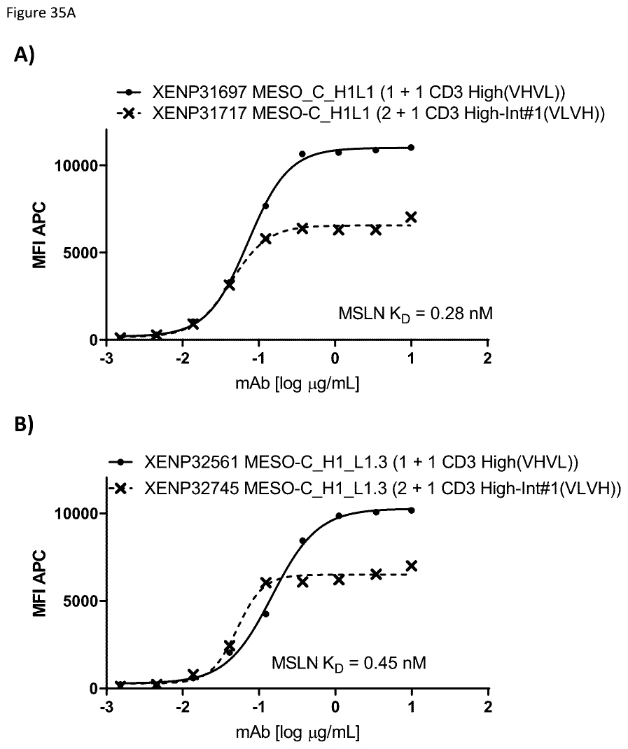

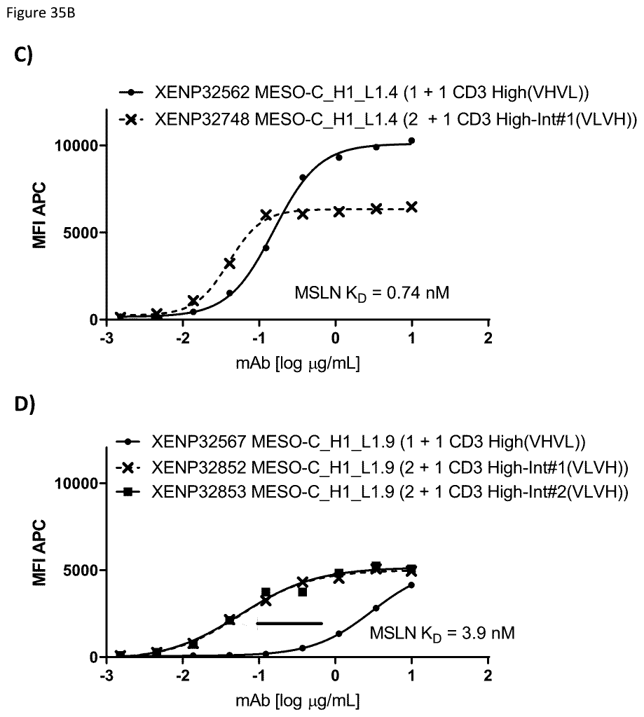

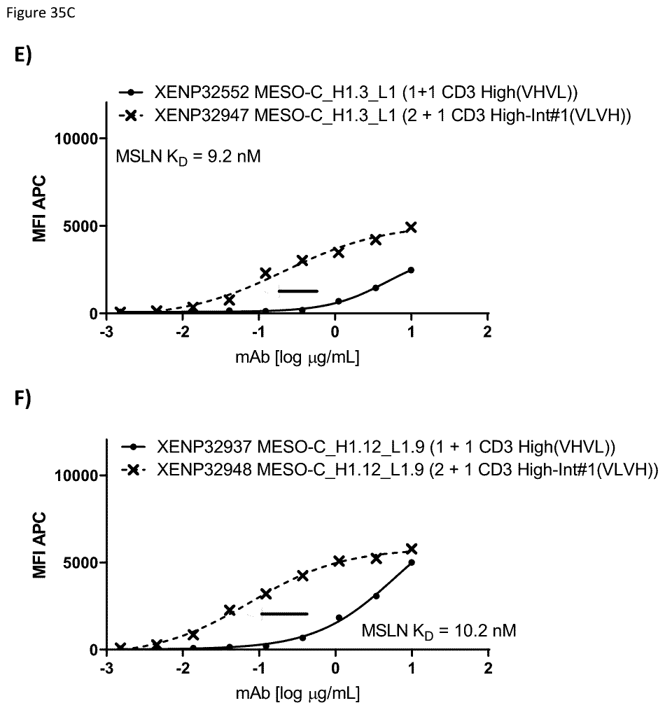

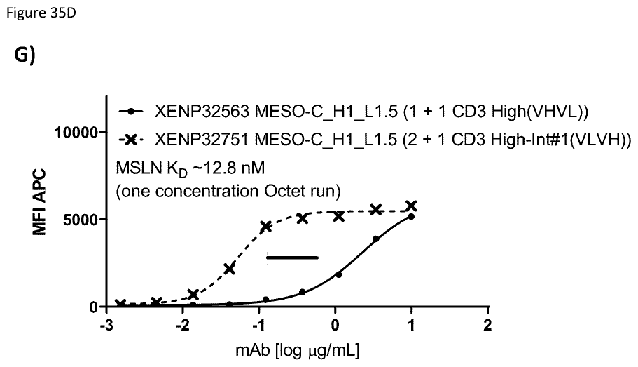

Next, the effect of MSLN binding affinity was investigated. 100,000 OVCAR8 (MSLN^{high}) cells were treated with the indicated concentrations of the indicated test articles. Binding was detected using A647-labelled anti-human Fc mAb, data for which are shown in FIG. 35. The data show that as the MSLN binding affinity is decreased, the 2+1 Fab2-scFv-Fc format provides more potent binding to OVCAR8 cells than the 1+1 Fab-scFv-Fc format. This indicates that reduction of MSLN binding affinity promotes avid binding by bsAbs in the 2+1 Fab2-scFv-Fc format to MSLN^{high} cells, as is readily observable for each of H1_L1.9 (3.9 nM K_{D}), H1.3_L1 (9.2 nM K_{D}), H1.12_L1.9 (10.2 nM K_{D}), and H1_L1.5 (˜12.8 nM K_{D}). Further, XENP32852 and XENP32853 which differ only in CD3 binding affinity have identical binding to OVCAR8 cells confirming that avid binding is due to the MSLN binding domain.

### 3B(b): Reducing MSLN Binding Affinity Enables Selectivity for Higher Antigen Density Cell Lines

Having determined that reducing MSLN binding affinity promoted avid binding in the 2+1 Fab2-scFv-Fc format to MSLN^{high} cells, the next step was to determine whether this subsequently conveys selective activity on higher density cell lines.

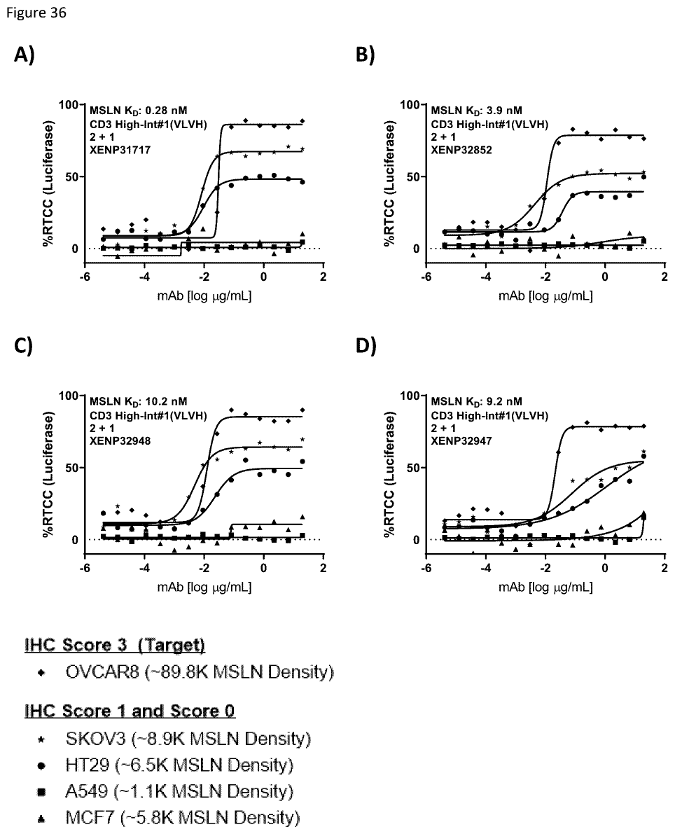

Accordingly in a first experiment, the 2+1 Fab2-scFv-Fc bsAbs investigated in Example 3B(a) were further probed in an RTCC assay. Cancer cells with varying MSLN densities (A549, HT29, MCF7, OVCAR8, and SKOV3) were transduced to constitutively express luciferase. Luciferase released from dead cancer cells rapidly degrades in assay media, so live target cells can be quantified based on luminescence readout. The luciferase-transduced cancer cells were incubated with T cells (effector:target ratio 10:1) for 24 hours. Test articles were added to the cells and incubated for 48 additional hours. Following incubation, Bio-Glo™ Luciferase reagent (PROMEGA™ Corporation, Madison, WI.) was added and plates were read with the Envision® Reader plate reader on luminescence setting. % RTCC was calculated by 1−(Raw Value/PBS AVG))*100. Data depicting dose-dependent induction of RTCC by the test articles on the various cells lines are depicted in FIG. 36. The data show that at high MSLN binding affinity, the 2+1 bsAbs induced potent RTCC on MSLN^{high} OVCAR8 cell lines as well as MSLN^{low} SKOV3 and HT29 cell lines. However, as MSLN binding affinity is reduced, the RTCC potency of the 2+1 bsAbs on the MSLN^{low} cell lines is reduced. However, there is room for further improvement in selectivity for MSLN^{med/high} cells.

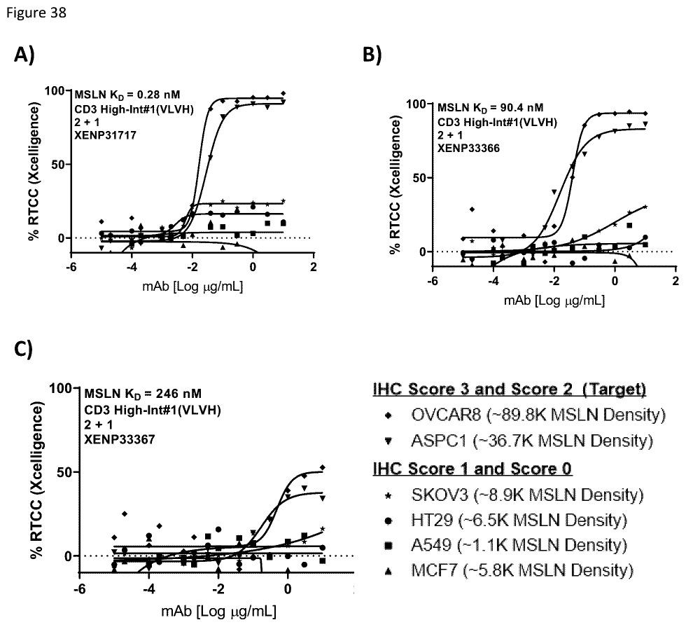

Accordingly in another set of experiments, 2+1 Fab2-scFv-Fc anti-MSLN×anti-CD3 bsAbs having further reduced MSLN affinity were explored. In one set of experiments, RTCC was assessed as described above using luciferase-transduced cancer cells. In a second experiment, RTCC was assessed using xCELLigence® Real Time Cell Analysis instrument (ACEA Biosciences, San Diego, CA). Data from each experiment are depicted in FIGS. 37-38. The data show that XENP33366 having 90.4 nM MSLN binding affinity enabled improved selectivity for MSLN^{high} OVCAR8, as indicated by reduced potency in killing of SKOV3 cells and more potent killing of OVCAR8. XENP33367 having 246 nM MSLN binding affinity demonstrated little to no RTCC activity on MSLN^{low} cells, but was also less potent on OVCAR8 than XENP33366. Notably, the data also show that even with the drastic reduction in MSLN binding affinity to enhance selectivity, RTCC activity on ASPC-1, a cancer cell line with ˜36K surface MSLN expression and a lower cancer IHC score (Score 2), was not compromised.

In yet another set of experiments, T cells were treated with a dose titration of the above test articles and various cancer cell lines (effector:target ratio 10:1) for 24 hours and assessed for proliferation, degranulation, and activation (as indicated by percentage CD3⁺ T cells respectively staining for Ki67, CD107a, and CD25), data for which are depicted in FIGS. 39-41. Notably, each of the three bispecific antibodies enabled proliferation in the presence of Score 3 and Score 2 cell lines, but not in the presence of Score 1 cell lines. Consistent with the RTCC data above, reduction in MSLN binding affinity reduced T cell degranulation and activation in the presence of Score 1 cell lines. Importantly even at drastically reduced MSLN binding affinity (i.e. for XENP33367), T cell proliferation, degranulation, and activation were not compromised for Score 3 and Score 2 cancer cell lines.

### 3C: Tuning CD3 Binding Affinity

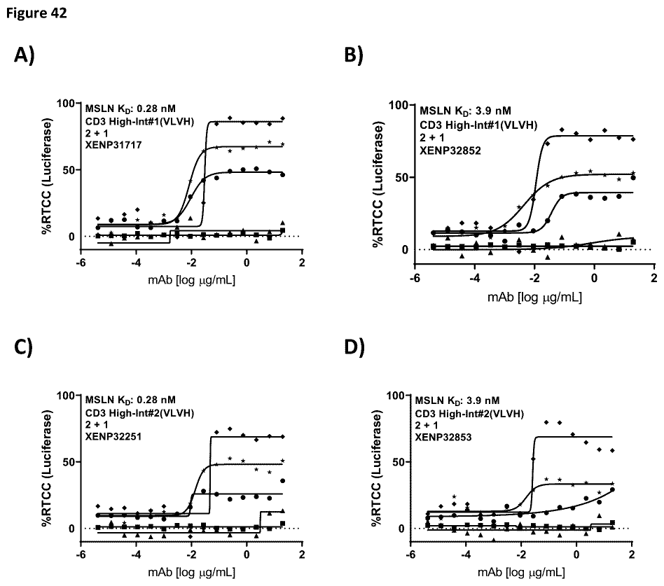

Another approach explored for optimizing the anti-MSLN×anti-CD3 bispecific antibodies was tuning CD3 binding affinity. Accordingly, anti-MSLN×anti-CD3 bsAbs in the 2+1 Fab2-scFv-Fc format having either CD3 High-Int #1 or CD3 High-Int #2 binding domains were investigated in an RTCC assay. RTCC was assessed as described above using luciferase-transduced cancer cells, data for which are depicted in FIG. 42. Collectively, the data show that reducing the CD3 binding affinity may also convey selective targeting to high expressing cell lines (even with higher affinity MSLN binding).

While exemplary embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A heterodimeric antibody comprising:
a) a first monomer comprising from N-terminus to C-terminus, a VH1-CH1-linker 1-scFv-linker 2-CH2-CH3,
wherein VH1 is a first variable heavy domain, scFv is an anti-CD3 scFV, linker 1 and linker 2 are a first domain linker and second domain linker, respectively, and CH2-CH3 is a first Fc domain;
b) a second monomer comprising from N-terminus to C-terminus a VH2-CH1-hinge-CH2-CH3, wherein VH2 is a second variable heavy domain and CH2-CH3 is a second Fc domain; and
c) a common light chain comprising a variable light domain and a light chain constant domain;
wherein the first variable heavy domain and the variable light domain form a first MSLN binding domain, and the second variable heavy domain and the variable light domain form a second MSLN binding domain.

2. A heterodimeric antibody according to claim 1, wherein the first and second MSLN binding domains each comprise the vhCDR1-3 and vlCDR1-3 of any of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

3. A heterodimeric antibody according to claim 1, wherein the first and second variable heavy domain each comprise a variable heavy domain of a MSLN binding domain, and the variable light domain each comprise a variable light domain of the MSLN binding domain, wherein the MSLN binding domain is any of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

4. A heterodimeric antibody according to any one of claims 1 to 3, wherein the scFv comprises the vhCDR1-3 and the vlCDR1-3 of any of the following CD3 binding domains: H1.32_L1.47, H1.30_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

5. A heterodimeric antibody according to any one of claims 1 to 3, wherein the scFv comprises the variable heavy domain and variable light domain of any of the following CD3 binding domains: H1.32_L1.47, H1.30_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

6. A heterodimeric antibody according to any one of claims 1 to 5, wherein the scFv comprises an scFv variable heavy domain, an scFv variable light domain and an scFv linker that connects the scFv variable heavy domain and the scFv variable light domain.

7. A heterodimeric antibody according to claim 6, wherein the scFv variable light domain is attached to the C-terminus of the CH1 of the first monomer using the first domain linker and the scFv variable heavy domain is covalently attached to the N-terminus of the first Fc domain using the second domain linker.

8. A heterodimeric antibody according to any one of claims 1 to 6, wherein the scFv variable heavy domain is attached to the C-terminus of the CH1 of the first monomer using the first domain linker and the scFv variable light domain is covalently attached to the N-terminus of the first Fc domain using the second domain linker.

9. A heterodimeric antibody according to any one of claims 1 to 8, wherein the scFv linker is a charged scFv linker.

10. A heterodimeric antibody according to any one of claims 1 to 9, wherein the first and second Fc domains are variant Fc domains.

11. A heterodimeric antibody according to claim 10, wherein the first and second Fc domains comprise a set of heterodimerization variants selected from the group consisting of those depicted in FIGS. 1A-1E.

12. A heterodimeric antibody according to claim 11, wherein the set of heterodimerization variants selected is from the group consisting of S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering.

13. A heterodimeric antibody according to claim 11 or 12, wherein the first and second monomers further comprise one or more ablation variants.

14. A heterodimeric antibody according to claim 13, wherein the one or more ablation variants are E233P/L234V/L235A/G236del/S267K, wherein numbering is according to EU numbering.

15. A heterodimeric antibody according to any of claims 10 to 14, wherein one of the first or second monomer further comprises one or more pI variant.

16. A heterodimeric antibody according to claim 15, wherein the one or more pI variant are N208D/Q295E/N384D/Q418E/N421D, wherein numbering is according to EU numbering.

17. A heterodimeric antibody according to any one claims 1 to 16, wherein the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267 K, and wherein numbering is according to EU numbering.

18. A heterodimeric antibody according to claim 17, wherein the scFv linker is a charged scFv linker having the amino acid sequence (GKPGS)4.

19. A heterodimeric antibody according to claim 17 or 18, wherein the first and second variant Fc domains each further comprise amino acid variants 428/434S, wherein numbering is according to EU numbering.

20. A heterodimeric antibody comprising:
a) a first monomer comprising from N-terminus to C-terminus, a V_{H}1-CH1-linker 1-scFv-linker 2-CH2-CH3,
wherein scFv is an anti-CD3 scFV and CH2-CH3 is a first Fc domain;
b) a second monomer comprising from N-terminus to C-terminus a V_{H}1-CH1-hinge-CH2-CH3, wherein CH2-CH3 is a second Fc domain; and
c) a common light chain comprising V_{L}-CL;
wherein the first variant Fc domain comprises amino acid variants S364K/E357Q,
wherein the second variant Fc domain comprises amino acid variants L368D/K370S,
wherein the first and second variant Fc domains each comprises amino acid variants E233P/L234V/L235A/G236del/S267K,
wherein the hinge-CH2-CH3 of the second monomer comprises amino acid variants N208D/Q295E/N384D/Q418E/N421D,
wherein said VH and VL comprise the variable heavy domain and the variable light domain of a MSLN binding domain selected from: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21), wherein the anti-CD3 scFv comprises the variable heavy domain and the variable light domain of a CD3 binding domain selected from H1.32_L1.47, H1.30_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F), and wherein numbering is according to EU numbering.

21. A heterodimeric antibody according to claim 20, wherein the scFv comprises a charged scFv linker having the amino acid sequence (GKPGS)4.

22. A heterodimeric antibody according to claim 21 or 22, wherein the first and second variant Fc domains each further comprise amino acid variants 428/434S.

23. A heterodimeric antibody according to claim 1 selected from the following: XENP33366, XENP32948, XENP32852, XENP32947, XENP32751, XENP31063, XENP31067, XENP31705, XENP31451, XENP31452, XENP31713, XENP31064, XENP31068, XENP31709, ENP32744, XENP32747, XENP32750, XENP32851, XENP31453, XENP31454, XENP31717, XENP32745, XENP32748, XENP32949, XENP32950, XENP32951, XENP33367, XENP33460, XENP33461, XENP33462, XENP33463, XENP33464, XENP33465, XENP33466, XENP33467, XENP33968, XENP33970, XENP33972, XENP34155, XENP34156, XENP34157, XENP34158, XENP31455, XENP31456, XENP32249, XENP32250, XENP32251, XENP32746, XENP32749, XENP32752, XENP32853, XENP33974, XENP33975, XENP33976, XENP33977, XENP33978, XENP33979, XENP33980, XENP34151, XENP34152, XENP34153, XENP34154, XENP34159, XENP34160, XENP34161, and XENP34162.

24. A nucleic acid composition comprising nucleic acids encoding the first and second monomers and the common light chain of the antibody according to any of claims 1 to 23.

25. An expression vector composition comprising:
a) a first expression vector comprising the first nucleic acid according to claim 24;
b) a second expression vector comprising the second nucleic acid according to claim 24; and
c) a third expression vector comprising the third nucleic acid according to claim 24.

26. A host cell transformed with an expression vector according to claim 25.

27. A method of treating a MSLN associated cancer comprising administering to a patient in need thereof an antibody according to any of claims 1 to 23.

28. A heterodimeric antibody comprising:
a) a first monomer comprising:
i) an anti-CD3 scFv comprising a first variable light domain, an scFv linker and a first variable heavy domain; and
ii) a first Fc domain, wherein the scFv is covalently attached to the N-terminus of the first Fc domain using a domain linker;
b) a second monomer comprising a VH2-CH1-hinge-CH2-CH3 monomer, wherein VH is a second variable heavy domain and CH2-CH3 is a second Fc domain; and
c) a light chain comprising a second variable light domain,
wherein the second variable heavy domain and the second variable light domain form a MSLN binding domain.

29. A heterodimeric antibody according to claim 28, wherein the MSLN binding domain comprises the vhCDR1-3 and vlCDR1-3 of any of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_HIL1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

30. A heterodimeric antibody according to claim 28, wherein the second heavy variable domain comprises a heavy variable domain and the second light variable domain comprises a variable light domain of any of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

31. A heterodimeric antibody according to any one of claims 28 to 30, wherein the anti-CD3 scFv comprises the vhCDR1-3 and the vlCDR1-3 of any of the following CD3 binding domains: H1.32_L1.47, H1.30_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

32. A heterodimeric antibody according to claim 31, wherein the anti-CD3 scFv comprises the variable heavy domain and variable light domain of any of the following CD3 binding domains: H1.30_L1.47, H1.32_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

33. A heterodimeric antibody according to any one of claims 28 to 32, wherein the first variable light domain is covalently attached to the N-terminus of the first Fc domain using a domain linker.

34. A heterodimeric antibody according to any one of claims 28 to 32, wherein the first variable heavy domain is covalently attached to the N-terminus of the first Fc domain using a domain linker.

35. A heterodimeric antibody according to any one of claims 28 to 34, wherein the scFv linker is a charged scFv linker.

36. A heterodimeric antibody according to any one of claims 28 to 35, wherein the first and second Fc domains are variant Fc domains.

37. A heterodimeric antibody according to claim 36, wherein the first and second Fc domains comprise a set of heterodimerization variants selected from the group consisting of those depicted in FIGS. 1A-1E.

38. A heterodimeric antibody according to claim 37, wherein the set of heterodimerization variants selected is from the group consisting of S364K/E357Q: L368D/K370S; S364K: L368D/K370S; S364K: L368E/K370S; D401K: T411E/K360E/Q362E; and T366W: T366S/L368A/Y407V, wherein numbering is according to EU numbering.

39. A heterodimeric antibody according to claim 37 or 38, wherein the first and second Fc domains further comprise one or more ablation variants.

40. A heterodimeric antibody according to claim 39, wherein the one or more ablation variants are E233P/L234V/L235A/G236del/S267K, wherein numbering is according to EU numbering.

41. A heterodimeric antibody according to any of claims 28 to 30, wherein one of the first or second monomer comprises one or more pI variants.

42. A heterodimeric antibody according to claim 41, wherein the one or more pI variants are N208D/Q295E/N384D/Q418E/N421D, wherein numbering is according to EU numbering.

43. A heterodimeric antibody according to any of claims 28 to 42, wherein the first monomer comprises amino acid variants S364K/E357Q/E233P/L234V/L235A/G236del/S267K,
wherein the second monomer comprises amino acid variants L368D/K370S/N208D/Q295E/N384D/Q418E/N421D/E233P/L234V/L235A/G236del/S267 K, and
wherein numbering is according to EU numbering.

44. A heterodimeric antibody according to claim 43, wherein the scFv linker is a charged scFv linker having the amino acid sequence (GKPGS)₄.

45. A heterodimeric antibody according to claim 43 or 44, wherein the first and second monomers each further comprise amino acid variants 428/434S.

46. A heterodimeric antibody according to claim 28, selected from the following heterodimeric antibodies: XENP16248, XENP16833, XENP31697, XENP32550, XENP32551, XENP32552, XENP32553, XENP32554, XENP32555, XENP32556, XENP32557, XENP32558, XENP32559, XENP32560, XENP32561, XENP32562, XENP32563, XENP32564, XENP32565, XENP32566, XENP32567, XENP32568, XENP32569, XENP32570, XENP32937, XENP32938, XENP32939, XENP32940, XENP32941, XENP32942, XENP32943, XENP32944, XENP32945, XENP32946, XENP33888, XENP33889, XENP30764, XENP30765, XENP31701, XENP33969, XENP33971, XENP33973, XENP16251, XENP16834, XENP16252, and XENP16835.

47. A heterodimeric antibody comprising:
a) a first monomer comprising from N-terminus to C-terminus, a scFv-linker-CH2-CH3,
wherein scFv is an anti-CD3 scFV and CH2-CH3 is a first Fc domain;
b) a second monomer comprising from N-terminus to C-terminus a V_{H}-CH1-hinge-CH2-CH3, wherein CH2-CH3 is a second Fc domain; and
c) a light chain comprising V_{L}-CL;
wherein the first variant Fc domain comprises amino acid variants S364K/E357Q,
wherein the second variant Fc domain comprises amino acid variants L368D/K370S,
wherein the first and second variant Fc domains each comprises amino acid variants E233P/L234V/L235A/G236del/S267K,
wherein the hinge-CH2-CH3 of the second monomer comprises amino acid variants N208D/Q295E/N384D/Q418E/N421D,
wherein the V_{H} and V_{L} form a MSLN binding domain comprising the variable heavy domain and the variable light domain, respectively, of a MSLN binding domain selected from: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21),
wherein the anti-CD3 scFv comprises the variable heavy domain and the variable light domain of a CD3 binding domain selected from H1.32_L1.47, H1.30_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F), and
wherein numbering is according to EU numbering.

48. A heterodimeric antibody according to claim 47, wherein the scFv comprises a charged scFv linker having the amino acid sequence (GKPGS)₄.

49. A heterodimeric antibody according to 47 or 48, wherein the first and second variant Fc domains each further comprise amino acid variants 428/434S, wherein numbering is according to EU numbering.

50. A nucleic acid composition comprising nucleic acids encoding the first and second monomers and the light chain of the antibody according to any of claims 28 to 49.

51. An expression vector comprising the nucleic acids according to claim 50.

52. A host cell transformed with an expression vector according to claim 51.

53. A method of treating a MSLN associated cancer comprising administering to a patient in need thereof an antibody according to any of claims 28 to 52.

54. A composition comprising a Mesothelin (MSLN) binding domain comprising the variable heavy complementary determining regions 1-3 (vhCDR1-3) and the variable light complementary determining regions (vlCDR1-3) of any of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_HIL1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

55. A composition comprising a Mesothelin (MSLN) binding domain comprising a variable heavy domain and a variable light domain of any of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

56. A nucleic acid composition comprising:
a) a first nucleic acid encoding a variable heavy domain comprising the variable heavy complementary determining regions 1-3 (vhCDR1-3) of a MSLN binding domain; and
b) a second nucleic acid encoding a variable light domain comprising the variable light complementary determining regions 1-3 (vlCDR1-3) of the MSLN binding domain, wherein the MSLN binding domain is one of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

57. A nucleic acid composition comprising:
a) a first nucleic acid encoding a variable heavy domain comprising the variable heavy domain of a MSLN binding domain; and
b) a second nucleic acid encoding a variable light domain comprising the variable light domain of the MSLN binding domain,
wherein the MSLN binding domain is any one of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

58. An expression vector composition comprising:
a) a first expression vector comprising the first nucleic acid according to claim 56 or 57; and
b) a second expression vector comprising a second nucleic acid according to claim 56 or 57.

59. A host cell comprising the expression vector composition according to claim 58.

60. A method of making an Mesothelin (MSLN) binding domain according to any one of claims 54 to 55 comprising culturing the host cell according to claim 59 under conditions wherein the MSLN binding domain is expressed, and recovering the MSLN binding domain.

61. An anti-MSLN antibody comprising an Mesothelin (MSLN) binding domain, the MSLN binding domain comprising the variable heavy complementary determining regions 1-3 (vhCDR1-3) and the variable light complementary determining regions (vlCDR1-3) of any of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

62. An anti-MSLN antibody comprising an Mesothelin (MSLN) binding domain, the MSLN binding domain comprising a variable heavy domain and a variable light domain of any of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

63. An anti-MSLN antibody comprising an Mesothelin (MSLN) binding domain selected from any one of the following MSLN binding domains: MESO-C[MSLN]_H1.3_L1.13, MESO-C[MSLN]_H1.12_L1.9, MESO-C[MSLN]_H1_L1.9, MESO-C[MSLN]_H1.3_L1, MESO-C[MSLN]_H1_L1.5, MESO-A[MSLN]_H1.1_L1, MESO-B[MSLN]_H0L0, MESO-C[MSLN]_H1L1, MESO-C[MSLN]_H1.1_L1, MESO-C[MSLN]_H1.2L1, MESO-C[MSLN]_H1.4_L1, MESO-C[MSLN]_H1.5_L1, MESO-C[MSLN]_H1.6_L1, MESO-C[MSLN]_H1.7_L1, MESO-C[MSLN]_H1.8_L1, MESO-C[MSLN]_H1.9_L1, MESO-C[MSLN]_H1_L1.1, MESO-C[MSLN]_H1_L1.2, MESO-C[MSLN]_H1_L1.3, MESO-C[MSLN]_H1_L1.4, MESO-C[MSLN]_H1_L1.6, MESO-C[MSLN]_H1_L1.7, MESO-C[MSLN]_H1_L1.8, MESO-C[MSLN]_H1_L1.10, MESO-C[MSLN]_H1.5_L1.3, MESO-C[MSLN]_H1.6_L1.3, MESO-C[MSLN]_H1.13_L1.9, MESO-C[MSLN]_H1.3_L1.14, MESO-C[MSLN]_H1.3_L1.15, MESO-C[MSLN]_H1.3_L1.16, MESO-C[MSLN]_H1.3_L1.17, MESO-C[MSLN]_H1.3_L1.18, MESO-C[MSLN]_H1.3_L1.9, MESO-C[MSLN]_H1.3_L1.5, MESO-C[MSLN]_H1_L1.20, MESO-C[MSLN]_H1.12_L1.20, MESO-C[MSLN]_H1.14_L1.14, MESO-C[MSLN]_H1.15_L1.14, MESO-C[MSLN]_H1.16_L1.14, MESO-C[MSLN]_H1_L1.19_, MESO-C[MSLN]_H1_L1.21, MESO-C[MSLN]_H1_L1.22, MESO-C[MSLN]_H1.12_L1.19, MESO-C[MSLN]_H1.12_L1.21, and MESO-C[MSLN]_H1.12_L1.22 (FIGS. 15-21).

64. An antibody according to any of claims 61 to 63, wherein the antibody comprises:
a) a first monomer comprising a first antigen binding domain and a first constant domain; and
b) a second monomer comprising a second antigen binding domain and a second constant domain,
wherein either of the first antigen binding domain or second antigen binding domain is the MSLN binding domain.

65. An antibody according to claim 64, wherein the first antigen binding domain and the second antigen binding domain bind different antigens.

66. An antibody according to claim 65, wherein the first antigen binding domain is the MSLN binding domain and the second antigen binding domain is a CD3 binding domain.

67. An antibody according to claim 66, wherein the CD3 binding domain comprises the vhCDR1-3, and vlCDR1-3 of any of the following CD3 binding domains: H1.32_L1.47, H1.30_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

68. An antibody according to 66, wherein the CD3 binding domain comprises the variable heavy domain and variable light domain of any of the following CD3 binding domains:
H1.32_L1.47, H1.30_L1.47, H1.89_L1.47, H1.90_L1.47, H1.33_L1.47, H1.31_L1.47, L1.47_H1.30, L1.47_H1.30, L1.47_H1.32, L1.47_H1.89, L1.47_H1.90, L1.47_H1.33, and L1.47_H1.31 (FIGS. 10A-10F).

69. An antibody according to any of claims 66 to 68, wherein the CD3 binding domain is an anti-CD3 scFv.

70. An antibody according to any of claims 64 to 69, wherein the first and second constant domains each comprise CH2-CH3.

71. An antibody according to any of claims 64 to 69, wherein the first and second constant domains each comprise CH1-hinge-CH2-CH3.

72. An antibody according to any of claims 64 to 71, where the first and second constant domains each are a variant constant domain.

73. An antibody according to any of claims 64 to 72, wherein the first and second monomers comprise a set of heterodimerization variants selected from the group consisting of those depicted in FIGS. 1A-1E.

74. An antibody according to claim 73, wherein the set of heterodimerization variants selected is from the group consisting of S364K/E357Q: L368D/K370S; S364K: L368D/K370S; S364K: L368E/K370S; D401K: T411E/K360E/Q362E; and T366W: T366S/L368A/Y407V.

75. An antibody according to claim 73 or 74, wherein the first and second monomers each further comprise one or more ablation variants.

76. An antibody according to claim 75, wherein the one or more ablation variants is E233P/L234V/L235A/G236del/S267K.

77. An antibody according to any of claims 73 to 76, wherein the at least one of the first or second monomer further comprises one or more pI variants.

78. An antibody according to claim 77, wherein one or more pI variants are N208D/Q295E/N384D/Q418E/N421D.

79. An antibody according to claim 69, wherein the scFv comprises a charged scFv linker.

80. A nucleic acid composition comprising nucleic acids encoding the first and second monomers according to any of claims 64 to 79.

81. An expression vector comprising the nucleic acids of claim 80.

82. A host cell transformed with an expression vector according to claim 81.

83. A method of making an anti-MSLN antibody comprising culturing the host cell according to claim 82 under conditions wherein the anti-MSLN antibody is expressed, and recovering the anti-MSLN antibody.

84. A method of treating a cancer comprising administering to a patient in need thereof an antibody according to any of claims 61 to 79.
